(19) United States
(12) Patent Application Publication
(10) Pub. No.: US 2005/0130989 A1
(43) Pub. Date: Jun. 16, 2005

(54) N-arylheteroaromatic products compositions containing them and use thereof

(75) Inventors: Alain Le-Brun, Vigneux (FR); Fabienne Thompson, Paris (FR); Gilles Tiraboschi, Chevilly Larue (FR); Joseph M. Salvino, Guilford, CT (US); Patrick Mailliet, Fontenay Sous Bois (FR)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Aventis Pharma S. A., Antony Cedex (FR)

(21) Appl. No.: 10/764,653

(22) Filed: Jan. 26, 2004

Related U.S. Application Data
(60) Provisional application No. 60/455,120, filed on Mar. 17, 2003.

(30) Foreign Application Priority Data
Jan. 28, 2003 (FR) 0300894

(51) Int. Cl. A61K 31/496; C07D 43/02

(52) U.S. Cl. 514/254.05; 544/371

(57) ABSTRACT

N-Arylheteroaromatic products, compositions containing them and use thereof. The present invention relates to novel chemical compounds, particularly to novel N-arylheteroaromatic products, to compositions containing them and to their use as medicinal products, in particular in oncology.

The present invention relates to novel chemical compounds, particularly novel N-arylheteroaromatic products, to compositions containing them and to their use as medicinal products.

More particularly, the invention relates to novel N-arylheteroaromatic products with anticancer activity, and in particular inhibitory activity on tubulin polymerization.

The N-arylheteroaromatic products concerned herein correspond to the following general formulae (Ia), (Ib) or (Ic):

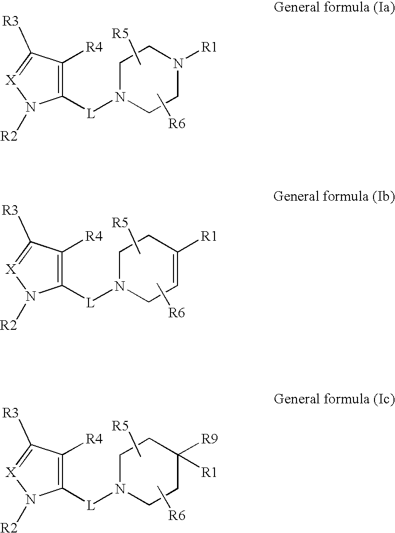

Van Wijngaarden et al. (U.S. Pat. No. 4,772,604, U.S. Pat. No. 4,874,770, EP 0 241 053) claim piperazine derivatives with antipsychotic properties. These patents have also been recalled in Chem. Abs., Vol. 108 (1988), p. 576, 221717q, in which they form the subject of the citation of two products, neither of which is disclosed in the abovementioned patents. These are

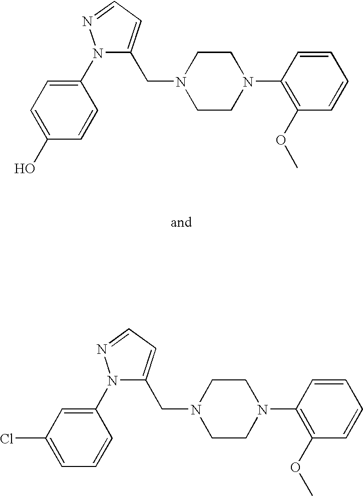

As may be seen on reading the patents from Van Wijngaarden et al., the products described and claimed are either 2,5-disubstituted pyrroles or 1,3- or 3,5-disubstituted pyrazoles. Thus, the two products described above, which are 1,5-disubstituted pyrazoles, can neither be found nor deduced from the content of these documents.

Patent application Ser. No. WO 01/19798 claims heterocyclic compounds that are useful as Factor Xa inhibitors for the treatment, for example, of thrombosis and for inhibiting the coagulation of biological samples. The products described are not included in the definition of the products according to the invention, with the exception of the following compound:

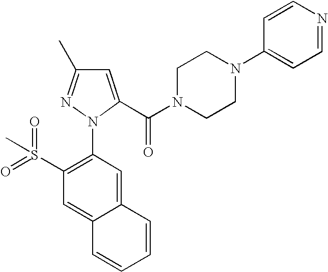

Ermondi et al., in Farmaco, 53, 519 (1998), discloses prazosine analogs, which are potential adrenoreceptor-α1 inhibitors. Only one prazosine analog is a 5-(4-heteroarylpiperazinocarbonyl)-1-phenylpyrazole:

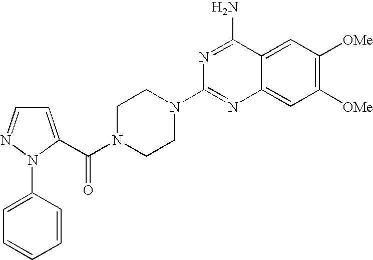

Baxter et al. (WO 01/5782) claim piperidine compounds having the formula below, for the treatment of diseases in which modulation of the chemokine receptors may be beneficial, such as pulmonary obstructions or rheumatoid arthritis.

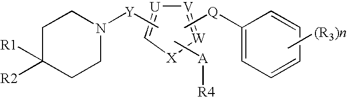

In no case can Q represent a single bond therein, and thus the products described in WO 01/5782 cannot be included in the present invention.

Now, surprisingly, it has been found that products corresponding to the general formula (I) below have considerable inhibitory activity on tubulin polymerization: 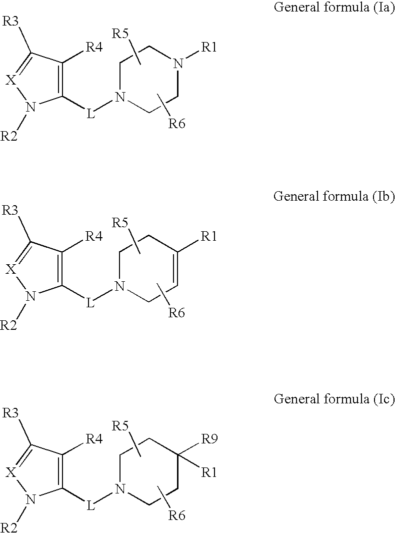
in which:
1) R1 and R2 are selected independently from the group consisting of aryl, heteroaryl, substituted aryl and substituted heteroaryl; R2 may also be chosen from the group C5-C7 cycloalkyl;
2) L is selected from the group consisting of C(R7)(R8), C═O, C═S, and C═N(R7);
3) R3 is selected from the group consisting of H, halogen, CF3, alkyl, substituted alkyl, alkylene, substituted alkylene, alkynyl, substituted alkynyl, cycloalkyl, cycloalkylene, heterocyclyl, substituted heterocyclyl, CO—R7, C(R7)=N—O(R8), COOH, CONH-aryl, CONH-heteroaryl, CONH—R7, CON(R7)(R8), CO—N(R7)-aryl, CO—N(R7)-heteroaryl, C(OR7)=NH, C[N(R7)(R8)]=NH, NH2, NH-aryl, NH-heteroaryl, NH(R7), N(R7)(R8), NH—CO—R7, N(R7)-CO-aryl, N(R7)-CO-heteroaryl, NH—SO2-R7, NH—SO2-aryl, NH—SO2-heteroaryl, NH—CH2-CO2(R7), NH—CH2-aryl, NH—CH2-heteroaryl, NH—COO—(C1-C4)alkyl, NH—CH2-(C2-C3)alkylene, NH—CH2-(C2-C3)alkynyl, N(R7)-N(R8)(R12), N—N═C(R7)(R8), CN, O—R7, O—CH2-aryl, O—CH2-heteroaryl, S—R7, SO—R7, SO₂—R7, aryl, heteroaryl, substituted cycloalkyl, substituted aryl and substituted heteroaryl;
4) R4 is selected from the group consisting of H, (C1-C3)alkyl, cyclopropyl, (C2-C3)alkylene, (C2-C3)alkynyl, O(C1-C3)alkyl, S—(C1-C3)alkyl, F, Cl and Br;
5) X is N or CH;
6) R5 and R6 are selected independently from the group consisting of H, (C1-C3)alkyl, oxo and halogen;
7) R7, R8 and R12 are independently selected from the group consisting of H, (C1-C3)alkyl and substituted (C1-C3)alkyl;
8) R9 is (C1-C3)alkyl;
   in racemic form, enriched in one enantiomer, enriched in one diastereoisomer, its tautomers, its prodrugs and its pharmaceutically acceptable salts, with the proviso that the product of formula (I) is not one of the following compounds:

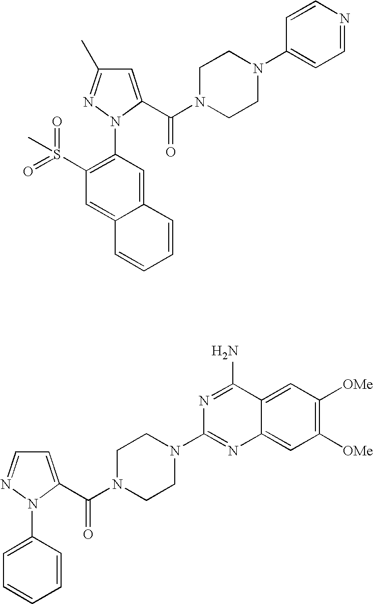

Products of general formula (Ia) or (Ib) are preferred.

Products for which X is N are preferred.

A preferred substituent R1 may be chosen from phenyl; phenyl substituted with at least one radical chosen from halogen, (C1-C3)alkyl, CON(R10)(R11), O—R10, S—R10 and N(R10)(R11), in which R10 and R11 are chosen independently from H, (C1-C3)alkyl and halogenated (C1-C3)alkyl; pyridyl; pyridyl substituted with at least one radical chosen from halogen, (C1-C3)-alkyl, CON(R10)(R11), O—R10, S—R10 and N(R10)(R11), in which R10 and R11 are chosen independently from H, (C1-C3)alkyl and halogenated (C1-C3)alkyl.

More preferably, R1 will be phenyl substituted with halogen or (C1-C3)alkyl, or (C1-C3)alkoxy, or carboxamide; 2- or 3-pyridyl; 2- or 3-pyridyl substituted with halogen or (C1-C3)alkyl.

Very preferably, R1 is phenyl substituted with a chloro radical, one or two methoxy radicals or a carboxamide radical.

When R1 is substituted phenyl, preferred substitution combinations may be chosen from 2,3-disubstituted phenyl, 2,5-disubstituted phenyl, 3-substituted phenyl, 3,5-disubstituted phenyl and 3,4-disubstituted phenyl, and more preferably from 3-substituted phenyl, 3,5-disubstituted phenyl and 3,4-disubstituted phenyl.

When R1 is substituted 2-pyridyl, preferred substitutions are chosen from 4- or 6-substituted 2-pyridyl or 4,6-disubstituted 2-pyridyl.

When R1 is substituted 3-pyridyl, preferred substitutions are 2- or 5-substituted 3-pyridyl.

A preferred substituent R2 may be chosen from phenyl, 3-pyridyl, phenyl substituted with at least one radical chosen from halogen, alkyl, O—R10, S—R10 and N(R10)(R11), in which R10 and R11 are independently chosen from H, (C1-C3)alkyl and halogenated (C1-C3)alkyl.

A preferred substituent R2 is chosen from unsubstituted phenyl and 3-pyridyl. Unsubstituted phenyl is more preferred.

Preferably, R3 is H or (C1-C3)alkyl, CF3, hydroxymethyl, amino, azetidino or pyrrolidino.

More preferably, R3 is H or a methyl, hydroxymethyl, CF3 or amino radical.

R4 is preferably H.

In general, products of general formula (Ia), (Ib) or (Ic) in accordance with the invention in which L is C(O) may be prepared by coupling a 1-aryl(heteroaryl)-pyrrole-2-carboxylic acid, or a 2-aryl(heteroaryl)pyrazole-3-carboxylic acid, of general formula (II), in which R2, R3 and R4 are defined as above, with, respectively, a piperazine derivative of general formula (IIIa) or a 1,2,3,6-tetrahydropyridine derivative (IIIb), in which R1 is defined as above, or a piperidine derivative of general formula (IIIc), in which R1 and R9 are defined as above, according to Scheme 1:

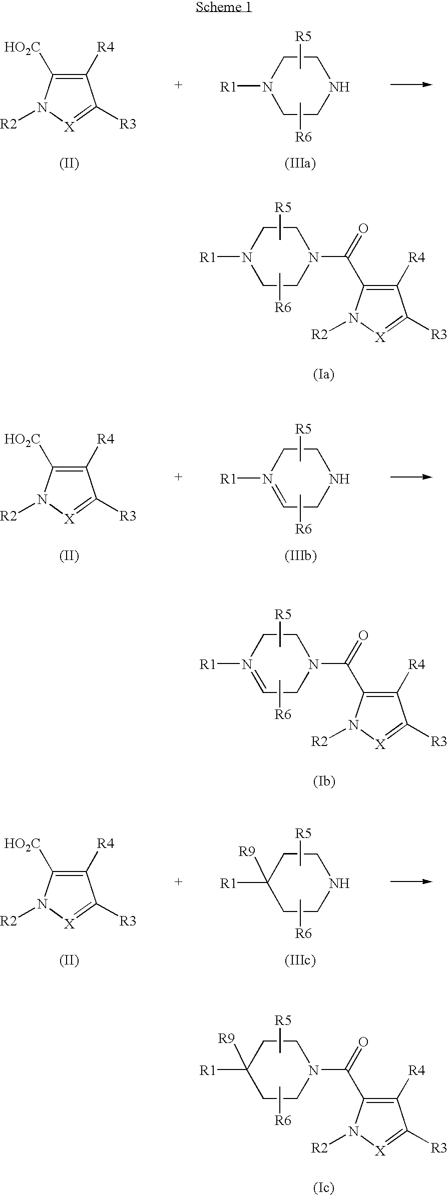

The coupling may be performed using the coupling methods known to those skilled in the art, in particular those consisting in activating the acid of general formula (II) in the form of chloride or anhydride, or any of the coupling methods developed for peptide synthesis.

In general, products of general formula (Ia), (Ib) or (Ic) in accordance with the invention in which L is C(O) may be prepared by coupling a methyl or ethyl ester of a 1-aryl(heteroaryl)pyrrole-2-carboxylic acid or of a 2-aryl(heteroaryl)-pyrazole-3-carboxylic acid, of general formula (II), in which R2, R3 and R4 are defined as above, with, respectively, a piperazine derivative of general formula (IIIa) or a 1,2,3,6-tetrahydropyridine derivative (IIIb), in which R1 is defined as above, or a piperidine derivative of general formula (IIIc), in which R1 and R9 are as defined above according to Scheme 1(a):

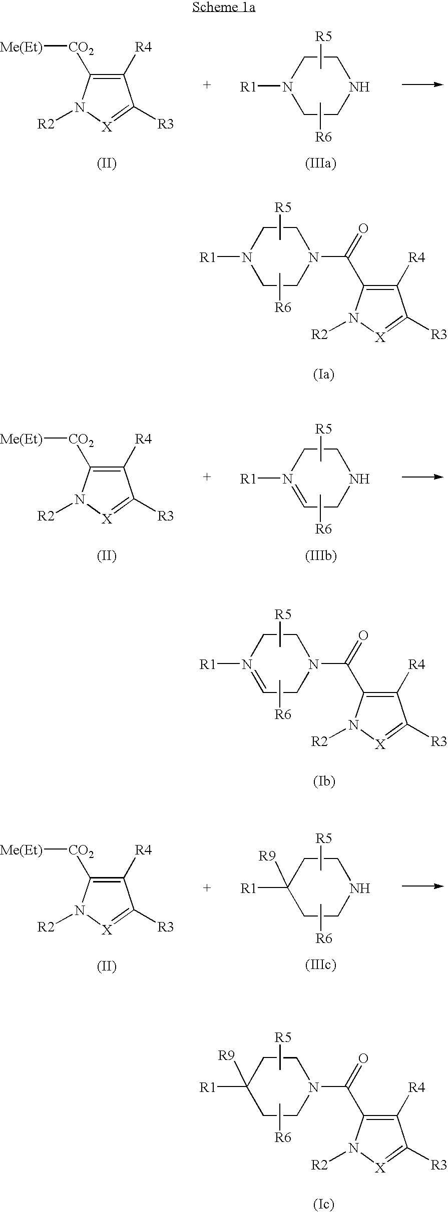

The coupling may be performed using the coupling methods known to those skilled in the art, in particular by activating the amine (IIIa), (IIIb) or (IIIc) with trimethylaluminum under the conditions described in Organic Synthesis 59, 49-53 (1980).

The acids or the methyl or ethyl esters of 1-aryl(heteroaryl)pyrrole-2-carboxylic or 2-aryl(heteroaryl)pyrazole-3-carboxylic acids of general formula (II) may be obtained according to the methods known to those skilled in the art, in particular the ortho-carboxylation of a pyrrole or pyrazole derivative, followed by the N-alkylation or N-arylation of the pyrrole or pyrazole according to Scheme 2; in the case of the pyrazoles, a readily separable mixture of N-1 and N-2 substituted products is generally obtained.

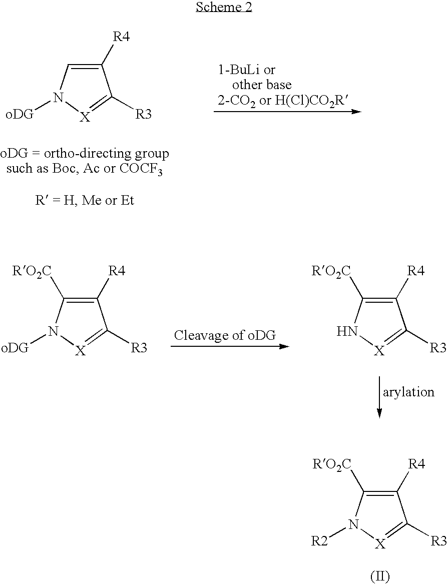

When X represents a nitrogen atom, the arylation, advantageously performed by Suzuki coupling, may be performed by working under the conditions described in Tetrahedron, 55, 12757 (1999).

When X represents a nitrogen atom, the arylation may very advantageously be performed by working under the conditions described by Buchwald in J. Amer. Chem. Soc., 123, 7727 (2001), with an aryl halide R2-Hal, by catalysis with cuprous iodide in the presence of cesium carbonate and 1,2-diaminocyclohexane.

In the case where R2 represents a phenyl radical, X represents a CH group and R4 represents a hydrogen atom, it is possible to perform the ortho-carboxylation of 1-phenylpyrrole-2-carboxylic acid directly, by working according to Tetrahedron, 49, 10278 (1993).

The groups R3 and/or R4, which are other than a hydrogen atom, the 1-aryl(heteroaryl)pyrrole-2-carboxylic or 2-aryl(heteroaryl)pyrazole-3-carboxylic acids of general formula (II) may also be introduced into 1-aryl(heteroaryl)pyrrole-2-carboxylic or 2-aryl(heteroaryl)pyrazole-3-carboxylic acids of general formula (II), in which R3 and/or R4 represent a hydrogen atom, by any of the conventional methods known to those skilled in the art.

Among these methods, mention may be made of the regioselective halogenation of the 1-aryl(heteroaryl)pyrrole-2-carboxylic or 2-aryl(heteroaryl)pyrazole-3-carboxylic acids, followed by substitution.

In the context of the invention, when X represents a nitrogen atom, the substitution of a halogen in position 3 of a 1-aryl-1H-pyrazole-5-carboxylic acid ester is found to be a method that is particularly advantageous for preparing a derivative of general formula (Ia) in which X represents a nitrogen atom, R4 represents a hydrogen atom and R3 represents alkylene, substituted alkylene, alkynyl, substituted alkynyl, aryl, substituted aryl, heteroaryl, substituted heteroaryl, NH2, NH-aryl, NH(R7), N(R7)(R8), NH—CO—R7, NH—CO-aryl, NH—SO2-aryl, NH—CH2-CO2R7, NH—CH2-aryl, N(R7)-N(R7)(R8), N—N═C(R7)(R8), CN, OR7, SR7, SO—R7 or SO2-R7, according to Scheme 2a.

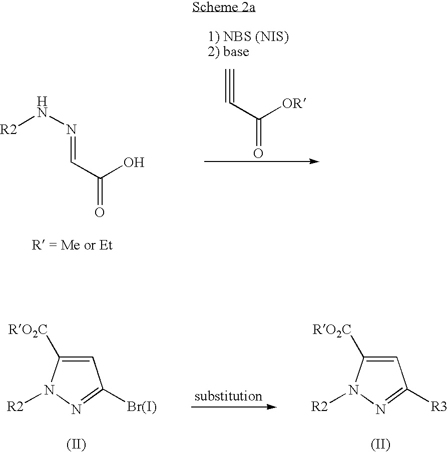

In the context of the invention, the substitution of ethyl 3-bromo-1-phenyl-1H-pyrazole-3-carboxylate, which may be prepared according to Tetrahedron Lett., 40, 2605 (1999), will be used in particular.

In the context of the invention, the substitutions of the bromine atom will advantageously be performed by heating for a few minutes at 120-150° C. in a microwave reactor, more particularly Suzuki and Heck carbon-carbon couplings or Buchwald aminations.

In the context of the invention, when X represents a nitrogen atom or a CH radical, the substitution of the bromine atom of a bromomethyl radical in position 3 of a 1-aryl-1H-pyrazole(pyrrole)-5-carboxylic acid ester is found to be a method that is particularly advantageous for preparing a derivative of general formula (Ia) in which X represents a nitrogen atom or a CH radical, CH, R4 represents a hydrogen atom and R3 represents an alkyl radical, according to Scheme 2b.

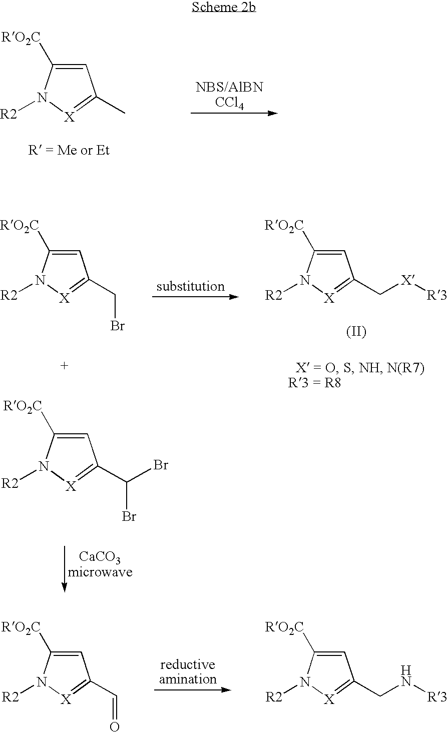

In the context of the invention, the substitutions of the bromine atom will advantageously be performed by heating for a few minutes at 120-150° C. in a microwave reactor.

In the case where X represents a nitrogen atom, R3 represents a hydrogen atom or a C1-C3 alkyl radical and R4 represents a hydrogen atom, it is advantageous to use the method for synthesizing 2-aryl(heteroaryl)pyrazole-3-carboxylic acids described in J. Het. Chem., 30, 307 (1993), starting with aryl(heteroaryl)hydrazines, by working according to Scheme 3:

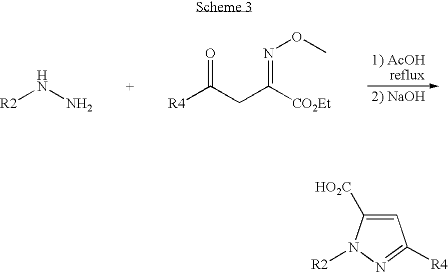

Starting with aryl(heteroaryl)hydrazines, it is also advantageously possible to prepare 2-aryl(heteroaryl)pyrazole-3-carboxylic esters, by working according to J. Het. Chem., 36, 217 (1999), which will then be saponified to the corresponding acids.

Another method for synthesizing 2-aryl(heteroaryl)pyrazole-3-carboxylic esters that is particularly advantageous in the context of the invention uses cycloaddition reactions, followed by oxidation of the intermediate adduct obtained with chloranil, of aryl(heteroaryl)hydrazones with a propionate, by working according to Tetrahedron, 36, 887 (1980).

Another method for synthesizing 1-aryl(heteroaryl)pyrrole-5-carboxylic acid esters that is particularly advantageous in the context of the invention uses the reaction of an aryl(heteroaryl)amine with 2,5-dimethoxytetrahydrofuran, by working according to Heterocycles, 53, 2160 (2000).

The piperazine derivatives of general formula (IIIa), in which R1, R5 and R6 are defined as above are either commercially available or are prepared according to the standard methods known to those skilled in the art.

Among these methods, the N1-aryl(heteroaryl)ation, according to Scheme 4, of piperazines bearing a protecting group on the nitrogen in position 4, is particularly advantageous in the context of the invention:

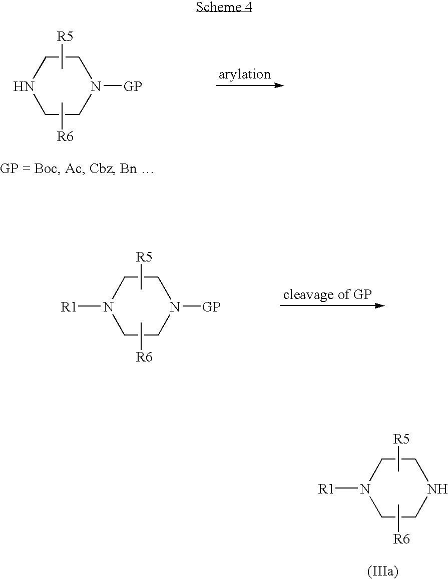

The aryl(heteroaryl)ation reaction of piperazines, generally of Hartwig/-Büchwald type, may be performed by working under the conditions described in Biorg. Med. Chem. Lett., 11, 1375 (2001) or in Biorg. Med. Chem., 10, 3817 (2002).

Another method for synthesizing aryl(heteroaryl)piperazines that is particularly advantageous in the context of the invention, when R5 and R6 represent hydrogen atoms, consists in reacting an aryl(heteroaryl)amine with a bis(2-hydroxy- or 2-haloethyl)amine, at a temperature above 100-120° C., according to Scheme 5:

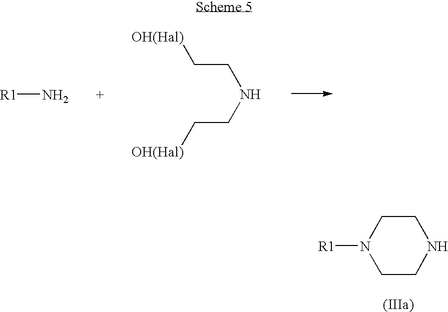

It is particularly advantageous to perform the process in the presence of microwaves under the conditions described in Synth. Comm., 28, 1175 (1998), or in Tetrahedron Lett, 38, 6875 (1997).

The 1,2,3,6-tetrahydropyridine derivatives (IIIb), in which R1, R5 and R6 are defined as above, are either commercially available or are prepared according to the conventional methods known to those skilled in the art.

Among these methods, the action according to Scheme 6, of an organometallic aryl(heteroaryl) derivative, such as an organomagnesium reagent, an organolithium reagent or an organocerium reagent, on a piperid-4-one derivative in which the nitrogen atom is substituted with a protecting group, is particularly advantageous.

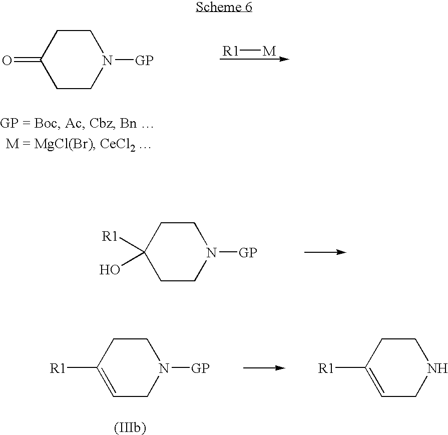

The process may be performed in particular under the conditions described in J. Med. Chem., 38, 1998 (1995) or in EP 306764 or in J. Med. Chem., 28, 311 (1985).

When R5 and R6 represent hydrogen atoms, Suzuki coupling of the pinacol ester of N-Boc-1,2,3,6-tetrahydropyridyl-4-boronic acid with an aryl or heteroaryl halide, preferably a bromide or an iodide, under the conditions described in Tetrahedron Lett, 41, 3705 (2000), according to Scheme 7, is particularly advantageous in the context of the invention. It is understood that the Boc protecting group may be replaced with any other protecting group that is compatible with the reaction conditions and that the pinacol boronic ester may also be replaced with any other boronic, acid or ester derivative that is compatible with said conditions.

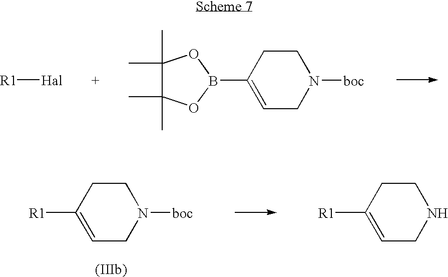

The piperidine derivatives, in which R1, R6, R7 and R9 are defined as above, are prepared according to the conventional methods known to those skilled in the art.

Among these methods, the action, according to Scheme 8, of an organometallic alkyl derivative, such as an organomagnesium reagent, an organolithium reagent or an organocerium reagent, on a piperid4-one derivative in which the nitrogen atom is substituted with a protecting group, followed by the action of an aryl or heteroaryl derivative, in the presence of an acid catalyst, of Lewis acid or superacid type according to Olah, is particularly advantageous in the context of the invention.

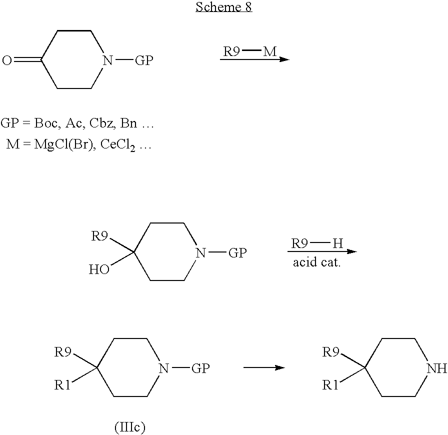

The process may be performed in particular under the conditions described in J. Med. Chem., 41, 5320 (1998) or in Tetrahedron Lett., 41, 8853 (2000).

In general, products of general formula (Ia), (Ib) or (Ic) in accordance with the invention in which L is CH₂ may be prepared by reducing a compound of general formula (Ia), (Ib) or (Ic), respectively, in which L is C(O), by any of the reduction methods known to those skilled in the art, for instance the Clemmensen or Wolff-Kishner methods, by working according to Scheme 9:

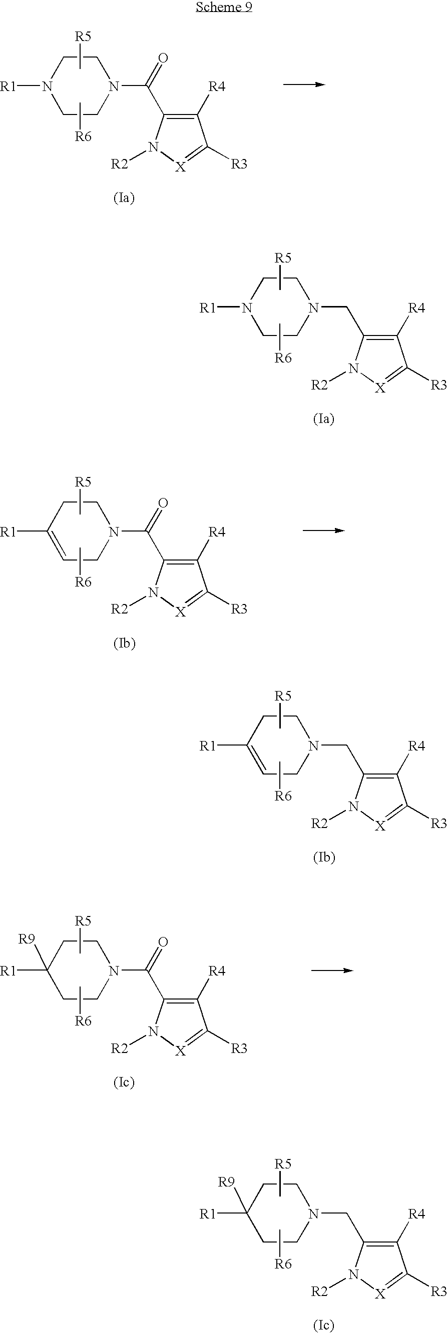

In general, products of general formula (Ia), (Ib) or (Ic) in accordance with the invention in which L is CH₂ may also be prepared from the esters of the products of general formula (II), by using the various methods known to those skilled in the art, according to the reaction sequences of Scheme 10:

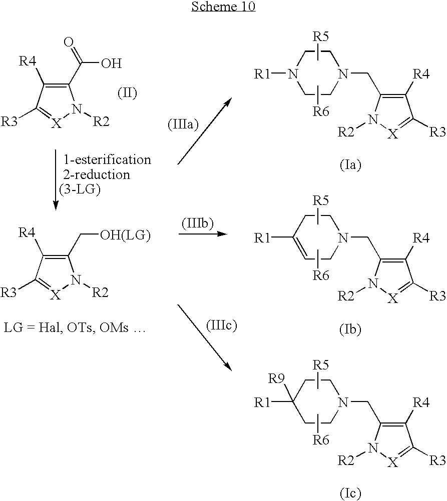

In general, products of general formula (Ia), (Ib) or (Ic) in accordance with the invention in which L is CR7R8, with R7 and/or R8 other than a hydrogen atom, may also be prepared from the products of general formula (II), or esters thereof, using the various methods known to those skilled in the art, according to the reaction sequences of Scheme 11:

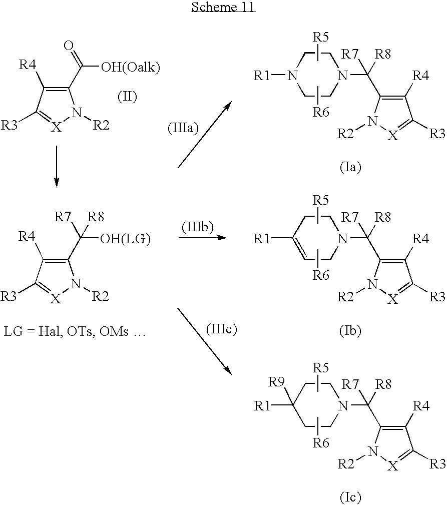

In general, products of general formula (Ia), (Ib) or (Ic) in accordance with the invention in which L is C(S) may be prepared by thionation of a compound of general formula (Ia), (Ib) or (Ic), respectively, in which L is C(O), by any of the reduction methods known to those skilled in the art, by working according to Scheme 12:

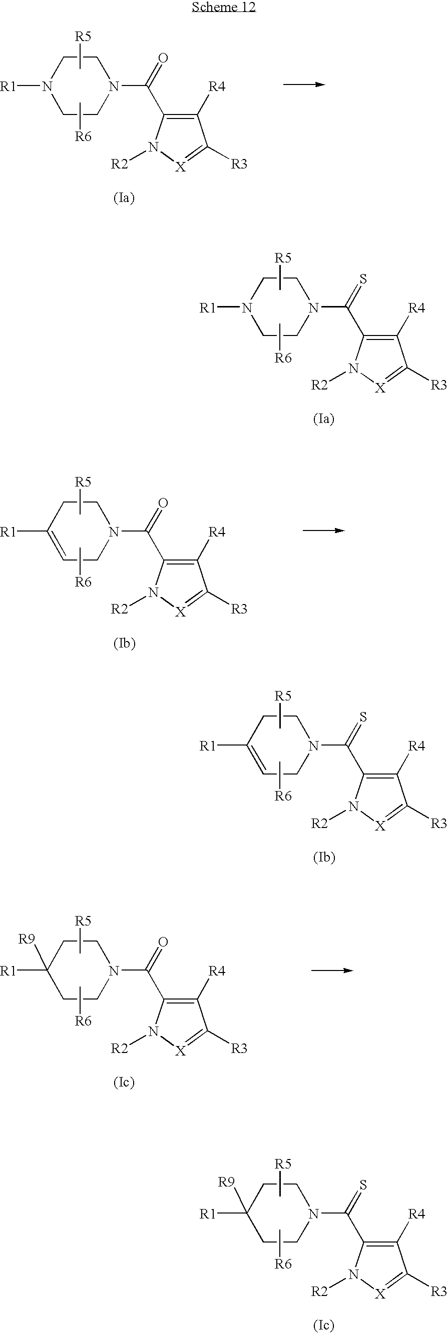

It is particularly advantageous in the context of the invention to perform the thionation using a Lawesson reagent, by working according to Bull. Soc. Chim. Belg., 87, 293 (1978).

In general, products of general formula (Ia), (Ib) or (Ic) in accordance with the invention in which L is C(NH) may be prepared from the nitrites derived from the products of general formula (II), using the various methods known to those skilled in the art, according to the reaction sequences of Scheme 13:

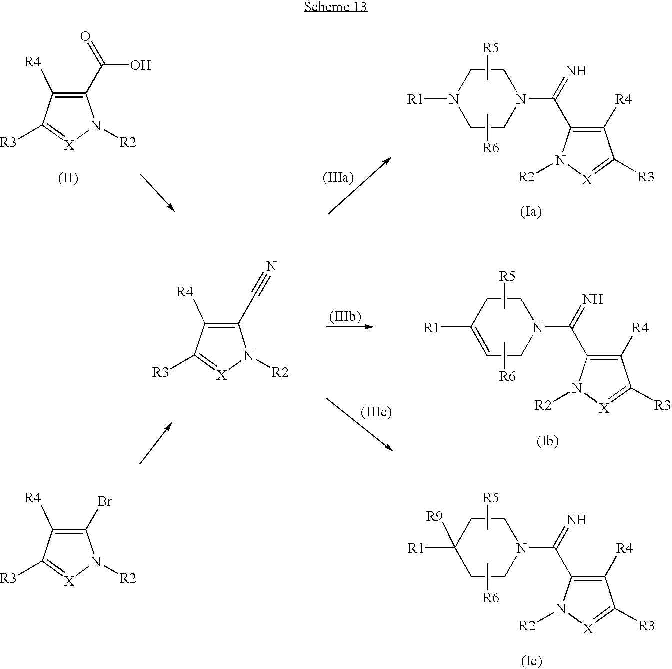

It is generally necessary to activate the nitrile, which is relatively unreactive, either with aluminum chloride, by working according to J. Chem. Soc. 1947, 1110; or with cuprous iodide, by working according to Tetrahedron Lett., 34, 6395 (1993); or by converting the nitrile into an imino ether prior to the reaction with the piperazine—or 1,2,3,6-terahydropyridine or piperidine—derivative, by working according to Eur. J. Med. Chem., 24, 427 (1989).

In general, products of general formula (Ia) or (Ic) in accordance with the invention in which L is C(NR7), with R7 identical to or different than a hydrogen atom, may be prepared from the products of general formula (Ia) or (Ic), respectively, in which L is C(O) and/or C(S), by using the various methods known to those skilled in the art, according to the reaction sequences of Scheme 14.

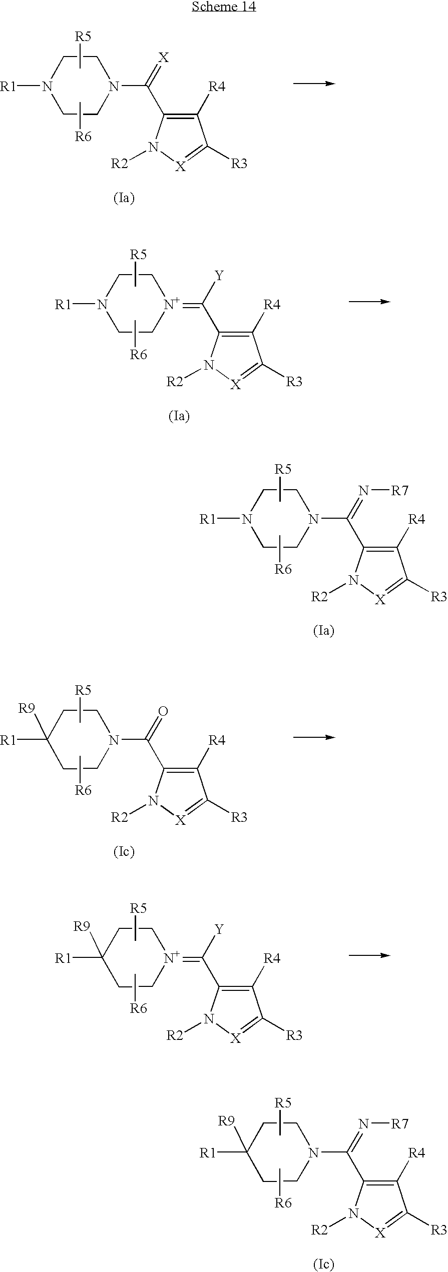

In the context of the invention, when X is an oxygen atom, it is particularly advantageous to successively react oxalyl chloride, which leads to an intermediate in which Y is a chlorine atom, and then an amine R7-NH2, by working according to Pol. J. Chem., 58, 117 (1984), and in the case where X is a sulfur atom, to first react methyl iodide, which leads to an intermediate in which Y is a methylthio radical, and then an amine R7-NH₂, by working according to Eur. J. Med. Chem, 12, 365 (1977).

More specifically and more advantageously in the context of the invention, products of general formula (Ia) in accordance with the invention in which L is C(O), X is N, R3 is methyl, R6 and R7 are H and R1 and R2 are defined as above, may be prepared by coupling between a 1-phenylpyrazole-3-carboxylic acid and a piperazine derivative according to Reaction Scheme 15:

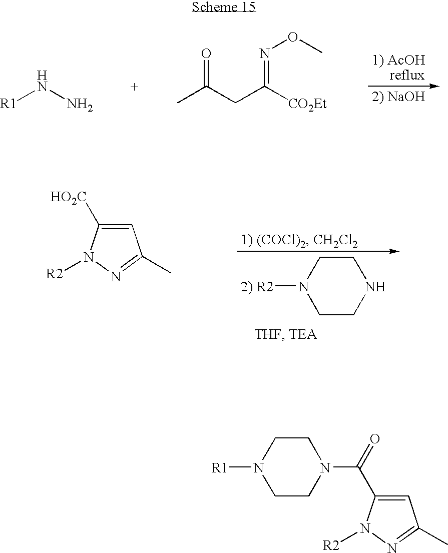

In this scheme, a phenylhydrazine, optionally in salified form, is condensed with an α-methyloxime of an α,γ-diketo ester in acidic medium to give a mixture of 5-methyl-2-phenyl-2H-pyrazole-3-carboxylic and 5-methyl-1-phenyl-1H-pyrazole-3-carboxylic acids. The 5-methyl-2-phenyl-2H-pyrazole-3-carboxylic acid is isolated and then used in an amidation reaction between the preactivated carboxylic function of the pyrazole and an amine such as a 4-arylpiperazine in basic medium to give a product in accordance with the invention.

The 5-alkyl-2-phenyl-2H-pyrazole-3-carboxylic and 5-alkyl-1-phenyl-1H-pyrazole-3-carboxylic acids may be obtained and isolated under the conditions described by Ashton, in J. Het Chem., 30, 307 (1993).

More specifically and more advantageously in the context of the invention, products in accordance with the invention may also be prepared on a solid phase, according to Reaction Scheme 16:

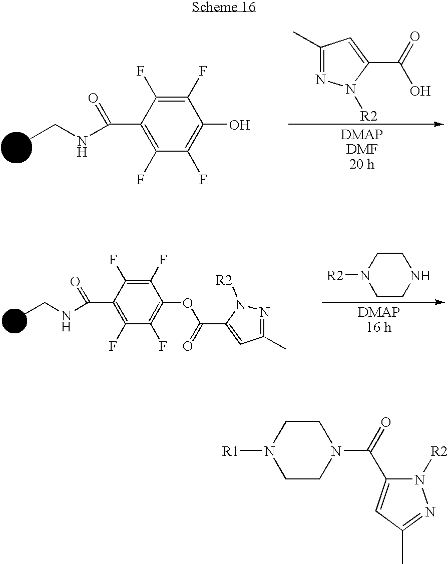

More specifically and more advantageously in the context of the invention, products of general formula (Ia) in accordance with the invention in which L is C(O), X is N, R3 is other than H or methyl, R6 and R7 are H and R1 and R2 are as defined above, may be prepared from a product of general formula (Ia) in which R3 is a bromine atom or a bromomethyl radical or a formyl radical, according to Reaction Schemes 17, 18 and 19:

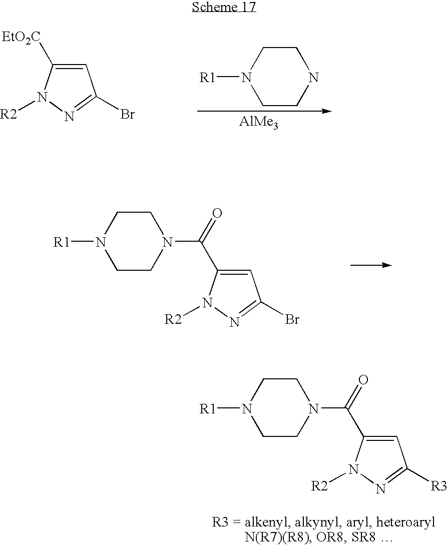

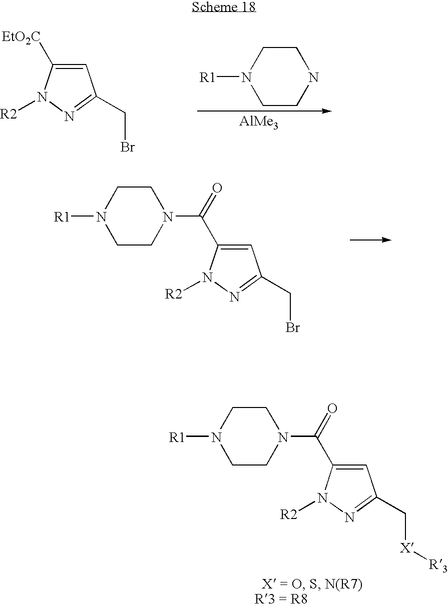

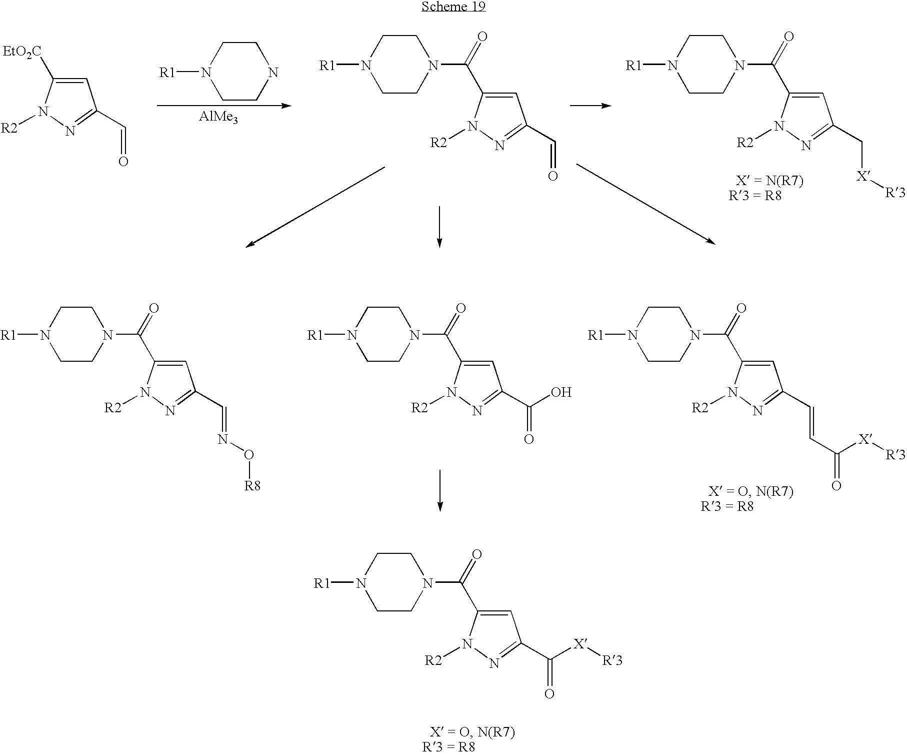

It is also advantageous, in the content of the invention, to modify, at the final stage(s) of the synthesis, the substituents borne by the radicals R1 and/or R2, which represent substituted aryl or heteroaryl groups, via standard methods known to those skilled in the art, for instance, in a nonlimiting manner, the reduction of a nitro radical to an amino radical, the alkylation of a phenol or thiophenol radical to a phenyl ether or thioether, the hydrolysis of a cyano radical to a carboxyl or carboxamide radical, the acylation of an amino radical to an amide, the esterification or amidation of a carboxyl radical.

The general synthetic methods presented in Schemes 1 to 16 illustrate, in a nonlimiting manner, the possible preparations of the compounds of the invention. Many other synthetic routes may be used, in particular those described in:
Comprehensive Heterocyclic Chemistry, 5 (Part 4A), by A. Katritsky et al. (Pergamon Press);
Advanced Organic Chemistry ‘Progress in Pyrazole Chemistry”, 6, 347-429 (1966) by A. N. Kost et al.;
Journal of Heterocyclic Chemistry ”Synthesis of Pyrazoles and condensed Pyrazoles”, 36, 321-332 (1999) by M. Kenzi et al.;
Organic Chemistry “The Chemistry of pyrroles”, 34 (1977) by A. R. Jones et al. (Academic Press);
Organic Chemistry in Monographs “Chemistry of Pyrroles, 15 (1974) by A. Gossauer (Springer Verlag).

The examples below illustrate, in a nonlimiting manner, the products of the invention.

General Conditions:

1. The reactions using microwaves were performed on a Personal Chemistry Emrys™ Optimizer machine in Emrys™ process vials of 0.5-2.0 ml or 2.0-5.0 ml.
2. With the exception of particular conditions explicitly described—for instance for the Example 3 library products—the LC/MS analyses are performed under the conditions below:
   X Terra RP₁₈ 2.1×50 mm 3.5 μm
   oven at 40° C., flow rate=0.7 ml/min, injection volume V=10 μl

eluent A: H₂O+0.1% HCOOH pH=2 B: CH₃CN

| time (min) | A % | B % |
|---|---|---|
| 0.0 | 95 | 5 |
| 5.0 | 5 | 95 |
| 6.5 | 5 | 95 |
| 7.0 | 95 | 5 |
| 9.0 | 95 | 5 |

MS/ES positive and negative mode detection CV50V, m/z 50-1500
DAD λ=200 to 400 nm
ELSD T evaporation=75° C., T nebulization=80° C., flow rate=1 I/min
3. With the exception of particular conditions explicitly described, the purifications by preparative LC/MS are performed under the conditions below:
   X Terra RP₁₈30×100 mm 5 μm column
   eluents:
   i) pH=5 A: aqueous 20 mM solution of ammonium hydrogen carbonate+HCOOH (up to pH=5)/B: CH₃CN, or

ii) pH=9 A: aqueous 20 mM solution of ammonium hydrogen carbonate+aqueous ammonia (up to pH=9). B: CH₃CN

| time (min) | flow rate (ml/min) | A % | B % |
|---|---|---|---|
| 0.0 | 10 | 70 | 30 |
| 3.0 | 30 | 50 | 50 |
| 4.0 | 30 | 40 | 60 |
| 11.0 | 30 | 0 | 100 |
| 12.5 | 30 | 0 | 100 |
| 12.9 | 20 | 0 | 100 |

detection: positive and negative mode MS/ES CV=20V, m/z 100-1100

### EXAMPLE 1

### [4-(3-Chlorophenyl)piperazin-1-yl](5-methyl-2-phenyl-2H-pyrazol-3-yl)methanone

A solution of 250 μl of oxalyl chloride in 1 ml of dichloromethane is added at 0° C. to a solution of 387 mg of 5-methyl-2-phenyl-2H-pyrazole-3-carboxylic acid, which may be obtained according to J. Het. Chem., 30, 307 (1993), in 4 ml of dichloromethane. One drop of dimethylformamide is added to this reaction mixture. After stirring for 3 hours at a temperature close to 20° C., the reaction mixture is concentrated to dryness, taken up in 5 ml of tetrahydrofuran and then added slowly to a solution of 413 mg of 1-(3-chlorophenyl)piperazine and 402 μl of triethylamine in 5 ml of tetrahydrofuran. After stirring for 18 hours at room temperature, the reaction mixture is concentrated to dryness. The brown paste obtained is taken up in 20 ml of ethyl acetate and then washed three times with 10 ml of distilled water. The organic phase is dried over magnesium sulfate and then concentrated to dryness under reduced pressure. After purification on silica (eluent: cyclohexane/ethyl acetate), 402 mg of [4-(3-chlorophenyl)piperazin-1-yl](5-methyl-2-phenyl-2H-pyrazol-3-yl)methanone are collected in the form of a white powder, the characteristics of which are as follows:
IR spectrum (KBr): 2835; 1634; 1593; 1500; 1445; 1236; 1003; 944; 762 and 692 cm⁻¹
¹H NMR spectrum (300 MHz, (CD₃)₂SO d6, δ in ppm): 2.30 (s: 3H); 2.88 (mt: 2H); 3.17 (mt: 2H); 3.36 (mt: 2H); 3.69 (mt: 2H); 6.53 (s: 1H); 6.83 (dd, J=8 and 1.5 Hz: 1H); 6.86 (dd, J=8 and 1.5 Hz: 1H); 6.92 (t, J=1.5 Hz: 1H); 7.23 (t, J=8 Hz: 1H); 7.36 (tt, J=7 and 2 Hz: 1H); from 7.40 to 7.55 (mt: 4H).

### EXAMPLE 2

### [4-(3,4-Dimethylphenyl)piperazin-1-yl](5-methyl-2-phenyl-2H-pyrazol-3-yl)methanone

By working in a manner similar to that for the synthesis of [4-(3-chlorophenyl)piperazin-1-yl](5-methyl-2-phenyl-2H-pyrazol-3-yl)methanone, but replacing the 1-(3-chlorophenyl)piperazine with 400 mg of 1-(3,4-dimethylphenyl)piperazine, 285 mg of [4-(3,4-dimethylphenyl)piperazin-1-yl]-(5-methyl-2-phenyl-2H-pyrazol-3-yl)methanone are obtained in the form of a pale yellow solid, the characteristics of which are as follows:
IR spectrum (CCl₄): 2921; 2862; 2819; 1646; 1504; 1473; 1446; 1365; 1236; 1002 and 692 cm⁻¹
¹H NMR spectrum (300 MHz, (CD3)2SO d6, δ in ppm): 2.12 (s: 3H); 2.17 (s: 3H); 2.30 (s: 3H); 2.71 (mt: 2H); 3.01 (mt: 2H); from 3.25 to 3.40 (mt: 2H); 3.68 (mt: 2H); 6.52 (s: 1H); 6.59 (dd, J=8 and 2 Hz: 1H); 6.70 (d, J=2 Hz: 1H); 6.97 (d, J=8 Hz: 1H); 7.36 (tt, J=7 and 2 Hz: 1H); from 7.40 to 7.55 (mt: 4H).

### EXAMPLE 3

Example 3 below shows an application of the use of the general synthetic route presented in Scheme 16. In this case, N-phenylpiperazine and 5-methyl-2-phenyl-2H-pyrazole-3-carboxylic acid may be substituted with radicals as defined above, to obtain products in accordance with the invention.

Synthesis of the Library

100 μl of a molar solution of N,N-diisopropylcarbodiimide in DMF, 100 μl of a decimolar solution of N-dimethylaminopyridine in DMF and then finally 200 μl of a solution at 0.5 mol/l in DMF of the corresponding pyrazole acids are added to a suspension of 50 mg of tetrafluorophenyl resin (IRORI, unisphere 200; substitution 0.99 mmol/g) in 0.6 ml of dimethylformamide. The reaction mixtures are stirred at room temperature for 18 hours and then filtered. The resins are then washed three times with 1 ml of DMF and then alternatively four times with 1 ml of CH₂Cl₂ and 4 times with 1 ml of methanol, and then finally twice with 1 ml of CH₂Cl₂. The resins are dried in ambient air.

A solution containing 0.5 mol/l of substituted phenylpiperazine and 0.75 mol/l of triethylamine is added to a suspension of 50 mg of resin obtained beforehand in 0.9 ml of dimethylformamide. The reaction medium is stirred for 18 hours and then filtered. The residual resin is washed twice with 0.5 ml of dimethylformamide. The filtrate is concentrated to dryness and the oils obtained are purified by high performance liquid chromatography coupled to a mass spectrometer (LC/MS).

Purification by LC/MS

The products were purified by LC/MS using a Waters FractionLynx system composed of a Waters model 600 gradient pump, a Waters model 515 regeneration pump, a Waters Reagent Manager dilution pump, a Waters model 2700 autoinjector, two Rheodyne LabPro model valves, a Waters model 996 diode array detector, a Waters model ZMD mass spectrometer and a Gilson model 204 fraction collector. The system was controlled by the Waters FractionLynx software. The separation was performed alternately on two Waters Symmetry columns (C₁₈, 5 μm, 19×50 mm, catalogue reference 186000210), one column undergoing regeneration with a 95/5 (v/v) water/acetonitrile mixture containing 0.07% (v/v) of trifluoroacetic acid, while the other column was performing separation. The columns were eluted using a linear gradient of from 5% to 95% of acetonitrile containing 0.07% (v/v) of trifluoroacetic acid in water containing 0.07% (v/v) of trifluoroacetic acid, at a flow rate of 10 ml/min. At the separation column outlet, a thousandth of the effluent is separated out using an LC Packing Accurate device, diluted with methanol at a flow rate of 0.5 ml/min and sent to the detectors, in a proportion of 75% to the diode array detector and the remaining 25% to the mass spectrometer. The rest of the effluent (999/1000) is sent to the fraction collector where the flow is discarded as long as the mass of the expected product is not detected by the FractionLynx software. The molecular formulae of the expected products are supplied to the FractionLynx software, which initiates collection of the product when the detected mass signal corresponds to the [M+H]⁺ and/or [M+Na]⁺ ion. In certain cases, depending on the analytical LC/MS results, when an intense ion corresponding to [M+2H]⁺⁺ was detected, the value corresponding to half the calculated molecular mass (MW/2) is also supplied to the FractionLynx software. Under these conditions, collection is also initiated when the mass signal of the [M+2H]⁺⁺ and/or [M+Na+H]⁺⁺ ion is (are) detected. The products were collected in tarred glass tubes. After collection, the solvents were evaporated off, in a Savant AES 2000 or Genevac HT8 centrifuge evaporator and the masses of products were determined by weighing the tubes after evaporation of the solvents.

The LC/MS analyses were performed on a Micromass model LCT machine connected to an HP 1100 machine. The abundance of the products was measured using an HP G1315A diode array detector over a wavelength range of 200-600 nm and a Sedex 65 light-scattering detector. The mass spectra were acquired over a range from 180 to 800. The data were analyzed using the Micromass MassLynx software. The separation was performed on a Hypersil BDS C18, 3 μm (50×4.6 mm) column, eluting with a linear gradient of from 5% to 90% of acetonitrile containing 0.05% (v/v) of trifluoroacetic acid (TFA) in water containing 0.05% (v/v) of TFA, over 3.5 minutes at a flow rate of 1 ml/min. The total analysis time, including the volume reequilibration period, is 7 minutes.

The products of Examples 3/1 to 3/153 were obtained using a protocol according to Example 3.

### EXAMPLE 4

### (4-Phenyl-1,2,3,6-tetrahydropyridin-1-yl)(5-methyl-2-phenyl-2H-pyrazol-3-yl)-methanone

316 mg of 1-(3-dimethylaminopropyl)-3-ethylcarbodiimide hydrochloride (EDCl) and 20 mg of 1-hydroxybenzotriazole hydrate (HOBT) are added to a solution of 300 mg of 5-methyl-2-phenyl-2H-pyrazole-3-carboxylic acid, which may be obtained according to J. Het. Chem., 30, 307 (1993) in 15 ml of dichloromethane. After stirring for 10 minutes at room temperature, 230 μl of triethylamine (TEA) and 323 mg of 4-phenyl-1,2,3,6-tetrahydropyridine hydrochloride are added and this reaction mixture is then stirred for 36 hours at room temperature. After addition of 10 ml of water, the organic phase is separated out by settling of the phases, and then washed with water, dried over magnesium sulfate and concentrated under reduced pressure. After purification by flash chromatography on a column of silica (60; 35-70 μm), eluting with a mixture of cyclohexane and ethyl acetate (75/25 by volume), 300 mg of (4-phenyl-1,2,3,6-tetrahydropyridin-1-yl)(5-methyl-2-phenyl-2H-pyrazol-3-yl)methanone are obtained in the form of a white foam, the characteristics of which are as follows:
Melting point (Kofler)=68° C.
Mass spectrum (EI): m/z=343 (M⁺)

### EXAMPLE 5

### [4-(3-Chlorophenyl)piperazin-1-yl](2-phenyl-2H-pyrazol-3-yl)methanone

The process is performed in a manner similar to that of Example 1, but starting with 500 mg of 2-phenyl-2H-pyrazol-5ylcarboxylic acid, which may be prepared according to Heterocycles, 23, 943 (1985), of 575 mg of 1-(3-chlorophenyl)piperazine and 342 μl of oxalyl chloride in 20 ml of dichloromethane, to give, after purification by flash chromatography on a column of silica (60; 35-70 μm), eluting with a mixture of dichloromethane and methanol (98/2 by volume), 680 mg of [4-(3-chlorophenyl)piperazin-1-yl](2-phenyl-2H-pyrazol-3-yl)methanone in the form of a very viscous pale yellow oil, the characteristics of which are as follows:
Mass spectrum (EI): m/z=366 (M⁺)

### EXAMPLE 6

### [4-(3-Chlorophenyl)piperazin-1-yl](1-phenyl-1H-pyrrol-2-yl)methanone

The process is performed in a manner similar to that of Example 1, but starting with 187 mg of 1-phenyl-1H-pyrrol-2ylcarboxylic acid, which may be prepared according to Tetrahedron, 49, 10271 (1993), 216 mg of 1-(3-chlorophenyl)piperazine and 128 μl of oxalyl chloride in 20 ml of dichloromethane, to give, after purification by flash chromatography on a column of silica (60; 35-70 μm), eluting with a mixture of dichloromethane and methanol (98/2 by volume) followed by recrystallization from diisopropyl ether, 200 mg of [4-(3-chlorophenyl)piperazin-1-yl](1-phenyl-1H-pyrrol-2-yl)methanone in the form of white crystals, the characteristics of which are as follows:
Mass spectrum (EI): m/z=365 (M⁺)
Melting point (Kofler)=105° C.

### EXAMPLE E2

### [4-(3,5-Dichlorophenyl)piperazin-1-yl](5-methyl-2-phenyl-2H-pyrazol-3-yl)methanone

By performing the process in a manner similar to that for the synthesis of [4-(3-chlorophenyl)piperazin-1-yl](5-methyl-2-phenyl-2H-pyrazol-3-yl)methanone (Example1), but replacing the 1-(3-chlorophenyl)piperazine with 486 mg of 1-(3,5-dichlorophenyl)piperazine, 147 mg of [4-(3,5-dichlorophenyl)piperazin-1-yl](5-methyl-2-phenyl-2H-pyrazol-3-yl)methanone are obtained in the form of a white solid, the characteristics of which are as follows:
IR spectrum (KBr): 2924; 2852; 1629; 1587; 1554; 1502; 1463; 1288; 1241; 982; 963; 794; 764; 691 and 672 cm⁻¹
¹H NMR spectrum (400 MHz, (CD₃)₂SO d6, δ in ppm): 2.31 (s: 3H); 2.98 (unresolved complex: 2H); 3.25 (unresolved complex: 2H); 3.37 (unresolved complex: 2H); 3.67 (unresolved complex: 2H); 6.54 (s: 1H); from 6.85 to 7.00 (mt: 3H); 7.37 (tt, J=7.5 and 1.5 Hz: 1H); 7.45 (broad d, J=7.5 Hz: 2H); 7.50 (broad t, J=7.5 Hz: 2H).

### EXAMPLE E3

### [4-(3-Dimethylaminophenyl)piperazin-1-yl](5-methyl-2-phenyl-2H-pyrazol-3-yl)methanone.

Step 1: 1.6 g of bis-(2-chloroethyl)amine hydrochloride and 2.86 g of sodium carbonate are added to a solution of 1.2 g of N,N-dimethylbenzene-1,3-diamine, which may be obtained according to J. Org. Chem., 57, 5254 (1992), in 15 ml of n-butanol. After refluxing for 18 hours, 50 ml of dichloromethane and 40 ml of water are added, the organic phase is separated out by settling of the phases and is then washed with 40 ml of water, dried over magnesium sulfate and concentrated under reduced pressure. 2.35 g of dimethyl-(3-piperazin-1-ylphenyl)amine are thus obtained in the form of a viscous brown oil, which is used without further purification in the following step, and the characteristics of which are as follows:
Mass spectrum IC m/z=206 MH⁺ base peak

Step 2: 316 mg of 1-(3-dimethylaminopropyl)-3-ethylcarbodiimide hydrochloride (EDCl), 20 mg of 1-hydroxybenzotriazole hydrate (HOBT) and 0.48 g of dimethyl(3-piperazin-1-ylphenyl)amine are added to a solution of 303 mg of 5-methyl-2-phenyl-2H-pyrazole-3-carboxylic acid, which may be obtained according to J. Het. Chem., 30, 307 (1993) in 15 ml of dichloromethane, and this reaction mixture is stirred for 36 hours at room temperature. After adding 20 ml of water, the organic phase is separated out by settling of the phases and is then washed with water, dried over magnesium sulfate and concentrated under reduced pressure. After purification by flash chromatography on a column of silica (60; 35-70 μm), eluting with a mixture of cyclohexane and ethyl acetate (60/40 by volume), 140 mg of [4-(3-dimethylaminophenyl)-piperazin-1-yl](5-methyl-2-phenyl-2H-pyrazol-3-yl)methanone are obtained in the form of a white foam, the characteristics of which are as follows:
¹H NMR spectrum (400 MHz, (CD₃)₂SO d6, δ in ppm): 2.31 (s: 3H); 2.76 (unresolved complex: 2H); 2.87 (s: 6H); 3.08 (unresolved complex: 2H); 3.33 (unresolved complex: 2H); 3.70 (unresolved complex: 2H); 6.18 (t, J=2 Hz: 1H); 6.22 (dd, J=8.5 and 2 Hz: 1H); 6.26 (dd, J=8.5 and 2 Hz: 1H); 6.54 (s: 1H); 7.02 (t, J=8.5 Hz: 1H); 7.38 (tt, J=7.5 and 1.5 Hz: 1H); from 7.45 to 7.55 (mt: 4H).

### EXAMPLE E4

### [4-(6-Chloropyrid-2-yl)piperazin-1-yl](5-methyl-2-phenyl-2H-pyrazol-3-yl)-methanone

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 745 mg of 1-(6-chloro-2-pyridyl)piperazine, which may be obtained according to U.S. Pat. No. 4,078,063, 900 mg of [4-(6-chloropyrid-2-yl)piperazin-1-yl](5-methyl-2-phenyl-2H-pyrazol-3-yl)methanone are obtained in the form of a white foam, the characteristics of which are as follows:
¹H NMR spectrum (300 MHz, (CD₃)₂SO d6, δ in ppm): 2.31 (s: 3H); 3.22 (unresolved complex: 2H); 3.36 (unresolved complex: 2H); 3.51 (unresolved complex: 2H); 3.66 (unresolved complex: 2H); 6.54 (s: 1H); 6.71 (d, J=7.5 Hz: 1H); 6.76 (d, J=8 Hz: 1H); 7.36 (broad t, J=7 Hz: 1H); from 7.40 to 7.55 (mt: 4H); 7.58 (broad dd, J=8 and 7 Hz: 1H).

### EXAMPLE E5

### [4-(3-Nitrophenyl)piperazin-1-yl](5-methyl-2-phenyl-2H-pyrazol-3-yl)-methanone

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 683.8 mg of 1-(3-nitrophenyl)piperazine, which may be obtained according to J. Med. Chem., 32, 1052 (1989), 500 mg of [4-(3-nitrophenyl)piperazin-1-yl](5-methyl-2-phenyl-2H-pyrazol-3-yl)methanone are obtained in the form of a yellow solid, the characteristics of which are as follows:
Melting point (Kofler): 142° C.
¹H NMR spectrum (300 MHz, (CD₃)₂SO d6, δ in ppm): 2.31 (broad s: 3H); 3.00 (unresolved complex: 2H); 3.28 (unresolved complex: 2H); 3.42 (unresolved complex: 2H); 3.73 (unresolved complex: 2H); 6.56 (s: 1H); from 7.30 to 7.65 (mt: 9H).

### EXAMPLE E6

### [4-(3-Bromophenyl)piperazin-1-yl](5-methyl-2-phenyl-2H-pyrazol-3-yl)methanone

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 1.25 g of 1-(3-bromophenyl)piperazine, which may be obtained according to Chem. Pharm. Bull. 50, 453 (2002), 1.65 g of [4-(3-bromophenyl)piperazin-1-yl](5-methyl-2-phenyl-2H-pyrazol-3-yl)methanone are obtained in the form of a white solid, the characteristics of which are as follows:
Melting point (Kofler): 138° C.
¹H NMR spectrum (300 MHz, (CD₃)₂SO d6, δ in ppm): 2.30 (s: 3H); 2.87 (unresolved complex: 2H); 3.17 (unresolved complex: 2H); 3.38 (unresolved complex: 2H); 3.69 (unresolved complex: 2H); 6.54 (s: 1H); 6.89 (dd, J=8.5 and 2 Hz: 1H); 6.97 (broad dd, J=8.5 and 1.5 Hz: 1H); 7.06 (broad s: 1H); 7.17 (t, J=8.5 Hz: 1H); 7.37 (tt, J=7.5 and 1.5 Hz: 1H); from 7.40 to 7.55 (mt: 4H).

### EXAMPLE E7

### [4-(3-Chlorophenyl)piperazin-1-yl](5-methyl-2-phenyl-2H-pyrazol-3-yl)-methanethione

212 mg of Lawesson's reagent are added to a solution of 0.2 g of [4-(3-chlorophenyl)piperazin-1-yl](5-methyl-2-phenyl-2H-pyrazol-3-yl)methanone, obtained in Example 1, in 10 ml of toluene. After refluxing for 1 hour 30 minutes and adding 10 ml of ethyl acetate, the organic phase is separated out by settling of the phases and is then washed with 30 ml of saturated sodium bicarbonate solution and dried over magnesium sulfate, and then concentrated under reduced pressure. After purification by flash chromatography on a column of silica (60; 35-70 μm), eluting with a mixture of cyclohexane and ethyl acetate (80-20 by volume), 130 mg of [4-(3-chlorophenyl)piperazin-1-yl](5-methyl-2-phenyl-2H-pyrazol-3-yl)methanethione are obtained in the form of a white foam, the characteristics of which are as follows:
Melting point: (Kofler)=95-98° C.
¹H NMR spectrum (300 MHz, (CD₃)₂SO d6 with addition of a few drops of CD₃COOD d4, δ in ppm): 2.29 (broad s: 3H); from 2.80 to 3.70 (broad unresolved complex: 6H); from 4.00 to 4.50 (broad unresolved complex: 2H); 6.42 (broad s: 1H); from 6.70 to 6.90 (mt: 3H); 7.21 (broad t, J=8 Hz: 1H); 7.33 (broad t, J=7 Hz: 1H); from 7.40 to 7.55 (mt: 4H).

### EXAMPLE E8

### [4-(6-Methoxypyrid-2-yl)piperazin-1-yl](5-methyl-2-phenyl-2H-pyrazol-3-yl)-methanone

169 mg of sodium methoxide are added to a solution of 300 mg of [4-(6-chloropyrid-2-yl)piperazin-1-yl](5-methyl-2-phenyl-2H-pyrazol-3-yl)methanone, described in Example E4, in 10 ml of methanol. After refluxing for 48 hours and concentrating under reduced pressure, the crude mixture is purified by flash chromatography on a column of silica (60; 35-70 μm), eluting with a mixture of cyclohexane and ethyl acetate (70-30 by volume), to give 9 mg of [4-(6-methoxypyrid-2-yl)piperazin-1-yl](5-methyl-2-phenyl-2H-pyrazol-3-yl)-methanone in the form of a white solid, the characteristics of which are as follows:
¹H NMR spectrum (300 MHz, (CD₃)₂SO d6, δ in ppm): 2.30 (broad s: 3H); 3.20 (unresolved complex: 2H); 3.33 (unresolved complex: 2H); 3.49 (unresolved complex: 2H); 3.66 (unresolved complex: 2H); 3.77 (s: 3H); 6.08 (d, J=7.5 Hz: 1H); 6.30 (d, J=8 Hz: 1H); 6.54 (s: 1 H); 7.37 (broad t, J=7.5 Hz: 1H); from 7.40 to 7.55 (mt: 5H).

### EXAMPLE E9

### [4-(3-Aminophenyl)piperazin-1-yl](5-methyl-2-phenyl-2H-pyrazol-3-yl)methanone

9.5 mg of 10% of palladium-on-charcoal are added to a solution of 350 mg of [4-(3-nitrophenyl)piperazin-1-yl](5-methyl-2-phenyl-2H-pyrazol-3-yl)methanone, described in Example E5, in 10 ml of absolute ethanol, and hydrogen is introduced at a pressure of about 1 bar and at a temperature in the region of 20° C. After reaction for 20 hours, the catalyst is filtered off and the filtrate is concentrated under reduced pressure. The residue is purified by flash chromatography on a column of silica (60; 35-70 μm), eluting with a mixture of cyclohexane and ethyl acetate (30/70 by volume), and 265 mg of [4-(3-aminophenyl)piperazin-1-yl](5-methyl-2-phenyl-2H-pyrazol-3-yl)methanone are thus obtained in the form of a white solid, the characteristics of which are as follows:
Melting point (Kofler): 158° C.

Mass spectrum (EI): m/z=361 (M+)

¹H NMR spectrum (300 MHz, (CD₃)₂SO d6, δ in ppm): 2.30 (broad s: 3H); 2.69 (unresolved complex: 2H); 2.98 (unresolved complex: 2H); 3.33 (unresolved complex: 2H); 3.67 (unresolved complex: 2H); 4.90 (broad s: 2H); from 6.00 to 6.10 (mt: 3H); 6.52 (s: 1H); 6.85 (broad t, J=8 Hz: 1H); 7.37 (tt, J=7 and 1.5 Hz: 1H); from 7.30 to 7.55 (mt: 4H).

### EXAMPLE E10

### [4-(3-Cyanophenyl)piperazin-1-yl](5-methyl-2-phenyl-2H-pyrazol-3-yl)-methanone

69 mg of sodium cyanide, 285 mg of tetrakis(triphenylphosphine)palladium and 140 mg of aluminum oxide are added to a solution of 0.5 g of [4-(3-bromophenyl)piperazin-1-yl](5-methyl-2-phenyl-2H-pyrazol-3-yl)methanone, described in Example E6, in 20 ml of toluene. After refluxing for 20 hours and adding 50 ml of ethyl acetate, the organic phase is separated out by settling of the phases and then washed with twice 50 ml of water and dried over magnesium sulfate. After purification by flash chromatography on a column of silica (60; 35-70, μm), eluting with a mixture of cyclohexane and ethyl acetate (60/40 by volume), 125 mg of [4-(3-cyanophenyl)piperazin-1-yl](5-methyl-2-phenyl-2H-pyrazol-3-yl)methanone are thus obtained in the form of a white solid, the characteristics of which are as follows:
Melting point (Kofler): 136° C.
Mass spectrum (EI): m/z=371 (M+)

¹H NMR spectrum (300 MHz, (CD₃)₂SO d6, δ in ppm): 2.31 (s: 3H); 2.96 (unresolved complex: 2H); 3.24 (unresolved complex: 2H); 3.37 (unresolved complex: 2H); 3.70 (unresolved complex: 2H); 6.54 (s: 1H); from 7.15 to 7.55 (mt: 9H).

### EXAMPLE E11

### [4-(3-Trifluoromethyloxyphenyl)piperazin-1-yl](5-methyl-2-phenyl-2H-pyrazol-3-yl)methanone

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 462 mg of 1-(3-trifluoromethyloxyphenyl)piperazine, which may be obtained according to J. Med. Chem., 22, 554 (1979), 425 mg of [4-(3-trifluoromethyloxyphenyl)-piperazin-1-yl](5-methyl-2-phenyl-2H-pyrazol-3-yl)methanone are obtained in the form of a white solid, the characteristics of which are as follows:
Melting point (Kofler): 107° C.
Mass spectrum (EI): m/z=430 (M+)

### EXAMPLE E12

### [4-(1,3-Benzodioxol-5-yl)piperazin-1-yl](5-methyl-2-phenyl-2H-pyrazol-3-yl)-methanone

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 606 mg of 1-(1,3-benzodioxol-5-yl)piperazine, which may be obtained according to Tetrahedron Lett., 39, 617 (1998), 920 mg of [4-(1,3-benzodioxol-5-yl)-piperazin-1-yl](5-methyl-2-phenyl-2H-pyrazol-3-yl)methanone are obtained in the form of a white solid, the characteristics of which are as follows:
Melting point (Kofler): 128° C.
Mass spectrum (EI): m/z=390 (M+)

### EXAMPLE E13

### [4-(3-Hydroxyphenyl)piperazin-1-yl](5-methyl-2-phenyl-2H-pyrazol-3-yl)-methanone

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 171 mg of 1-(3-hydroxyphenyl)piperazine, 258 mg of [4-(3-hydroxyphenyl)piperazin-1-yl](5-methyl-2-phenyl-2H-pyrazol-3-yl)methanone are obtained in the form of a solid, the characteristics of which are as follows:
¹H NMR spectrum (300 MHz, (CD₃)₂SO d6, δ in ppm): 2.30 (s: 3H); 2.73 (unresolved complex: 2H); 3.03 (unresolved complex: 2H); from 3.25 to 3.40 (unresolved complex: 2H); 3.67 (unresolved complex: 2H); from 6.20 to 6.30 (mt: 2H); 6.32 (broad d, J=8.5 Hz: 1H); 6.52 (s: 1H); 6.98 (t, J=8.5 Hz: 1H); 7.36 (tt, J=7 and 1.5 Hz: 1H); from 7.40 to 7.55 (mt: 4H); 9.17 (s: 1H).

### EXAMPLE E14

### [4-(3-chlorophenyl)piperazin-1-yl](5-methyl-2-phenyl-2H-pyrazol-3-yl)methane

Step 1: 195 mg of O,N-dimethylhydroxylamine hydrochloride and 300 μl of triethylamine are added to a solution of 404 mg of 5-methyl-2-phenyl-2H-pyrazole-3-carboxylic acid, 360 mg of 1-(3-dimethylaminopropyl)-3-ethyl-carbodiimide hydrochloride and 270 mg of 1-hydroxybenzotriazole in 10 ml of dichloromethane. After stirring for 96 hours at a temperature in the region of 20°C., the medium is diluted with 20 ml of dichloromethane, the phases are separated by settling and the organic phase is washed with 20 ml of 1N hydrochloric acid solution and then three times with 15 ml of distilled water. After drying over magnesium sulfate, concentrating under reduced pressure and purifying by flash chromatography on a column of silica (60; 35-70 μm), eluting with a mixture of dichloromethane and methanol (gradient of from 100% to 90% dichloromethane by volume), 360 mg of N-methoxy-N-methyl-5-methyl-2-phenyl-2H-pyrazole-3-carboxamide are obtained, and used directly in the following step.

Step 2: A solution of 350 mg of N-methoxy-N-methyl-5-methyl-2-phenyl-2H-pyrazole-3-carboxamide in 1.2 ml of ethyl ether is added, at a temperature in the region of −60° C., to a suspension of 69 mg of lithium aluminum hydride in 3.2 ml of ethyl ether. After the temperature has risen by about 5° C., a solution of 315 mg of sodium hydrogen sulfate in 1.1 ml of distilled water is added. After dilution with 20 ml of ethyl ether, the organic phase is separated out by settling of the phases and then washed twice with 1N hydrochloric acid solution, at a temperature in the region of 0° C., twice with saturated sodium hydrogen carbonate solution, once with saturated sodium chloride solution, dried over magnesium sulfate and concentrated under reduced pressure. The concentrate is dissolved in 5 ml of dichloromethane and 200 mg of manganese dioxide are added. After stirring for 18 hours at a temperature in the region of 20° C., the medium is concentrated under reduced pressure and then taken up in 20 ml of ethyl acetate, filtered in the presence of Celite and then concentrated under reduced pressure to give 266 mg of 5-methyl-2-phenyl-2H-pyrazole-3-carboxaldehyde in the form of a foam, which is used without further purification in the following step after control by LC/MS analysis.

Step 3: 55.8 mg of 5-methyl-2-phenyl-2H-pyrazole-3-carboxaldehyde, 17 μl of acetic acid and 380 mg of powdered 3 Å molecular sieves are added to a solution of 39 mg of 1-(3-chlorophenyl)piperazine in 10 ml of acetonitrile. After stirring for 2 hours at a temperature in the region of 20° C., 18.9 mg of sodium cyanoborohydride are added. After stirring for 48 hours at a temperature in the region of 20° C., 200 μl of distilled water are added. After filtration, concentration under reduced pressure and then purification by LC/MS, 13 mg of [4-(3-chlorophenyl)piperazin-1-yl](5-methyl-2-phenyl-2H-pyrazol-3-yl)methane trifluoroacetate are obtained in the form of a white solid, the characteristics of which are as follows:
IR spectrum: 1679; 1596; 1502; 1456; 1206; 1137; 945; 800; 721 and 699 cm⁻¹
¹H NMR spectrum (400 MHz, (CD₃)₂SO d6, at a temperature of 373 K, δ in ppm): 2.27 (s: 3H); 2.69 (unresolved complex: 4H); from 3.10 to 3.35 (unresolved complex: 4H); 3.75 (broad s: 2H); 6.33 (s: 1H); 6.80 (broad d, J=8 Hz: 1H); 6.88 (dd, J=8 and 2 Hz: 1 H); 6.92 (mt: 1H); 7.22 (t, J=8 Hz: 1H); 7.42 (broad t, J=7.5 and 1.5 Hz: 1H); 7.52 (broad t, J=7.5 Hz: 2H); 7.62 (broad d, J=7.5 Hz: 2H).

### EXAMPLE E15

### [4-(Isoquinolin-1-yl)piperazin-1-yl](5-methyl-2-phenyl-2H-pyrazol-3-yl)-methanone hydrochloride

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 100 mg of 1-(piperazin-1-yl)isoquinoline, which may be obtained according to patent WO 2002002568, 93 mg of [4-(isoquinolin-1-yl)piperazin-1-yl](5-methyl-2-phenyl-2H-pyrazol-3-yl)methanone hydrochloride are isolated in the form of a white solid, the characteristics of which are as follows:
Melting point (Kofler): 128° C.
Mass spectrum (EI): m/z=433 (M+)

### EXAMPLE E16

### [4-(4-Chloro-3-methylphenyl)piperazin-1-yl](5-methyl-2-phenyl-2H-pyrazol-3-yl)methanone

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 99.8 mg of 1-(4-chloro-3-methylphenyl)piperazine, which may be obtained from (4-chloro-3-methylphenyl)amine by working as described in Step 1 of Example E3, 70 mg of [4-(4-chloro-3-methylphenyl)piperazin-1-yl](5-methyl-2-phenyl-2H-pyrazol-3-yl)methanone are obtained in the form of a white solid, the characteristics of which are as follows:
Melting point (Kofler): 110° C.
Mass spectrum (EI): m/z=394 (M+)

### EXAMPLE E17

### (5-Methyl-2-phenyl-2H-pyrazol-3-yl)(4-quinolin-4-ylpiperazin-1-yl)methanone

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 100 mg of 4-(piperazin-1-yl)quinoline, which may be obtained according to J. Het. Chem., 33, 415 (1996), 100 mg of (5-methyl-2-phenyl-2H-pyrazol-3-yl)[4-(quinolin-4-yl)piperazin-1-yl]methanone are obtained in the form of a white foam, the characteristics of which are as follows:
Mass spectrum (EI): m/z=397 (M+)

### EXAMPLE E18

### N-{3-[4-(5-methyl-2-phenyl-2H-pyrazole-3-carbonyl)piperazin-1-yl]phenyl}-acetamide

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 800 mg of N-(3-piperazin-1-ylphenyl)acetamide, which may be obtained according to Tetrahedron Lett., 35, 7331 (1994), 1 g of N-{3-[4-(5-methyl-2-phenyl-2H-pyrazole-3-carbonyl)piperazin-1-yl]phenyl}acetamide is obtained in the form of an amorphous beige-colored solid, the characteristics of which are as follows:
Mass spectrum (EI): m/z=403 (M+)

¹H NMR spectrum (300 MHz, (CD₃)₂SO d6, δ in ppm): 2.03 (s: 3H); 2.31 (s: 3H); 2.76 (unresolved complex: 2H); 3.05 (unresolved complex: 2H); 3.33 (unresolved complex: 2H); 3.71 (unresolved complex: 2H); 6.53 (s: 1H); 6.58 (broad dd, J=8 and 2 Hz: 1H); 7.03 (broad d, J=8 Hz: 1H); 7.13 (t, J=8 Hz: 1H); 7.18 (broad s: 1H); from 7.40 to 7.55 (mt: 4H); 7.36 (tt, J=7 and 1.5 Hz: 1H); 9.79 (broad s: 1H).

### EXAMPLE E19

### (5-Methyl-2-phenyl-2H-pyrazol-3-yl)(2,3,5,6-tetrahydro-[1,2′]bipyrazinyl-4-yl)-methanone

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 51 mg of 2,3,5,6-tetrahydro[1,2′]bipyrazine, which may be obtained according to J. Med. Chem., 21, 536 (1978), 70 mg of (5-methyl-2-phenyl-2H-pyrazol-3-yl)-(2,3,5,6-tetrahydro-[1,2′]bipyrazinyl-4-yl)methanone are obtained in the form of a white solid, the characteristics of which are as follows:
Mass spectrum (EI): m/z=348 (M+)
Melting point (Kofler): 129° C.

### EXAMPLE E20

### [4-(3,5-Dimethoxyphenyl)piperazin-1-yl](5-methyl-2-phenyl-2H-pyrazol-3-yl)-methanone

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 467 mg of 1-(3,5-dimethoxyphenyl)piperazine, 727 mg of [4-(3,5-dimethoxyphenyl)-piperazin-1-yl](5-methyl-2-phenyl-2H-pyrazol-3-yl)methanone are obtained in the form of a solid, the characteristics of which are as follows:
¹H NMR spectrum (300 MHz, (CD₃)₂SO d6, δ in ppm): 2.31 (s: 3H); 2.80 (unresolved complex: 2H); 3.10 (unresolved complex: 2H); from 3.25 to 3.40 (unresolved complex: 2H); 3.68 (unresolved complex: 2H);
3.71 (s: 6H); from 5.95 to 6.05 (mt: 3H); 6.53 (s: 1H); 7.38 (broad t, J=7 Hz: 1H); from 7.40 to 7.55 (mt: 4H).

### EXAMPLE E21

### (5-Methyl-2-pyrid-2-yl-2H-pyrazol-3-yl)(4-phenylpiperazin-1-yl)methanone

Step 1: 2.26 ml of diisopropylethylamine, 2.96 g of HATU and 1.19 ml of 1-phenylpiperazine are added to a solution of 818 mg of 5-methyl-1H-pyrazole-3-carboxylic acid in 10 ml of DMF. After stirring for 2 hours at room temperature, the reaction mixture is poured into 100 ml of saturated aqueous sodium chloride solution and extracted with ethyl acetate. The organic phase is dried over magnesium sulfate and concentrated under reduced pressure. After purification by flash chromatography on a column of silica (40-63 μm), eluting with a mixture of dichloromethane, methanol and aqueous ammonia (95/5/0.5 by volume), 3.30 g of an orange oil are obtained. The product is taken up in 210 ml of dichloromethane in the presence of 22 g of Dowex® 50WX8 50-100 mesh resin, and the suspension is stirred for 1 hour at room temperature, filtered, rinsed with dichloromethane and suction-filtered. The resin is then taken up in 200 ml of a 9/1 mixture of methanol and aqueous ammonia, left in contact overnight and then filtered and rinsed. Concentration of the filtrate gives 1.39 g of (5-methyl-1H-pyrazol-3-yl)(4-phenylpiperazin-1-yl)methanone in the form of a pale yellow solid, the characteristics of which are as follows:
Mass spectrum (ES): m/z=343 (MH⁺)

Step 2: 100 mg of product from Step 1, 14 mg of cuprous iodide, 2.0 ml of 1,4-dioxane, 38 mg of trans-1,2-diaminocyclohexane, 169 mg of cesium carbonate, 88 mg of 2-bromopyridine and 20 mg of 1-ethyl-3-methylimidazolium chloride are placed in a microwave reactor and then subjected to the microwave field for 15 minutes at 140° C. 60 mg of cuprous iodide and 40 mg of 2-bromopyridine are added and the mixture is subjected to the microwave field for a further 15 minutes at 140° C. The reaction mixture is poured into 50 ml of water and extracted with ethyl acetate. The organic phase is dried over magnesium sulfate and concentrated under reduced pressure. After purification by flash chromatography on a column of silica (30-60 μm), eluting with a mixture of toluene, ethyl acetate and triethylamine (80/20/0.1 by volume), 52 mg of (5-methyl-2-pyrid-2-yl-2H-pyrazol-3-yl)(4-phenylpiperazin-1-yl)methanone are obtained in the form of a pale yellow solid, the characteristics of which are as follows:
Mass spectrum (EI): m/z=347 (M⁺)
¹H NMR spectrum (400 MHz, (CD₃)₂SO d6, δ in ppm): 2.63 ppm (s, 3H); 3.20 ppm (m, 4H); 3.80 ppm (m, 2H); 4.08 ppm (m, 2H); 6.62 ppm (s, 1H); 6.81 ppm (bt, J=8 Hz, 1H); 6.97 ppm (bd, J=8 Hz, 2H); 7.23 ppm (bt, J=8 Hz, 2H); 7.44 ppm (ddd, J=1.5-7.5-8.5 Hz, 1H); 7.85 ppm (bd, J=8.5 Hz, 1H); 8.03 ppm (ddd, J=2-7.5-8.5 Hz, 1H); 8.54 ppm (dm, J=5 Hz).

### EXAMPLE E22

### [4-(3-Chlorophenyl)piperazin-1-yl](5-methyl-2-pyrid-2-yl-2H-pyrazol-3-yl)-methanone

Step 1: 14.7 ml of a 2M solution of trimethylaluminum in toluene are added, at 25° C., to a solution of 4.325 g of 1-(3-chlorophenyl)piperazine in 60 ml of toluene, followed by addition, after 10 minutes, of 2.26 g of ethyl 5-methyl-1H-pyrazole-3-carboxylate. The reaction mixture is stirred for 6 hours at 60° C. and then poured into 100 ml of aqueous 1M sodium potassium tartrate solution and extracted with ethyl acetate. The organic phase is dried over magnesium sulfate and concentrated under reduced pressure. After purification by flash chromatography on a column of silica (40-63 μm), eluting with a mixture of ethyl acetate and triethylamine (98/2 by volume), 3.22 g of [4-3-chlorophenyl)piperazin-1-yl](5-methyl-2H-pyrazol-3-yl)methanone are obtained in the form of a pale yellow solid, the characteristics of which are as follows:
Mass spectrum (EI): m/z=304 (M⁺)

Step 2: The process is performed in a manner similar to that of Step 2 of Example E21, starting with 100 mg of the product of Step 1 of the present example, to give, after purification by flash chromatography on a column of silica (30-60 μm), eluting with a mixture of toluene, ethyl acetate and triethylamine (80/20/0.1 by volume), 24 mg of [4-(3-chlorophenyl)piperazin-1-yl](5-methyl-2-pyrid-2-yl-2H-pyrazol-3-yl)methanone in the form of a yellow oil, the characteristics of which are as follows:
LC/MS analysis: tr=4.12; m/z=382 (MH⁺)
¹H NMR spectrum (400 MHz, CDCl₃, δ in ppm): 2.37 (s, 3H); 3.08 ppm (m, 2H); 3.31 ppm (m, 2H); 3.45 ppm (m, 2H); 3.94 ppm (m, 2H); 6.28 ppm (s, 1H); 6.71 ppm (dd, J=2.5-8.5 Hz, 1H); 6.84 ppm (m, 2H); 7.15 ppm (m, 2H); 7.78 ppm (td, J=8-1.5 Hz, 1H); 7.88 ppm (bd, J=8 Hz, 1H); 8.26 ppm (bd, J=5 Hz, 1H).

### EXAMPLE E23

### 3-[4-(5-Methyl-2-phenyl-2H-pyrazole-3-carbonyl)piperazin-1-yl]benzamide

By working in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 11.13 g of 3-(piperazin-1-yl)benzamide, which may be obtained according to patent WO 98/00400, 11.5 g of 3-[4-(5-methyl-2-phenyl-2H-pyrazole-3-carbonyl)-piperazin-1-yl]benzamide are obtained in the form of an off-white solid, the characteristics of which are as follows:
Mass spectrum (EI): m/z=389 (M+)
Melting point (Kofler): 186° C.
¹H NMR spectrum (300 MHz, (CD₃)₂SO d6, δ in ppm): 2.31 (s: 3H); 2.89 (unresolved complex: 2H); 3.17 (unresolved complex: 2H); 3.38 (unresolved complex: 2H); 3.72 (unresolved complex: 2H); 6.54 (s: 1H); 7.04 (broad d, J=8 Hz: 1H); from 7.20 to 7.55 (mt: 9H); 7.89 (unresolved complex: 1H).

### EXAMPLE E24

### [4-(Biphenyl-3-yl)piperazin-1-yl)(5-methyl-2-phenyl-2H-pyrazol-3-yl)methanone

The process is performed in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 250.9 mg of 1-(biphenyl-3-yl)piperazine, which may be prepared according to patent WO 01/021604. 93 mg of [4-(biphenyl-3-ylpiperazin-1-yl)(5-methyl-2-phenyl-2H-pyrazol-3-yl)methanone are obtained in the form of a white lacquer, the characteristics of which are as follows:
Mass spectrum (EI): m/z=422 (M+)

### EXAMPLE E25

### [4-(3-Phenylmethyloxyphenyl)piperazin-1-yl](5-methyl-2-phenyl-2H-pyrazol-3-yl)methanone

By working in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 149 mg of 1-(3-benzyloxyphenyl)piperazine, which may be obtained from (3-phenylmethyloxyphenyl)amine by working as described in Step 1 of Example E3, 121 mg of [4-(3-phenylmethyloxyphenyl)piperazin-1-yl](5-methyl-2-phenyl-2H-pyrazol-3-yl)methanone are obtained in the form of a white solid, the characteristics of which are as follows:
Mass spectrum (EI): m/z=452(M+)
¹H NMR spectrum (300 MHz, (CD₃)₂SO d6, δ in ppm): 2.31 (s: 3H); 2.81 (unresolved complex: 2H); 3.11 (unresolved complex: 2H); 3.33 (unresolved complex: 2H); 3.68 (unresolved complex: 2H); 5.07 (s: 2H); from 6.40 to 6.55 (mt: 3H); 6.53 (s: 1H); 7.13 (t, J=8 Hz: 1H); from 7.25 to 7.55 (mt: 10H).

### EXAMPLE E26

### [4-(3-Methanesulfonylphenyl)piperazin-1-yl](5-methyl-2-phenyl-2H-pyrazol-3-yl)methanone

By working in a manner similar to that for the synthesis of [4-3-chlorophenyl)piperazin-1-yl](5-methyl-2-phenyl-2H-pyrazol-3-yl)methanone (Example 1), but replacing the 1-(3-chlorophenyl)piperazine with 150.2 mg of 1-(3-methanesulfonylphenyl)piperazine, which may be obtained according to patent WO 01/046145, 18 mg of [4-(3-methanesulfonylphenyl)piperazin-1-yl](5-methyl-2-phenyl-2H-pyrazol-3-yl)methanone are obtained in the form of a white solid, the characteristics of which are as follows:
Mass spectrum (EI): m/z=424 (M+)

### EXAMPLES E27 and E28

### tert-Butyl and ethyl esters of 3-[4-(5-methyl-2-phenyl-2H-pyrazole-3-carbonyl)piperazin-1-yl]benzoic acid

By working in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 1.5 g of a mixture (20/80) of the tert-butyl and ethyl esters of 3-(piperazin-1-yl)benzoic acid, which may be obtained according to patent WO 98/02432, 0.3 g of tert-butyl 3-[4-(5-methyl-2-phenyl-2H-pyrazole-3-carbonyl)piperazin-1-yl]benzoate 30 is obtained in the form of an off-white solid, the characteristics of which are as follows:
Mass spectrum (EI): m/z=446 (M+)
Melting point (Kofler): 144° C.
¹H NMR spectrum (300 MHz, (CD₃)₂SO d6, δ in ppm): 1.54 (s: 9H); 2.30 (s: 3H); 2.84 (unresolved complex: 2H); 3.15 (unresolved complex: 2H); 3.38 (unresolved complex: 2H); 3.71 (unresolved complex: 2H); 6.54 (s: 1H); 7.14 (mt: 1H); from 7.35 to 7.55 (mt: 8H). and 1 g of the ethyl ester of 3-[4-(5-methyl-2-phenyl-2H-pyrazole-3-carbonyl)piperazin-1-yl]benzoic acid are obtained in the form of a white solid, the characteristics of which are as follows:
Mass spectrum (EI): m/z=418 (M+)
Melting point (Kofler): 134° C.

### EXAMPLE E29

### [4-(1,3-Benzodioxol-4-yl)piperazin-1-yl](5-methyl-2-phenyl-2H-pyrazol-3-yl-methanone

By working in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 0.5 g of 1-(1,3-benzodioxol-4-yl)piperazine, which may be obtained according to J. Med. Chem., 45, 4128 (2002), 0.59 g of [4-(1,3-benzodioxol-4-yl)piperazin-1-yl])(5-methyl-2-phenyl-2H-pyrazol-3-yl)methanone is obtained in the form of a white solid, the characteristics of which are as follows:
Mass spectrum (EI): m/z=390 (M+)
Melting point (Kofler): 131° C.

### EXAMPLE E30

### [4-(1,3-Benzodioxol-4-yl)piperazin-1-yl](5-methyl-2-m-tolyl-2H-pyrazol-3-yl)methanone

By working in a manner similar to that for the synthesis of [4-3-chlorophenyl)piperazin-1-yl](5-methyl-2-phenyl-2H-pyrazol-3-yl)methanone (Example 1), but replacing the 1-(3-chlorophenyl)piperazine with 0.5 g of 1-(1,3-benzodioxol-4-yl)piperazine, which may be obtained according to J. Med. Chem., 45, 4128 (2002), and replacing the 5-methyl-2-phenyl-2H-pyrazole-3-carboxylic acid with 5-methyl-2-m-tolyl-2H-pyrazole-3-carboxylic acid, which may be obtained according to J. Het. Chem., 30, 307 (1993), 0.73 g of [4-(1,3-benzodioxol-4-yl)piperazin-1-yl](5-methyl-2-m-tolyl-2H-pyrazol-3-yl)methanone is obtained in the form of a white solid, the characteristics of which are as follows:
Mass spectrum (EI): m/z=404 (M+)
Melting point (Kofler): 132° C.

### EXAMPLE E31

### (5-Methyl-2-phenyl-2H-pyrazol-3-yl)[4-(4-methylpyrid-2-yl)piperazin-1-yl]methanone

By working in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 287 mg of 1-(4-methylpyrid-2-yl)piperazine, 154 mg of (5-methyl-2-phenyl-2H-pyrazol-3-yl)[4-(4-methylpyrid-2-yl)piperazin-1-yl]methanone are obtained in the form of a yellow gum, the characteristics of which are as follows:
Mass spectrum (EI): m/z=361 (M+)

### EXAMPLE E32

### [4-(3-Phenylmethylaminophenyl)piperazin-1-yl](5-methyl-2-phenyl-2H-pyrazol-3-yl)methanone hydrochloride.

Step 1: 0.93 ml of triethylamine and 0.78 ml of benzoyl chloride are added, in the region of 0° C., to a solution of 1.6 g of 3-(4-phenylmethylpiperazin-1-yl)phenylamine, which may be obtained according to patent WO 02/090327, in 50 ml of dichloromethane. After 72 hours in the region of room temperature and addition of 50 ml of water, the organic phase is separated out by settling of the phases, washed with twice 50 ml of water, with 50 ml of saturated sodium chloride solution and dried over magnesium sulfate and then concentrated under reduced pressure. After purification by flash chromatography on a column of silica (60; 35-70 μm), eluting with a mixture of cyclohexane and ethyl acetate (70-30 by volume), 1.2 g of N-(3-(piperazin-1-yl)phenyl]benzamide are obtained in the form of an amorphous brown solid, the characteristics of which are as follows:

| | | | |
|---|---|---|---|
| Mass spectrum IE | m/z = 281 | M^{+.} | |
| | m/z = 239 | (M - C₂H₄N)^{+.} | base peak |
| | m/z = 105 | C₇H₅O⁺ | |
| | m/z = 77 | C₆H₅⁺ | |

Step 2: 106 mg of lithium aluminum hydride are added to a solution of 0.5 g of N-[3-(piperazin-1-yl)phenyl]benzamide in 20 ml of tetrahydrofuran, and the mixture is refluxed for 20 hours. After addition of 1 ml of ethyl acetate, 1 ml of water, 1 ml of 1N sodium hydroxide and 1 ml of water, is thus obtained; the insoluble material is removed by filtration and, after separation of the phases by settling, washing with 25 ml of saturated aqueous sodium chloride solution and drying over magnesium sulfate, 0.5 g of phenylmethyl-[3-(piperazin-1-yl-)phenyl]amine in the form of an orange-colored oil, the characteristics of which are as follows:

| | | | |
|---|---|---|---|
| Mass spectrum IC | m/z = 282 | M₁H⁺ | |
| | m/z = 268 | MH⁺ | |
| | m/z = 178 | C₁₀H₁₆N₃⁺ | base peak |

Step 3: By working in a manner similar to that for the synthesis of [4-(3-chlorophenyl)piperazin-1-yl](5-methyl-2-phenyl-2 H-pyrazol-3-yl)methanone (Example 1), but replacing the 1-(3-chlorophenyl)piperazine with 0.5 g of phenylmethyl-[3-(piperazin-1-yl)phenyl]amine, 200 mg of [4-(3-phenylmethylaminophenyl)piperazin-1-yl](5-methyl-2-phenyl-2 H-pyrazol-3-yl)methanone hydrochloride are isolated in the form of an amorphous white solid, the characteristics of which are as follows:
Mass spectrum (EI): m/z=451 (M+)

### EXAMPLE E33

### [4-(5-Chloro-3-pyridyl)piperazin-1-yl](5-methyl-2-phenyl-2H-pyrazol-3-yl)methanone

Step 1: A suspension of 0.74 g of 3,5-dichloropyridine, 2.98 g of 1-tert-butoxycarbonylpiperazine, 7.33 g of cesium carbonate, 0.687 9 of tris(dibenzylideneacetone)dipalladium (0) and 1.96 g of 2-dicyclohexylphosphino-2′-(N,N-dimethylamino)biphenyl in 450 ml of 1,2-dimethoxyethane is maintained at 90° C. for 100 hours. The medium is concentrated under reduced pressure and then taken up in 50 ml of dichloromethane and filtered in the presence of Celite. After concentration under reduced pressure and purification by flash chromatography on a column of silica (60; 35-70 μm), eluting with a mixture of cyclohexane and ethyl acetate (gradient of from 100% to 50% of cyclohexane by volume), 715 mg of 1-tert-butoxycarbonyl4-(5-chloro-3-pyridyl)piperazine are obtained in the form of an oil, the characteristics of which are as follows:
¹H NMR spectrum (300 MHz, (CD₃)₂SO d6, δ in ppm): 1.43 (s: 9H); 3.26 (broad t, J=5 Hz: 4H); 3.47 (broad t, J=5 Hz: 4H); 7.45 (mt: 1H); 8.01 (d, J=2 Hz: 1H); 8.29 (d, J=2.5 Hz: 1H).

Step 2: A suspension of 680 mg of 1-tert-butoxycarbonyl-4-(5-chloro-3-pyridyl)piperazine in 1.8 ml of 5N hydrochloric acid solution is heated at 60° C. for 3 hours. The medium is concentrated under reduced pressure and then diluted with 20 ml of dichloromethane. After addition of 5 ml of normal sodium hydroxide solution, the organic phase is separated out by settling of the phases and then washed with water, dried over magnesium sulfate and concentrated under reduced pressure to give 450 mg of 1-(5-chloro-3-pyridyl)piperazine, which is used without further purification in the following step.

Step 3: By working in a manner similar to that for the synthesis of [4-(3-chlorophenyl)piperazin-1-yl](5-methyl-2-phenyl-2H-pyrazol-3-yl)methanone (Example1), but replacing the 1-(3-chlorophenyl)piperazine with 429 mg of 1-(5-chloro-3-pyridyl)piperazine, 286 mg of [4-(5-chloro-3-pyridyl)piperazin-1-yl](5-methyl-2-phenyl-2H-pyrazol-3-yl)methanone are obtained in the form of a solid, the characteristics of which are as follows:
IR spectrum (KBr): 2921; 2853; 1641; 1574; 1502; 1445; 1363; 1234; 1002; 996; 946; 763 and 693 cm⁻¹
¹H NMR spectrum (300 MHz, (CD₃)₂SO d6, δ in ppm): 2.31 (s: 3H); 3.01 (unresolved complex: 2H); 3.28 (unresolved complex: 2H); 3.40 (unresolved complex: 2H); 3.70 (unresolved complex: 2H); 6.54 (s: 1H); from 7.35 to 7.55 (mt: 4H); 7.37 (tt, J=7.5 and 1.5 Hz: 1H); 7.41 (mt: 1H); 8.02 (d J=2Hz: 1H); 8.23 (d, J=2.5Hz: 1H).

### EXAMPLE E34

### [4-(3-Methylaminophenyl)piperazin-1-yl](5-methyl-2-phenyl-2H-pyrazol-3-yl)-methanone

Step 1: 0.93 ml of triethylamine and 0.54 ml of methyl chloroformate are added, in the region of 0° C., to a solution of 1.6 g of 3-(4-phenylmethylpiperazin-1-yl)phenylamine, which may be obtained according to patent WO 02/090327, in 50 ml of dichloromethane. After 72 hours in the region of room temperature and addition of 50 ml of water, the organic phase is separated out by settling of the phases, washed with twice 50 ml of water and with 50 ml of saturated sodium chloride solution and then dried over magnesium sulfate and concentrated under reduced pressure. After purification by flash chromatography on a column of silica (60; 35-70 μm), eluting with a mixture of cyclohexane and ethyl acetate (70-30 by volume), 0.6 g of methyl [3-(4-phenylmethylpiperazin-1-yl)phenyl]carbamate is obtained in the form of an orange-colored oil, the characteristics of which are as follows:
Mass spectrum (EI): m/z=325(M+)

Step 2: By working in a manner similar to that of Step 2 of Example E34, but replacing the N-[3-(piperazin-1-yl)phenyl]benzamide with 1.5 g of [3-(4-phenylmethylpiperazin-1-yl)phenyl]carbamate, 1.3 g of [3-(4-phenylmethylpiperazin-1-yl)phenyl]methylamine are obtained in the form of an orange-colored oil, the characteristics of which are as follows:

| | | | |
|---|---|---|---|
| Mass spectrum IE | m/z = 281 | M^{+.} | base peak |
| | m/z = 266 | (M - CH₃)^{+.} | |
| | m/z = 190 | (M - C₇H₇)⁺ | |
| | m/z = 135 | C₈H₁₁N₂⁺ | |
| | m/z = 91 | C₇H₇⁺ | |

Step 3: 1.16 g of ammonium formate and 53 mg of 20% palladium-on-charcoal are added to a solution of 1.3 g of [3-(4-phenylmethylpiperazin-1-yl)phenyl]methylamine in 65 ml of methanol under an inert atmosphere. After refluxing for 4 hours, the catalyst is filtered off on Celite and the filtrate is concentrated under reduced pressure. 20 ml of water and 1 ml of 1 N sodium hydroxide are added, and the mixture is then extracted with 3 times 25 ml of ethyl acetate. The combined organic phases are washed with water, dried over magnesium sulfate and then concentrated under reduced pressure. After purification by flash chromatography on a column of silica (60; 35-70 μm), eluting with a mixture of dichloromethane and methanol (90-10 by volume), 0.8 g of methyl-[3-(piperazin-1-yl)phenyl]amine is obtained in the form of an orange-colored oil, the characteristics of which are as follows:

| | | | |
|---|---|---|---|
| Mass spectrum IE | m/z = 191 | M^{+.} | |
| | m/z = 149 | (M - C₂H₄N)^{+.} | base peak |

Step 4: By working in a manner similar to that for the synthesis of [4-(3-chlorophenyl)piperazin-1-yl](5-methyl-2-phenyl-2H-pyrazol-3-yl)methanone, but replacing the 1-(3-chlorophenyl)piperazine with 0.5 g of methyl[3-(piperazin-1-yl)phenyl])amine, 0.31 g of [4-(3-methylaminophenyl)piperazin-1-yl](5-methyl-2-phenyl-2H-pyrazol-3-yl)methanone is obtained in the form of a beige-colored foam, the characteristics of which are as follows: Mass spectrum (EI): m/z=375(M+)

### EXAMPLE E35

### Methyl 3-hydroxy-2-{3-[4-(5-methyl-2-phenyl-2H-pyrazole-3-carbonyl)piperazin-1-yl]benzoylamino)propionate

Step 1: 763 mg of potassium hydroxide pellets are added to a solution of 4.4 g of ethyl 3-[4-(5-methyl-2-phenyl-2H-pyrazole-3-carbonyl)piperazin-1-yl]-benzoate, described in Example E28, in 75 ml of distilled water and 150 ml of methanol. After 20 hours at room temperature, the reaction mixture is concentrated under reduced pressure and the residue is acidified with 5N hydrochloric acid to pH 5. After filtration of the solid formed, 3.9 g of 3-[4-(5-methyl-2-phenyl-2H-pyrazole-3-carbonyl)piperazin-1-yl]benzoic acid are thus obtained in the form of a pale yellow solid, the characteristics of which are as follows:
Melting point (Kofler): 206° C.
¹H NMR spectrum (300 MHz, (CD₃)₂SO d6, δ in ppm): 2.30 (s: 3H); 2.86 (unresolved complex: 2H); 3.15 (unresolved complex: 2H); 3.38 (unresolved complex: 2H); 3.71 (unresolved complex: 2H); 6.54 (s: 1H); 7.14 (broad d, J=8.5 Hz: 1H); from 7.25 to 7.55 (mt: 8H); from 12.60 to 13.20 (very broad unresolved complex: 1H).

Step 2: 316 mg of 1-(3-dimethylaminopropyl)-3-ethylcarbodiimide hydrochloride (EDCl) and 223 mg of 1-hydroxybenzotriazole hydrate (HOBT) are added to a solution of 586 mg of 3-[4-(5-methyl-2-phenyl-2H-pyrazole-3-carbonyl)piperazin-1-yl]benzoic acid in 25 ml of dichloromethane. After stirring for 10 minutes at room temperature, 211 μl of triethylamine (TEA) and 233 mg of methyl D,L-2-amino-3-hydroxypropionate hydrochloride are added and this reaction mixture is then stirred for 20 hours at room temperature. After addition of 50 ml of dichloromethane and 50 ml of water, the organic phase is separated out by settling of the phases and then washed with water, dried over magnesium sulfate and concentrated under reduced pressure. After purification by flash chromatography on a column of silica (60; 35-70 μm), eluting with pure ethyl acetate, 520 mg of methyl 3-hydroxy-2-{3-[4-(5-methyl-2-phenyl-2H-pyrazole-3-carbonyl)piperazin-1-yl]benzoylamino)propionate are obtained in the form of a beige-colored foam, the characteristics of which are as follows:
Mass spectrum (EI): m/z=491 (M⁺)
¹H NMR spectrum (300 MHz, (CD₃)₂SO d6, δ in ppm): 2.31 (s: 3H); 2.90 (unresolved complex: 2H); 3.19 (unresolved complex: 2H); 3.40 (unresolved complex: 2H); 3.67 (s: 3H); 3.73 (unresolved complex: 2H); 3.80 (broad t, J=5.5 Hz: 2H); 4.55 (mt: 1H); 5.06 (very broad t, J=5.5 Hz: 1H); 6.54 (s: 1H); 7.08 (mt: 1H); from 7.25 to 7.55 (mt: 8H); 8.51 (d, J=7.5 Hz: 1H).

### EXAMPLE E36

### [4-(3-Chlorophenyl)piperazin-1-yl](5-methyl-2-pyrazin-2-yl-2H-pyrazol-3-yl)-methanone

The process is performed in a manner similar to that of Step 2 of Example E22, starting with 150 mg of product from Step 1 of Example E22 and 85 mg and then 39 mg of 2-chloropyrazine, to give, after purification by flash chromatography on a column of silica (30-60 μm), eluting with a mixture of dichloromethane, methanol and aqueous ammonia (98/2/0.1 by volume), 4 mg of [4-(3-chlorophenyl)piperazin-1-yl](5-methyl-2-pyrazin-2-yl-2 H-pyrazol-3-yl)methanone in the form of a yellow resin, the characteristics of which are as follows:
LC/MS analysis: tr=3.90; m/z=383 (MH⁺)
1H NMR spectrum (400 MHz, CDCl₃, δ in ppm): 2.40 ppm (s, 3H); 3.11 ppm (m, 2H); 3.21 ppm (m, 2H); 3.50 ppm (m, 2H); 3.97 ppm (m, 2H); 6.32 ppm (s, 1H); 6.80 ppm (bd, J=8.5 Hz, 1H); 6.89 ppm (m, 2H); 7.19 ppm (t, J=8.5Hz, 1H); 8.23 ppm (bs, 1H); 8.44 ppm (d, J=2.5 Hz, 1H); 9.24 ppm (bs, 1H)

### EXAMPLE E37

### [4-(3-Chlorophenyl)piperazin-1-yl](5-methyl-2-thiazol-2-yl-2H-pyrazol-3-yl)-methanone

The process is performed in a manner similar to that of Step 2 of Example E22, starting with 150 mg of the product from Step 1 of Example E22 and 121 mg and then 56 mg of 2-bromothiazole, to give, after purification by flash chromatography on a column of silica (30-60 μm), eluting with a mixture of toluene, ethyl acetate and triethylamine (80/20/0.1 by volume), 5 mg of [4-(3-chlorophenyl)piperazin-1-yl](5-methyl-2-thiazol-2-yl-2H-pyrazol-3-yl)methanone in the form of a yellow oil, the characteristics of which are as follows:
LC/MS analysis: tr=4.16; m/z=388 (MH⁺)
¹H NMR spectrum (400 MHz, CDCl₃, δ in ppm): 2.36 ppm (s, 3H); 3.08 ppm (m, 2H); 3.32 ppm (m, 2H); 3.43 ppm (m, 2H); 3.97 ppm (m, 2H); 6.30 ppm (s, 1H); 6.78 ppm (bd, J=8.5 Hz, 1H); 6.87 ppm (m, 2H); 7.06 ppm (d, J=3.5 Hz, 1H); 7.18 ppm (t, J=8.5 Hz, 1H); 7.45 ppm (d, J=3.5 Hz, 1H)

### EXAMPLE E38

### 2-{5-[4-(3-Chlorophenyl)piperazine-1-carbonyl]-3-methylpyrazol-1-yl}nicotinonitrile

The process is performed in a manner similar to that of Step 2 of Example E22, starting with 150 mg of the product from Step 1 of Example E22 and 68 mg and then 32 mg of 2-chloro-3-cyanopyridine, to give, after purification by flash chromatography on a column of silica (30-60 μm), eluting with a mixture of toluene, ethyl acetate and triethylamine (80/20/0.1 by volume), 7 mg of 2-{5-[4-(3-chlorophenyl)piperazine-1-carbonyl]-3-methylpyrazol-1-yl}-nicotinonitrile in the form of a white solid, the characteristics of which are as follows:
LC/MS analysis: tr=4.03; m/z=408 (MH⁺)
¹H NMR spectrum (400 MHz, CDCl₃, δ in ppm): 2.43 ppm (s, 3H); 3.19 ppm (m, 2H); 3.29 ppm (m, 2H); 3.66 ppm (m, 2H); 3.91 ppm (m, 2H); 6.38 (s, 1H); 6.82 ppm (bd, J=8.5Hz, 1H); 6.89 ppm (m, 2H); 7.21 ppm (t, J=8.5 Hz, 1H); 7.31 ppm (dd, J=5-7.5 Hz, 1H); 8.13 ppm (dd, J=2-7.5 Hz, 1H); 8.52 ppm (dd, J=2-5 Hz, 1H).

### EXAMPLE E39

### {4-[3-(1-Hydroxyethyl)phenyl]piperazin-1-yl}(5-methyl-2-phenyl-2H-pyrazol-3-yl)methanone hydrochloride

Step 1: 2.99 g of 3-bromoacetophenone, 317 mg of (R)(+)-2,2′-bis(diphenyl-phosphino)-1,1′-binaphthyl, 114 mg of palladium acetate and 1.59 g of sodium tert-butoxide are added to a solution of 2.78 g of N-phenylmethylpiperazine in 100 ml of toluene. After heating at 80° C. for 20 hours, the insoluble material is filtered off, 25 ml of ethyl acetate and 25 ml of water are added and the organic phase is separated out by settling of the phases and then washed with water, dried over magnesium sulfate and concentrated under reduced pressure. After purification by flash chromatography on a column of silica (60; 35-70 μm), eluting with a mixture of cyclohexane and ethyl acetate (20-70 by volume), 0.4 g of 1-[3-(4-phenylmethylpiperazin-1-yl)phenyl]ethanone is obtained in the form of an orange-colored oil, the characteristics of which are as follows:
Mass spectrum (EI): m/z=294(M+)

Step 2: By working in a manner similar to that of Step 3 of Example 34, but replacing the [3-(4-phenylmethylpiperazin-1-yl)phenyl]methylamine with 0.7 g of 1-[3-(4-phenylmethylpiperazin-1-yl)phenyl]ethanone, 0.3 g of 1-[3-(piperazin-1-yl)phenyl]ethanol is obtained in the form of an orange-colored oil, the characteristics of which are as follows:
Mass spectrum (EI): m/z=294(M+)

Step 3: By working in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 0.3 g of 1-(3-(piperazin-1-yl)phenyl]ethanol, 0.15 g of {4-[3-(1-hydroxyethyl)phenyl]-piperazin-1-yl}-(5-methyl-2-phenyl-2H-pyrazol-3-yl)methanone hydrochloride is isolated in the form of an amorphous off-white solid, the characteristics of which are as follows:
Mass spectrum (EI): m/z=390(M+)

### EXAMPLE E40

### N-(2-Hydroxyethyl)-3-[4-(5-methyl-2-phenyl-2H-pyrazole-3-carbonyl)piperazin-1-yl]benzamide

Working in a manner similar to that for the synthesis of methyl 3-hydroxy-2-{3-[4-(5-methyl-2-phenyl-2H-pyrazole-3-carbonyl)piperazin-1-yl]benzoylamino}-propionate of Step 2 of Example E35, but replacing the D,L-2-amino-3-hydroxypropionic acid with 62 μl of ethanolamine, 0.36 g of N-(2-hydroxy-ethyl)-3-[4-(5-methyl-2-phenyl-2H-pyrazole-3-carbonyl)piperazin-1-yl]benzamide is obtained in the form of a beige-colored foam, the characteristics of which are as follows:
Mass spectrum (EI): m/z=433(M+)
¹H NMR spectrum (300 MHz, (CD₃)₂SO d6, δ in ppm): 2.31 (s: 3H); 2.87 (unresolved complex: 2H); 3.17 (unresolved complex: 2H); 3.33 (mt: 2H); 3.39 (unresolved complex: 2H); 3.52 (mt: 2H); 3.73 (unresolved complex: 2H); 4.73 (t, J=5.5 Hz: 1H); 6.54 (s: 1H); 7.04 (mt: 1H); from 7.20 to 7.55 (mt: 8H); 8.37 (broad t, J=5.5 Hz: 1H).

### EXAMPLE E41

### [4-(Isoquinolin-4-yl)piperazin-1-yl)](5-methyl-2-phenyl-2H-pyrazol-3-yl)-methanone

By working in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 213.3 mg of 4-piperazin-1-ylisoquinoline, which may be obtained according to patent DE 19900544, 320 mg of [4-(isoquinolin-4-yl)piperazin-1-y])(5-methyl-2-phenyl-2H-pyrazol-3-yl)methanone are obtained in the form of a white solid, the characteristics of which are as follows:
Melting point (Kofler): 166° C.
Mass spectrum (EI): m/z=397(M+)

### EXAMPLE E42

### [4-(3-Chlorophenyl)piperazin-1-yl][2-(2,4-difluorophenyl)-5-methyl-2H-pyrazol-3-yl]methanone

Step 1: 3.35 g of methyl hydroxylamine hydrochloride are added to a solution of 5.28 g of ethyl 2,4-dioxovalerate in 35 ml of DMF and 35 ml of ethanol, followed by addition of 9.99 g of sodium acetate trihydrate. After stirring for 2 hours at 60° C., the reaction mixture is filtered and the filtrate concentrated. The oil obtained is taken up in isopropyl ether and the organic phase is washed with saturated aqueous sodium dihydrogen phosphate solution, dried over magnesium sulfate and concentrated under reduced pressure. After purification by flash chromatography on a column of silica (40-63 μm), eluting, with a mixture of heptane and ethyl acetate (40/60 by volume), 2.13 g of ethyl 2,4-dioximinovalerate A are obtained in the form of a colorless liquid, the characteristics of which are as follows:
Mass spectrum (EI): m/z=216 (M⁺)

Step 2: 5.6 ml of a 2M solution of trimethylaluminum in toluene are added, at 25° C., to a solution of 1.65 g of 1-(3-chlorophenyl)piperazine in 45 ml of toluene, followed by addition, at 60° C., of a solution of 1.21 g of dioxime A (Step 1 of the present example) in 10 ml of toluene. The reaction medium is stirred for 1 hour at 75° C. and then poured into 100 ml of aqueous 1M sodium potassium tartrate solution and extracted with ethyl acetate. The organic phase is dried over magnesium sulfate and concentrated under reduced pressure. After purification by flash chromatography on a column of silica (40-63 μm), eluting with a mixture of heptane and ethyl acetate (80/20 by volume), 1.15g of N-[4-(3-chlorophenyl)piperazinyl]-2,4-dioximinovaleramide B are obtained in the form of a yellow oil, the characteristics of which are as follows:
Mass spectrum (ES): m/z=367 (MH⁺)

Step 3: 108 mg of 2,4-difluorophenylhydrazine hydrochloride are added, at 25° C., to a solution of 110 mg of amide B in 0.6 ml of acetic acid and 0.3 ml of methylglycol. The reaction medium is stirred for 3.5 hours at 100° C. and then concentrated. After purification by flash chromatography on a column of silica (40-63 μm), eluting with a mixture of heptane and ethyl acetate (70/30 by volume), 101 mg of [4-(3-chlorophenyl)piperazin-1-yl][2-(2,4-difluorophenyl)-5-methyl-2H-pyrazol-3-yl]methanone are obtained in the form of a pale yellow solid, the characteristics of which are as follows:
LC/MS analysis: tr=4.34; m/z=417 (MH⁺)
¹H NMR spectrum (400 MHz, (CD₃)₂SO d6, δ in ppm): 2.29 ppm (s, 3H); 3.18 ppm ( m, 4H); 3.66 ppm (m, 4H); 6.60 ppm (s, 1H); 6.82 (dd, J=2-8.5 Hz, 1H); 6.91 ppm (dd, J=2-8.5 Hz, 1H); 6.97 ppm (t, J=2 Hz, 1H); 7.22 ppm (m, 2H); 7.45 ppm (ddd, J=2.5-9-11 Hz, 1H); 7.61 ppm (dt, 6-9Hz, 1H).

### EXAMPLE E43

### [4-(3-Chlorophenyl)piperazin-1-yl][5-methyl-2-(2,3,5,6-tetrafluorophenyl)-2H-pyrazol-3-yl]methanone

The process is performed in a manner similar to that of Example E42, starting with 75 mg of amide B, obtained from Step 2 of Example E42, and 109 mg of 2,3,5,6-tetrafluorophenylhydrazine in the presence of 76 mg of paratoluenesulfonic acid monohydrate, for 9 hours at 100° C., to give, after purification by flash chromatography on a column of silica (40-63 μm), eluting with a mixture of heptane and ethyl acetate (80/20 by volume), 14 mg of [4-(3-chlorophenyl)piperazin-1-yl][5-methyl-2-(2,3,5,6-tetrafluorophenyl)-2H-pyrazol-3-yl]methanone in the form of a yellow solid, the characteristics of which are as follows:
LC/MS analysis: tr=4.51; m/z=453 (MH⁺)
¹H NMR spectrum (400 MHz, (CD₃)₂SO d6, δ in ppm): 2.32 ppm (s, 3H); 3.22 ppm (m, 4H); 3.66 ppm (m, 2H); 3.77 ppm (m, 2H); 6.79 ppm (s, 1H); 6.83 ppm (dd, 2.5-8.5 Hz, 1H); 6.92 ppm (dd, J=2.5-8.5 Hz, 1H); 6.98 ppm (t, J=2.5 Hz, 1H); 7.24 ppm (t, J=8.5 Hz, 1H); 8.10 ppm (m, 1H)

### EXAMPLE E44

### [4-(3-Chlorophenyl)piperazin-1-yl][2-(2,5-dichlorophenyl)-5-methyl-2H-pyrazol-3-yl]methanone

184 mg of 2,5-dichlorophenylhydrazine and 197 mg of para-toluenesulfonic acid monohydrate are added, at 25° C., to a solution of 190 mg of amide B, obtained in Step 2 of Example E42, in 1.5 ml of acetic acid. The reaction medium is stirred for 1.5 hours at 100° C. and then concentrated. After purification by flash chromatography on a column of silica (60; 40-63 μm), eluting with a mixture of heptane and ethyl acetate (80/20 by volume), 84 mg of [4-(3-chlorophenyl)piperazin-1-yl][2-(2,5-dichlorophenyl)-5-methyl-2H-pyrazol-3-yl]methanone are obtained in the form of a yellow solid, the characteristics of which are as follows:
LC/MS analysis: tr=4.62; m/z=449 (MH⁺)
¹H NMR spectrum (400 MHz, (CD₃)₂SO d6, δ in ppm): 2.29 ppm (s, 3H); 3.20 ppm (m, 4H); 3.65 ppm (m, 2H); 3.76 ppm (m, 2H); 6.64 ppm (s, 1H); 6.82 ppm (dd, J=2.5-8.5 Hz, 1H); 6.91 ppm (dd, J=2.5-8.5 Hz, 1H); 6.97 ppm (t, J=2.5 Hz, 1H); 7.23 ppm (t, J=8.5 Hz, 1H); 7.56 ppm (dd, 2.5-8.5 Hz, 1H); 7.62 ppm (d, J=2.5 Hz, 1H); 7.63 ppm (d, J=8.5 Hz).

### EXAMPLE E45

### [4-(3-Chlorophenyl)piperazin-1-yl](5-methyl-2-o-tolyl-2H-pyrazol-3-yl)methanone

55 mg of ortho-tolylhydrazine hydrochloride are added at 25° C. to a solution of 106 mg of amide B, obtained in Step 2 of Example E42 in 1.0 ml of acetic acid. The reaction medium is stirred for 2 hours at 100° C. and then concentrated. After purification by flash chromatography on a column of silica (30-60 μm), eluting with a mixture of heptane and ethyl acetate (80/20 by volume), 22 mg of [4-(3-chlorophenyl)piperazin-1-yl](5-methyl-2-o-tolyl-2H-pyrazol-3-yl)methanone are obtained in the form of a white solid, the characteristics of which are as follows:
LC/MS analysis: tr=4.31; m/z=495 (MH⁺)

### EXAMPLE E46

### (1-Phenyl-1H-pyrrol-2-yl)[4-(pyrid-3-yl)piperazin-1-yl]methanone hydrochloride

The process is performed in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 65.4 mg of 1-(pyrid-3-yl)piperazine, which may be obtained according to Chem. Pharm. Bull., 49, 1314 (2001), and replacing the 5-methyl-2-phenyl-2H-pyrazole-3-carboxylic acid with 75 mg of 1-phenyl-1H-pyrrole-2-carboxylic acid, which may be obtained according to Synth. Commun., 28, 443 (1998). 77 mg of (1-phenyl-1H-pyrrol-2-yl)[(4-(pyrid-3-yl)piperazin-1-yl]methanone hydrochloride are thus isolated in the form of a yellow solid, the characteristics of which are as follows:
Melting point (Kofler): 180° C.
Mass spectrum (EI): m/z=332 (M+)

### EXAMPLE E47

### [4-(3-Chlorophenyl)piperazin-1-yl][2-(2,5-dimethylphenyl)-5-methyl-2H-pyrazol-3-yl]methanone

The process is performed in a manner similar to that of Example E45, starting with 106 mg of amide B, obtained in Step 2 of Example E42, and 60 mg of 2,5-dimethylphenylhydrazine hydrochloride, to give, after purification by flash chromatography on a column of silica (30-60 μm), eluting with a mixture of heptane and ethyl acetate (80/20 by volume), 46 mg of [4-(3-chlorophenyl)piperazin-1-yl][2-(2,5-dimethylphenyl)-5-methyl-2H-pyrazol-3-yl]methanone in the form of a white solid, the characteristics of which are as follows:
LC/MS analysis: tr=4.48; m/z=409 (MH⁺)

### EXAMPLE E48

### [4-(3-Chlorophenyl)piperazin-1-yl](2-cyclohexyl-5-methyl-2H-pyrazol-3-yl)-methanone

The process is performed in a manner similar to that of Example E45, starting with 106 mg of amide B, obtained in Step 2 of Example E42, and 74 mg of cyclohexylhydrazine hydrochloride, for 6 hours at 100° C., to give, after purification by flash chromatography on a column of silica (30-60 μm), eluting with a mixture of heptane and ethyl acetate (80/20 by volume), 70 mg of [4-(3-chlorophenyl)piperazin-1-yl](2-cyclohexyl-5-methyl-2H-pyrazol-3-yl)methanone in the form of a white solid, the characteristics of which are as follows:
LC/MS analysis: tr=4.51; m/z=387 (MH⁺)

### EXAMPLE E49

### [4-(3-Chlorophenyl)piperazin-1-yl][5-methyl-2-(4-nitrophenyl)-2H-pyrazol-3-yl]-methanone

The process is performed in a manner similar to that of Example E45, starting with 165 mg of amide B, obtained in Step 2 of Example 42, and 105 mg of 4-nitrophenylhydrazine in the presence of 104 mg of para-toluenesulfonic acid monohydrate, for 4 hours at 100° C., to give, after purification by flash chromatography on a column of silica (40-63 μm), eluting with a mixture of heptane and ethyl acetate (80/20 by volume), 148 mg of [4-(3-chlorophenyl)piperazin-1-yl][5-methyl-2-(4-nitrophenyl)-2H-pyrazol-3-yl]methanone in the form of a yellow solid, the characteristics of which are as follows:
LC/MS analysis: tr=4.36; m/z=426 (MH⁺)
¹H NMR spectrum (400 MHz, (CD₃)₂SO d6, δ in ppm): 2.31 ppm (s, 3H); 3.11 ppm (m, 2H); 3.28 ppm (masked, 2Hz); 3.53 ppm (m, 2H); 3.74 ppm (m, 2H); 6.66 ppm (s, 1H); 6.81 ppm (dd, J=2.5-8.5 Hz, 1H); 6.88 ppm (dd, J=2.5-8.5 Hz, 1H); 6.94 ppm (t, J=2.5 Hz, 1H); 7.22 ppm (t, J=8.5 Hz, 1H); 7.70 and 8.33 ppm (AA′BB′ system, 4H).

### EXAMPLE E50

### [4-(3-Chlorophenyl)piperazin-1-yl][5-methyl-2-(4-trifluoromethylphenyl)-2H-pyrazol-3-yl]methanone

The process is performed in a manner similar to that of Example E45, starting with 165 mg of amide B, obtained in Step 2 of Example E42, and 120 mg of 4-(trifluoromethyl)phenylhydrazine in the presence of 104 mg of paratoluenesulfonic acid monohydrate, for 5 hours at 100° C., to give, after purification by flash chromatography on a column of silica (40-63 μm), eluting with a mixture of heptane and ethyl acetate (80/20 by volume), 98 mg of [4-(3-chlorophenyl)piperazin-1-yl][5-methyl-2-(4-trifluoromethylphenyl)-2H-pyrazol-3-yl]methanone in the form of a white solid, the characteristics of which are as follows:
LC/MS analysis: tr=4.61; m/z=449 (MH⁺)
¹H NMR spectrum (400 MHz, (CD₃)₂SO d6, δ in ppm): 2.31 ppm (s, 3H); 3.01 ppm (m, 2H); 3.23 ppm (m, 2H); 3.48 ppm (m, 2H); 3.72 ppm (m, 2H); 6.61 ppm (s, 1H); 6.82 ppm (dd, J=2.5-8.5 Hz, 1H); 6.87 ppm (dd, J=2.5-8.5 Hz, 1H); 6.93 ppm (t, J=2.5 Hz, 1H); 7.22 ppm (t, J=8.5 Hz, 1H); 7.66 and 7.85 ppm (AA′BB′ system, 4H).

### EXAMPLE E51

### [4-(3,5-Dimethoxyphenyl)piperazin-1-yl](1-phenyl-1H-pyrrol-2-yl)methanone hydrochloride

By working in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 89 mg of 1-(3,5-dimethoxyphenyl)piperazine and replacing the 5-methyl-2-phenyl-2H-pyrazole-3-carboxylic acid with 75 mg of 1-phenyl-1H-pyrrole-2-carboxylic, acid, which may be obtained according to Synth. Comm., 28, 443 (1998), 30 mg of [4-(3,5-dimethoxyphenyl)piperazin-1-yl](1-phenyl-1H-pyrrol-2-yl)methanone hydrochloride are isolated in the form of an amorphous white solid, the characteristics of which are as follows:
Mass spectrum (EI): m/z=391 (M+)

### EXAMPLE E52

### [4-(3-Chlorophenyl)piperazin-1-yl][5-methyl-2-(pyrid-3-yl)-2H-pyrazol-3-yl]methanone

By working in a manner similar to that for the synthesis of [4-(3-chlorophenyl)piperazin-1-yl](5-methyl-2-phenyl-2H-pyrazol-3-yl)methanone (Example 1), but replacing the 5-methyl-2-phenyl-2H-pyrazole-3-carboxylic acid with 203 mg of 5-methyl-2-(pyrid-3-yl)-2H-pyrazole-3-carboxylic acid, which may be obtained according to J. Het. Chem., 36, 217 (1999), 290 mg of [4-(3-chlorophenyl)piperazin-1-yl][5-methyl-2-(pyrid-3-yl)-2H-pyrazol-3-yl]methanone are obtained in the form of a beige-colored solid, the characteristics of which are as follows:
Melting point (Kofler): 124° C.
Mass spectrum (EI): m/z=381 (M+)

### EXAMPLE E53

### [4-(3,5-Dimethoxyphenyl)piperazin-1-yl][5-methyl-2-(pyrid-3-yl)-2H-pyrazol-3-yl]methanone

By working in a manner similar to that for the synthesis of [4-(3-chlorophenyl)piperazin-1-yl][5-methyl-2-(pyrid-3-yl)-2H-pyrazol-3-yl]methanone (Example 1), but replacing the 1-(3-chlorophenyl)piperazine with 602 mg of 1-(3,5-dimethoxyphenyl)piperazine, and 5-methyl-2-phenyl-2H-pyrazole-3-carboxylic acid with 600 mg of 5-methyl-2-pyrid-3-yl-2H-pyrazole-3-carboxylic acid, which may be obtained according to J. Het. Chem., 36, 217 (1999), 800 mg of [4-(3,5-dimethoxyphenyl)piperazin-1-yl][5-methyl-2-(pyrid-3-yl)-2H-pyrazol-3-yl]methanone are obtained in the form of a white solid, the characteristics of which are as follows:
Melting point (Kofler): 60° C.
Mass spectrum (EI): m/z=407 (M+)

### EXAMPLE E54

### [4-(4-Fluoro-3-pyridyl)piperazin-1-yl](5-methyl-2-phenyl-2H-pyrazol-3-yl)methanone

By working, in 3 steps, in a manner similar to that for the synthesis of [4-(5-chloro-3-pyridyl)piperazin-1-yl](5-methyl-2-phenyl-2H-pyrazol-3-yl)methanone (Example 33), but replacing in Step 1 of the synthesis the 3,5-dichloropyridine with 3.52 g of 5-bromo-2-fluoropyridine, 433 mg of [4-(4-fluoro-3-pyridyl)-piperazin-1-yl](5-methyl-2-phenyl-2H-pyrazol-3-yl)methanone are obtained, in Step 3 of the synthesis, in the form of a solid, the characteristics of which are as follows:
¹H NMR spectrum (300 MHz, (CD₃)₂SO d6, in ppm): 2.31 (s: 3H); 2.85 (unresolved complex: 2H); 3.13 (unresolved complex: 2H); 3.39 (unresolved complex: 2H); 3.72 (unresolved complex: 2H); 6.54 (s: 1H); 7.05 (dd, J=9 and 3 Hz: 1H); 7.37 (tt, J=7 and 1.5 Hz: 1H); from 7.35 to 7.60 (mt: 5H); 7.79 (dd, J=3 and 1.5 Hz: 1H).

### EXAMPLE E55

### 3-{5-[4-(3-Chlorophenyl)piperazine-1-carbonyl]-3-trifluoromethylpyrazol-1-yl}benzonitrile

By working in a manner similar to that for the synthesis of [4-(3-chloro-phenyl)piperazin-1-yl](5-methyl-2-phenyl-2H-pyrazol-3-yl)methanone (Example 1), but replacing the 5-methyl-2-phenyl-2H-pyrazole-3-carboxylic acid with 100 mg of 2-(3-cyanophenyl)-5-trifluoromethyl-2H-pyrazole-3-carboxylic acid, which may be obtained according to patent WO 02/000647, 72 mg of 3-{5-[4-(3-chlorophenyl)piperazine-1-carbonyl]-3-trifluoromethylpyrazol-1-yl}benzonitrile are obtained in the form of a white foam, the characteristics of which are as follows:
Mass spectrum (EI): m/z=459 (M+)

### EXAMPLE E56

### 3-{5-[4-(3,5-Dimethoxyphenyl)piperazine-1-carbonyl]-3-trifluoromethylpyrazol-1-yl}benzonitrile

By working in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 79 mg of 1-(3,5-dimethoxyphenyl)piperazine and replacing the 5-methyl-2-phenyl-2H-pyrazole-3-carboxylic acid with 100 mg of 2-(3-cyanophenyl)-5-trifluoromethyl-2H-pyrazole-3-carboxylic acid, which may be obtained according to patent WO 02/000647, 118 mg of 3-{5-[4-(3,5-dimethoxyphenyl)piperazine-1-carbonyl]-3-trifluoromethylpyrazol-1-yl}benzonitrile are obtained in the form of a white foam, the characteristics of which are as follows:
Mass spectrum (EI): m/z=485 (M+)

### EXAMPLE E57

### 3-[4-(1-Phenyl-1H-pyrrole-2-carbonyl)piperazin-1-yl]benzamide

By working in a manner similar to that for the synthesis of [4-(3-chlorophenyl)piperazin-1-yl](5-methyl-2-phenyl-2H-pyrazol-3-yl)methanone (Example1), but replacing the 1-(3-chlorophenyl)piperazine with 113.2 mg of 3-piperazin-1-ylbenzamide, which may be obtained according to patent WO 98/00400, and replacing the 5-methyl-2-phenyl-2H-pyrazole-3-carboxylic acid with 75 mg of 1-phenyl-1H-pyrrole-2-carboxylic acid, which may be obtained according to Synth. Comm., 28, 443 (1998), 65 mg of 3-[4-(1-phenyl-1H-pyrrole-2-carbonyl)piperazin-1-yl]benzamide are obtained in the form of an amorphous beige-colored solid, the characteristics of which are as follows:
Mass spectrum (EI): m/z=374 (M+)

### EXAMPLE E58

### [2-(3-Fluorophenyl)-5-methyl-2H-pyrazol-3-yl](4-pyrid-3-ylpiperazin-1-yl)methanone

By working in a manner similar to that for the synthesis of [4-(3-chlorophenyl)piperazin-1-yl](5-methyl-2-phenyl-2H-pyrazol-3-yl)methanone, but replacing the 1-(3-chlorophenyl)piperazine with 214 mg of 1-pyrid-3-ylpiperazine, which may be obtained according to Chemical and Pharmaceutical Bulletin, 49, 1314 (2001) and replacing the 5-methyl-2-phenyl-2H-pyrazole-3-carboxylic acid with 288.7 mg of 2-(3-fluorophenyl)-5-methyl-2H-pyrazole-3-carboxylic acid, which may be obtained according to J. Het Chem., 30, 304 (1993), 280 mg of [2-(3-fluorophenyl)-5-methyl-2H-pyrazol-3-yl](4-pyrid-3-ylpiperazin-1-yl)methanone are obtained in the form of a white powder, the characteristics of which are as follows:
Melting point (Kofler): 132° C.
Mass spectrum (EI): m/z=365 (M+)

### EXAMPLE E59

### [4-(4-Bromo-3-chlorophenyl)piperazin-1-yl](5-methyl-2-phenyl-2H-pyrazol-3-yl)methanone

140 mg of N-bromosuccinimide and 6.5 mg of 2,2′-azobis(2-methylpropionitrile) are added to a solution of 300 mg of [4-(3-chlorophenyl)piperazin-1-yl](5-methyl-2-phenyl-2H-pyrazol-3-yl)methanone (Example 1) in 6 ml of carbon tetrachloride. The reaction mixture is refluxed under irradiation from a 250 W lamp (white light) for 3 hours and then filtered and concentrated under reduced pressure. After purification by flash chromatography on a column of silica (40-63 μm), eluting with a mixture of toluene and ethyl acetate (80/20 by volume), 350 mg of [4-(4-bromo-3-chlorophenyl)piperazin-1-yl](5-methyl-2-phenyl-2H-pyrazol-3-yl)methanone are obtained in the form of a pale yellow oil, the characteristics of which are as follows:
LC/MS analysis: tr=4.49; m/z=458 (MH⁺)
¹H NMR spectrum (400 MHz, (CD₃)₂SO d6, δ in ppm): 2.29 ppm (s, 3H); 2.89 ppm (m, 2H); 3.17 ppm (m, 2H); 3.34 ppm (m, 2H); 3.66 ppm (m, 2H); 6.51 ppm (s, 1H); 6.80 ppm (dl J=8.5 Hz, 1H); 7.09 ppm (bs, 1H); 7.35 ppm (bt, J=8 Hz, 1H); from 7.41 to 7.52 ppm (m, 5H).

### EXAMPLE E60

### (5-Hydroxymethyl-2-phenyl-2H-pyrazol-3-yl)[4-(3-chlorophenyl)piperazin-1l-yl]methanone

The process is performed in a manner similar to that of Step 3 of Example E73, starting with 0.3 g of ethyl 3-benzoyloxymethyl-1-phenyl-1-H-pyrazole-5-carboxylate, obtained in Step 2 of Example 73, and 537 mg of 1-(3-chlorophenyl)piperazine to give, after purification by flash chromatography on a column of silica (40-63 μm), eluting with a mixture of dichloromethane and ethyl acetate (80/20 by volume), 253 mg of (5-hydroxymethyl-2-phenyl-2H-pyrazol-3-yl)[4-(3-chlorophenyl)piperazin-1-yl]methanone in the form of a white solid, the characteristics of which are as follows:
Mass spectrum(ES): m/z=422 (MH⁺)

### EXAMPLE E61

### (5-Benzyloxy-2-pyrid-2-yl-2H-pyrazol-3-yl)[4-(3-chlorophenyl)piperazin-1-yl]methanone

Step 1: A solution of 4.58 g of N-tert-butoxycarbonyl anhydride (Boc₂O) in 40 ml of dichloromethane is added to a solution of 3.12 g of ethyl 5-hydroxy-1H-pyrazole-3-carboxylate, which may be prepared according to Chem. Pharm. Bull. 31(4) 1228 (1983), using toluene instead of benzene, in 40 ml of dichloromethane and 3.1 ml of triethylamine, cooled to 0° C. The reaction medium is stirred for 3 hours at room temperature and then washed with saturated aqueous sodium dihydrogen phosphate solution. The organic phase is dried over magnesium sulfate and concentrated under reduced pressure. After purification by flash chromatography on a column of silica (40-63 μm), eluting with a mixture of cyclohexane and ethyl acetate (80/20 by volume), 3.26 g of 1-tert-butyl 3-ethyl 5-hydroxypyrazole-1,3-dicarboxylate are obtained in the form of a white solid, the characteristics of which are as follows:
Mass spectrum (ES): m/z=257 (MH⁺)

Step 2: A solution of 0.37 ml of benzyl bromide in 3 ml of DMF is added to 796 mg of the product of Step 1 of the present Example and 1.11 g of cesium carbonate in 15 ml of DMF at −5° C. The reaction medium is stirred for 3 hours at 0° C. and then poured into saturated aqueous sodium dihydrogen phosphate solution and extracted with ethyl acetate. The organic phase is dried over magnesium sulfate and concentrated under reduced pressure. After purification by flash chromatography on a column of silica (40-63 μm), eluting with a mixture of heptane and ethyl acetate (90/10 by volume), 117 mg of 1-tert-butyl 3-ethyl 5-benzyloxypyrazole-1,3-dicarboxylate are obtained in the form of a white solid, the characteristics of which are as follows:
Mass spectrum (ES): m/z=347 (MH⁺)

Step 3: 3 ml of trifluoroacetic acid are added to 1.50 g of the product of Step 2 of the present Example in 12 ml of dichloromethane at room temperature. The reaction medium is stirred for 1 hour at room temperature, concentrated under reduced pressure and then purified by flash chromatography on a column of silica (40-63 μm), eluting with a mixture of dichloromethane and ethyl acetate (97/3 by volume), to give 340 mg of ethyl 5-benzyloxy-2H-pyrazole-3-carboxylate in the form of a white solid, the characteristics of which are as follows:
Mass spectrum (ES): m/z=247 (MH⁺)

Step 4: The process is performed in a manner similar to that of Step 2 of Example E42, starting with 320 mg of the product of Step 3 of the present example and 537 mg of 1-(3-chlorophenyl)piperazine to give, after purification by flash chromatography on a column of silica (40-63 μm), eluting with a mixture of dichloromethane and ethyl acetate (80/20 by volume), 173 mg of (5-benzyloxy-1H-pyrazol-3-yl)[4-(3-chlorophenyl)piperazin-1-yl]methanone in the form of a white solid, the characteristics of which are as follows:
Mass spectrum(ES): m/z=397 (MH⁺)

Step 5: 100 mg of the product from Step 4 of the present example, 10 mg of cuprous iodide, 2.0 ml of 1,4-dioxane, 30 μl of trans-1,2-diaminocyclohexane, 115 mg of cesium carbonate, 37 μl of 2-bromopyridine and 30 μl of 1-hexyl-3-methylimidazolium pentafluorophosphate are placed in a microwave reactor and then subjected to the microwave field for 15 minutes at 140° C. The mixture is filtered, rinsed with 0.5 ml of 1,4-dioxane, a further 10 mg of cuprous iodide, 30 μl of trans-1,2-diaminocyclohexane, 115 mg of cesium carbonate, 37 μl of 2-bromopyridine and 30 μl of 1-hexyl-3-methylimidazolium pentafluorophosphate are added and the mixture is then subjected to the microwave field for a further 15 minutes at 140° C. The reaction mixture is poured into 20 ml of water and extracted with ethyl acetate. The organic phase is dried over magnesium sulfate and concentrated under reduced pressure. After purification by flash chromatography on a column of silica (40-63 μm), eluting with a mixture of dichloromethane and ethyl acetate (80/20 by volume), 62 mg of (5-benzyloxy-2-pyrid-2-yl-2H-pyrazol-3-yl)[4-(3-chlorophenyl)piperazin-1-yl]methanone are obtained in the form of an orange gum, the characteristics of which are as follows:
LC/MS analysis: tr=4.77; m/z=474 (MH⁺)
¹H NMR spectrum (400 MHz, (CD₃)₂SO d6, δ in ppm): 3.08 ppm (m, 2H); 3.31 ppm (masked, 4H); 3.74 ppm (mr, 2H); 5.30 ppm (s, 2H); 6.25 ppm (s, 1H); 6.81 ppm (dd, J=2.5-8.5 Hz, 1H); 6.88 ppm (dd, J=2.5-8.5 Hz, 1H); 6.94 ppm (t, J=2.5 Hz, 1H); 7.21 ppm (t, J=8.5 Hz, 1H); 7.26 ppm (ddd, J=1-5-7.5 Hz, 1H); 7.36 ppm (bt, J=8.5 Hz, 1H); 7.41 ppm (bt, J=8.5 Hz, 2H); 7.51 ppm (bd, J=8.5 Hz, 2H); 7.76 ppm (td, J=1-8.5 Hz, 1H); 7.96 ppm (ddd, J=2-7.5-8.5 Hz, 1H); 8.30 ppm (ddd, J=1-2-5 Hz, 1H).

### EXAMPLE E62

### (5-Methyl-2-phenyl-2H-pyrazol-3-yl)[4-(2-nitrophenyl)piperazin-1-yl]methanone

By working in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 414.5 mg of 1-(2-nitrophenyl)piperazine, 490 mg of (5-methyl-2-phenyl-2H-pyrazol-3-yl)[4-(2-nitrophenyl)piperazin-1-yl]methanone are obtained in the form of a yellow solid, the characteristics of which are as follows:
Melting point (Kofler): 127° C.
Mass spectrum (EI): m/z=391 (M+)

### EXAMPLE E63

### [4-(3,5-Dimethylphenyl)piperazin-1-yl](5-methyl-2-phenyl-2H-pyrazol-3-yl)methanone

By working in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 380.6 mg of 1-(3,5-dimethylphenyl)piperazine, 450 mg of [4-(3,5-dimethylphenyl)piperazin-1-yl](5-methyl-2-phenyl-2H-pyrazol-3-yl)methanone are obtained in the form of a white solid, the characteristics of which are as follows:
Melting point (Kofler): 109° C.
Mass spectrum (EI): m/z=374(M+)

### EXAMPLES E64 and E65

### [5-Bromo-2-(4-bromophenyl)-2H-pyrazol-3-yl][4-(3-chlorophenyl)piperazin-1-yl]methanone (EXAMPLE E64) and

### (5-Bromo-2-phenyl-2H-pyrazol-3-yl)[4-(3-chlorophenyl)piperazin-1-yl]-methanone (EXAMPLE E65)

Step 1: The synthesis of ethyl 5-bromo-2-phenyl-2H-pyrazole-3-carboxylate, according to Tetrahedron Lett., 40, 2605 (1999), starting with 1.64 g of (phenylhydrazono)acetic acid and 3.56 g of N-bromosuccinimide in 40 ml of DMF, followed by addition of 5.1 ml of ethyl propionate and 1.4 ml of triethylamine gives, after 2 successive purifications by flash chromatography on a column of silica (40-63 μm), eluting, respectively, with a mixture of heptane and ethyl acetate (90/10 by volume) and, for the fractions that are still impure, with another mixture of cyclohexane and acetone (95/5 by volume):
57 mg of ethyl 5-bromo-2-(4-bromophenyl)-2H-pyrazole-3-carboxylate, in the form of an orange-colored solid, the characteristics of which are as follows:
Mass spectrum (ES): m/z=374 (MH⁺)
¹H NMR spectrum (400 MHz, (CD₃)₂SO d6, δ in ppm): 1.16 ppm (t, J=7 Hz, 3H); 4.18 ppm (q, J=7 Hz, 2H); 7.24 ppm (s, 1H); 7.47 and 7.70 ppm (AA′BB′ system, 4H). and
520 mg of ethyl 5-bromo-2-phenyl-2H-pyrazole-3-carboxylate, in the form of an orange-colored solid, the characteristics of which are in accordance with those given in the literature.

Step 2: The process is performed in a manner similar to that of Step 2 of Example E42, starting with 192 mg of ethyl 5-bromo-2-phenyl-2H-pyrazole-3-carboxylate, containing 10 to 20% of ethyl 5-bromo-2-(4-bromophenyl)-2H-pyrazole-3-carboxylate, and 256 mg of 1-(3-chlorophenyl)piperazine, to give, after purification by flash chromatography on a column of silica (40-63 μm), eluting with a mixture of cyclohexane and ethyl acetate (80/20 by volume):
8 mg of [5-bromo-2-(4-bromophenyl)-2H-pyrazol-3-yl][4-(3-chlorophenyl)piperazin-1-yl]methanone (Example E64) in the form of a pale yellow solid, the characteristics of which are as follows:
LC/MS analysis: tr=4.78; m/z=523 (MH⁺)
¹H NMR spectrum (400 MHz, CDCl₃, δ in ppm): 2.84 ppm (m, 2H); 3.14 ppm (m, 2H); 3.32 ppm (m, 2H); 3.84 ppm (m, 2H); 6.58 ppm (s, 1H); 6.72 ppm (dd, J=2.5-8.5 Hz, 1H); 6.82 ppm (t, J=2.5 Hz, 1H); 6.90 ppm (dd, J=2.5-8.5 Hz, 1H); 7.18 ppm (t, J=8.5 Hz, 1H); 7.42 and 7.59 ppm (AA′BB′ system, 4H).
   and
108 mg of (5-bromo-2-phenyl-2H-pyrazol-3-yl)[4-(3-chlorophenyl)-piperazin-1-yl]methanone (Example E65) in the form of a white solid, the characteristics of which are as follows:
LC/MS analysis: tr=4.51; m/z=445 (MH⁺)

### EXAMPLE E66

### [4-(3-Chlorophenyl)piperazin-1-yl](2,5-diphenyl-2H-pyrazol-3-yl)methanone

22 mg of tetrakis(triphenylphosphine)palladium(0) and 0.5 ml of water are added to 90 mg of the product of Example E65, 35 mg of phenylboronic acid and 42 mg of sodium carbonate in 2 ml of DMF in a microwave reactor. The reaction mixture is subjected to the microwave field for 5 minutes at 140° C. and then poured into 10 ml of saturated aqueous sodium dihydrogen phosphate solution and extracted with ethyl acetate. The organic phase is dried over magnesium sulfate and concentrated under reduced pressure. After purification by flash chromatography on a column of silica (30-60 μm), eluting with a mixture of cyclohexane and acetone (80/20 by volume), 50 mg of [4-(3-chlorophenyl)piperazin-1-yl](2,5-diphenyl-2H-pyrazol-3-yl)methanone are obtained in the form of a yellow resin.
LC/MS analysis: tr=4.77; m/z=443 (MH⁺)

### EXAMPLE E67

### [4-(3-Chlorophenyl)piperazin-1-yl][2-phenyl-5-(pyrid-3-yl)-2H-pyrazol-3-yl]methanone

The process is performed in a manner similar to that of Example E66, starting with 74 mg of the product of Example E65 and 29 mg of pyridyl-3-boronic acid, to give, after purification by flash chromatography on a column of silica (30-60 μm), eluting with a mixture of dichloromethane and ethyl acetate (80/20 by volume), 35 mg of [4-(3-chlorophenyl)piperazin-1-yl][2-phenyl-5-(pyrid-3-yl)-2H-pyrazol-3-yl]methanone in the form of a white solid, the characteristics of which are as follows:
LC/MS analysis: tr=4.08; m/z=444 (MH⁺)

### EXAMPLE E68

### [4-(3-Chlorophenyl)piperazin-1-yl][2-phenyl-5-(thiophen-3-yl)-2H-pyrazol-3-yl]methanone

The process is performed in a manner similar to that of Example E66, starting with 74 mg of the product of Example E65 and 30 mg of thienyl-3-boronic acid, to give, after successive purifications by flash chromatography on a column of silica (30-60 μm), eluting with a mixture of heptane and ethyl acetate (90/10 by volume) and then by preparative HPLC/MS (H₂O pH=5/CH₃CN), 20.5 mg of [4-(3-chlorophenyl)piperazin-1-yl][2-phenyl-5-(thiophen-3-yl)-2H-pyrazol-3-yl])methanone in the form of a white powder, the characteristics of which are as follows:
LC/MS analysis: tr=5.04; m/z=449 (MH⁺)

### EXAMPLE E69

### [4-(3-Chlorophenyl)piperazin-1-yl][2-phenyl-5-(thiophen-2-yl)-2H-pyrazol-3-yl]methanone

The process is performed in a manner similar to that of Example E66, starting with 67 mg of the product of Example E65 and 27 mg of thienyl-2-boronic acid, to give, after successive purifications by flash chromatography on a column of silica (30-60 μm), eluting with a mixture of heptane and ethyl acetate (90/10 by volume) and then by preparative HPLC/MS (H₂O pH=5/CH₃CN), 27 mg of [4-(3-chlorophenyl)piperazin-1-yl][2-phenyl-5-(thiophen-2-yl)-2H-pyrazol-3-yl]methanone in the form of a white powder, the characteristics of which are as follows:
LC/MS analysis: tr=5.07; m/z=449 (MH⁺)

### EXAMPLE E70

### 5-[4-(3-Chlorophenyl)piperazine-1-carbonyl]-1-phenyl-1H-pyrazole-3-carboxaldehyde

Step 1: 2.50 g of calcium carbonate are added at room temperature to 9.68 g of ethyl 5-dibromomethyl-2-phenyl-2H-pyrazole-3-carboxylate, obtained in Step 1 of Example E73, in 260 ml of water. The reaction mixture is stirred at reflux for 7 hours, cooled, acidified to pH 1 by controlled addition of concentrated hydrochloric acid and extracted with ethyl acetate. The organic phase is dried over magnesium sulfate and concentrated under reduced pressure. After purification by flash chromatography on a column of silica (40-63 μm), eluting with a mixture of dichloromethane and heptane (70/30 by volume), 5.06 g of ethyl 5-formyl-2-phenyl-2H-pyrazole-3-carboxylate are obtained in the form of a white powder, the characteristics of which are as follows:
Mass spectrum (ES): m/z=245 (MH⁺)

Step 2: The process is performed in a manner similar to that of Step 1 of Example E22, starting with 63 mg of ethyl 5-formyl-2-phenyl-2H-pyrazole-3-carboxylate and 101 mg of 1-(3-chlorophenyl)piperazine, to give, after purification by flash chromatography on a column of silica (30-60 μm), eluting with a mixture of heptane and ethyl acetate (70/30 by volume), 60 mg of 5-[4-(3-chlorophenyl)piperazine-1-carbonyl]-1-phenyl-1H-pyrazole-3-carboxaldehyde in the form of a colorless oil, the characteristics of which are as follows:
LC/MS analysis: tr=4.47; m/z=395 (MH⁺)

### EXAMPLE E71

### [4-(3,5-Dimethoxyphenyl)piperazin-1-yl](5-isopropyl-2-phenyl-2H-pyrazol-3-yl)methanone

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 193.2 mg of 1-(3,5-dimethoxyphenyl)piperazine and by replacing the 5-methyl-2-phenyl-2H-pyrazole-3-carboxylic acid with 200 mg of 5-isopropyl-2-phenyl-2H-pyrazole-3-carboxylic acid, 300 mg of [4-(3,5-dimethoxyphenyl)piperazin-1-yl](5-isopropyl-2-phenyl-2H-pyrazol-3-yl)methanone are obtained in the form of a white powder, the characteristics of which are as follows:
Melting point (Kofler): 116° C.
Mass spectrum (EI): m/z=434 (M+)

### EXAMPLE E72

### [4-(3-Chloro-4-fluorophenyl)piperazin-1-yl](5-methyl-2-phenyl-2H-pyrazol-3-yl)methanone

By performing the process, in 3 steps, in a manner similar to that for the synthesis of [4-(3-chloro-3-pyridyl)piperazin-1-yl](5-methyl-2-phenyl-2H-pyrazol-3-yl)methanone (Example 33), but replacing in Step 1 of the synthesis of the 3,5-dichloropyridine with 404 mg of 3-chloro-4-fluorobromobenzene, 213 mg of [4-(3-chloro-4-fluorophenyl)piperazin-1-yl](5-methyl-2-phenyl-2H-pyrazol-3-yl)methanone are obtained, in Step 3 of the synthesis, in the form of a solid, the characteristics of which are as follows:
Spectre IR: 2924; 2839; 1647; 1501; 1221; 1003; 996; 771; 730 and 693 cm⁻¹
¹H NMR spectrum (300 MHz, (CD₃)₂SO d6, δ in ppm): 2.31 (s: 3H); 2.82 (unresolved complex: 2H); 3.118 (unresolved complex: 2H); from 3.25 to 3.40 (unresolved complex: 2H); 3.69 (unresolved complex: 2H); 6.53 (s: 1 H); 6.89 (dt, J=9 and 3.5 Hz: 1H); 7.05 (dd, J=6 and 3 Hz: 1H); 7.26 (t, J=9 Hz: 1H); 7.36 (broad t, J=7.5 Hz: 1H); from 7.40 to 7.55 (mt: 4H).

### EXAMPLE E 73

### [4-(3,5-Dimethoxyphenyl)piperazin-1-yl](3-hydroxymethyl-1-phenyl-1H-pyrazol-5-yl)methanone

Step 1: 13 g of ethyl 3-methyl-1-phenyl-1H-pyrazole-5-carboxylate, which may be obtained according to J. Het Chem 1999, 36(1), 217-220, are dissolved in 250 ml of carbon tetrachloride in a 2 L photochemical reactor, followed by successive addition of 12.45 g of N-bromosuccinimide and 0.65 g of 2,2′-azobis(2-methylpropionitrile). The mixture is irradiated for 4 hours using a Hanovia lamp, 2.67 g of N-bromosuccinimide are then added and the mixture is irradiated for a further 2 hours. After cooling to room temperature, the insoluble material formed is filtered off, washed twice with 50 ml of carbon tetrachloride, and the combined filtrates are concentrated under reduced pressure. The orange oil obtained is purified by flash chromatography on a column of silica (60; 35-70 μm), eluting with toluene, to give, by collecting the fractions eluted between 600 and 1200 ml, 8 g of ethyl 3-bromomethyl-1-phenyl-1H-pyrazole-5-carboxylate in the form of a yellow powder, which is used without further purification in the following step.

Step 2: 14.22 g of ethyl 3-bromomethyl-1-phenyl-1H-pyrazole-5-carboxylate, obtained as in the preceding step, are dissolved in 170 ml of dimethylformamide in a 500 ml three-necked flask under an argon atmosphere, followed by addition of 7.95 g of sodium benzoate, and the mixture is heated at 50° C. for 3 hours. After cooling and concentrating under reduced pressure, the residue is poured into 200 ml of water and then extracted with 3 times 100 ml of ethyl acetate. The combined organic phases are washed with saturated aqueous ammonium chloride solution, dried over magnesium sulfate and concentrated under reduced pressure. 15.5 g of ethyl 3-benzoyloxymethyl-1-phenyl-1H-pyrazole-5-carboxylate are thus obtained in the form of a beige-colored powder, which is used without further purification in the following step.

Step 3: 2.1 g of (3,5-dimethoxyphenyl)piperazine are dissolved in 8 ml of dry toluene at 30° C., in a 500 ml three-necked flask under an argon atmosphere, followed by dropwise addition of 4.5 ml of a 2 m solution of trimethylaluminum in toluene, and the mixture is stirred for 30 minutes. After cooling to about 20° C., 1.06 g of ethyl 3-benzoyloxymethyl-1-phenyl-1H-pyrazole-5-carboxylate obtained in the preceding step, dissolved in 20 ml of toluene, are added. After heating at 60° C. for 7 hours, the reaction medium is poured into 70 ml of aqueous 1M sodium potassium tartrate solution and then extracted with 3 times 50 ml of ethyl acetate. The combined organic phases are washed with 50 ml of aqueous 1M sodium potassium tartrate solution, dried over magnesium sulfate and concentrated under reduced pressure. The orange oil obtained is purified by flash chromatography on a column of silica (60; 35-70 μm), eluting with a mixture of dichloromethane and of methanol (99.5/0.5 by volume), and 0.8 g of [4-(3,5-dimethoxyphenyl)piperazin-1-yl](5-hydroxymethyl-2-phenyl-2H-pyrazol-3-yl)methanone is thus obtained in the form of a pale beige-colored foam, the characteristics of which are as follows:
Mass spectrum (EI): m/z=422 (M⁺)

### EXAMPLE E74

### [4-(3-Difluoromethoxyphenyl)piperazin-1-yl](5-methyl-2-phenyl-2H-pyrazol-3-yl)methanone hydrochloride

Step1: A mixture of 500.1 mg of 1-boc piperazine and 598.8 mg of commercial 3-difluoromethoxybromobenzene is placed in 20 ml of toluene in a 50 ml three-necked flask under an inert atmosphere of argon, followed by addition of 56.85 mg of (R)(+)-2,2′-bis(diphenylphosphino)-1,1′-binaphthyl and 20.4 mg of palladium(II) acetate. The reaction mixture is stirred at reflux for 16 hours. After cooling to 20° C., the reaction mixture is diluted with water (20 ml) and then extracted with ethyl acetate (2×30 ml). The organic extracts are combined, dried over magnesium sulfate, filtered and evaporated under reduced pressure. The compound obtained is purified by chromatography on silica gel (AIT cartridge, ref. FC-25 Si-BP-SUP, 20-40 μm, dichloromethane eluent, flow rate of 20 ml/min). The fractions containing the expected compound are combined and then evaporated under reduced pressure. 253 mg of tert-butyl 4-(3-difluoromethoxyphenyl)piperazine-1-carboxylate are thus isolated, the characteristics of which are as follows:
LC/MS: RT=4.18 min, M+H⁺ 329.31 (Micromass machine, LCT model, connected to an HP 1100 machine, HP G1315A diode array detector (200-600 nm), Sedex 65 light-scattering detector; data analyzed with the Micromass MassLynx software; separation on a Hypersil BDS C18, 3 μm (50×4.6 mm) column, eluting with a linear gradient of from 5% to 90% of acetonitrile containing 0.05% (v/v) of trifluoroacetic acid (TFA) in water containing 0.05% (v/v) TFA, over 3.5 minutes at a flow rate of 1 ml/min).

Step 2: A solution of 253 mg of tert-butyl 4-(3-difluoromethoxyphenyl)-piperazine-1-carboxylate in a mixture of 1016 μl of dioxane and 963 μl of hydrochloric acid is placed in a 10 ml round-bottomed flask. The reaction mixture is stirred at 20° C. for 48 hours. The solid formed is filtered off, washed with 10 ml of diisopropyl ether and dried under reduced pressure. 189 mg of 1-(3-difluoromethoxyphenyl)piperazine hydrochloride are thus isolated, and are used without purification for the following step.

Step 3: A solution of 144.4 mg of 5-methyl-2-phenyl-2H-pyrazole-3-carboxylic acid, which may be obtained according to J. Het. Chem., 30, 307 (1993), in 11 ml of dichloromethane is placed in a 50 ml three-necked flask under an inert atmosphere of argon, followed by successive addition of 189 mg of 1-(3-difluoromethoxyphenyl)piperazine hydrochloride, 106.1 mg of 1-hydroxy-benzotriazole, 150.6 mg of 1-(3-dimethylaminopropyl)-3-ethylcarbodiimide hydrochloride and then 331 μl of triethylamine (331 μl). The reaction mixture is stirred at 20° C. for 48 hours and then diluted with dichloromethane (20 ml) and water (20 ml), the phases are separated by settling and the organic phase is extracted (30 ml of dichloromethane). The organic extracts are combined, dried over magnesium sulfate, filtered and evaporated under reduced pressure. The compound obtained is purified by chromatography on silica gel (AIT cartridge, ref. FC 25-Si-HP, 15-35 μm, eluent: 80/20 to 60/40 cyclohexane/ethyl acetate over 60 minutes, flow rate of 7 ml/min). The fractions containing the expected compound are combined and then evaporated under reduced pressure. The evaporation residue is taken up in a mixture of ethyl ether (12 ml) and 2N hydrochloric acid/ethyl ether (500 μl) and then triturated until a solid is obtained, which is filtered off, washed (5 ml) and dried under reduced pressure. 199 mg of [4-(3-difluoromethoxy-phenyl)piperazin-1-yl](5-methyl-2-phenyl-2H-pyrazol-3-yl)methanone hydrochloride are thus isolated in the form of a white powder, the characteristics of which are as follows:
Melting point (Kofler): 127° C.

### EXAMPLE E75

### [4-(3-Chlorophenyl)piperazin-1-yl][5-(2-methylimidazol-1-ylmethyl)-2-phenyl-2H-pyrazol-3-yl]methanone

Step 1: 133 mg of 2-methylimidazole are added to 100 mg of ethyl 5-bromomethyl-2-phenyl-2H-pyrazole-3-carboxylate, obtained in Step 1 of Example E73, in 1.5 ml of THF in a microwave reactor. The reaction mixture is subjected to the microwave field for 10 minutes at 120° C., poured into 20 ml of saturated aqueous sodium bicarbonate solution and extracted with ethyl acetate. The organic phase is dried over magnesium sulfate and concentrated under reduced pressure. After purification by flash chromatography on a column of silica (30-60 μm), eluting with a mixture of ethyl acetate and triethylamine (90/10 by volume), 74 mg of ethyl 5-(2-methylimidazol-1-ylmethyl)-2-phenyl-2H-pyrazole-3-carboxylate are obtained in the form of a colorless oil, the characteristics of which are as follows:
Mass spectrum (ES): m/z=311 (MH⁺)

Step 2: The process is performed in a manner similar to that of Step 1 of Example E22, starting with 114 mg of the product of Step 1 of the present example and 145 mg of 1-(3-chlorophenyl)piperazine, to give, after purification by flash chromatography on a column of silica (30-60 μm), eluting with a mixture of ethyl acetate and triethylamine (90/10 by volume), 21 mg of [4-(3-chlorophenyl)piperazin-1-yl[]5-(2-methylimidazol-1-ylmethyl)-2-phenyl-2H-pyrazol-3-yl]methanone in the form of a yellow oil, the characteristics of which are as follows:
LC/MS analysis: tr=2.85; m/z=461 (MH⁺)

### EXAMPLE E76

### [4-(3-Chlorophenyl)piperazin-1-yl](2-phenyl-5-phenylaminomethyl-2H-pyrazol-3-yl)methanone

Step 1: The process is performed in a manner similar to that of Step 1 of Example E75, starting with 100 mg of the product of Step 1 of Example E73 and 151 mg of aniline, to give, after purification by flash chromatography on a column of silica (30-60 μm), eluting with a mixture of toluene, ethyl acetate and triethylamine (80/20/0.1 by volume), 79 mg of ethyl 2-phenyl-5-phenylaminomethyl-2H-pyrazole-3-carboxylate in the form of a pale pink oil, the characteristics of which are as follows:
Mass spectrum (ES): m/z=322 (MH⁺)

Step 2: The process is performed in a manner similar to that of Step 2 of Example E75, starting with 79 mg of the product of Step 1 of present example and 170 mg of 1-(3-chlorophenyl)piperazine, to give, after purification by flash chromatography on a column of silica (30-60 μm), eluting with a mixture of toluene, ethyl acetate and triethylamine (70/30/0.1 by volume), 39 mg of [4-(3-chlorophenyl)piperazin-1-yl](2-phenyl-5-phenylaminomethyl-2H-pyrazol-3-yl)-methanone in the form of a pale yellow oil, the characteristics of which are as follows:
LC/MS analysis: tr=4.51; m/z=472 (MH⁺)

### EXAMPLE E77

### [4-(2-Bromo-5-chlorophenyl)piperazin-1-yl](5-methyl-2-phenyl-2H-pyrazol-3-yl)methanone

The process is performed in a manner similar to that of Example E59, starting with 300 mg of [4-(3-chlorophenyl)piperazin-1-yl](5-methyl-2-phenyl-2H-pyrazol-3-yl)methanone in 3 ml of carbon tetrachloride, 210 mg of N-bromosuccinimide, 15 mg of benzoyl peroxide and 65 mg of potassium carbonate, to give, after successive purifications by flash chromatography on a column of silica (40-63 μm), eluting with a mixture of toluene and ethyl acetate (8/2 by volume) and then by preparative HPLC/MS (H₂O pH=9/CH₃CN), 16 mg of [4-(2-bromo-5-chlorophenyl)piperazin-1-yl](5-methyl-2-phenyl-2H-pyrazol-3-yl)methanone in the form of a cream-colored solid, the characteristics of which are as follows:
LC/MS analysis: tr=4.45; m/z=459 (MH⁺)
¹H NMR spectrum (400 MHz, (CD₃)₂SO d6, δ in ppm): 2.28 ppm (s, 3H); 2.68 ppm (m, 2H); 2.93 ppm (m, 2H); 3.40 ppm (m, 2H); 3.70 ppm (m, 2H); 6.53 ppm (s, 1H); 7.40 ppm (tl, J=8 Hz, 1H); 7.44 ppm (dl, J=8 Hz, 2H); 7.50 ppm (tl, J=8 Hz, 2H); 7.62 ppm (d, J=8.5 Hz, 1H); 7.05 ppm (d, J=2.5 Hz, 1H); 7.09 ppm (dd, J=2.5-8.5 Hz, 1H).

### EXAMPLE E 78

### [4-(3,5-Dimethoxyphenyl)piperazin-1-yl](3-dibromomethyl-1-phenyl-1H-pyrazol-5-yl)methanone

Step 1: By working as in Step 1 of Example 73, but collecting the fractions eluted between 150 and 550 ml, 9 g of ethyl 3-dibromomethyl-1-phenyl-1H-pyrazole-5-carboxylate are obtained in the form of an orange-colored oil, which is used without further purification for the following step.

Step 2: 0.52 ml of a 2M solution of trimethylaluminum in toluene is added, at 25° C., to a solution of 195 mg of 1-(3,5-dimethoxyphenyl)piperazine in 2 ml of toluene, followed by addition, at 60° C., of a solution of 162 mg of the product of Step 1 of the present example in 4 ml of toluene. The reaction medium is stirred for 2.5 hours at 65° C. and then for 1.5 hours at 80° C., after which is it poured into 10 ml of aqueous 1M sodium potassium tartrate solution and extracted with ethyl acetate. The organic phase is dried over magnesium sulfate and concentrated under reduced pressure. After purification by flash chromatography on a column of silica (40-63 μm), eluting with a mixture of dichloromethane and ethyl acetate (90/10 by volume), 235 mg of (5-dibromomethyl-2-phenyl-2H-pyrazol-3-yl)[4-(3,5-dimethoxyphenyl)piperazin-1-yl]methanone are obtained in the form of a pale yellow solid, the characteristics of which are as follows:
LC/MS analysis: tr=4.36; m/z=563 (MH⁺)
¹H NMR spectrum (400 MHz, (CD₃)₂SO d6, δ in ppm): 2.83 ppm (m, 2H); 3.10 ppm (m, 2H); 3.37 (masked, 2H); 3.66 ppm (m, 2H); 3.68 ppm (s, 6H); 5.99 ppm (bs, 1H); 6.02 ppm (bs, 2H); 6.99 ppm (s, 1H); 7.42 ppm (s, 1H); 7.46 ppm (m, 3H); 7.54 ppm (tl, J=8Hz, 2H).

### EXAMPLE E79

### [4-(2,4-Dibromo-5-chlorophenyl)piperazin-1-yl](5-methyl-2-phenyl-2H-pyrazol-3-yl)methanone

The product is obtained at the same time as that of Example E77 after preparative HPLC/MS purification, in the form of a pale yellow oil, the characteristics of which are as follows:
LC/MS analysis: tr=4.74; m/z=537 (MH⁺)
¹H NMR spectrum (400 MHz, (CD₃)₂SO d6, δ in ppm): 2.28 ppm (s,3H); 2.71 ppm (m, 2H); 2.94 ppm (m, 2H); 3.40 ppm (m, 2H); 3.70 ppm (m, 2H); 6.53 ppm (s, 1H); 7.23 ppm (s, 1H); 7.40 ppm (tl, J=8, 1H); 7.44 ppm (dl, J=8 Hz, 2H); 7.51 ppm (tl, J=8 Hz, 2H); 8.01 ppm (s, 1H).

### EXAMPLE E80

### (5-Benzyloxymethyl-2-phenyl-2H-pyrazol-3-yl)[4-(3-chlorophenyl)piperazin-1-yl]methanone

Step 1: 35 mg of 50% sodium hydride in oil are added to a solution of 87 mg of benzyl alcohol in 0.5 ml of DMF. The reaction mixture is stirred for 30 minutes at room temperature, followed by addition of a solution of 200 mg of ethyl 5-bromomethyl-2-phenyl-2H-pyrazole-3-carboxylate, obtained in Step 1 of Example E73, in 1.5 ml of DMF. The reaction mixture is stirred for 4 hours at room temperature and then poured into 50 ml of saturated aqueous sodium dihydrogen phosphate solution and extracted with ethyl acetate. The organic phase is dried over magnesium sulfate and concentrated under reduced pressure. After purification by flash chromatography on a column of silica (30-60 μm), eluting with a mixture of ethyl acetate and triethylamine (90/10 by volume), 52 mg of ethyl 5-benzyloxymethyl-2-phenyl-2H-pyrazole-3-carboxylate are obtained in the form of a colorless oil, the characteristics of which are as follows:
Mass spectrum (ES): m/z=337 (MH⁺)

Step 2: The process is performed in a manner similar to that of Step 2 of Example E75, starting with 52 mg of the product of Step 1 of the present example and 122 mg of 1-(3-chlorophenyl)piperazine, for 1.5 hours at 60° C., to give, after purification by flash chromatography on a column of silica (30-60 μm), eluting with a mixture of dichloromethane and ethyl acetate (95/5 by volume), 54 mg of (5-benzyloxymethyl-2-phenyl-2H-pyrazol-3-yl)[4-(3-chlorophenyl)piperazin-1-yl]methanone in the form of a pale yellow oil, the characteristics of which are as follows:
LC/MS analysis: tr=4.63; m/z=487 (MH⁺)

### EXAMPLES E81 and E82

### [5-Bromo-2-(4-bromophenyl)-2H-pyrazol-3-yl][4-(3,5-dimethoxyphenyl)-piperazin-1-yl]methanone (EXAMPLE E81) and

### (5-Bromo-2-phenyl-2H-pyrazol-3-yl)[4-(3,5-dimethoxyphenyl)piperazin-1-y]-methanone (EXAMPLE E82)

The process is performed in a manner similar to that of Step 2 of Examples E64 and E65, starting with 1.18 g of ethyl 5-bromo-2-phenyl-2H-pyrazole-3-carboxylate, obtained in Step 1 of Examples E64 and E65 and containing 10% to 20% of ethyl 5-bromo-2-(4-bromophenyl)-2H-pyrazole-3-carboxylate, and 1.87 g of 1-(3,5-dimethoxyphenyl)piperazine, to give, after purification by flash chromatography on a column of silica (40-63 μm), eluting with a mixture of cyclohexane and ethyl acetate (70/30 by volume):
58 mg of [5-bromo-2-(4-bromophenyl)-2H-pyrazol-3-yl][4-(3,5-dimethoxyphenyl)piperazin-1-yl]methanone (Example E81), in the form of an ochre-colored solid, the characteristics of which are as follows:
LC/MS analysis: tr=4.47; m/z=549 (MH⁺)
¹H NMR spectrum (300 MHz, (CD₃)₂SO d6, δ in ppm): 2.95 ppm (m, 2H); 3.14 ppm (m, 2H); 3.44 ppm (m, 2H); 3.65 ppm (m, 2H); 3.70 ppm (s, 6H); 6.00 ppm (t, J=2 Hz, 1H); 6.05 ppm (d, J=2 Hz, 2 H); 6.98 ppm (s, 1H); 7.42 and 7.77 ppm (systeme AA′BB′, 4H).
   and
1.80 g of (5-bromo-2-phenyl-2H-pyrazol-3-yl)[4-(3,5-dimethoxyphenyl)-piperazin-1-yl]methanone (Example E82), in the form of an orange-colored resin, the characteristics of which are as follows:
LC/MS analysis: tr=4.17; m/z=471 (MH⁺)

### EXAMPLE E83

### Methyl N-{3-[4-(5-methyl-2-phenyl-2H-pyrazole-3-carbonyl)piperazin-1-yl]-phenyl}succinamate

424 mg of 1-(3-dimethylaminopropyl)-3-ethylcarbodiimide hydrochloride (EDCl), 297 mg of 1-hydroxybenzotriazole hydrate (HOBT) and 264 mg of succinic acid monomethyl ester are added to a solution of 726 mg of [4-(3-aminophenyl)piperazin-1-yl](5-methyl-2-phenyl-2H-pyrazol-3-yl)methanone, described in Example E9, in 35 ml of dichloromethane. The reaction mixture is stirred for 20 hours at room temperature. After addition of 50 ml of dichloromethane and 50 ml of water, the organic phase is separated out by settling of the phases and then washed with water, dried over magnesium sulfate and concentrated under reduced pressure. After purification by flash chromatography on a column of silica (60; 35-70 μm), eluting with pure ethyl acetate, 650 mg of methyl N-{3-[4-(5-methyl-2-phenyl-2H-pyrazole-3-carbonyl)piperazin-1-yl]phenyl}succinamate are obtained in the form of a white foam, the characteristics of which are as follows:
Mass spectrum (EI): m/z=475 (M⁺)

### EXAMPLE 84

### Parallel Synthesis of EXAMPLES E84, E105, E108 and 109

E84 [4-(3-Isopropoxyphenyl)piperazin-1-yl](5-methyl-2-phenyl-2H-pyrazol-3-yl)methanone

E105 Methyl {3-[4-(5-methyl-2-phenyl-2H-pyrazole-3-carbonyl)piperazin-1-yl]-phenoxy}acetate hydrochloride

E108 [4-(3-Butoxyphenyl)piperazin-1-yl](5-methyl-2-phenyl-2H-pyrazol-3-yl)-methanone hydrochloride

E109 [4-(3-Ethoxyphenyl)piperazin-1-yl](5-methyl-2-phenyl-2H-pyrazol-3-yl)methanone hydrochloride

A solution of 100 mg of [4-(3-hydroxyphenyl)piperazin-1-yl](5-methyl-2-phenyl-2H-pyrazol-3-yl)methanone, prepared in Example 13, in 1000 μl of dimethylformamide (1000 μl) is placed in 4 glass reactors (13×100 mm) identified from 1 to 4, equipped with a magnetic stirrer and placed under an inert atmosphere of argon, followed by addition of 12.4 mg of sodium hydride to each reactor. Each tube is stirred at 20° C. for 1.5 hours, followed by addition of the halo derivatives, i.e., respectively, 38.87 μl of 2-bromopropane to tube 1, 39.19 μl of methyl bromoacetate to tube 2, 44.67 μl of 1-bromobutane to tube 3 and 26.7 μl of iodoethane to tube 4. After 1 hour at 20° C., analysis by thin-layer chromatography (1/1 cyclohexane/ethyl acetate) shows that reactions 2, 3 and 4 are complete, whereas the reaction in tube 1 no longer proceeds. A further 12.4 mg of sodium hydride and 38.87 μl of 2-bromopropane are thus added to reactor 1. After reaction for a further 30 minutes, analysis by thin layer chromatography (1/1 cyclohexane/ethyl acetate) shows that reaction 1 is complete. The contents of reactors 1 to 4 are transferred into 4 glass tubes of 36×100, each reactor tube being rinsed with ethyl acetate (15 ml) and water (15 ml), and then transferred onto the liquid-liquid extraction platform. The following protocol is applied to the three reaction mixtures: decantation of the two phases, separation of the heavy and light extracts and then extraction of the heavy phases with ethyl acetate (2×10 ml), reunification of the organic extracts. After drying and evaporating, the compounds are isolated and are purified by chromatography on silica gel (prefilled cartridge, diameter 26 mm, height 135 mm, SiO₂ 15-40 μm), eluting with mixtures of cyclohexane and ethyl acetate (E84: 90-10 by volume at 10 ml/min; E105: 80-20 by volume at 10 ml/min , E108: 80-20 by volume at 10 ml/min; E109: 75-25 by volume at 10 ml/min). After evaporation of the fractions containing the expected compound,
either the expected compound is isolated directly, and 80.3 mg of [4-(3-isopropoxyphenyl)piperazin-1-yl](5-methyl-2-phenyl-2H-pyrazol-3-yl)-methanone E84 are thus obtained, the characteristics of which are as follows:
¹H NMR spectrum (300 MHz, (CD₃)₂SO d6, chemical shift in ppm): 1.25 (d, J=6.5 Hz: 6H); 2.30 (s: 3H); 2.80 (unresolved complex: 2H); 3.09 (unresolved complex: 2H); 3.33 (unresolved complex: 2H); 3.68 (unresolved complex: 2H); 4.57 (mt: 1H); from 6.30 to 6.40 (mt: 2H); 6.45 (broad d, J=8.5 Hz: 1H); 6.53 (s: 1H); 7.10 (t, J=8.5 Hz: 1H); 7.36 (broad t, J=7 Hz: 1H); from 7.40 to 7.55 (mt: 4H).
or the compound obtained is taken up in a mixture of ethyl ether (10 ml) and 2N hydrochloric acid/ethyl ether (150 μl) and is triturated until a solid appears. After filtration, washing with ethyl ether (5 ml) and drying, the corresponding hydrochlorides are isolated, and the following are thus obtained: 81.4 mg of methyl {3-[4-(5-methyl-2-phenyl-2H-pyrazole-3-carbonyl)piperazin-1-yl]phenoxy}acetate hydrochloride E105, the characteristics of which are as follows:
¹H NMR spectrum (300 MHz, (CD₃)₂SO d6, chemical shift in ppm): 2.30 (s: 3H); 2.82 (unresolved complex: 2H); 3.12 (unresolved complex: 2H); 3.35 (unresolved complex: 2H); 3.69 (unresolved complex: 2H); 3.71 (s: 3H); 4.75 (s: 2H); 6.37 (dd large, J=8.5 and 2 Hz: 1H); 6.44 (broad t, J=2 Hz: 1H); 6.52 (dd large, J=8.5 and 2 Hz: 1H); 6.53 (s: 1H); 7.12 (t, J=8.5 Hz: 1H); 7.37 (broad t, J=7 Hz: 1H); from 7.40 to 7.55 (mt: 4H).

81.4 mg of [4-(3-butoxyphenyl)piperazin-1-yl](5-methyl-2-phenyl-2H-pyrazol-3-yl)methanone hydrochloride E108, the characteristics of which are as follows:
¹H NMR spectrum (300 MHz, (CD₃)₂SO d6, chemical shift in ppm): 0.95 (t, J=7 Hz: 3H); 1.44 (mt: 2H); 1.68 (mt: 2H); 2.31 (s: 3H); 2.81 (unresolved complex: 2H); 3.10 (unresolved complex: 2H); 3.35 (unresolved complex: 2H); 3.69 (unresolved complex: 2H); 3.93 (t, J=6.5 Hz: 2H); from 6.35 to 6.45 (mt: 2H); 6.47 (broad d, J=8.5 Hz: 1H); 6.53 (s: 1H); 7.11 (t, J=8.5 Hz: 1H); of 7.35 to 7.55 (mt: 4H); 7.37 (broad t, J=7 Hz: 1H).
Melting point (Kofler)=125° C.

75.5 mg of [4-(3-ethoxyphenyl)piperazin-1-yl](5-methyl-2-phenyl-2H-pyrazol-3-yl)methanone hydrochloride E109, the characteristics of which are as follows:
¹H NMR spectrum (300 MHz, (CD₃)₂SO d6, chemical shift in ppm): 1.32 (t, J=7 Hz: 3H); 2.31 (s: 3H); 2.81 (unresolved complex: 2H); 3.10 (unresolved complex: 2H); from 3.25 to 3.45 (mt: 2H); 3.69 (unresolved complex: 2H); 3.99 (q, J=7 Hz: 2H); from 6.35 to 6.45 (mt: 2H); 6.47 (broad d, J=8.5 Hz: 1H); 6.53 (s: 1H); 7.11 (t, J=8.5 Hz: 1H); of 7.35 to 7.55 (mt: 4H); 7.37 (broad t, J=7 Hz: 1H).

### EXAMPLE E85

### [4-(3,5-Dimethoxyphenyl)piperazin-1-yl][2-phenyl-5-(thiophen-3-yl)-2H-pyrazol-3-yl]methanone

The process is performed in a manner similar to that of Example E66, starting with 150 mg of the product of Example E82 and 57.5 mg of thienyl-3-boronic acid, by microwave reaction for 3 minutes at 140° C., to give, after purification by flash chromatography on a column of silica (30-60 μm), eluting with a mixture of heptane and ethyl acetate (70/30 by volume), 126 mg of [4-3,5-dimethoxyphenyl)piperazin-1-yl][2-phenyl-5-(thiophen-3-yl)-2H-pyrazol-3-yl]-ethanone in the form of an ochre-colored solid, the characteristics of which are as follows:
LC/MS analysis: tr=4.36; m/z=475 (MH⁺)

### EXAMPLE E86

### [4-(3,5-Dimethoxyphenyl)piperazin-1-yl][2-phenyl-5-(E-propen-2-yl)-2H-pyrazol-3-yl]methanone

The process is performed in a manner similar to that of Example E66, starting with 150 mg of the product of Example E82 and 39 mg of trans-propenylboronic acid, by microwave reaction for 3 minutes to 140° C., to give, after purification by flash chromatography on a column of silica (30-60 μm), eluting with a mixture of heptane and ethyl acetate (70/30 by volume), 85 mg of [4-(3,5-dimethoxyphenyl)piperazin-1-yl][2-phenyl-5-(E-propen-2-yl)-2H-pyrazol-3-yl]methanone in the form of a pale yellow solid, the characteristics of which are as follows:
LC/MS analysis: tr=4.15; m/z=433 (MH⁺)
¹H NMR spectrum (300 MHz, (CD₃)₂SO d6, δ in ppm): 1.88 ppm (d, J=5 Hz, 3H); 2.79 ppm (m, 2H); 3.09 ppm (m, 2H); 3.33 (masked, 2H); 3.65 ppm (m, 2H); 3.68 ppm (s, 6H); 5.99 ppm (t, J=2 Hz, 1H); 6.01 ppm (d, J=2 Hz, 2H); 6.42 ppm (m, 2H); 6.86 ppm (s, 1H); 7.37 ppm (m, 1H); from 7.42 to 7.53 ppm (m, 5H).

### EXAMPLE E87

### [4-(3,5-Dimethoxyphenyl)piperazin-1-yl]-{5-[E-2-(4-fluorophenyl)vinyl]-2-phenyl-2H-pyrazol-3-yl}methanone

The process is performed in a manner similar to that of Example E66, starting with 150 mg of the product of Example E82 and 75 mg of trans-2-(4-fluorophenyl)vinylboronic acid, by microwave reaction for 3 minutes at 140° C., to obtain, after purification by flash chromatography on a column of silica (30-60 μm), eluting with a mixture of heptane and ethyl acetate (70/03 by volume), 118 g of [4-(3,5-dimethoxyphenyl)piperazin-1-yl]-{5-[E-2-(4-fluoro-phenyl)vinyl]-2-phenyl-2H-pyrazol-3-yl}methanone are obtained in the form of a pale yellow solid, the characteristics of which are as follows:
LC/MS analysis: tr=4.60; m/z=513 (MH⁺)
¹H NMR spectrum (300 MHz, (CD₃)₂SO d6, δ in ppm): 2.83 ppm (m, 2H); 3.10 ppm (m, 2H); 3.38 ppm (masked, 2H); 3.68 ppm (m, 2H); 3.69 ppm (s, 6H); 5.99 ppm (t, J=2 Hz, 1H); 6.03 ppm (d, J=2 Hz, 2H); 7.05 ppm (s, 1H); 7.23 ppm (t, J=8.5 Hz, 2H); 7.20 ppm (d, J=16.5 Hz, 1H); 7.34 ppm (d, J=16.5 Hz, 1H); 7.40 ppm (m, 1H); 7.50 ppm (m, 4H); 7.68 ppm ( dd, J=5-8.5 Hz, 2H).

### EXAMPLE E88

### [4-(3,5-Dimethoxyphenyl)piperazin-1-yl][5-(4-fluorophenyl)-2-phenyl-2H-pyrazol-3-yl]methanone

The process is performed in a manner similar to that of Example E66, starting with 150 mg of the product of Example E82 and 66 mg of 4-fluorophenylboronic acid, by microwave reaction for 3 minutes at 140° C., to obtain, after purification by flash chromatography on a column of silica (30-60 μm), eluting with a mixture of heptane and ethyl acetate (70/30 by volume), 133 mg of [4-(3,5-dimethoxyphenyl)piperazin-1-yl][5-(4-fluorophenyl)-2-phenyl-2H-pyrazol-3-yl]methanone in the form of a beige-colored solid, the characteristics of which are as follows:
LC/MS analysis: tr=4.51; m/z=487 (MH⁺)

### EXAMPLE E89

### [4-(3,5-Dimethoxyphenyl)piperazin-1-yl][2-phenyl-5-(4-trifluoromethylphenyl)-2H-pyrazol-3-yl]methanone

The process is performed in a manner similar to that of Example E66, starting with 150 mg of the product of Example E82 and 86 mg of 4-(trifluoromethyl)phenylboronic acid, by microwave reaction for 3 minutes at 140° C., to obtain, after purification by flash chromatography on a column of silica (30-60 μm), eluting with a mixture of heptane and ethyl acetate (70/30 by volume), 147 mg of [4-(3,5-dimethoxyphenyl)piperazin-1-yl][2-phenyl-5-(4-trifluoromethylphenyl)-2H-pyrazol-3-yl]methanone in the form of a yellow solid, the characteristics of which are as follows:
LC/MS analysis: tr=4.76; m/z=537 (MH⁺)

### EXAMPLE E90

### [4-(3,5-Dimethoxyphenyl)piperazin-1-yl][5-(furan-3-yl)-2-phenyl-2H-pyrazol-3-yl]methanone

The process is performed in a manner similar to that of Example E66, starting with 150 mg of the product of Example E82 and 51 mg of furyl-3-boronic acid, by microwave reaction for 3 minutes at 140° C., to obtain, after purification by flash chromatography on a column of silica (30-60 μm), eluting with a mixture of heptane and ethyl acetate (70/30 by volume), 137 mg of [4-(3,5-dimethoxyphenyl)piperazin-1-yl][5-(furan-3-yl)-2-phenyl-2H-pyrazol-3-yl]-methanone in the form of a pale yellow solid, the characteristics of which are as follows:
LC/MS analysis: tr=4.17; m/z=459 (MH⁺)

### EXAMPLE E91

### [4-(3,5-Dimethoxyphenyl)piperazin-1-yl][2-phenyl-5-(1H-pyrrol-2-yl)-2H-pyrazol-3-yl]methanone

The process is performed in a manner similar to that of Example E66, starting with 153 mg of the product of Example E82 and 105 mg of 1-(t-butoxycarbonyl)pyrrole-2-boronic acid, by microwave reaction for 3 minutes at 140° C., to obtain, after purification by flash chromatography on a column of silica (30-60 μm), eluting with a mixture of heptane and ethyl acetate (70/30 by volume), 68 mg of [4-(3,5-dimethoxyphenyl)piperazin-1-yl][2-phenyl-5-(1H-pyrrol-2-yl)-2H-pyrazol-3-yl]methanone in the form of a pale yellow solid, the characteristics of which are as follows:
LC/MS analysis: tr=4.11; m/z=458 (MH⁺)
¹H NMR spectrum (300 MHz, (CD₃)₂SO d6, δ in ppm): 2.80 ppm (m, 2H); 3.10 ppm (m, 2H); 3.36 ppm (masked, 2H); 3.67 ppm (m, 2H); 3.68 ppm (s, 6H); 5.99 ppm (t, J=2 Hz, 1H); 6.02 ppm (d, J=2 Hz, 2 H); 6.12 ppm (m, 1H); 6.51 ppm (m, 1H); 6.83 ppm (m, 1H); 6.92 ppm (s, 1H); 7.39 ppm (m, 1H); 7.52 ppm (m, 4H); 11.35 ppm (bs, 1H).

### EXAMPLE E92

### [4-(3,5-Dimethoxyphenyl)piperazin-1-yl](2-phenyl-2H-pyrazol-3-yl)methanone

36 μl of pyrrolidine, 11 mg of sodium tert-butoxide and 0.20 ml of THF are added to 50 mg of the product of Example E82, 10.5 mg of tris(dibenzylideneacetone)dipalladium(0) and 42 mg of 2-dicyclohexylphosphino-2′-(N,N-dimethylamino)biphenyl in 0.30 ml of THF in a microwave reactor. The reaction mixture is subjected to the microwave field for 10 minutes at 80° C. and then poured into 10 ml of saturated aqueous sodium dihydrogen phosphate solution and extracted with ethyl acetate. The organic phase is dried over magnesium sulfate and concentrated under reduced pressure. After purification by preparative HPLC/MS (H₂O pH=9/CH₃CN), 6 mg of [4-(3,5-dimethoxyphenyl)piperazin-1-yl](2-phenyl-2H-pyrazol-3-yl)methanone are obtained in the form of a pale yellow powder, the characteristics of which are as follows:
LC/MS analysis: tr=3.65; m/z=393 (MH⁺)
¹H NMR spectrum (300 MHz, (CD₃)₂SO d6, δ in ppm): 2.78 ppm (m, 2H); 3.08 ppm (m, 2H); 3.31 ppm (masked, 2H); 3.69 ppm (m, 2H); 3.70 ppm (s, 6H); 5.99 ppm (t, J=2 Hz, 1H); 6.01 ppm (d, J=2 Hz, 2H); 6.73 ppm (d, J=2 Hz, 1H); 7.40 ppm (m, 1H); from 7.46 to 7.55 ppm (m, 4H); 7.81 ppm (d, J=2 Hz, 1H).

### EXAMPLE E93

### [4-(3,5-Dimethoxyphenyl)piperazin-1-yl][2-phenyl-5-(pyrrolidin-1-yl)-2H-pyrazol-3-yl]methanone

36 μl of pyrrolidine, 11 mg of sodium tert-butoxide and 0.20 ml of THF are added to 50 mg of the product of Example E82, 10.5 mg of tris(dibenzylideneacetone)dipalladium(0) and 42 mg of 2-(di-tert-butyl-phosphino)biphenyl in 0.30 ml of THF in a microwave reactor. The reaction mixture is subjected to the microwave field for 10 minutes at 80° C. and then poured into 10 ml of saturated aqueous sodium dihydrogen phosphate solution and extracted with ethyl acetate. The organic phase is dried over magnesium sulfate and concentrated under reduced pressure. After purification by preparative HPLC/MS, (H₂O pH=9/CH₃CN), 23 mg of [4-(3,5-dimethoxyphenyl)piperazin-1-yl][2-phenyl-5-(pyrrolidin-1-yl)-2H-pyrazol-3-yl]methanone are obtained in the form of a pale yellow powder, the characteristics of which are as follows:
LC/MS analysis: tr=4.13; m/z=462 (MH⁺)

### EXAMPLE E94

### 5-[4-(3,5-Dimethoxyphenyl)piperazine-1-carbonyl]-1-phenyl-1H-pyrazole-3-carboxaldehyde oxime E

The process is performed in a manner similar to that of Step 1 of Example E95, starting with 440 mg of the product of Example E100 and 80 mg of hydroxylamine hydrochloride, to give 401 mg of 5-[4-(3,5-dimethoxyphenyl)piperazine-1-carbonyl]-1-phenyl-1H-pyrazole-3-carboxaldehyde oxime E in the form of a white foam, the characteristics of which are as follows:
LC/MS analysis: tr=3.99; m/z=436 (MH⁺)
¹H NMR spectrum (400 MHz, (CD₃)₂SO d6, δ in ppm): 2.81 ppm (m,2H); 3.12 ppm (m, 2H); 3.36 ppm (m, 2H); 3.68 ppm (m, 2 H); 3.70 ppm (s, 6H); 5.98 ppm (t, J=2 Hz, 1H); 6.02 ppm (d, J=2 Hz, 2H); 6.92 ppm (s, 1H); from 7.38 to 7.46 ppm (m, 5H); 8.15 ppm (s, 1H); 11.46 ppm (bs, 1H).

### EXAMPLE E95

### 5-[4-(3,5-Dimethoxyphenyl)piperazine-1-carbonyl]-1-phenyl-1H-pyrazole-3-carboxaldehyde oxime Z

Step 1: 94 mg of hydroxyamine hydrochloride are added, at 0° C., to 300 mg of the product of Step 1 of Example E70 in 4 ml of ethanol and 120 μl of pyridine. The reaction mixture is stirred for 1 hour at room temperature, poured into 10 ml of saturated aqueous sodium dihydrogen phosphate solution and extracted with ethyl acetate. The organic phase is dried over magnesium sulfate and concentrated under reduced pressure. After purification by flash chromatography on a column of silica (40-63 μm), eluting with a mixture of dichloromethane and ethyl acetate (90/10 by volume), 115 mg of ethyl 5Z-oximino-2-phenylpyrazole-3-carboxylate are obtained in the form of a white solid, the characteristics of which are as follows:
¹H NMR spectrum (300 MHz, (CD₃)₂SO d6, δ in ppm): 1.17 ppm (t, J=7 Hz, 3H); 4.19 (q, J=7 Hz, 2H); 7.63 ppm (s, 1H); 7.50 ppm (m, 5 Hz); 7.60 ppm (s, 1H); 11.90 ppm (bs, 1H).

Step 2: The process is performed in a manner similar to that of Step 1 of Example E22, starting with 183 mg of the product of Step 1 of the present example and 330 mg of 1-(3,5-dimethoxyphenyl)piperazine, for 5 hours at 80° C., to give, after purification by preparative HPLC/MS (H₂O pH=9/CH₃CN), 37 mg of 5-[4-(3,5-dimethoxyphenyl)piperazine-1-carbonyl]-1-phenyl-1H-pyrazole-3-carboxaldehyde oxime Z, in the form of a white powder, the characteristics of which are as follows:
LC/MS analysis: tr=3.63; m/z=436 (MH⁺)
¹H NMR spectrum (400 MHz, (CD₃)₂SO d6, δ in ppm): 2.94 ppm (m, 2H); 3.10 ppm (m, 2H); 3.37 ppm (masked, 2H); 3.68 ppm (m, 2H); 3.69 ppm (s, 6H); 5.99 ppm (bs, 1H); 6.02 ppm (bs, 2H); from 7.42 to 7.57 ppm (m, 5H); 7.28 ppm (s, 1H); 7.60 ppm (s, 1H); 11.8 ppm (bs, 1H).

### EXAMPLE E96

### [4-(3,5-Dimethoxyphenyl)piperazin-1-yl][5-(morpholin-4-yl)-2-phenyl-2H-pyrazol-3-yl]methanone

The process is performed in a manner similar to that of Example E93, starting with 100 mg of the product of Example E82 and 116 μl of morpholine in 1,2-dimethoxyethane (DME), by microwave reaction for 10 minutes at 80° C., to give, after purification by preparative HPLC/MS (H₂O pH=9/CH₃CN), 91 mg of [4-(3,5-dimethoxyphenyl)piperazin-1-yl][5-(morpholin-4-yl)-2-phenyl-2H-pyrazol-3-yl]methanone in the form of a pale yellow solid, the characteristics of which are as follows:
LC/MS analysis: tr=3.80; m/z=478 (MH⁺)

### EXAMPLE E97

### 3-[4-(5-Methyl-2-pyrid-3-yl-2H-pyrazole-3-carbonyl)piperazin-1-yl]benzamide

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 278 mg of 3-(piperazin-1-yl)benzamide, which may be obtained according to patent WO 98/00400 and 5-methyl-2-phenyl-2H-pyrazole-3-carboxylic acid, with 223.5 mg of 5-methyl-2-pyrid-3-yl-2H-pyrazole-3-carboxylic acid, which may be obtained according to J. Het. Chem., 36, 217 (1999), 300 mg of 3-[4-(5-methyl-2-pyrid-3-yl-2H-pyrazole-3-carbonyl)piperazin-1-yl]benzamide are obtained in the form of a pale yellow foam, the characteristics of which are as follows:
Mass spectrum (EI): m/z=390 (M+)

### EXAMPLE E98

### (5-Benzylamino-2-phenyl-2H-pyrazol-3-yl)[4-(3,5-dimethoxyphenyl)piperazin-1-yl]methanone

The process is performed in a manner similar to that of Example E93, starting with 100 mg of the product of Example E82, 21 mg of tris(dibenzylideneacetone)dipalladium(0), and 64 mg of 2-(di-tert-butyl-phosphino)biphenyl in 0.60 ml of DME, to which are added 116 μl of benzylamine, 30.5 mg of sodium tert-butoxide and 0.40 ml of DME. The reaction mixture is subjected to the microwave field for 5 minutes at 90° C., 10 mg of tris(dibenzylideneacetone)dipalladium(0) are added and the mixture is subjected to the microwave field for a further 3 minutes at 100° C. After work-up and purification by preparative HPLC/MS (H₂O pH=9/CH₃CN), 38 mg of (5-benzylamino-2-phenyl-2H-pyrazol-3-yl)[4-(3,5-dimethoxyphenyl)-piperazin-1-yl]methanone are obtained in the form of a white powder, the characteristics of which are as follows:
LC/MS analysis: tr=4.19; m/z=498 (MH⁺)

### EXAMPLE E99

### (5-Amino-2-phenyl-2H-pyrazol-3-yl)[4-(3,5-dimethoxyphenyl)piperazin-1-yl]methanone

Step 1: The process is performed in a manner similar to that of Example E98, starting with 100 mg of the product of Example E82 and 142 μl of benzophenoneimine. The reaction mixture is subjected to the microwave field for 12 minutes at 80° C., 10 mg of tris(dibenzylideneacetone)dipalladium(0) and 100 μl of DME are added and the mixture is subjected to the microwave field for a further 2 minutes at 90° C. After work-up and purification by flash chromatography on a column of silica (40-63 μm), eluting with a mixture of heptane and ethyl acetate (70/30 by volume), 66 mg of [5-(benzhydrylidene-amino)-2-phenyl-2H-pyrazol-3-yl][4-(3,5-dimethoxyphenyl)piperazin-1-yl]-methanone are obtained in the form of a yellow solid, the characteristics of which are as follows:
Mass spectrum (EI): m/z=571 (M⁺)

Step 2: 131 mg of ammonium formate and 60 mg of 10% palladium-on-charcoal are added to 66 mg of the product of Step 1 of the present example in 2.5 ml of methanol, in a microwave reactor. The reaction mixture is subjected to the microwave field for 3 minutes at 100° C., filtered, concentrated under reduced pressure and then purified by flash chromatography on a column of silica (30-60 μm), eluting with a mixture of dichloromethane and ethyl acetate (70/30 by volume), to give 29 mg of (5-amino-2-phenyl-2H-pyrazol-3-yl)[4-(3,5-dimethoxyphenyl)piperazin-1-yl]methanone in the form of a pale yellow solid, the characteristics of which are as follows:
LC/MS analysis: tr=3.30; m/z=408 (MH⁺)

### EXAMPLE E100

### 5-[4-(3,5-Dimethoxyphenyl)piperazine-1-carbonyl]-1-phenyl-1H-pyrazole-3-carboxaldehyde

27 mg of calcium carbonate and 2 ml of water are added to 150 mg of the product of Example E78 in 2 ml of 1,4-dioxane, in a microwave reactor. The reaction mixture is subjected to the microwave field for 10 minutes at 120° C., acidified to pH 5 by controlled addition of aqueous 1M hydrochloric acid solution and extracted with ethyl acetate. The organic phase is dried over magnesium sulfate and concentrated under reduced pressure. After purification by flash chromatography on a column of silica (30-60 μm), eluting with a mixture of dichloromethane and ethyl acetate (95/5 by volume), 98 mg of 5-[4-(3,5-dimethoxyphenyl)piperazine-1-carbonyl]-1-phenyl-1H-pyrazole-3-carboxaldehyde are obtained in the form of a white solid, the characteristics of which are as follows:
LC/MS analysis: tr=3.73; m/z=421 (MH⁺)

### EXAMPLE E101

### [4-(3-Chlorophenyl)piperazin-1-yl](5-phenoxymethyl-2-phenyl-2H-pyrazol-3-yl)methanone

Step 1: 0.58 ml of 1M sodium hydroxide is added to a solution of 68 mg of phenol in 1.5 ml of acetone. The reaction mixture is stirred for 45 minutes at room temperature and then treated with a solution of 150 mg of ethyl 5-bromomethyl-2-phenyl-2H-pyrazole-3-carboxylate, obtained in Step 1 of Example E73, in 1.5 ml of acetone. The reaction mixture is stirred for 5 hours at room temperature and then poured into 20 ml of saturated aqueous sodium dihydrogen phosphate solution and extracted with ethyl acetate. The organic phase is dried over magnesium sulfate and concentrated under reduced pressure. After purification by flash chromatography on a column of silica (30-60 μm), eluting with a mixture of dichloromethane and heptane (70/30 by volume) and then with a mixture of dichloromethane, ethyl acetate and acetic acid (70/30/1 by volume), 73 mg of ethyl 5-phenoxymethyl-2-phenyl-2H-pyrazole-3-carboxylate are obtained in the form of a colorless oil, the characteristics of which are as follows:
Mass spectrum (ES): m/z=323 (MH⁺)

Step 2: The process is performed in a manner similar to that of Step 2 of Example E75, starting with 73 mg of the product of Step 1 of the present example and 179 mg of 1-(3-chlorophenyl)piperazine, for 3 hours at 60° C., to give, after purification by flash chromatography on a column of silica (30-60 μm), eluting with a mixture of dichloromethane and ethyl acetate (95/5 by volume), 78 mg of [4-(3-chlorophenyl)piperazin-1-yl](5-phenoxymethyl-2-phenyl-2H-pyrazol-3-yl)methanone in the form of a white solid, the characteristics of which are as follows:
LC/MS analysis: tr=4.59; m/z=473 (MH⁺)

### EXAMPLE E102

### [4-(3-Chlorophenyl)piperazin-1-yl](2-phenyl-5-phenylsulfanylmethyl-2H-pyrazol-3-yl)methanone

Step 1: A solution of 73 mg of potassium tert-butoxide in 1 ml of THF is added to a solution of 66 μl of thiophenol in 2 ml of THF, cooled to 5° C. The reaction mixture is stirred for 10 minutes at 10° C. and then treated with a solution of 200 mg of ethyl 3-bromomethyl-1-phenyl-1H-pyrazole-5-carboxylate, obtained in Step 1 of Example E73, in 2 ml of THF. The reaction mixture is stirred for 16 hours at room temperature and then poured into 50 ml of saturated aqueous sodium dihydrogen phosphate solution and extracted with ethyl acetate. The organic phase is dried over magnesium sulfate and concentrated under reduced pressure. After purification by flash chromatography on a column of silica (40-63 μm), eluting with a mixture of toluene and ethyl acetate (97/3 by volume), 235 mg of ethyl 2-phenyl-5-phenylsulfanylmethyl-2H-pyrazole-3-carboxylate are obtained in the form of a pale yellow oil, the characteristics of which are as follows:
Mass spectrum (ES): m/z=339 (MH³⁰ )

Step 2: The process is performed in a manner similar to that of Step 2 of Example E75, starting with 100 mg of the product of Step 1 of the present example and 232 mg of 1-(3-chlorophenyl)piperazine, for 2 hours at 60° C., to give, after purification by flash chromatography on a column of silica (30-60 μm), eluting with a mixture of dichloromethane and ethyl acetate (98/2 by volume), 114 mg of [4-(3-chlorophenyl)piperazin-1-yl](2-phenyl-5-phenylsulfanylmethyl-2H-pyrazol-3-yl)methanone are obtained in the form of a pale yellow solid, the characteristics of which are as follows:
LC/MS analysis: tr=4.65; m/z=489 (MH⁺)

### EXAMPLE E103

### {4-[3-(2-Hydroxyethylamino)phenyl]piperazin-1-yl}-(5-methyl-2-phenyl-2H-pyrazol-3-yl)methanone

Starting with 361 mg of [4-(3-aminophenyl)piperazin-1-yl](5-methyl-2-phenyl-2H-pyrazol-3-yl)methanone, described in Example 9, in 2 ml of toluene, 211 μl of triethylamine and 158 μl of 2-iodoethanol are added. After refluxing for 20 hours, 25 ml of ethyl acetate and 25 ml of water are added and the organic phase is separated out by settling and then washed with twice 25 ml of water, dried over magnesium sulfate and concentrated under reduced pressure. After purification by flash chromatography on a column of silica (60; 35-70 μm), eluting with a mixture of cyclohexane and ethyl acetate (80-20 by volume), 0.2 g of {4-[3-(2-hydroxyethylamino)phenyl]piperazin-1-yl}-(5-methyl-2-phenyl-2H-pyrazol-3-yl)methanone is obtained in the form of an orange-colored foam, the characteristics of which are as follows:
Mass spectrum (EI): m/z=405 (M⁺)
¹H NMR spectrum (300 MHz, (CD₃)₂SO d6, δ in ppm): 2.31 (s: 3H); 2.71 (unresolved complex: 2H); 3.02 (unresolved complex: 2H); 3.06 (q, J=6 Hz: 2H); 3.32 (unresolved complex: 2H); 3.54 (broad q, J=6 Hz: 2H); 3.68 (unresolved complex: 2H); 4.65 (broad t, J=6 Hz: 1H); 5.28 (t, J=6 Hz: 1H); from 6.05 to 6.15 (mt: 3H); 6.52 (s: 1H); 6.91 (broad t, J=8 Hz: 1H); 7.38 (broad t, J=7.5 Hz: 1H); from 7.40 to 7.55 (mt: 4H).

### EXAMPLE E104

### (5-Methyl-2-phenyl-2H-pyrazol-3-yl)[4-(thiophen-3-yl)piperazin-1-yl]-methanone

Step 1: 5.2 g of 1-(3-dimethylaminopropyl)-3-ethylcarbodiimide hydrochloride (EDCl), 3.3 g of 1-hydroxybenzotriazole hydrate (HOBT) and 5 g of 5-methyl-2-phenyl-2H-pyrazole-3-carboxylic acid are added to a solution of 4.6 g of tert-butyl piperazine-1-carboxylate in 100 ml of dichloromethane. The reaction mixture is stirred for 20 hours at room temperature. After addition of 50 ml of dichloromethane and 50 ml of saturated aqueous sodium bicarbonate solution, the organic phase is separated out by settling of the phases, and then dried over magnesium sulfate and concentrated under reduced pressure. 8.1 g of tert-butyl 4-(5-methyl-2-phenyl-2H-pyrazole-3-carbonyl)piperazine-1-carboxylate are thus obtained in the form of a white solid, the characteristics of which are as follows:
Melting point (Kofler): 150° C.
¹H NMR spectrum (300 MHz, (CD₃)₂SO d6, δ in ppm): 1.40 (s: 9H); 2.29 (s: 3H); 3.03 (unresolved complex: 2H); 3.23 (unresolved complex: 2H); 3.32 (unresolved complex: 2H); 3.54 (unresolved complex: 2H); 6.50 (s: 1H); from 7.30 to 7.55 (mt: 5H).

Step 2: 27 ml of a 4N solution of hydrochloric acid in dioxane are added dropwise to a solution of 8 g of tert-butyl 4-(5-methyl-2-phenyl-2H-pyrazole-3-carbonyl)piperazine-1-carboxylate in 50 ml of dichloromethane. After reaction for 20 hours and concentration under reduced pressure, the residue is taken up in 1N sodium hydroxide solution to pH 10, extracted with 50 ml of ethyl acetate, dried over sodium sulfate and concentrated under reduced pressure. 2.96 g of (5-methyl-2-phenyl-2H-pyrazol-3-yl)(piperazin-1-yl)methanone are thus obtained in the form of a white solid, the characteristics of which are as follows:
Melting point (Kofler): 138° C.
¹H NMR spectrum (300 MHz, (CD₃)₂SO d6, δ in ppm): of 2.20 to 2.35 (mt: 2H); 2.29 (s: 3H); 2.60 (broad t, J=4.5 Hz: 2H); 3.06 (broad t, J=4.5 Hz: 2H); 3.48 (broad t, J=4.5 Hz: 2H); 6.45 (s: 1H); from 7.30 to 7.55 (mt: 5H).

Step 3: 121 mg of 3-bromothiophene, 46 mg of (R)(+)-2,2′-bis(diphenylphosphino)-1,1′-binaphthyl, 17 mg of palladium acetate and 71 mg of sodium tert-butoxide are added to a solution of 200 mg of (5-methyl-2-phenyl-2H-pyrazol-3-yl)(piperazin-1-yl)methanone in 6 ml of toluene. After heating at 90° C. for 20 hours, the insoluble material is filtered off, 50 ml of ethyl acetate and the organic phase is separated out by settling of the phases and then washed with 3 times 10 ml of water, dried over magnesium sulfate and concentrated under reduced pressure. After purification by flash chromatography on a column of silica (60; 35-70 μm), eluting with a mixture of cyclohexane and ethyl acetate (70-30 by volume), 18 mg of (5-methyl-2-phenyl-2H-pyrazol-3-yl)[4-(thiophen-3-yl)piperazin-1-yl]methanone are obtained in the form of an amorphous yellow solid, the characteristics of which are as follows:
¹H NMR spectrum (300 MHz, (CD₃)₂SO d6, δ in ppm): 2.31 (s: 3H); 2.74 (unresolved complex: 2H); 3.01 (unresolved complex: 2H); 3.34 (unresolved complex: 2H); 3.69 (unresolved complex: 2H); 6.33 (dd, J=3 and 1.5 Hz: 1H); 6.52 (s: 1H); 6.93 (dd, J=5.5 and 1.5 Hz: 1H); 7.37 (tt, J=7.5 and 1.5 Hz: 1H); from 7.40 to 7.55 (mt: 4H); 7.42 (dd, J=5.5 and 3 Hz: 1H).

### EXAMPLE E105

This example is described with Example E84.

### EXAMPLE E106

### [4-(3,5-Dimethoxyphenyl)piperazin-1-yl][5-(3-hydroxypyrrolidin-1-yl)-2-phenyl-2H-pyrazol-3-yl]methanone

The process is performed in a manner similar to that of Example E98, starting with 100 mg of the product of Example E82 and 90 μl of 3-pyrrolidinol, by microwave reaction for 5 minutes at 90° C., to give, after purification by preparative HPLC/MS (H₂O pH=9/CH₃CN), 48 mg of [4-(3,5-dimethoxyphenyl)piperazin-1-yl][5-(3-hydroxypyrrolidin-1-yl)-2-phenyl-2H-pyrazol-3-yl]-methanone in the form of a white powder, the characteristics of which are as follows:
LC/MS analysis: tr=3.47; m/z=478 (MH⁺)

### EXAMPLE E107

### 1-{3-[4-(5-Methyl-2-phenyl-2H-pyrazole-3-carbonyl)piperazin-1-yl]phenyl}ethanone

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 82 mg of 1-[3-(piperazin-1-yl)phenyl]ethanone, which may be obtained according to patent WO 02/088107, 21.8 mg of 1-{3-[4-(5-methyl-2-phenyl-2H-pyrazole-3-carbonyl)piperazin-1-yl]phenyl}ethanone are obtained in the form of a white solid, the characteristics of which are as follows:
Melting point (Kofler): 134° C.
Mass spectrum (EI): m/z=388 (M+)

### EXAMPLE E108

This example is described with Example E84

### EXAMPLE E109

This example is described with Example E84

### EXAMPLE E110

### N-(2-Methylaminoethyl)-3-[4-(5-methyl-2-phenyl-2H-pyrazole-3-carbonyl)piperazin-1-yl]benzamide

Step 1: 763 mg of potassium hydroxide pellets are added to a solution of 4.4 g of ethyl 3-[4-(5-methyl-2-phenyl-2H-pyrazole-3-carbonyl)piperazin-1-yl]-benzoate, described in Example E28, in 75 ml of distilled-water and 150 ml of methanol. After 20 hours at room temperature, the reaction mixture is concentrated under reduced pressure and the residue is acidified to pH 5 with 5N hydrochloric acid. After filtering off the solid formed, 3.9 g of 3-[4-5-methyl-2-phenyl-2H-pyrazole-3-carbonyl)piperazin-1-yl]benzoic acid are thus obtained in the form of a pale yellow solid, the characteristics of which are as follows:
Melting point (Kofler): 206° C.
Mass spectrum (EI): m/z=390 (M⁺)

Step 2: 211 mg of 1-(3-dimethylaminopropyl)-3-ethylcarbodiimide hydrochloride (EDCl), 148 mg of 1-hydroxybenzotriazole hydrate (HOBT) and 175 mg of tert-butyl (2-aminoethyl)methylcarbamate are added to a solution of 390.5 mg of 3-[4-(5-methyl-2-phenyl-2H-pyrazole-3-carbonyl)piperazin-1-yl]benzoic acid in 17 ml of dichloromethane. The reaction mixture is stirred for 72 hours at room temperature. After addition of 25 ml of dichloromethane and 25 ml of water, the organic phase is separated out by settling of the phases and then washed with 25 ml of saturated aqueous sodium bicarbonate solution, dried over magnesium sulfate and concentrated under reduced pressure. After purification by flash chromatography on a column of silica (60; 35-70 μm), eluting with a mixture of cyclohexane and ethyl acetate (70-30 by volume), 500 mg of tert-butyl methyl(2-{3-[4-(5-methyl-2-phenyl-2H-pyrazole-3-carbonyl)piperazin-1-yl]benzoylamino}ethyl)carbamate are obtained in the form of a beige-colored foam, the characteristics of which are as follows:
Mass spectrum (EI): m/z=546 (M⁺)

Step 3: 1 ml of a 4N solution of hydrochloric acid in dioxane is added dropwise to a solution of 440 mg of tert-butyl methyl-(2-{3-[4-(5-methyl-2-phenyl-2H-pyrazole-3-carbonyl)piperazin-1-yl]benzoylamino}ethyl)carbamate in 1 ml of dioxane. After reaction for 20 hours and concentration under reduced pressure, 440 mg of N-(2-methylaminoethyl)-3-[4-(5-methyl-2-phenyl-2H-pyrazole-3-carbonyl)piperazin-1-yl]benzamide are thus obtained in the form of an amorphous yellow solid, the characteristics of which are as follows:
Mass spectrum (EI): m/z=446 (M+)
¹H NMR spectrum (300 MHz, (CD₃)₂SO d6, δ in ppm): 2,32 (s: 3H); 2,60 (t, J=5 Hz: 3H); 2,93 (unresolved complex: 2H); 3,09 (mt: 2H); 3,20 (unresolved complex: 2H); 3,40 (mt: 2H); 3,58 (unresolved complex: 2H); 3,73 (unresolved complex: 2H); 6,55 (s: 1 H); 7,08 (broad d, J=8 Hz: 1H); from 7,25 à 7,55 (mt: 8H); 8,77 (broad t, J=5 Hz: 1H); 8,89 (unresolved complex: 2H).

### EXAMPLE E111

### (5-Methyl-2-phenyl-2H-pyrazol-3-yl)[4-(3,4,5-trifluorophenyl)piperazin-1-yl]methanone

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 200 mg of 1-(3,4,5-trifluorophenyl)piperazine, which may be obtained from 3,4,5-trifluorobromobenzene by working in a manner similar to that for the synthesis of 1-[3-(4-benzylpiperazin-1-yl)phenyl]ethanone of Step 1 of Example E39—and the characteristics of which are as follows: ¹H NMR spectrum (300 MHz, (CD₃)₂SO d6, δ in ppm): 3.19 (unresolved complex: 4H); 3.43 (broad t, J=5.5 Hz: 4H); 6.96 (dd, J=12 and 6.5 Hz: 2H); 9.23 (unresolved complex: 2H).—, 115 mg of (5-methyl-2-phenyl-2H-pyrazol-3-yl)[4-(3,4,5-trifluorophenyl)piperazin-1-yl]methanone are obtained in the form of a white solid, the characteristics of which are as follows:
Melting point (Kofler): 204° C.
Mass spectrum (EI): m/z=400 (M+)

### EXAMPLE E 112

### Ethyl E-3-{5-[4-(3,5-dimethoxyphenyl)piperazine-1-carbonyl]-1-phenyl-1H-pyrazol-3-yl}acrylate

195 mg of (carbethoxymethylene)triphenylphosphorane are added, at room temperature, to a solution of 196 mg of 5-[4-(3,5-dimethoxyphenyl)piperazine-1-carbonyl]-1-phenyl-1H-pyrazole-3-carboxaldehyde, obtained in Example E100, in 3 ml of THF. After stirring for 12 hours at 50° C., the reaction mixture is concentrated under reduced pressure and purified by flash chromatography on a column of silica (40-63 μm), eluting with a mixture of dichloromethane and ethyl acetate (95/5 by volume), to give 130 mg of ethyl E-3-{5-[4-(3,5-dimethoxyphenyl)piperazine-1-carbonyl]-1-phenyl-1H-pyrazol-3-yl]acrylate in the form of a white solid, the characteristics of which are as follows:
LC/MS analysis: tr=4.16; m/z=491 (MH⁺)

### EXAMPLE E113

### 3-[4-(5-Methoxymethoxymethyl-2-phenyl-2H-pyrazole-3-carbonyl)piperazin-1-yl]benzamide

Step 1: 455 mg of sodium hydride at 60% in liquid petroleum jelly and 1.15 g of chloromethoxymethane are added, in the region of 0° C., to a solution of 1.4 g of ethyl 5-hydroxymethyl-2-phenyl-2H-pyrazole-3-carboxylate, which is obtained as a side product in the synthesis of Example 71, in 35 ml of tetrahydrofuran. After reaction for 20 hours at room temperature, 50 ml of water and 50 ml of ethyl acetate are added and the organic phase is separated out by settling of the phases, washed with water, dried over magnesium sulfate and concentrated under reduced pressure. After purification by flash chromatography on a column of silica (60; 35-70 μm), eluting with pure dichloromethane, 1.1 g of ethyl 5-methoxymethoxymethyl-2-phenyl-2H-pyrazole-3-carboxylate are obtained in the form of an orange-colored oil, the characteristics of which are as follows.
Mass spectrum (EI): m/z=290 (M+)

Step 2: 6 ml of water and 250 mg of potassium hydroxide are added to a solution of 1.1 g of ethyl 5-methoxymethoxymethyl-2-phenyl-2H-pyrazole-3-carboxylate in 12 ml of ethanol. After 20 hours at room temperature, the mixture is concentrated under reduced pressure, taken up in aqueous 1N hydrochloric acid solution to pH 1 and extracted with 3 times 25 ml of dichloromethane. The organic phase is separated out by settling of the phases, washed with 25 ml of water, dried over magnesium sulfate and concentrated under reduced pressure. 500 mg of 5-methoxymethoxymethyl-2-phenyl-2H-pyrazole-3-carboxylic acid are thus obtained in the form of an orange-colored oil, the characteristics of which are as follows:
Mass spectrum (EI): m/z=262 (M+)

Step 3: By performing the process in a manner similar to that for the synthesis of [4-(3-chlorophenyl)piperazin-1-yl](5-methyl-2-phenyl-2H-pyrazol-3-yl)methanone, but replacing the 1-(3-chlorophenyl)piperazine with 556 mg of 3-piperazin-1-ylbenzamide, which may be obtained according to patent WO 98/00400, and by replacing the 5-methyl-2-phenyl-2H-pyrazole-3-carboxylic acid with 525 mg of 5-methoxymethoxymethyl-2-phenyl-2H-pyrazole-3-carboxylic acid, 900 mg of 3-[4-(5-methoxymethoxymethyl-2-phenyl-2H-pyrazole-3-carbonyl)piperazin-1-yl]benzamide are obtained in the form of an amorphous beige-colored solid, the characteristics of which are as follows:
Mass spectrum (EI): m/z=449 (M+)

### EXAMPLE E114

### 3-{5-[4-(3,5-Dimethoxyphenyl)piperazine-1-carbonyl]-1-phenyl-1H-pyrazol-3-yl}thiophene-2-carboxaldehyde

The process is performed in a manner similar to that of Example E66, starting with 86 mg of the product of Example E82 and 40 mg of 2-formyl-3-thiopheneboronic acid. After microwave reaction for 3 minutes at 140° C., 6 mg of tetrakis(triphenylphosphine)palladium(0) and 0.2 ml of DMF are added and the mixture is subjected to the microwave field for a further 1 minute at 140° C., to give, after work-up and successive purifications by flash chromatography on a column of silica (30-60 μm), eluting with a mixture of heptane and ethyl acetate (6/4 by volume) and then by preparative HPLC/MS (H₂O pH=9/CH₃CN), 33 mg of 3-{5-[4-(3,5-dimethoxyphenyl)piperazine-1-carbonyl]-1-phenyl-1H-pyrazol-3-yl}thiophene-2-carboxaldehyde in the form of a pale yellow powder, the characteristics of which are as follows:
LC/MS: tr=4.63; m/z=503 (MH⁺)

### EXAMPLE E116

### N-Methyl-3-[4-(5-methyl-2-phenyl-2H-pyrazole-3-carbonyl)piperazin-1-yl]-benzamide

Step 1: A solution of 0.86 ml of oxalyl chloride is added, at 0° C., to a solution of 3.1 g tert-butyl 4-(3-carboxyphenyl)piperazine-1-carboxylate, which may be obtained according to patent GB 2 327 609, in 15 ml of dioxane. One drop of dimethylformamide is added to this reaction mixture. After stirring for 2 hours at a temperature in the region of 20° C., the reaction mixture is added dropwise to 50 ml of aqueous 40% methylamine solution. After stirring for 1 hour at room temperature, the reaction mixture is taken up in 200 ml of dichloromethane and then washed twice with 50 ml of distilled water. The organic phase is dried over magnesium sulfate and then concentrated to dryness under reduced pressure. 1.5 g of tert-butyl 4-(3-methylcarbamoylphenyl)piperazine-1-carboxylate are thus obtained in the form of an orange-colored oil, the characteristics of which are as follows:
¹H NMR spectrum (300 MHz, (CD₃)₂SO d6, δ in ppm): 1.45 (s: 9H); 2.78 (d, J=4.5 Hz: 3H); 3.16 (broad t, J=5.5 Hz: 4H); 3.49 (broad t, J=5.5 Hz: 4H); 7.11 (broad dt, J=7.5 and 2 Hz: 1H); from 7.20 to 7.35 (mt: 2H); 7.39 (broad s: 1H); 8.32 (broad q, J=4.5 Hz: 1H).

Step 2: 5.9 ml of a 4N solution of hydrochloric acid in dioxane are added dropwise to a solution of 1.5 g of tert-butyl 4-(3-methylcarbamoylphenyl)-piperazine-1-carboxylate in 5.8 ml of dioxane. After reaction for 6 hours at room temperature and concentration under reduced pressure, 950 mg of N-methyl-3-(piperazin-1-yl)benzamide hydrochloride are thus obtained in the form of an amorphous brown solid, the characteristics of which are as follows:
1H NMR spectrum (300 MHz, (CD₃)₂SO d6, δ in ppm): 2.79 (d, J=4.5 Hz: 3H); 3.15 (unresolved complex: 4H); 3.44 (broad t, J=5.5 Hz: 4H); 7.15 (mt: 1H); from 7.25 to 7.40 (mt: 2H); 7.44 (broad s: 1H); 8.42 (broad q, J=4.5 Hz: 1H); 9.17 (unresolved complex: 2H).

Step 3: By performing the process in a manner similar to that for the synthesis of [4-(3-chlorophenyl)piperazin-1-yl](5-methyl-2-phenyl-2H-pyrazol-3-yl)-methanone (Example 1), but replacing the 1-(3-chlorophenyl)piperazine with 584 mg of N-methyl-3-(piperazin-1-yl)benzamide, 450 mg of N-methyl-3-[4-(5-methyl-2-phenyl-2H-pyrazole-3-carbonyl)piperazin-1-yl]benzamide are obtained in the form of a beige-colored solid, the characteristics of which are as follows:
Melting point (Kofler): 180° C.
Mass spectrum (EI): m/z=403 (M+)

### EXAMPLE E117

### [4-(3,5-Dimethoxyphenyl)piperazin-1-yl](2-phenyl-5-trifluoromethyl-2H-pyrazol-3-yl)methanone

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine by 244.5 mg of 1-(3,5-dimethoxyphenyl)piperazine and replacing the 5-methyl-2-phenyl-2H-pyrazole-3-carboxylic acid with 256 mg of 2-phenyl-5-trifluoromethyl-2H-pyrazole-3-carboxylic acid, which may be obtained according to patent WO 03/024222, 340 mg of [4-(3,5-dimethoxyphenyl)piperazin-1-yl](2-phenyl-5-trifluoromethyl-2H-pyrazol-3-yl)methanone are obtained in the form of a white solid, the characteristics of which are as follows:
Melting point (Kofler): 84° C.
Mass spectrum (EI): m/z=460 (M+)

### EXAMPLE E118

### [4-(3-Chlorophenyl)piperazin-1-yl](2-phenyl-5-trifluoromethyl-2H-pyrazol-3-yl)-methanone

By performing the process in a manner similar to that for the synthesis of [4-(3-chlorophenyl)piperazin-1-yl](5-methyl-2-phenyl-2H-pyrazol-3-yl)methanone (Example 1), but replacing the 5-methyl-2-phenyl-2H-pyrazole-3-carboxylic acid with 256 mg of 2-phenyl-5-trifluoromethyl-2H-pyrazole-3-carboxylic acid, which may be obtained according to patent WO 03/024222, 240 mg of [4-3-chlorophenyl)piperazin-1-yl](2-phenyl-5-trifluoromethyl-2H-pyrazol-3-yl)methanone are obtained in the form of a white solid, the characteristics of which are as follows:
Melting point (Kofler): 160° C.
Mass spectrum (EI): m/z=434 (M+)

### EXAMPLE E119

### 3-[4-(2-Phenyl-5-trifluoromethyl-2H-pyrazole-3-carbonyl)piperazin-1-yl]benzamide

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 306 mg of 3-(piperazin-1-yl)benzamide, which may be obtained according to patent WO 98/00400, and replacing the 5-methyl-2-phenyl-2H-pyrazole-3-carboxylic acid with 256 mg of 2-phenyl-5-trifluoromethyl-2H-pyrazole-3-carboxylic acid, which may be obtained according to patent WO 03/024222, 260 mg of 3-[4-(2-phenyl-5-trifluoromethyl-2H-pyrazole-3-carbonyl)piperazin-1-yl]benzamide are obtained in the form of a white solid, the characteristics of which are as follows:
Melting point (Kofler): 160° C.
Mass spectrum (EI): m/z=443 (M+)

### EXAMPLE E120

### (5-Aminomethyl-2-phenyl-2H-pyrazol-3-yl)[4-(3,5-dimethoxyphenyl)piperazin-1-yl]methanone

Step 1: 32 mg of sodium azide are added to a solution of 150 mg of ethyl 5-bromomethyl-2-phenyl-2H-pyrazole-3-carboxylate, obtained in Step 1 of Example E73, in 2 ml of DMSO at room temperature. The reaction mixture is stirred for 2 hours at room temperature and then for 1 hour at 50° C., and is finally poured onto 50 ml of water and extracted with ethyl acetate. The organic phase is dried over magnesium sulfate and concentrated under reduced pressure to give 135 mg of ethyl 5-azidomethyl-2-phenyl-2H-pyrazole-3-carboxylate in the form of a pale yellow oil, the characteristics of which are as follows:
TLC analysis: 7/3 heptane/EtOAc, Rf=0.35
Mass spectrum (ES): m/z=272 (MH+)

Step 2: The process is performed in a manner similar to that of Step 2 of Example E94, starting with 135 mg of the product of Step 1 of the present example and 221 mg of 1-(3,5-dimethoxyphenyl)piperazine, for 6 hours at 60° C., to give, after purification by flash chromatography on a column of silica (30-60 μm), eluting with a mixture of dichloromethane and ethyl acetate (95/5 by volume), 129 mg of (5-azidomethyl-2-phenyl-2H-pyrazol-3-yl)[4-(3,5-dimethoxyphenyl)piperazin-1-yl]methanone in the form of a white solid, the characteristics of which are as follows:
Mass spectrum (ES): m/z=448 (MH⁺)

Step 3: 90 mg of triphenylphosphine are added to a solution of 129 mg of the product of Step 2 of the present example in 3 ml of THF at room temperature. The reaction mixture is stirred for 6 hours at room temperature, 0.6 ml of water is then added and the resulting mixture is stirred for 40 hours at room temperature, concentrated to dryness under reduced pressure and taken up in dichloromethane. The organic phase is washed with aqueous 1M hydrochloric acid solution and then with saturated aqueous sodium bicarbonate solution, dried over magnesium sulfate and concentrated under reduced pressure to give 84 mg of (5-aminomethyl-2-phenyl-2H-pyrazol-3-yl)[4-(3,5-dimethoxyphenyl)piperazin-1-yl]methanone in the form of a white solid, the characteristics of which are as follows:
LC/MS analysis: tr=2.63; m/z=422 (MH⁺)

### EXAMPLE E121

### Allyl {5-[4-(3,5-Dimethoxyphenyl)piperazine-1-carbonyl]-1-phenyl-1H-pyrazol-3-ylmethyl}carbamate

23 μl of allyl isocyanate are added, at 5° C., to a solution of 100 mg of (5-aminomethyl-2-phenyl-2H-pyrazol-3-yl)[4-(3,5-dimethoxyphenyl)piperazin-1-yl]methanone, obtained in Example 120, in 1 ml of dichloromethane and 33 μl of triethylamine. The reaction mixture is stirred for 1.5 hours at 5° C. and then for 18 hours at 20° C., after which it is poured into 50 ml of saturated aqueous sodium dihydrogen phosphate solution and extracted with ethyl acetate. The organic phase is dried over magnesium sulfate and concentrated under reduced pressure. After purification by flash chromatography on a column of silica (30-60 μm), eluting with a mixture of dichloromethane and ethyl acetate (60/40 by volume), 48 mg of allyl {5-[4-(3,5-dimethoxyphenyl)piperazine-1-carbonyl]-1-phenyl-1H-pyrazol-3-ylmethyl}carbamate are obtained in the form of a white solid, the characteristics of which are as follows:
LC/MS analysis: tr=4.71; m/z=506 (MH⁺)

### EXAMPLE E122

### Ethyl {5-[4-(3,5-Dimethoxyphenyl)piperazine-1-carbonyl]-1-phenyl-1H-pyrazol-3-ylmethyl}carbamate

The process is performed in a manner similar to that of Example E121, starting with 100 mg of (5-aminomethyl-2-phenyl-2H-pyrazol-3-yl)[4-(3,5-dimethoxyphenyl)piperazin-1-yl]methanone, obtained in Example 120, and 21 μl of ethyl isocyanate, to give, after purification by flash chromatography on a column of silica (30-60 μm), eluting with a mixture of dichloromethane and ethyl acetate (60/40 by volume), 74 mg of ethyl {5-[4-(3,5-dimethoxyphenyl)piperazine-1-carbonyl]-1-phenyl-1H-pyrazol-3-ylmethyl}-carbamate, in the form of a white solid, the characteristics of which are as follows:
LC/MS analysis: tr=4.62; m/z=494 (MH⁺)

### EXAMPLE E123

### [4-(3,5-Dimethoxyphenyl)piperazin-1-yl][5-(2-fluoroethoxymethyl)-2-phenyl-2H-pyrazol-3-yl]methanone

Step 1: The process is performed in a manner similar to that of Step 1 of Example E80, starting with 34 μl of 2-fluoroethanol and 150 mg of ethyl 5-bromomethyl-2-phenyl-2H-pyrazole-3-carboxylate, obtained in Step 1 of Example E73, to give, after purification by flash chromatography on a column of silica (30-60 μm), eluting with a mixture of heptane and ethyl acetate (80/20 by volume), 66 mg of ethyl 5-(2-fluoroethoxymethyl)-2-phenyl-2H-pyrazole-3-carboxylate in the form of a pale yellow oil, the characteristics of which are as follows:
Mass spectrum (ES): m/z=293 (MH⁺)

Step 2: The process is performed in a manner similar to that of Step 2 of Example E80, starting with 66 mg of the product of Step 1 of the present example and 151 mg of 1-(3,5-dimethoxyphenyl)piperazine for 4 hours at 60° C., to give, after purification by flash chromatography on a column of silica (30-60 μm), eluting with a mixture of dichloromethane and ethyl acetate (60/40 by volume), 86 mg of [4-(3,5-dimethoxyphenyl)piperazin-1-yl][5-(2-fluoroethoxymethyl)-2-phenyl-2H-pyrazol-3-yl]methanone in the form of a pale yellow oil, the characteristics of which are as follows:
LC/MS analysis: tr=3.70; m/z=469 (MH⁺)

### EXAMPLE E124

### [5-(Cyclopentylhydroxymethyl)-2-phenyl-2H-pyrazol-3-yl][4-(3,5-dimethoxy-phenyl)piperazin-1-yl]methanone

A solution of 100 mg of 5-[4-(3,5-dimethoxyphenyl)piperazine-1-carbonyl]-1-phenyl-1H-pyrazole-3-carboxaldehyde, obtained in Example E100, in 1.5 ml of THF is added, at room temperature, to 0.24 ml of a 1N solution of cyclopentylmagnesium bromide in THF, at room temperature. After stirring for two hours at room temperature, the reaction mixture is poured into 5 ml of saturated aqueous ammonium chloride solution and extracted with ether. The organic phase is dried over magnesium sulfate and concentrated under reduced pressure. After purification by flash chromatography on a column of silica (30-60 μm), eluting with a mixture of dichloromethane and ethyl acetate (60/40 by volume), 18.5 mg of [5-(cyclopentylhydroxymethyl)-2-phenyl-2H-pyrazol-3-yl][4-(3,5-dimethoxyphenyl)piperazin-1-yl]methanone are obtained in the form of a colorless resin, the characteristics of which are as follows:
LC/MS analysis: tr=3.27; m/z=423 (MH⁺)

### EXAMPLE E125

### [4-(3,5-Dimethoxyphenyl)piperazin-1-yl][5-(1-hydroxypropyl)-2-phenyl-2H-pyrazol-3-yl]methanone

The process is performed in a manner similar to that of Example E124, starting with 0.595 ml of a 1N solution of ethylmagnesium bromide in THF and 100 mg of 5-[4-(3,5-dimethoxyphenyl)piperazine-1-carbonyl]-1-phenyl-1H-pyrazole-3-carboxaldehyde, obtained in Example E100, in 1.5 ml of THF, to give, after purification by flash chromatography on a column of silica (30-60 μm), eluting with a mixture of dichloromethane and ethyl acetate (60/40 by volume), 36 mg of [4-(3,5-dimethoxyphenyl)piperazin-1-yl][5-(1-hydroxypropyl)-2-phenyl-2H-pyrazol-3-yl]methanone in the form of a colorless resin, the characteristics of which are as follows:
LC/MS analysis: tr=3.53; m/z=451 (MH⁺)

### EXAMPLE E126

### Methyl E-3-{5-[4-(3,5-dimethoxyphenyl)piperazine-1-carbonyl]-1-phenyl-1H-pyrazol-3-yl}acrylate

0.205 ml of a 1N solution of lithium aluminum hydride in THF is added, at room temperature, to a solution of 100 mg of 5-[4-(3,5-dimethoxyphenyl)piperazine-1-carbonyl]-1-phenyl-1H-pyrazole-3-carboxaldehyde, obtained in Example E100, in 1 ml of THF and 1 ml of methanol. After stirring for 6 hours at room temperature, the reaction mixture is poured into 50 ml of saturated aqueous sodium dihydrogen phosphate solution and extracted with ethyl acetate. The organic phase is dried over magnesium sulfate and concentrated under reduced pressure. After purification by flash chromatography on a column of silica (30-60 μm), eluting with a mixture of dichloromethane and ethyl acetate (95/5 by volume), 49 mg of methyl E-3-{5-[4-(3,5-dimethoxyphenyl)piperazine-1-carbonyl]-1-phenyl-1H-pyrazol-3-yl}acrylate are obtained in the form of a white solid, the characteristics of which are as follows:
LC/MS analysis: tr=3.95; m/z=477 (MH⁺)
¹H NMR spectrum (300 MHz, (CD₃)₂SO d6, δ in ppm): 2.81 ppm (m, 2H); 3.08 ppm (m, 2H); 3.33 ppm (masked, 2H); 3.67 ppm (m, 2H); 3.68 ppm (s, 6H); 3.75 ppm (s, 3H); 5.99 ppm (bs, 1H); 6.02 ppm (bs, 2H); 6.70 ppm (d, J=16 Hz, 1H); 7.28 ppm (s, 1H); from 7.40 to 7.58 ppm (m, 6H).

This product may also be obtained in a manner similar to that of Example E112 with (carbomethoxymethylene)triphenylphosphorane.

### EXAMPLE E127

### [4-(3,5-Dimethoxyphenyl)piperazin-1-yl][5-(1-hydroxyethyl)-2-phenyl-2H-pyrazol-3-yl]methanone

0.5 ml of THF and a solution of 50 mg of 5-[4-(3,5-dimethoxyphenyl)-piperazine-1-carbonyl]-1-phenyl-1H-pyrazole-3-carboxaldehyde, obtained in Example E100, in 1 ml of THF are added at room temperature to 87 μl of a 1.5M solution of methyllithium in THF. After stirring for 20 hours at room temperature, the reaction mixture is poured into 5 ml of saturated aqueous ammonium chloride solution and extracted with ether. The organic phase is dried over magnesium sulfate and concentrated under reduced pressure. After purification by flash chromatography on a column of silica (30-60 μm), eluting with a mixture of dichloromethane and ethyl acetate (60/40 by volume), 6 mg of [4-(3,5-dimethoxyphenyl)piperazin-1-yl][5-(1-hydroxyethyl)-2-phenyl-2H-pyrazol-3-yl]methanone are obtained in the form of a pale yellow solid, the characteristics of which are as follows:
LC/MS analysis: tr=3.38; m/z=437 (MH⁺)

### EXAMPLE E128

### 3-Hydroxy-N-{3-[4-(5-methyl-2-phenyl-2H-pyrazole-3-carbonyl)piperazin-1-yl]-phenyl}propionamide

1 ml of a 1M solution of diethylaluminum chloride in hexane and 72 mg oxetan-2-one are added, at 0° C., to a solution of 362 mg of [4-(3-amino-phenyl)piperazin-1-yl](5-methyl-2-phenyl-2H-pyrazol-3-yl)methanone, described in Example E9, in 20 ml of dichloromethane. After 20 minutes at 0° C. and 3 hours at room temperature, the mixture is cooled again to about 0° C. and 1 ml of 1N hydrochloric acid solution is added. The reaction mixture is then neutralized with saturated sodium bicarbonate solution, extracted with 3 times 50 ml of dichloromethane and washed with 50 ml of water. After drying over magnesium sulfate and concentrating under reduced pressure, followed by purification by flash chromatography on a column of silica (60; 35-70 μm), eluting with a mixture of dichloromethane and methanol (90-10 by volume), 20 mg of 3-hydroxy-N-{3-[4-(5-methyl-2-phenyl-2H-pyrazole-3-carbonyl)piperazin-1-yl]phenyl}propionamide are obtained in the form of an amorphous beige-colored solid, the characteristics of which are as follows:
Mass spectrum (EI): m/z=433 (M⁺)
¹H NMR spectrum (300 MHz, (CD₃)₂SO d6, δ in ppm): 2.32 (s: 3H); 2.45 (t, J=6.5 Hz: 2H); 2.76 (unresolved complex: 2H); 3.06 (unresolved complex: 2H); 3.36 (unresolved complex: 2H); 3.70 (mt: 4H); 4.65 (unresolved complex: 1H); 6.53 (s: 1H); 6.58 (broad d, J=8 Hz: 1H); 7.04 (broad d, J=8 Hz: 1H); 7.12 (t, J=8 Hz: 1H); 7.24 (broad s: 1H); 7.36 (broad t, J=7.5 Hz: 1H); from 7.40 to 7.55 (mt: 4H); 9.75 (broad s: 1H).

### EXAMPLE E129

### [5-(Azetidin-1-yl)-2-phenyl-2H-pyrazol-3-yl][4-(3,5-dimethoxyphenyl)piperazin-1-yl]methanone

The process is performed in a manner similar to that of Example E98, starting with 100 mg of the product of Example E82 and 72 μl of azetidine, by microwave reaction for 5 minutes at 90° C., to give, after purification by preparative HPLC/MS (H₂O pH=9/CH₃CN), 23.5 mg of [5-(azetidin-1-yl)-2-phenyl-2H-pyrazol-3-yl][4-(3,5-dimethoxyphenyl)piperazin-1-yl]methanone in the form of a white powder, the characteristics of which are as follows:
LC/MS analysis: tr=4.05; m/z=448 (MH⁺)

### EXAMPLE E130

### (5-Allylamino-2-phenyl-2H-pyrazol-3-yl)[4-(3,5-dimethoxyphenyl)piperazin-1-yl]methanone

The process is performed in a manner similar to that of Example E98, starting with 100 mg of the product of Example E82 and 80 μl of allylamine, by microwave reaction for 3 minutes at 100° C. 10 mg of tris(dibenzylideneacetone)dipalladium(0), 80 μl of allylamine and 0.20 ml of DME are added and the mixture is subjected to the microwave field for a further 3 minutes at 100° C., to give, after purification by preparative HPLC/MS (H₂O pH=9/CH₃CN), 33 mg of (5-allylamino-2-phenyl-2H-pyrazol-3-yl)[4-(3,5-dimethoxyphenyl)piperazin-1-yl]methanone in the form of a pale yellow powder, the characteristics of which are as follows:
LC/MS analysis: tr=4.22; m/z=448 (MH⁺)

### EXAMPLE E131

### [4-(3,5-Dimethoxyphenyl)piperazin-1-yl][5-(hydroxyphenylmethyl)-2-phenyl-2H-pyrazol-3-yl]methanone

The process is performed in a manner similar to that of Example E124, starting with 0.36 ml of a 1N solution phenylmagnesium bromide in THF and 100 mg of 5-[4-(3,5-dimethoxyphenyl)piperazine-1-carbonyl]-1-phenyl-1H-pyrazole-3-carboxaldehyde, obtained in Example E100, in 1.5 ml of THF, to give, after purification by flash chromatography on a column of silica (30-60 μm), eluting with a mixture of dichloromethane and ethyl acetate (60/40 by volume), 81 mg of [4-(3,5-dimethoxyphenyl)piperazin-1-yl][5-(hydroxyphenyl-methyl)-2-phenyl-2H-pyrazol-3-yl]methanone in the form of a white solid, the characteristics of which are as follows:
LC/MS analysis: tr=3.86; m/z=499 (MH⁺)

### EXAMPLE E132

### [4-(3-Hydroxymethylphenyl)piperazin-1-yl](5-methyl-2-phenyl-2H-pyrazol-3-yl)methanone

Step 1: 3.6 ml of a 4N solution of hydrochloric acid in dioxane are added dropwise to a solution of 850 mg of tert-butyl 4-(3-hydroxymethylphenyl)piperazine-1-carboxylate, which may be obtained according to patent WO 00/015609, in 4 ml of dioxane. After reaction for 20 hours, the precipitate formed is filtered off and then washed with 20 ml of petroleum ether. 770 mg of [3-(piperazin-1-yl)phenyl]methanol hydrochloride are thus obtained in the form of an amorphous brown solid, the characteristics of which are as follows:
Mass spectrum (EI): m/z=192 (M+)

Step 2: By performing the process in a manner similar to that for the synthesis of [4-(3-chlorophenyl)piperazin-1-yl](5-methyl-2-phenyl-2H-pyrazol-3-yl)-methanone, but replacing the 1-(3-chlorophenyl)piperazine with 265 mg of [3-(piperazin-1-yl)phenyl]methanol hydrochloride, 250 mg of [4-(3-hydroxy-methylphenyl)piperazin-1-yl](5-methyl-2-phenyl-2H-pyrazol-3-yl)methanone are obtained in the form of a beige-colored foam, the characteristics of which are as follows:
Mass spectrum (EI): m/z=376 (M+)

### EXAMPLE E133

### 3-[4-(2-Phenyl-2H-pyrazole-3-carbonyl)piperazin-1-yl]benzamide

By performing the process in a manner similar to that for the synthesis of [4-(3-chlorophenyl)piperazin-1-yl](5-methyl-2-phenyl-2H-pyrazol-3-yl)methanone (Example 1), but replacing the 1-(3-chlorophenyl)piperazine with 556 mg of 3-(piperazin-1-yl)benzamide, which may be obtained according to patent WO 98/00400 and replacing the 5-methyl-2-phenyl-2H-pyrazole-3-carboxylic acid with 376.4 mg of 2-phenyl-2H-pyrazole-3-carboxylic acid, 530 mg of 3-[4-(2-phenyl-2H-pyrazole-3-carbonyl)piperazin-1-yl]benzamide are obtained in the form of an amorphous white solid, the characteristics of which are as follows:
Mass spectrum (EI): m/z=375 (M+)

### EXAMPLE E134

### 3-[4-(5-Hydroxymethyl-2-phenyl-2H-pyrazole-3-carbonyl)piperazin-1-yl]benzamide

1.17 ml of a 4N solution of hydrochloric acid in dioxane are added to a solution of 840 mg of 3-[4-(5-methoxymethoxymethyl-2-phenyl-2H-pyrazole-3-carbonyl)piperazin-1-yl]benzamide, obtained in Example E113, in 30 ml of absolute ethanol. After reaction for 20 hours at room temperature and concentration under reduced pressure, the residue is taken up in 20 ml of water and aqueous 10% sodium bicarbonate solution to pH 8. The solid formed is filtered off and washed with twice 25 ml of water and then with 2 ml of absolute ethanol. 555 mg of 3-[4-(5-hydroxymethyl-2-phenyl-2H-pyrazole-3-carbonyl)piperazin-1-yl]benzamide are thus obtained in the form of an off-white solid, the characteristics of which are as follows:
Melting point (Kofler): 216° C.
Mass spectrum (EI): m/z=405 (M+)

### EXAMPLE E135

### (5-Cyanomethyl-2-phenyl-2H-pyrazol-3-yl)[4-(3,5-dimethoxyphenyl)piperazin-1-yl]methanone

Step 1: 32 mg of potassium cyanide are added to a solution of 150 mg of ethyl 5-bromomethyl-2-phenyl-2H-pyrazole-3-carboxylate, obtained in Step 1 of Example E73, in 2 ml of DMSO at room temperature. The reaction mixture is stirred for 4 hours at 20° C. and then poured into 50 ml of saturated aqueous sodium dihydrogen phosphate solution and extracted with ethyl acetate. The organic phase is dried over magnesium sulfate and concentrated under reduced pressure. After purification by flash chromatography on a column of silica (30-60 μm), eluting with a mixture of heptane and ethyl acetate (70/30 by volume), 102 mg of ethyl 5-cyanomethyl-2-phenyl-2H-pyrazole-3-carboxylate are thus obtained in the form of a colorless oil, the characteristics of which are as follows:
Mass spectrum (ES): m/z=256 (MH⁺)

Step 2: The process is performed in a manner similar to that of Step 2 of Example E80, starting with 100 mg of the product of Step 1 of the present example and 174 mg of 1-(3,5-dimethoxyphenyl)piperazine, for 4 hours at 60° C., to give, after purification by flash chromatography on a column of silica (30-60 μm), eluting with a mixture of dichloromethane and ethyl acetate (80/20 by volume), 106 mg of (5-cyanomethyl-2-phenyl-2H-pyrazol-3-yl)[4-(3,5-dimethoxyphenyl)piperazin-1-yl]methanone in the form of a colorless oil, the characteristics of which are as follows:
LC/MS analysis: tr=3.64; m/z=432 (MH⁺)

### EXAMPLE E136

### [4-(3-Difluoromethoxyphenyl)piperazin-1-yl](5-methyl-2-phenyl-2H-pyrazol-3-yl)methanone hydrochloride

### Step 1: 4-(3-Difluoromethoxyphenyl)piperazine-1-carboxylic acid tert-butoxide

A mixture of commercial 1-boc piperazine (500.1 mg, 2.685 mmol) and of commercial 3-difluoromethoxybromobenzene (598.8 mg,. 2.685 mmol) in toluene (20 ml) is placed in a 50 ml three-necked flask under an inert atmosphere of argon, followed by addition of the ligand (R)(+)-2,2′-bis(diphenylphosphino)-1,1′-binaphthyl (56.850 mg, 91.2 μmol) and palladium(ii) acetate (20.4 mg, 91.2 μmol). The reaction mixture is stirred and refluxed for 16 hours. After cooling to 20° C., the reaction mixture is diluted with water (20 ml) and then extracted with ethyl acetate (2×30 ml). The organic extracts are combined, dried over magnesium sulfate, filtered and evaporated under reduced pressure. The compound obtained is purified by chromatography on silica gel (AIT cartridge, ref. FC-25 Si-BP-SUP, 20-40 μm, dichloromethane eluent, flow rate of 20 ml/min). The fractions containing the expected compound are combined and then evaporated under reduced pressure. The expected 4-(3-difluoromethoxyphenyl)piperazine-1-carboxylic acid tert-butoxide (253 mg) is isolated, the characteristics of which are as follows:
LC/MS analysis: tr=4.18 min, M+H⁺ 329.31

### Step 2: 1-(3-Difluoromethoxyphenyl)piperazine hydrochloride

A solution of 4-(3-difluoromethoxyphenyl)piperazine-1-carboxylic acid tert-butoxide (253 mg, 3.8 mmol) is placed in a mixture of dioxane (1016 μl) and hydrochloric acid (963 μl) in a 10 ml round-bottomed flask. The reaction mixture is stirred at 20° C. for 48 hours. The solid formed is filtered off, washed (diisopropyl ether, 10 ml) and dried under reduced pressure. The 1-(3-difluoromethoxyphenyl)piperazine hydrochloride (189 mg) is isolated, identified, characterized and used without further purification for the following step.

### Step 3: [4-(3-Difluoromethoxyphenyl)piperazin-1-yl](5-methyl-2-phenyl-2H-pyrazol-3-yl)methanone hydrochloride

A solution of 5-methyl-2-phenyl-2H-pyrazole-3-carboxylic acid, which may be obtained according to J. Het. Chem., 30, 307 (1993), (144.4 mg, 714 μmol) in dichloromethane (11 ml) is placed in a 50 ml three-necked flask under an inert atmosphere of argon, followed by successive addition of 1-(3-difluoromethoxyphenyl)piperazine hydrochloride (189 mg, 714 μmol), 1-hydroxybenzotriazole (106.1 mg, 785 μmol), 1-(3-dimethylaminopropyl)-3-ethyl-carbodiimide hydrochloride (150.6 mg, 785 μmol) and then triethylamine (331 μl). The reaction mixture is stirred for 48 hours at 20° C. and then diluted with dichloromethane (20 ml) and water (20 ml), the phases are separated by settling and the aqueous phase is extracted (30 ml of dichloromethane). The organic extracts are combined, dried over magnesium sulfate, filtered and evaporated under reduced pressure. The compound obtained is purified by chromatography on silica gel (AIT cartridge, ref. FC 25-Si-HP, 15-35 μm, 80/20 to 60/40 cyclohexane/ethyl acetate eluent over 60 minutes, flow rate of ml/min). The fractions containing the expected compound are combined and then evaporated under reduced pressure. The evaporation residue is taken up in a mixture of ethyl ether (12 ml) and 2N hydrochloric acid/ethyl ether (500 μl) and then triturated until a solid is obtained, which is filtered off, washed (5 ml) and dried under reduced pressure. The [4-(3-difluoromethoxyphenyl)piperazin-1-yl](5-methyl-2-phenyl-2H-pyrazol-3-yl)methanone hydrochloride (199 mg) is isolated, the characteristics of which are as follows:
Melting point 127° C. (Kofler).

### EXAMPLE E137

### N-{{3-{[4-(3,5-Dimethoxyphenyl)piperazin-1-yl]carbonyl}-2-phenyl-2H-pyrazin-5-yl}glycine tert-butyl ester

The process is performed in a manner similar to that of Example E98, starting with 100 mg of the product of Example E82 and 145 μl of glycine tert-butyl ester, by microwave reaction for 5 minutes at 100° C. 21 mg of tris-(dibenzylideneacetone)dipalladium(0), 145 μl of glycine tert-butyl ester and 0.20 ml of DME are added and the mixture is then subjected to the microwave field for a further 5 minutes at 120° C. and then for 5 minutes at 140° C., to give, after purification by preparative HPLC/MS (H₂O pH=9/CH₃CN), 2.2 mg of tert-butyl ester of N-{{3-{[4-(3,5-dimethoxyphenyl)piperazin-1-yl]carbonyl}-2-phenyl-2H-pyrazin-5-yl}glycine in the form of a white powder, the characteristics of which are as follows:
LC/MS analysis: tr=4.23; m/z=476 (MH⁺)

### EXAMPLE E138

### [4-(3,5-dimethoxyphenyl)piperazin-1-yl][5-(piperid-1-yl)-2-phenyl-2H-pyrazol-3-yl)methanone

The process is performed in a manner similar to that of Example E98, starting with 100 mg of the product of Example E82 and 105 μl of piperidine, by microwave reaction for 5 minutes at 90° C., to give, after purification by preparative HPLC/MS (H₂O pH=9/CH₃CN), 12 mg of [4-(3,5-dimethoxyphenyl)piperazin-1-yl][5-(piperid-1-yl)-2-phenyl-2H-pyrazol-3-yl)methanone in the form of a white powder, the characteristics of which are as follows:
LC/MS analysis: tr=4.57; m/z=522 (MH⁺)

### EXAMPLE E139

### [4-(3,5-dimethoxyphenyl)piperazin-1-yl](4,5-difluoro-2-phenyl-2H-pyrazol-3-yl)methanone

0.30 ml of a 1.7N solution of tert-butyllithium in THF is added to a solution of 100 mg of (5-bromo-2-phenyl-2H-pyrazol-3-yl)[4-(3,5-dimethoxyphenyl)-1-yl]methanone (Example E82) in 2 ml of THF, cooled to −78° C. The reaction medium is stirred for 30 minutes at −78° C. and then treated with a solution of 87 mg of N-fluorobenzenesulfonimide in 1 ml of THF. The reaction medium is stirred for 1.5 hours at −78° C. and then for 16 hours at room temperature, poured into 50 ml of saturated aqueous sodium dihydrogen phosphate solution and extracted with ethyl acetate. The organic phase is dried over magnesium sulfate and concentrated under reduced pressure. After purification by flash chromatography on a column of silica (30-60 μm), eluting with a mixture of toluene and ethyl acetate (90/10 by volume), 15 mg of [4-(3,5-dimethoxyphenyl)piperazin-1-yl](4,5-difluoro-2-phenyl-2H-pyrazol-3-yl)-methanone are obtained in the form of a yellow oil, the characteristics of which are as follows:
LC/MS analysis: tr=4.26; m/z=429 (MH⁺)

### EXAMPLE E140

### [4-(5-hydroxypyrid-3-yl)piperazin-1-yl](5-methyl-2-phenyl-2H-pyrazol-3-yl)-methanone

### Step 1: 3-Benzyloxy-5-bromopyridine

A solution of 5-bromopyrid-3-ol (1 g, 5.74 mmol) in dimethylformamide (15 ml) is placed in a 50 ml three-necked flask under an inert atmosphere of argon, followed by addition, at 20° C., of potassium carbonate (794.3 mg, 5.74 mmol) and benzyl bromide (687 μl, 5.74 mmol). The reaction mixture is stirred at 20° C. for 16 hours and then diluted with ethyl acetate (150 ml) and water (150 ml). After separation of the phases by settling, the organic phase is extracted with 2 portions of ethyl acetate (50 ml). The organic extracts are combined, dried over magnesium sulfate, filtered and evaporated under reduced pressure. The compound obtained is purified by chromatography on silica gel (FC-50-Si-BP-Support cartridge, 20-40 μm, dichloromethane eluent, flow rate of 20 ml/min). The fractions containing the expected compound are combined and then evaporated under reduced pressure to give 350 mg of 3-benzyloxy-5-bromopyridine, the characteristics of which are as follows:
Mass spectrum: (IE) m/z=263 M⁺; m/z=91 C₇H₇⁺ base peak

### Step 2: tert-Butyl 4-(5-benzyloxypyrid-3-yl)piperazine-1-carboxylate

A mixture of 1-boc piperazine (246.8 mg, 1.325 mmol) and of 3-benzyloxy-5-bromopyridine (350 mg, 1.325 mmol) in toluene (10 ml) is placed in a 50 ml three-necked flask under an inert atmosphere of argon, followed by addition of the ligand (R)(+)-2,2′-bis(diphenylphosphino)-1,1′-binaphthyl (28.05 mg, 45.05 μmol) and palladium(ii) acetate (10.10 mg, 45.05 μmol). The reaction mixture is stirred and refluxed for 16 hours. After cooling to 20° C., the reaction mixture is diluted with water (30 ml) and then extracted with ethyl acetate (2×30 ml). The organic extracts are combined, dried over magnesium sulfate, filtered and evaporated under reduced pressure. The crystalline compound obtained is taken up in ethyl ether (20 ml) and then triturated, filtered off and dried under reduced pressure. The tert-butyl 4-(5-benzyloxypyrid-3-yl)piperazine-1-carboxylate, 280.1 mg, is isolated, the characteristics of which are as follows:
LC/MS analysis: tr=3.04 min.; m/z=370.34, (M+H⁺)

### Step 3: 1-(5-Benzyloxypyrid-3-yl)piperazine hydrochloride

A solution of tert-butyl 4-(5-benzyloxypyrid-3-yl)piperazine-1-carboxylate (280.1 mg, 0.758 μmol) in dioxane (1 ml) is placed in a 50 ml round-bottomed flask and is then stirred at 22° C. while adding hydrochloric acid (948 μl). The reaction mixture is stirred at 22° C. for 16 hours. The yellow solid formed is filtered off, rinsed with diisopropyl ether (15 ml) and then dried under reduced pressure. The 1-(5-benzyloxypyrid-3-yl)piperazine hydrochloride (280 mg, 98%) is isolated and used without further purification for the following step.

### Step 4: [4-(5-Benzyloxypyrid-3-yl)piperazin-1-yl](5-methyl-2-phenyl-2H-pyrazol-3-yl)methanone

A solution of 5-methyl-2-phenyl-2H-pyrazole-3-carboxylic acid, which may be obtained according to J. Het Chem., 30, 307 (1993), (150 mg, 742 μmol) in dichloromethane (11 ml) is placed in a 50 ml three-necked flask under an inert atmosphere of argon, followed by successive addition of 1-(5-benzyloxypyrid-3-yl)piperazine hydrochloride (226.9 mg, 742 μmol), 1-hydroxybenzotriazole (110.3 mg, 816 μmol), 1-(3-dimethylaminopropyl)-3-ethylcarbodiimide hydrochloride (156.5 mg, 742 μmol) and then triethylamine (344 μl). The reaction mixture is stirred at 20° C. for 16 hours and is then diluted with dichloromethane (20 ml) and water (10 ml), the phases are separated by settling and the aqueous phase is extracted (30 ml of dichloromethane). The organic extracts are combined, dried over magnesium sulfate, filtered and evaporated under reduced pressure. The compound obtained is purified by chromatography on silica gel (AIT cartridge, ref. FC 25-Si-HP, 15-35μm, eluent: 100% dichloromethane to 90/10 dichloromethane/methanol over 60 minutes, flow rate of 7 ml/min). The fractions containing the expected compound are combined and then evaporated under reduced pressure and [4-(5-benzyloxypyrid-3-yl)piperazin-1-yl](5-methyl-2-phenyl-2Hpyrazol-3-yl)methanone (268 mg) is isolated, the characteristics of which are as follows:
LC/MS analysis: tr=2.90 min; m/z=454.24 (M+H⁺

### Step 4: [4-(5-Hydroxypyrid-3-yl)piperazin-1-yl](5-methyl-2-phenyl-2H-pyrazol-3-yl)methanone

A solution of [4-(5-benzyloxypyrid-3-yl)piperazin-1-yl](5-methyl-2-phenyl-2H-pyrazol-3-yl)methanone (268 mg; 591 μmol) in ethanol (22.5 ml) is placed in a 50 ml round-bottomed flask, ammonium formate (231.1 mg, 3.664 mmol) and palladium-on-charcoal (94.3 mg, 88.6 μmol) are then added and the reaction mixture is maintained at 80° C. for 3.5 hours. After cooling to 20° C., the catalyst is removed by filtration through Celite and the filtrate is then evaporated under reduced pressure. The compound obtained is purified by chromatography on silica gel (cartridge 26 mm in diameter, height 135 mm, 20 g of 15-40 μm silica, eluent of 100% dichloromethane to 80/20 dichloromethane/methanol over 60 minutes, flow rate of 10 ml/min). The fractions containing the expected compound are combined and then evaporated under reduced pressure, to give a compound that is purified again by chromatography on silica gel (cartridge 26 mm in diameter, height 135 mm, 20 g of 15-40 μm silica, eluent of 100% ethyl acetate to 80/20 ethyl acetate/methanol over 60 minutes, flow rate of 10 ml/min). The fractions containing the expected compound are combined and then evaporated under reduced pressure to give [4-(5-hydroxypyrid-3-yl)piperazin-1-yl](5-methyl-2-phenyl-2H-pyrazol-3-yl)methanone (154 mg), the characteristics of which are as follows:
Melting point: 128° C. (Kofler).

Among the products obtained, the products that are particularly preferred are:
[4-(3-Chlorophenyl)piperazin-1-yl](5-methyl-2-phenyl-2H-pyrazol-3-yl)methanone (Example 1)
[4-(3,4-Dimethylphenyl)piperazin-1-yl](5-methyl-2-phenyl-2H-pyrazol-3-yl)-methanone (Example 2)
[4-(3,5-Dichlorophenyl)piperazin-1-yl](5-methyl-2-phenyl-2H-pyrazol-3-yl)-methanone (Example E2)
[4-(Quinolin-4-yl)piperazin-1-yl](5-methyl-2-phenyl-2H-pyrazol-3-yl)methanone (Example E17)
[4-(3-Chlorophenyl)piperazin-1-yl](5-hydroxymethyl-2-phenyl-2H-pyrazol-3-yl)methanone (Example E60)
[4-(3,4-Methylenedioxyphenyl)piperazin-1-yl](5-methyl-2-phenyl-2H-pyrazol-3-yl)methanone (Example E30)
[4-(3,5-Dimethoxyphenyl)piperazin-1-yl](5-methyl-2-phenyl-2H-pyrazol-3-yl)-methanone (Example E20)
[4-(3,5-Dimethylphenyl)piperazin-1-yl](5-methyl-2-phenyl-2H-pyrazol-3-yl)-methanone (Example E63)
[4-(3-Difluoromethoxyphenyl)piperazin-1-yl](5-methyl-2-phenyl-2H-pyrazol-3-yl)methanone (Example E74)
[4-(3-Chlorophenyl)piperazin-1-yl][5-(2-methylimidazol-1-yl-methyl)-2-phenyl-2H-pyrazol-3-yl]methanone (Example E75)
[4-(3,5-Dimethoxyphenyl)piperazin-1-yl][5-(1H-pyrrol-2-yl)methyl-2-phenyl-2H-pyrazol-3-yl]methanone (Example E91)
[4-(3,5-Dimethoxyphenyl)piperazin-1-yl][5-(pyrrolidin-1-yl)methyl-2-phenyl-2H-pyrazol-3-yl]methanone (Example E93)
[4-(3-Carboxamidophenyl)piperazin-1-yl](5-trifluoromethyl-2-phenyl-2H-pyrazol-3-yl)methanone (Example E119)
[5-(Azetidin-1-yl)-2-phenyl-2H-pyrazol-3-yl][4-(3,5-dimethoxyphenyl)piperazin-1-yl]methanone (Example E129)
[4-(3-Carboxamidophenyl)piperazin-1-yl](2-phenyl-2H-pyrazol-3-yl)methanone (Example E133)
[4-(3-Carboxamidophenyl)piperazin-1-yl](5-hydroxymethyl-2-phenyl-2H-pyrazol-3-yl)methanone (Example E134)
[4-(3-Carboxamidophenyl)piperazin-1-yl](5-methyl-2-phenyl-2H-pyrazol-3-yl)methanone (Example E23)
[4-(3,5-Dimethoxyphenyl)piperazin-1-yl](5-hydroxymethyl-2-phenyl-2H-pyrazol-3-yl)methanone (Example E73)
(5-Amino-2-phenyl-2H-pyrazol-3-yl)[4-(3,5-dimethoxyphenyl)piperazin-1-yl]-methanone (Example E99).

Among the products that are particularly preferred, the following products are preferred:
[4-(3-Carboxamidophenyl)piperazin-1-yl](5-methyl-2-phenyl-2H-pyrazol-3-yl)methanone (Example E23)
[4-(3,5-Dimethoxyphenyl)piperazin-1-yl](5-hydroxymethyl-2-phenyl-2H-pyrazol-3-yl)methanone (Example E73)
(5-Amino-2-phenyl-2H-pyrazol-3-yl)[4-(3,5-dimethoxyphenyl)piperazin-1-yl]methanone (Example E99).

A product in accordance with the invention may be used for the manufacture of a medicinal product that is useful for treating a pathological condition, in particular a cancer.

The present invention also relates to therapeutic compositions containing a compound according to the invention, in combination with a pharmaceutically acceptable excipient depending on the chosen mode of administration. The pharmaceutical composition may be in solid or liquid form or in the form of liposomes.

Among the solid compositions that may be mentioned are powders, gelatin capsules and tablets. Among the oral forms, solid forms protected against the acidic medium of the stomach may also be included. The supports used for the solid forms consist in particular of mineral supports such as phosphates or carbonates, or organic supports such as lactose, celluloses, starch or polymers. The liquid forms consist of solutions, suspensions or dispersions. They contain, as dispersive support, either water or an organic solvent (ethanol, NMP or the like) or mixtures of surfactants and solvents or of complexing agents and solvents.

The liquid forms will preferably be injectable and, as a result, will have a formulation that is acceptable for such a use.

Acceptable routes of administration by injection include intravenous, intraperitoneal, intramuscular and subcutaneous routes, the intravenous route being preferred.

The administered dose of the compounds of the invention will be adapted by the practitioner depending on the route of administration to the patient and the condition of said patient.

The compounds of the present invention may be administered alone or as a mixture with other anticancer agents. Among the possible combinations, that may be mentioned are:
alkylating agents and especially cyclophosphamide, melphalan, ifosfamide, chlorambucil, busulfan, thiotepa, prednimustine, carmustine, lomustine, semustine, steptozotocin, decarbazine, temozolomide, procarbazine and hexamethylmelamine;
platinum derivatives especially such as cisplatin, carboplatin or oxaliplatin;
antibiotic agents especially such as bleomycin, mitomycin or dactinomycin;
antimicrotubule agents especially such as vinblastine, vincristine, vindesine, vinorelbine or taxoids (paclitaxel and docetaxel);
anthracyclines especially such as doxorubicin, daunorubicin, idarubicin, epirubicin, mitoxantrone or losoxantrone;
group I and 11 topoisomerases such as etoposide, teniposide, amsacrine, irinotecan, topotecan and tomudex;
fluoropyrimidines such as 5-fluorouracil, UFT or floxuridine;
cytidine analogues such as 5-azacytidine, cytarabine, gemcitabine, 6-mercaptomurine or 6-thioguanine;
adenosine analogs such as pentostatin, cytarabine or fludarabine phosphate;
methotrexate and folinic acid;
various enzymes and compounds such as L-asparaginase, hydroxyurea, trans-retinoic acid, suramin, dexrazoxane, amifostine, herceptin and estrogen and androgen hormones;
antivascular agents such as combretastatin or colchicine derivatives and prodrugs thereof.

It is also possible to combine the compounds of the present invention with a radiation treatment. These treatments may be administered simultaneously, separately or sequentially. The treatment will be adapted to the patient to be treated by the practitioner.

A product of the invention may be useful for inhibiting the in vitro polymerization of tubulin.

Evaluation of the Inhibition of Tubulin Polymerization

Tubulin is purified from pig brain according to published methods (Shelanski et al., 1973, Proc. Natl. Acad. Sci. USA, 70, 765-768. Weingarten et al., 1975, Proc. Natl. Acad. Sci. USA, 72, 1858-1862). Briefly, the brains are ground and centrifuged in an extraction buffer. The tubulin contained in the supernatant of the extract undergoes two successive cycles of polymerization at 37° C. and depolymerization at 4° C., before being separated from the MAPs (Microtubule Associated Proteins) by chromatography on a phosphocellulose P11 column (Whatman). The tubulin thus isolated is more than 95% pure. It is stored in a buffer known as RB/2 30% glycerol, the composition of which is 50 mM MES-NaOH [2-(N-morpholino)ethanesulfonic acid], pH 6.8; 0.25 mM MgCl₂; 0.5 mM EGTA; 30% glycerol (v/v), 0.2 mM GTP (guanosine-5′-triphosphate).

The polymerization of tubulin to microtubules is monitored by turbidimetry as follows: the tubulin is adjusted to a concentration of 10 μm (1 mg/ml) in RB/2 30% glycerol buffer to which 1 mM GTP and 6 mM MgCl₂ are added. The polymerization is triggered by an increase in temperature from 6° C. to 37° C. in a cuvette with an optical path length of 1 cm, placed in a UVIKON 931 spectrophotometer (Kontron) equipped with a thermostatically maintained cuvette holder. The increase in the turbidity of the solution is monitored at 350 nm.

The products are dissolved at 10 mM in DMSO and added at variable concentrations (0.5 to 10 μm) to the tubulin solution before polymerization. The IC₅₀ value is defined as the concentration of product that inhibits the rate of polymerization by 50%. A product whose IC₅₀ value is less than or equal to 25 μm is considered as being very active.

A product in accordance with the invention may be useful for inhibiting the in vitro proliferation of tumoral cells.

Test for Determining the Inhibition of Proliferation of the Human Colon Tumor Line HCT116

The proliferation of HCT116 cells is evaluated by measuring the incorporation of [¹⁴C]-thymidine in the following manner. The HCT 116 cells (obtained from the ATCC) are cultured in a DMEM medium (Gibco) containing 10% fetal calf serum and antibiotics (1% penicillin, 1% streptomycin). To perform the proliferation test, the cells are inoculated in cytostar 96-well microplates (Amersham), at a rate of 5000 cells per well. [¹⁴C]-thymidine (0.1 μCi/well) and the products to be evaluated are then added. Variable concentrations of products up to 10 μm are used; the DMSO (solvent used to dissolve the products) should not exceed 0.5% in the medium. 48 hours after incubation at 37° C., the radioactivity incorporated into the cells is measured by counting the plate in a TRI-LUX counter (Wallac). The IC₅₀ is defined as the concentration of product that reduces the radioactivity by 50% compared with an untreated control. A product whose IC₅₀ is less than 3 μm is considered cytotoxic.

Test for Determining the Inhibition of Vascularization

A test for determining the detachment of the endothelial cells was developed in order to select the products with regard to their “in vitro” activity. This test for determining the detachment of endothelial cells is characterized in that the endothelial cells, inoculated into plates whose bottom is covered with a binder preferably chosen from gelatin, fibronectin and vitronectin, after culturing, are supplemented with a medium containing the test compound, and the cells are then labeled with a fluorescent substance, the, cells which have become detached are removed by washing and the fluorescence of the remaining cells is counted in a fluorimeter.

This test consists in measuring the detachment of endothelial cells cultured on substrata based on a binder preferably chosen from fibronectin, vitronectin and gelatin. Preferably, a day after the inoculation of the cells in plates containing, for example, 96 wells, the culture medium is replaced with a medium containing the test compound in the absence of serum. The same preparation is prepared six times at three different concentrations (0.1, 0.3 and 0.6 μm) and the control six times without addition of antivascular product. After two hours of treatment with the test substance, the cells are labeled with calcein-AM (1.6 μg/ml) in the culture medium supplemented with 0.1% BSA. The cells that have become detached are removed by washing with the culture medium containing 0.1% bovine serum albumin; 100 μl of medium are added to each well. The fluorescence of the remaining cells is counted in a fluorimeter. The data obtained are expressed relative to the control (untreated cells).

The evaluation of the detachment of the endothelial cells in vitro is determined in the following manner. HDMEC cells (Human Dermal Microvascular Endothelial Cells, Promocell, c-122102) are cultured in an ECGM-MV medium that contains 5% fetal calf serum, growth factors (EGF 10 ng/ml, hydrocortisone 1 μg/ml, 0.4% growth supplement with heparin) and antibiotics (amphotericin 50 ng/ml, gentamycin 50 μg/ml). For the detachment test, the HDMECs are inoculated at a rate of 5 000 cells in clear-bottomed 96-well plates (Costar) precoated with fibronectin (10 μg/ml), vitronectin (1 μg/ml) or gelatin. Twenty-four hours later, the culture medium is replaced with ECGM-MV 0.1% BSA medium containing the products indicated. The concentrations tested are 0.1-0.3 and 1 μM for each product. After two hours of treatment, the cells are labeled for one hour with calcein (1.6 μg/ml, Molecular Probes) in ECGM-MV 0.1% BSA medium. The detached cells are then removed by washing with ECGM-MV 0.1% BSA medium; 100 μl of medium is added to each well. The fluorescence of the cells that remain attached to the substratum of the well is counted using a fluorimeter, Spectrafluor Plus (Tecan excitation 485 nm, and emission 535 nm). The data are the mean of six different samples and are expressed as the percentage of the control (untreated cells).

A cell detachment effect of greater than or equal to 15% is considered as significant.

Biological results

| | | | Inhibition of | Inhibition of | Percentage |
|---|---|---|---|---|---|
| | | | tubulin | HCT116 | detachment |
| Ex. | | Molar | polymerization | proliferation | of HDMEC at |
| No. | Structure | mass | IC 50 (μm) | IC 50 (μm) | 1 μm |
| 3/1 | 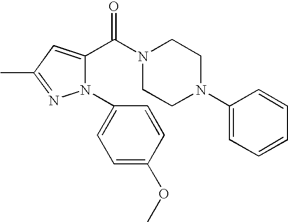 | 376.457 | 20 | | |
| 3/2 | 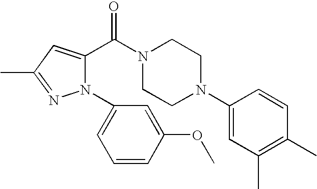 | 404.511 | | 0.312 | |
| 3/3 | 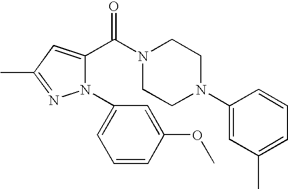 | 390.49 | 1.8 | 0.087 | |
| 3/4 | 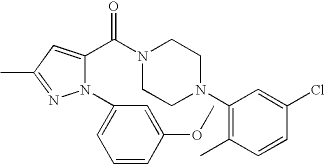 | 424.93 | 1.8 | 0.389 | |
| 3/5 | 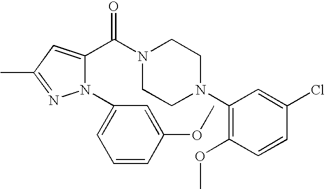 | 440.93 | 3 | 0.238 | |
| 3/6 | 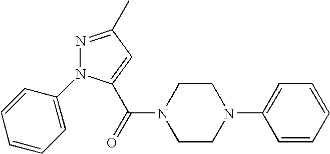 | 346.44 | 4.5 | 0.177 | |
| 3/7 | 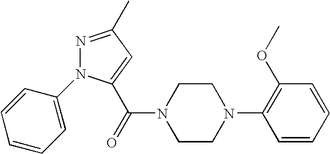 | 376.46 | 5.6 | 0.040 | |
| 3/8 | 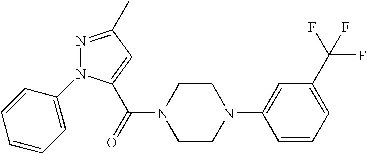 | 414.43 | 6.5 | 0.841 | |
| 3/9 | 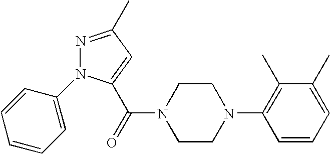 | 374.49 | 7 | 0.494 | |
| 1 | 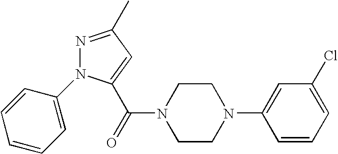 | 380.87 | 0.6 | 0.002 | 22 |
| 3/10 | 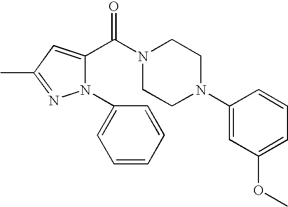 | 376.46 | 2 | 0.025 | |
| 3/11 | 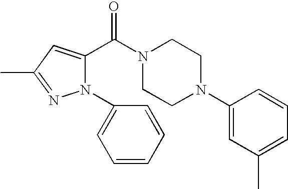 | 360.46 | 3 | 0.036 | |
| 3/12 | 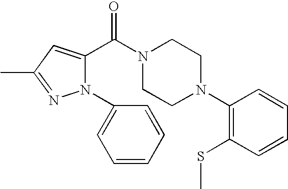 | 392.53 | 3 | O.145 | |
| 3/13 | 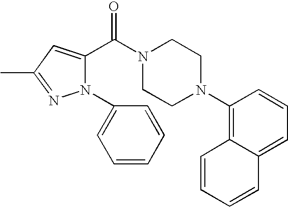 | 396.5 | 6 | 0.466 | |
| 3/14 | 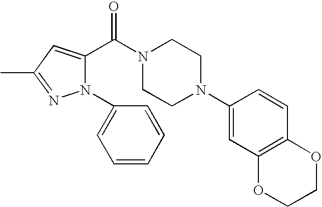 | 404.47 | 15 | 0.352 | |
| 3/15 | 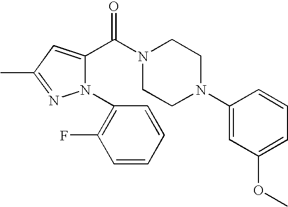 | 394.45 | 2 | 0.0040 | |
| 3/16 | 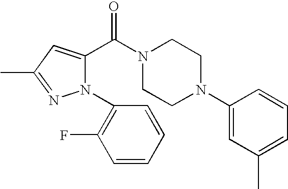 | 378.45 | 6 | 0.224 | |
| 3/17 | 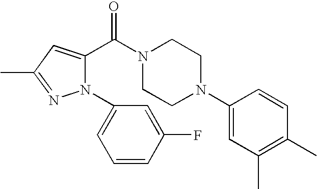 | 392.48 | 3.5 | 0.159 | |
| 3/18 | 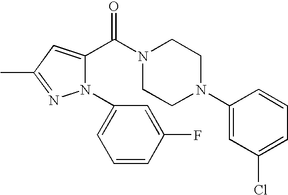 | 398.87 | 2 | 0.028 | |
| 3/19 | 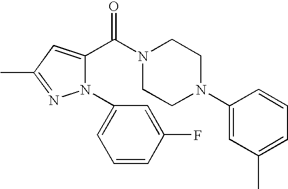 | 378.45 | 1.8 | 0.017 | |
| 3/20 | 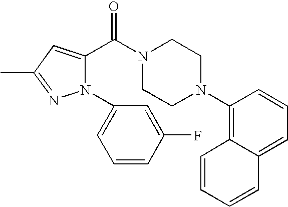 | 414.49 | 3 | 1.846 | |
| 3/21 | 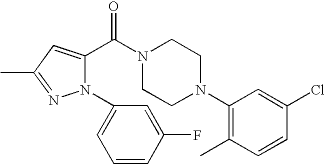 | 412.9 | 1.8 | 0.469 | |
| 3/22 | 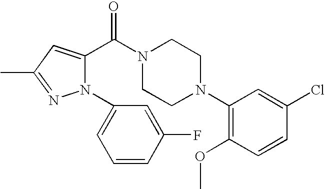 | 428.9 | 1.5 | 0.046 | |
| 3/23 | 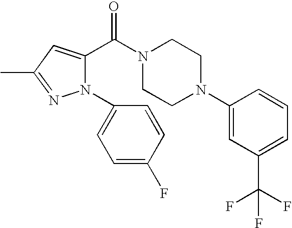 | 432.419 | 3 | 0.434 | |
| 3/24 | 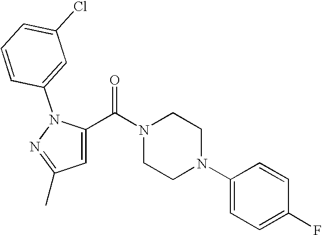 | 398.87 | 5 | 0.239 | |
| 3/25 | 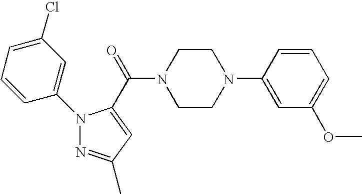 | 410.902 | 1.8 | 0.040 | |
| 3/26 | 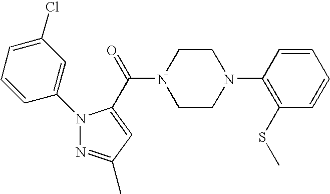 | 426.969 | 1.5 | 0.090 | |
| 3/27 | 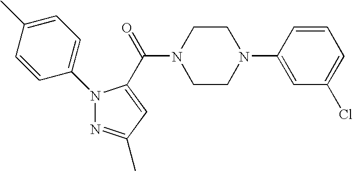 | 394.903 | 4.5 | 0.217 | |
| 3/28 | 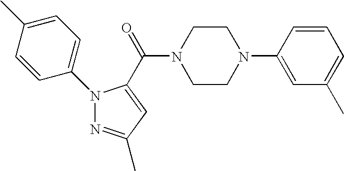 | 374.485 | 7 | 0.269 | |
| 3/29 | 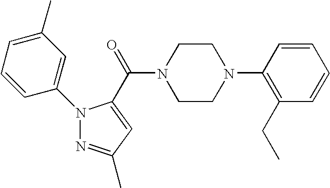 | 388.512 | 5 | 0.452 | |
| 3/30 | 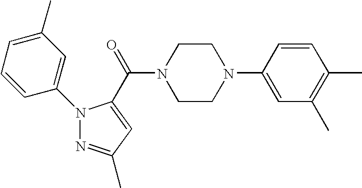 | 388.512 | 2.9 | 0.729 | |
| 3/31 | 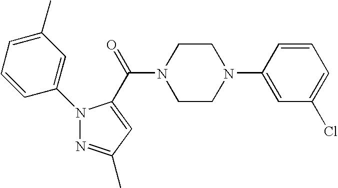 | 394.903 | 1.3 | 0.140 | |
| 3/32 | 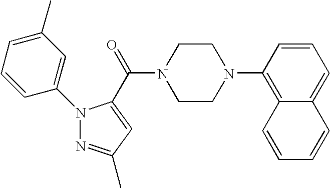 | 410.518 | 3 | 0.556 | |
| 3/33 | 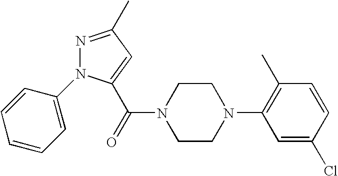 | 394.903 | 3 | 0.170 | |
| 3/34 | 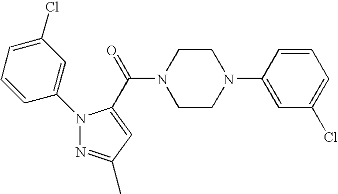 | 415.322 | 2.5 | 0.526 | |
| 3/35 | 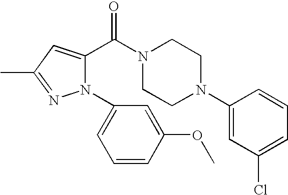 | 410.902 | 5 | 0.284 | |
| 3/36 | 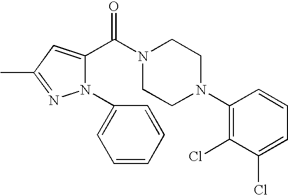 | 415.322 | 1.8 | 0.119 | |
| 3/37 | 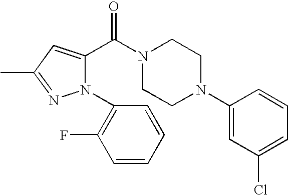 | 398.867 | 20 | 1.436 | |
| 3/38 | 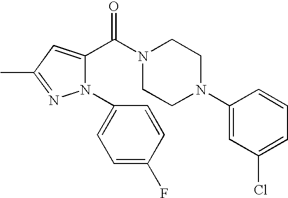 | 398.867 | 6 | 0.338 | |
| 3/39 | 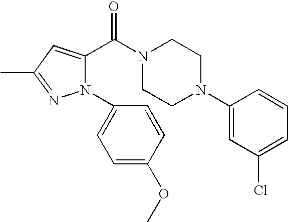 | 410.902 | 25 | 0.734 | |
| 3/40 | 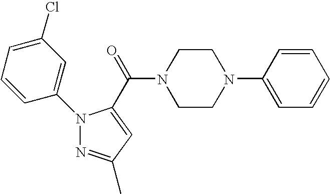 | 380.876 | 3 | 0.775 | |
| 3/41 | 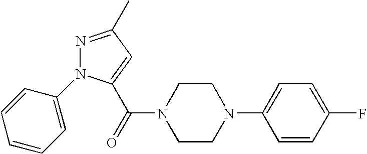 | 364.421 | 6.5 | 0.2940 | |
| 3/42 | 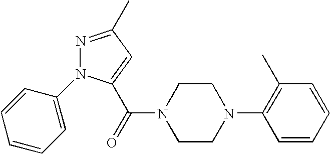 | 360.458 | 5 | 0.0850 | |
| 3/43 | 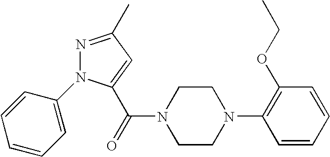 | 390.484 | 9 | 0.2290 | |
| 3/44 | 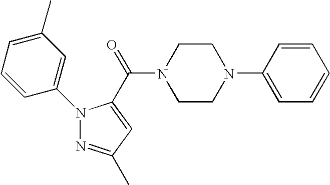 | 360.458 | 2 | 0.0470 | |
| 3/45 | 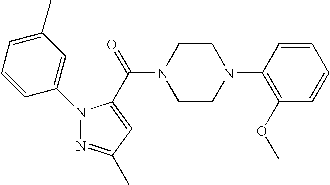 | 390.484 | 3 | 0.0730 | |
| 3/46 | 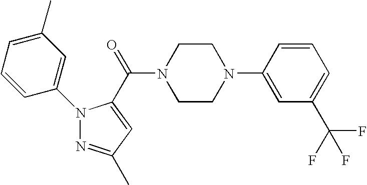 | 428.455 | 8 | 0.1970 | |
| 3/47 | 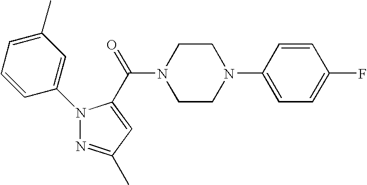 | 378.448 | 8 | 0.3120 | |
| 3/48 | 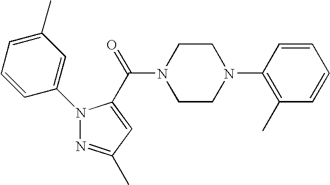 | 374.485 | 5 | 0.1180 | |
| 3/49 | 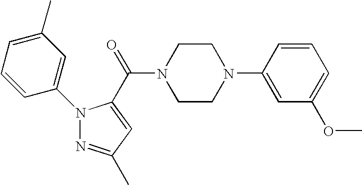 | 390.484 | 1 | 0.0079 | |
| 3/50 | 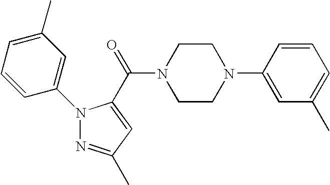 | 374.485 | 1.5 | 0.0061 | |
| 3/51 | 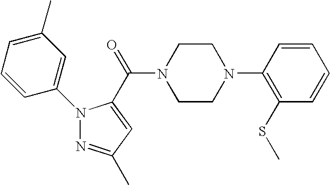 | 406.551 | 2 | 0.0370 | |
| 3/52 | 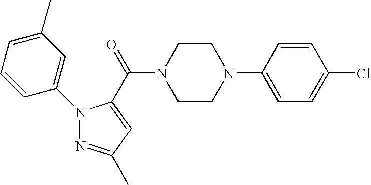 | 394.903 | 13 | 0.2560 | |
| 3/53 | 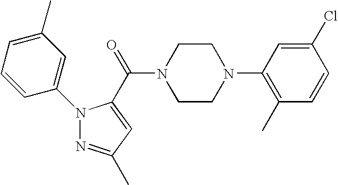 | 408.930 | 1.2 | 0.0520 | |
| 3/54 | 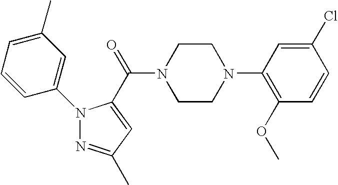 | 424.929 | 1 | 0.1170 | |
| 3/55 | 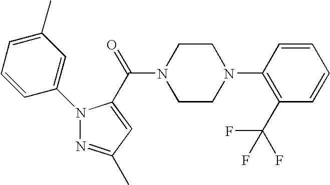 | 428.455 | 5.5 | 1.3500 | |
| 3/56 | 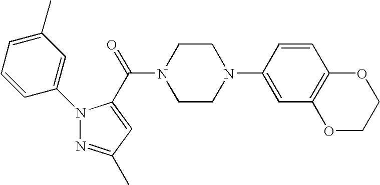 | 418.494 | 5 | 0.1390 | |
| 3/57 | 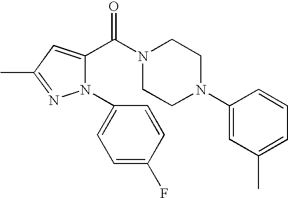 | 378.448 | 1.4 | 0.0150 | |
| 3/58 | 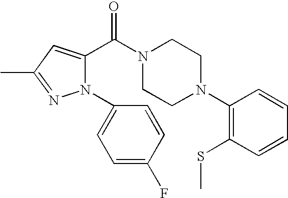 | 410.514 | 4.5 | 3.9370 | |
| 3/59 | 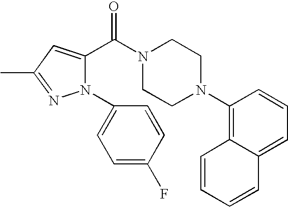 | 414.481 | 5.7 | 4.9750 | |
| 3/60 | 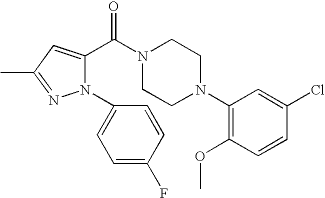 | 428.892 | 1.3 | 0.0086 | |
| 3/61 | 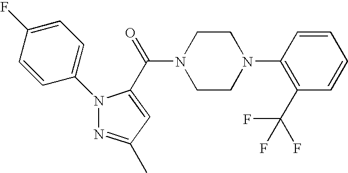 | 432.419 | 25 | | |
| 3/62 | 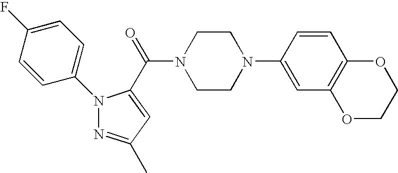 | 422.457 | 6 | 0.0710 | |
| 3/63 | 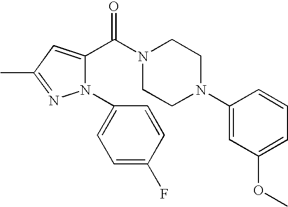 | 394.447 | 0.9 | 0.0190 | |
| 3/64 | 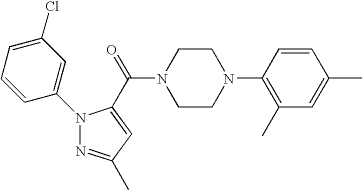 | 408.930 | 25 | | |
| 3/65 | 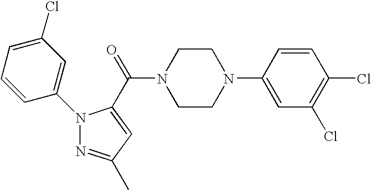 | 449.767 | 1.3 | 0.0730 | |
| 3/66 | 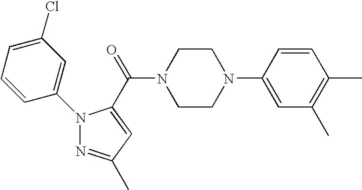 | 408.930 | 2 | 0.0730 | |
| 3/67 | 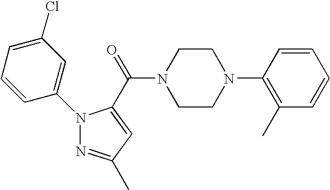 | 394.903 | 2.7 | 0.1270 | |
| 3/68 | 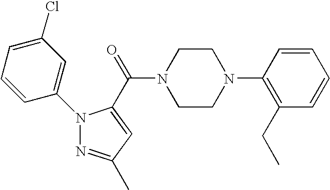 | 408.930 | 1.5 | 0.0360 | |
| 3/69 | 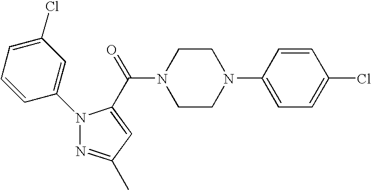 | 415.322 | 18 | 0.3470 | |
| 3/70 | 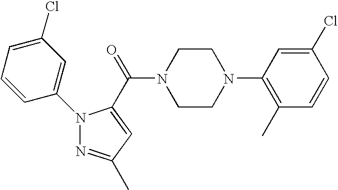 | 429.348 | 12.5 | | |
| 3/71 | 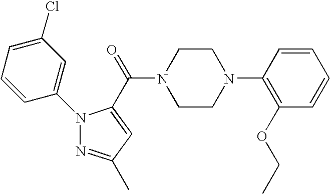 | 424.929 | 2.5 | 0.2200 | |
| 3/72 | 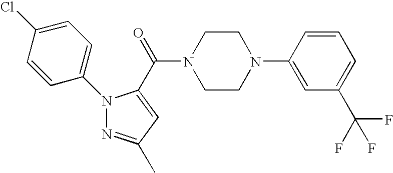 | 448.874 | 7 | 0.1430 | |
| 3/73 | 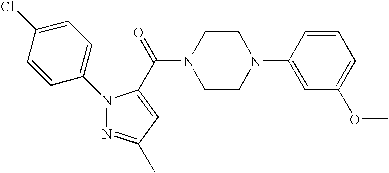 | 410.902 | 2.5 | 0.1110 | |
| 3/74 | 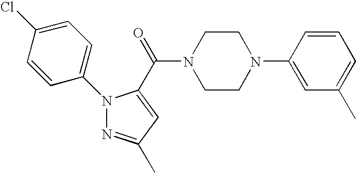 | 394.903 | 7.5 | 0.2980 | |
| 3/75 | 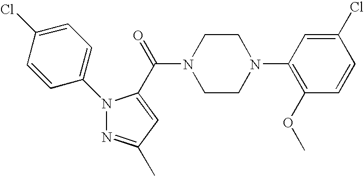 | 445.347 | 17 | 0.4680 | |
| 3/76 | 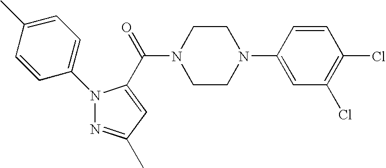 | 429.348 | 3 | 1.0810 | |
| 3/77 | 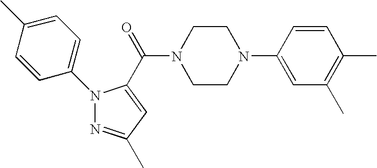 | 388.512 | 25 | | |
| 3/78 | 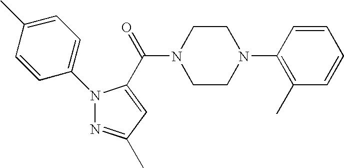 | 374.485 | 25 | | |
| 3/79 | 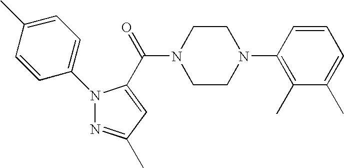 | 388.512 | 25 | | |
| 3/80 | 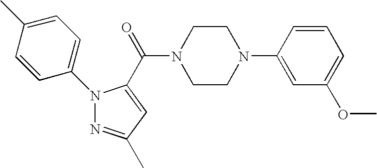 | 390.484 | 5 | 0.0780 | |
| 3/81 | 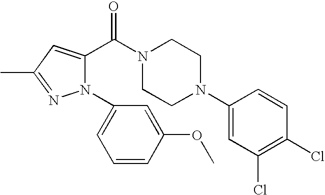 | 445.347 | 3.5 | 0.2950 | |
| 3/82 | 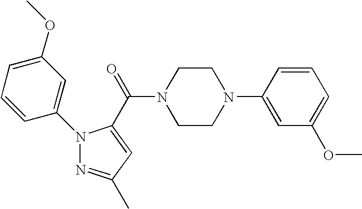 | 406.483 | 1.2 | 0.0083 | |
| 3/83 | 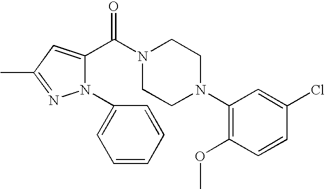 | 410.902 | 1.5 | 0.0066 | |
| 3/84 | 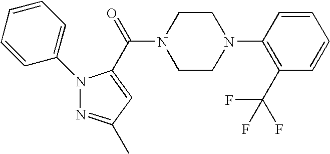 | 414.428 | 5.5 | 0.1760 | |
| 3/85 | 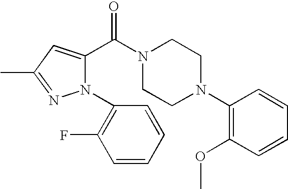 | 394.447 | 3 | 0.1890 | |
| 3/86 | 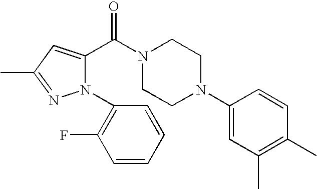 | 392.475 | 12.5 | 0.385 | |
| 3/87 | 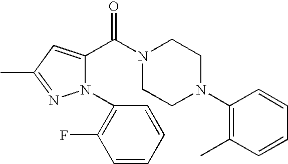 | 378.448 | 15 | 0.2380 | |
| 3/88 | 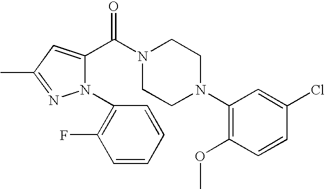 | 428.892 | 6 | 0.0605 | |
| 3/89 | 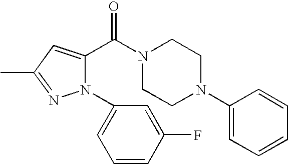 | 364.421 | 2.2 | 0.0140 | |
| 3/90 | 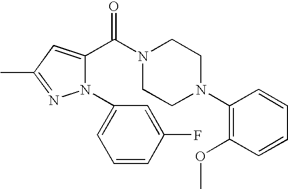 | 394.447 | 3 | 0.0330 | |
| 3/91 | 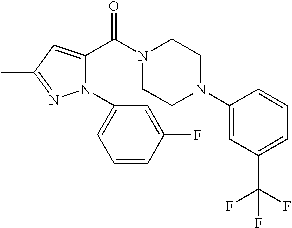 | 432.419 | 1.5 | 0.0140 | |
| 3/92 | 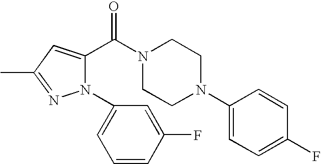 | 382.412 | 12.5 | 0.1580 | |
| 3/93 | 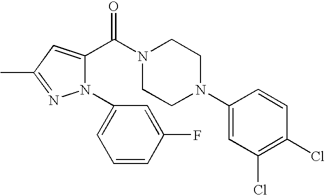 | 433.312 | 1.7 | 0.0320 | |
| 3/94 | 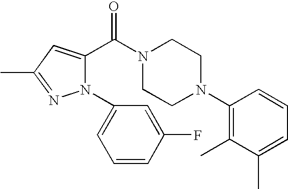 | 392.475 | 0.8 | 0.0320 | |
| 3/95 | 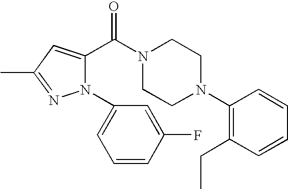 | 392.475 | 3 | 0.1640 | |
| 3/96 | 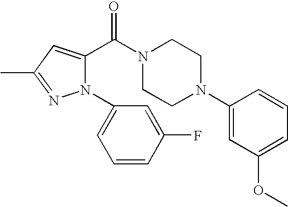 | 394.447 | 1.8 | 0.0036 | |
| 3/97 | 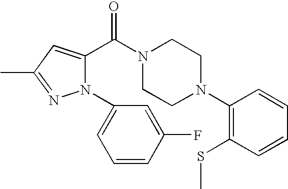 | 410.514 | 3 | 0.0830 | |
| 3/98 | 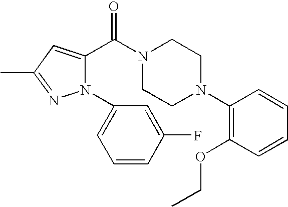 | 408.474 | 7 | 0.1570 | |
| 3/99 | 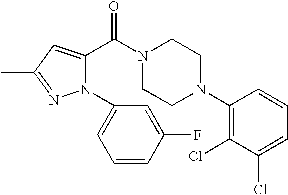 | 433.312 | 2 | 0.0410 | |
| 3/100 | 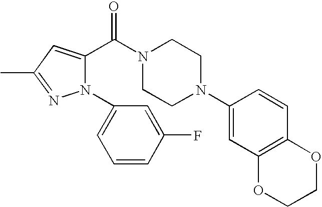 | 422.457 | <25 | | |
| 3/101 | 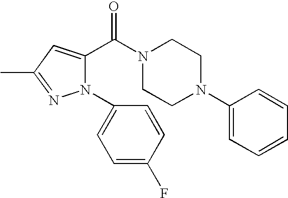 | 364.421 | 5 | 0.5690 | |
| 3/102 | 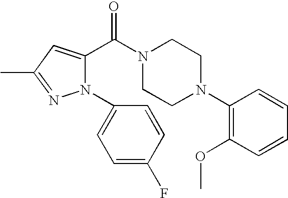 | 394.447 | 13 | 0.1810 | |
| 3/103 | 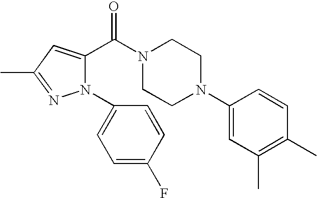 | 392.475 | 21 | | |
| 3/104 | 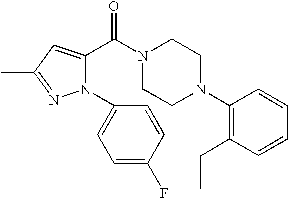 | 392.475 | 5 | 0.2850 | |
| 3/105 | 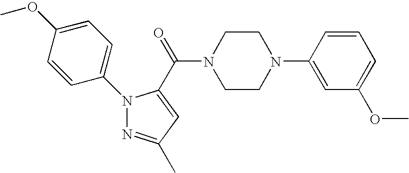 | 406.483 | 4.5 | 0.1180 | |
| 3/106 | 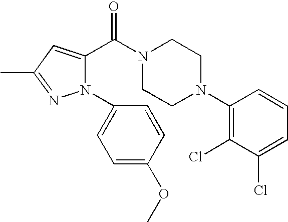 | 445.347 | 7 | 0.9850 | |
| 3/107 | 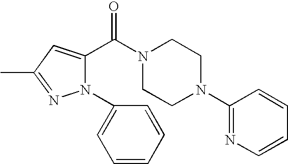 | 347.419 | 12.5 | | |
| 3/108 | 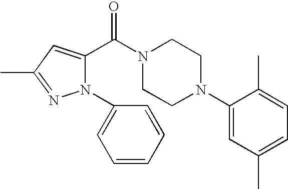 | 374.485 | 3.5 | | |
| 3/109 | 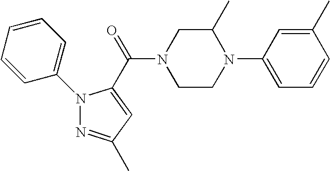 | 374.485 | 8 | 4.3300 | |
| 3/110 | 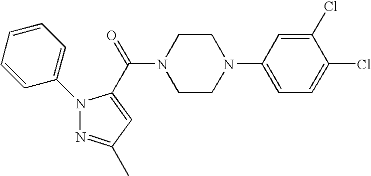 | 415.322 | | 0.2987 | |
| 2 | 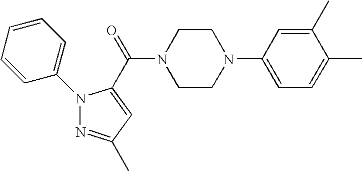 | 374.485 | 0.6 | 0.1600 | 16 |
| 3/111 | 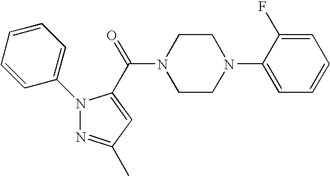 | 364.421 | 12.5 | | |
| 3/112 | 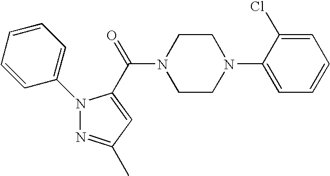 | 380.876 | 15 | 1.5240 | |
| 4 | 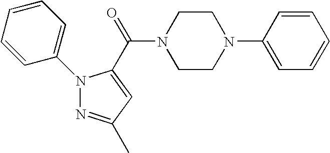 | 343.427 | 3 | 0.0588 | |
| 3/113 | 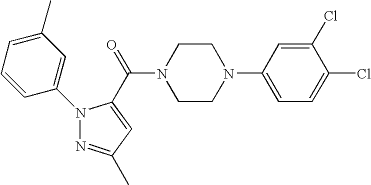 | 429.348 | 1.2 | 0.0750 | |
| 3/114 | 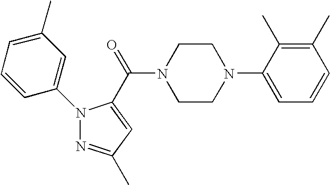 | 388.512 | 0.8 | | |
| 3/115 | 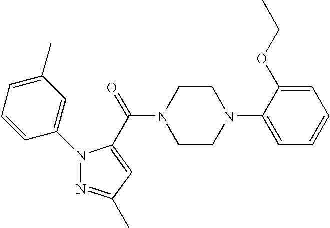 | 404.511 | 20 | | |
| 3/116 | 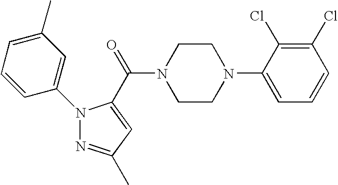 | 429.348 | 3 | 0.1410 | |
| 3/117 | 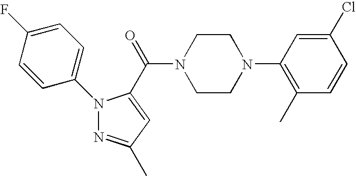 | 412.893 | 7 | | |
| 3/118 | 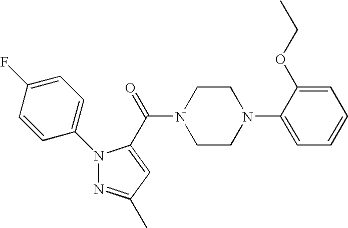 | 408.474 | 25 | | |
| 3/119 | 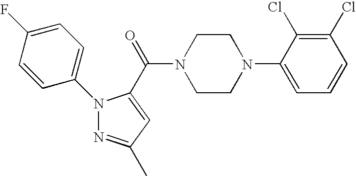 | 433.312 | 3.1 | 0.5620 | |
| 3/120 | 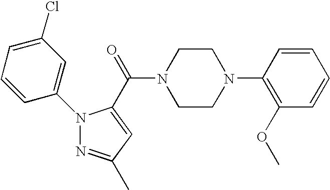 | 410.910 | | 0.040 | |
| 3/121 | 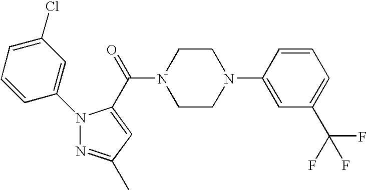 | 448.874 | 3.3 | 0.0510 | |
| 3/122 | 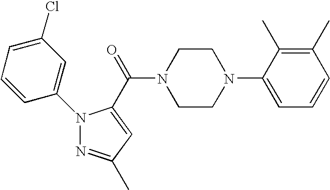 | 408.930 | 7.5 | | |
| 3/123 | 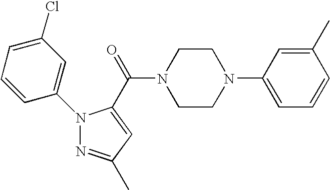 | 394.903 | 3.3 | 0.051 | |
| 3/124 | 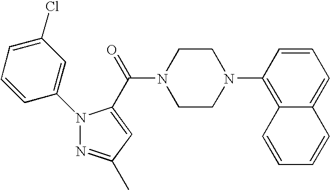 | 430.936 | 12 | | |
| 3/125 | 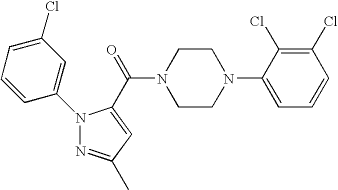 | 449.767 | 12.5 | 0.1480 | |
| 3/126 | 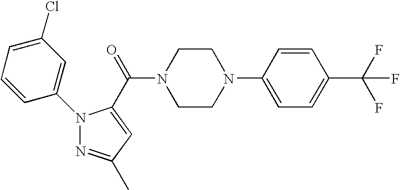 | 448.874 | 25 | 8.7350 | |
| 3/127 | 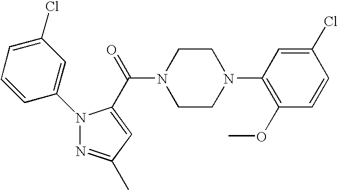 | 445.347 | 1.8 | | |
| 3/128 | 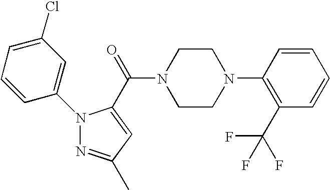 | 448.874 | 12 | 0.9010 | |
| 3/129 | 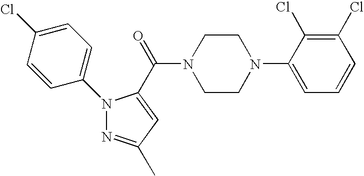 | 449.767 | <25 | 2.5580 | |
| 3/130 | 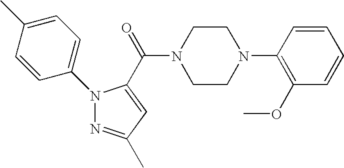 | 390.484 | <25 | | |
| 3/131 | 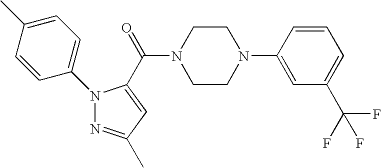 | 428.455 | 3.1 | 0.1350 | |
| 3/132 | 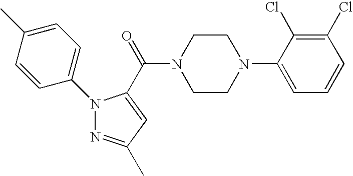 | 429.348 | 20 | 3.0980 | |
| 3/133 | 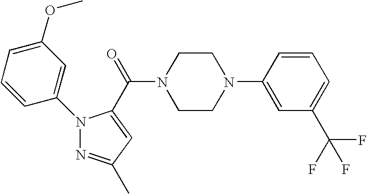 | 444.454 | 6.2 | 0.2140 | |
| 3/134 | 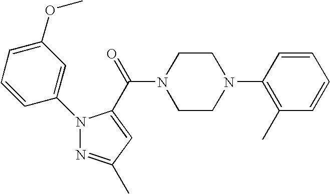 | 390.484 | 4.3 | | |
| 3/135 | 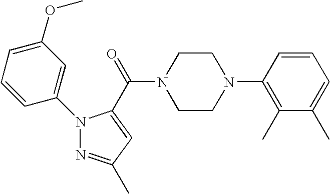 | 404.511 | 6 | 0.3210 | |
| 3/136 | 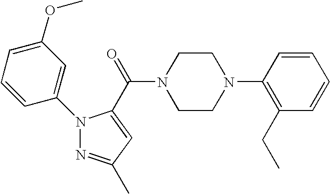 | 404.511 | 12.5 | 0.9320 | |
| 3/137 | 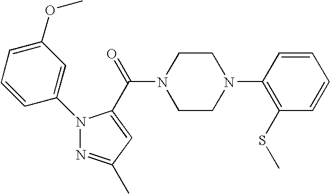 | 422.550 | 4.2 | | |
| 3/138 | 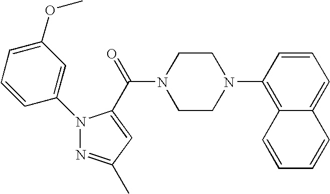 | 426.517 | <25 | 19.9550 | |
| 3/139 | 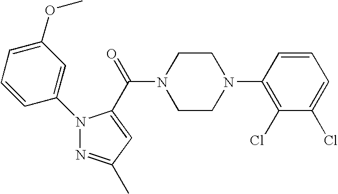 | 445.347 | <25 | 1.3490 | |
| 3/140 | 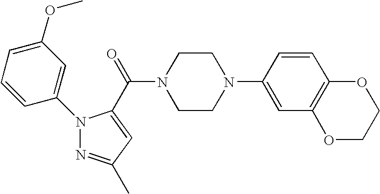 | 434.500 | <25 | 2.9680 | |
| 3/141 | 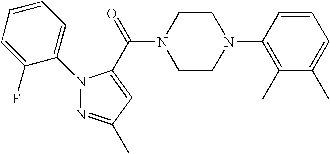 | 392.475 | 7 | 0.0660 | |
| 3/142 | 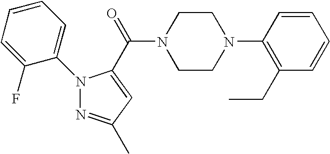 | 392.475 | 3.8 | 0.1100 | |
| 3/143 | 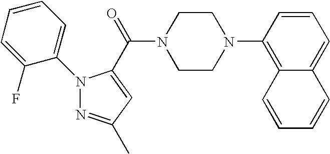 | 414.481 | 2.5 | 0.1400 | |
| 3/144 | 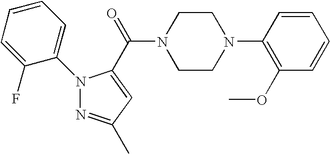 | 394.45 | 9.5 | 0.2550 | |
| 3/145 | 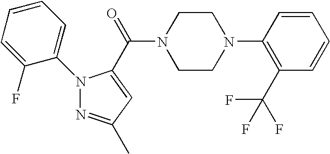 | 432.419 | 10 | 1.5060 | |
| 3/146 | 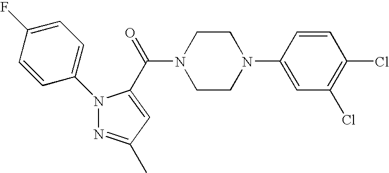 | 433.312 | 1.3 | 0.0430 | |
| 3/147 | 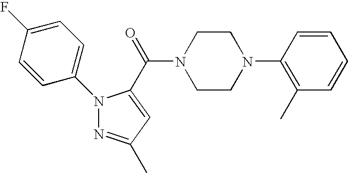 | 378.448 | 7 | 1.2920 | |
| 3/148 | 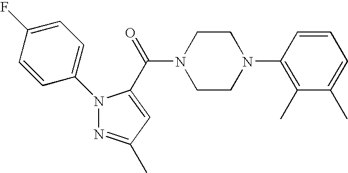 | 392.475 | 1.5 | 0.1980 | |
| 3/149 | 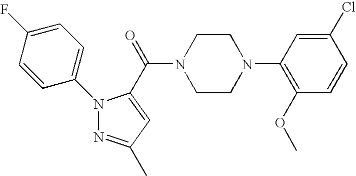 | 440.928 | 7.5 | | |
| 5 | 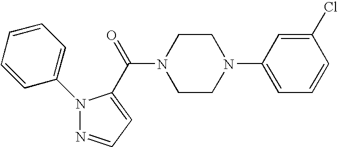 | 366.850 | 1.2 | 0.0041 | |
| 6 | 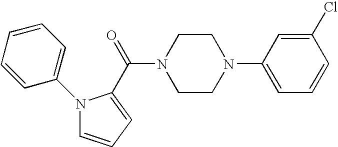 | 365.862 | 1.2 | 0.0382 | |
| 3/150 | 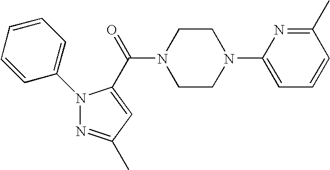 | 361.446 | 1.1 | 0.0055 | |
| 3/151 | 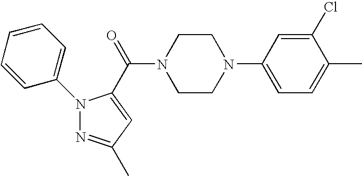 | 394.903 | 1.7 | 0.0221 | |
| 3/152 | 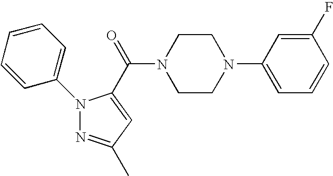 | 364.421 | 0.8 | 0.0063 | |
| 3/153 | 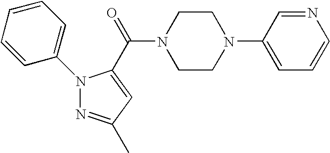 | 347.419 | 2.2 | 0.0224 | |
| E2 | 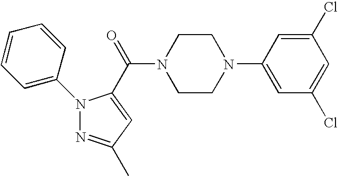 | 415.32 | 1.5 | 0.0001 | |
| E3 | 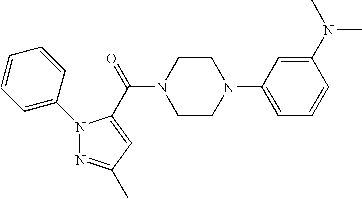 | 399.50 | 25 | | |
| E4 | 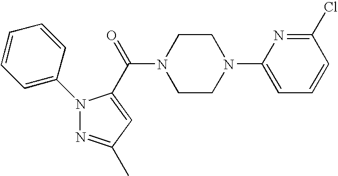 | 381.87 | 1.3 | 0.0047 | |
| E5 | 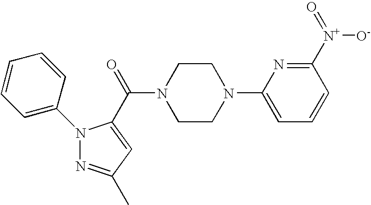 | 391.43 | 0.8 | 0.0030 | |
| E6 | 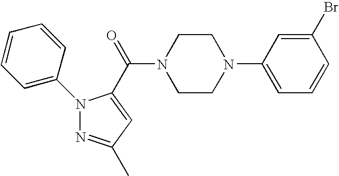 | 425.33 | 0.9 | 0.0012 | |
| E7 | 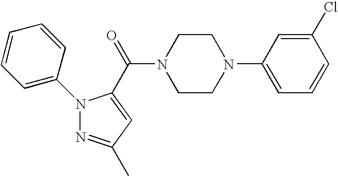 | 396.92 | 1.4 | 0.0110 | |
| E8 | 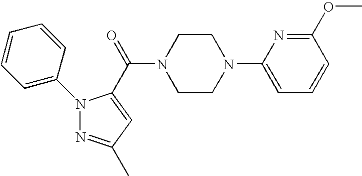 | 377.45 | 2 | | |
| E9 | 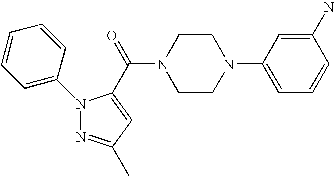 | 361.45 | 3 | 0.0531 | |
| E10 | 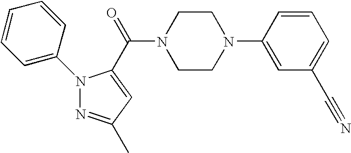 | 371.44 | 0.9 | 0.0049 | |
| E11 | 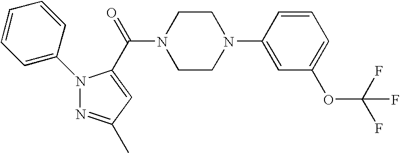 | 430.43 | 1 | 0.0120 | |
| E12 | 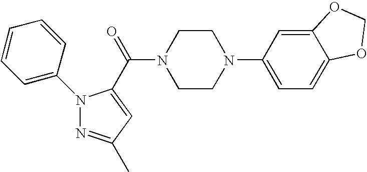 | 390.44 | 2.3 | 0.0031 | |
| E13 | 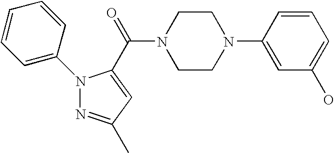 | 362.43 | 1 | 0.0142 | |
| E14 | 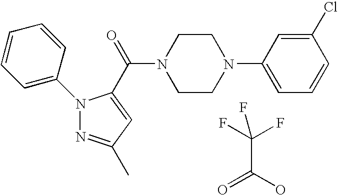 | 480.91 | 12 | 3.079 | |
| E15 | 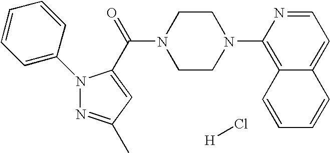 | 434.94 | 14 | 0.2387 | |
| E16 | 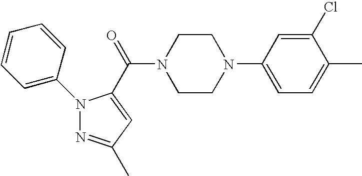 | 394.90 | 1.5 | 0.0390 | |
| E17 | 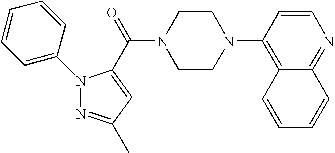 | 397.48 | 1.5 | 0.0085 | |
| E18 | 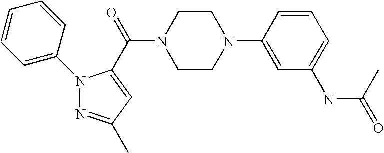 | 403.48 | 1.2 | 0.0595 | |
| E19 | 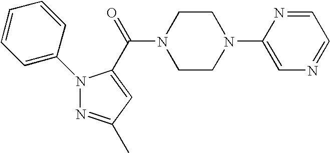 | 348.41 | 6 | 0.040 | |
| E20 | 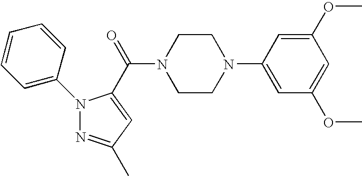 | 406.48 | 0.29 | 0.0015 | |
| E21 | 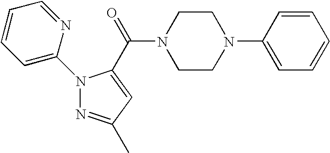 | 347.42 | 9.5 | 0.1982 | |
| E22 | 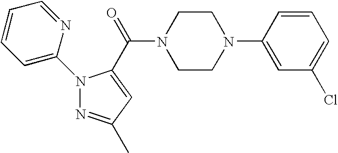 | 381.87 | 1.9 | 0.0064 | |
| E23 | 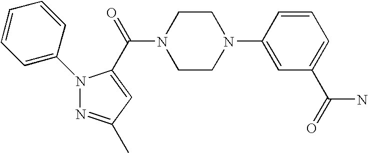 | 389.46 | 0.32 | 0.010 | |
| E24 | 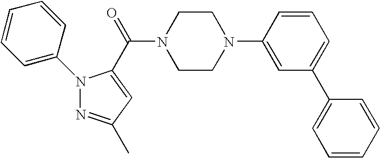 | 422.53 | 1.7 | 0.3119 | |
| E25 | 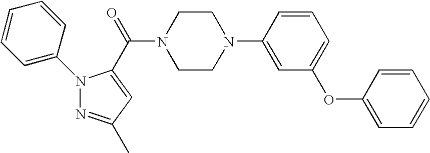 | 438.53 | 6 | 0.5602 | |
| E26 | 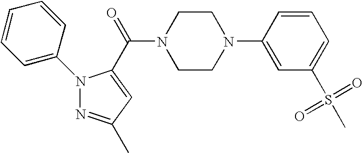 | 424.52 | 6.4 | 0.1351 | |
| E27 | 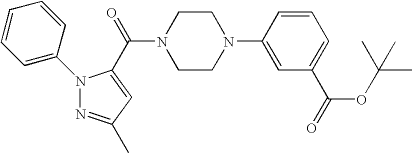 | 446.55 | 22.5 | | |
| E28 | 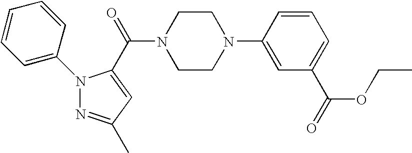 | 418.49 | 3.4 | 0.0606 | |
| E29 | 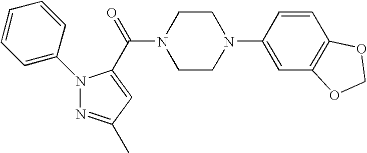 | 390.44 | 0.46 | 0.0095 | |
| E30 | 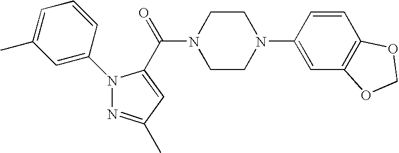 | 404.47 | 0.42 | 0.0021 | |
| E31 | 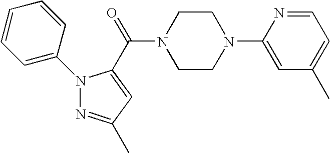 | 361.45 | 1.5 | 0.0208 | |
| E32 | 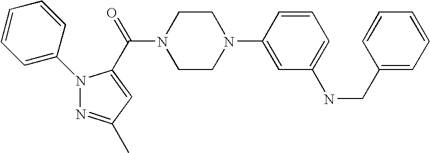 | 451.47 | 3.1 | 0.5135 | |

| | | | | |
|---|---|---|---|---|
| E33 | 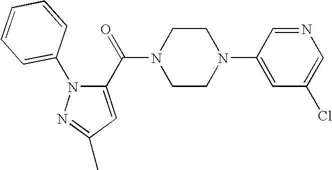 | 381.87 | 0.9 | 0.0089 |
| E34 | 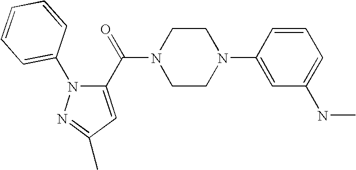 | 375.47 | 0.8 | 0.0140 |
| E35 | 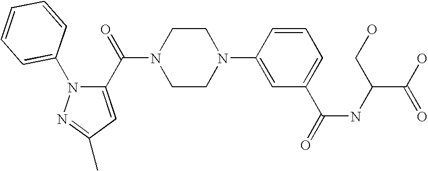 | 477.52 | 5.5 | |
| E36 | 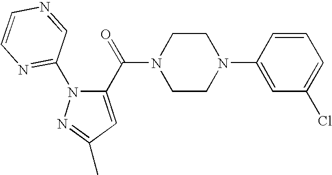 | 382.85 | 20 | 0.9388 |
| E37 | 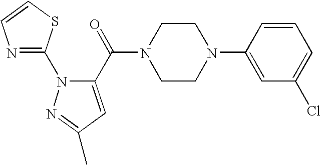 | 387.89 | 2 | 0.0377 |
| E38 | 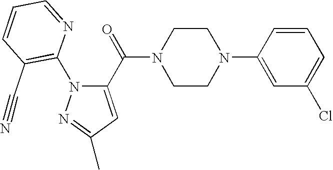 | 406.88 | 4 | 0.0196 |
| E39 | 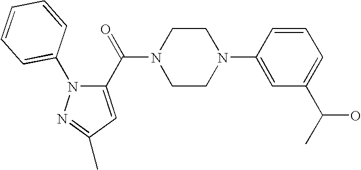 | 390.48 | 2.5 | 0.0719 |
| E40 | 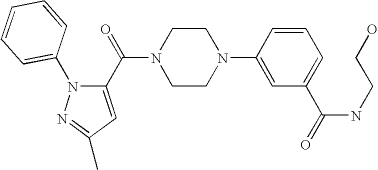 | 433.51 | 1.6 | 0.1185 |
| E41 | 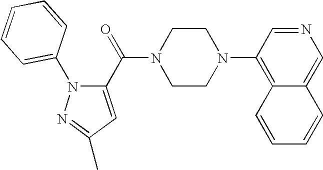 | 397.48 | 4.5 | 0.0894 |
| E42 | 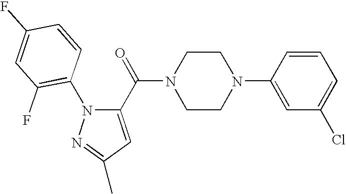 | 416.86 | 2.09 | 0.0502 |
| E43 | 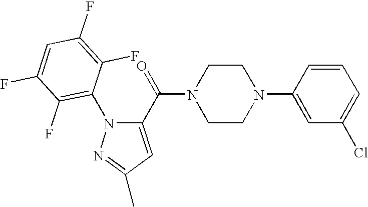 | 452.84 | 2.17 | 1.4635 |
| E44 | 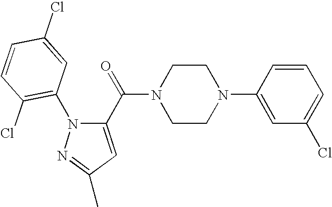 | 449.77 | 1.68 | 0.2357 |
| E45 | 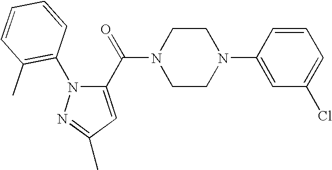 | 394.90 | 0.99 | 0.0105 |
| E46 | 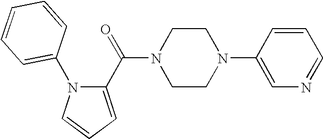 | 332.41 | 14.5 | 1.2360 |
| E47 | 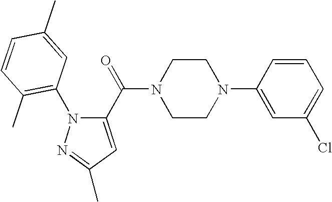 | 408.93 | 1.55 | |
| E48 | 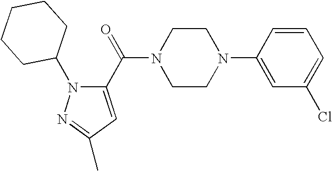 | 386.92 | 3.26 | 0.4895 |
| E49 | 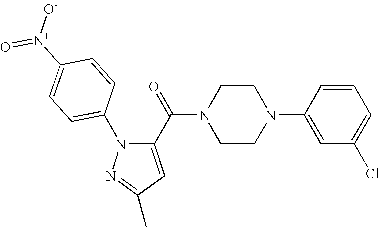 | 425.87 | 20.2 | |
| E50 | 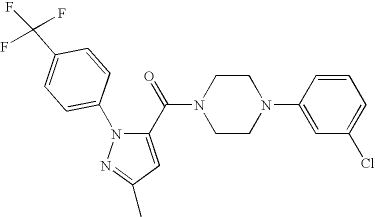 | 448.87 | 25 | |
| E51 | 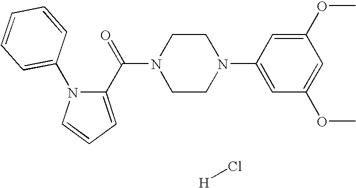 | 427.93 | 1.3 | 2.2980 |
| E52 | 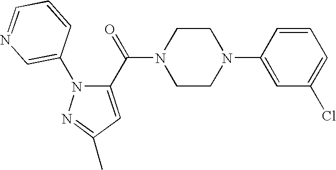 | 381.87 | 1.41 | 0.0102 |
| E53 | 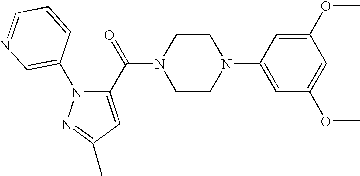 | 407.47 | 0.62 | 0.0111 |
| E54 | 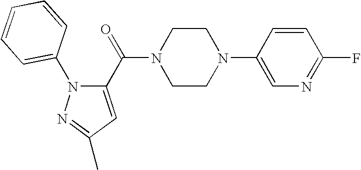 | 365.41 | 14.2 | 1.0370 |
| E55 | 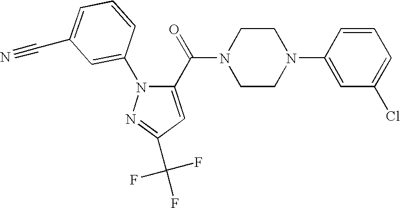 | 459.86 | 3.6 | 1.3370 |
| E56 | 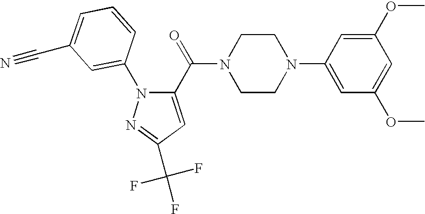 | 485.46 | 1.1 | 0.1450 |
| E57 | 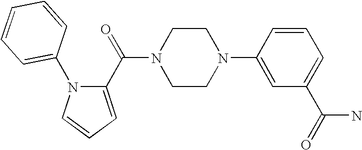 | 374.44 | 2.0 | 0.0898 |
| E58 | 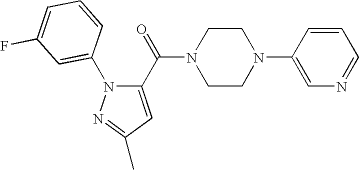 | 365.41 | 1 | 0.0411 |
| E59 | 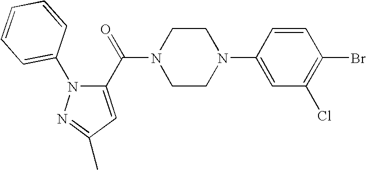 | 459.77 | 2.1 | 0.1040 |
| E60 | 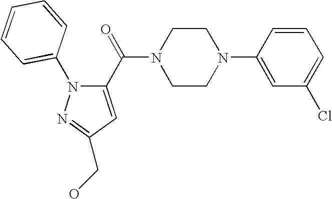 | 396.88 | 1.37 | 0.0117 |
| E61 | 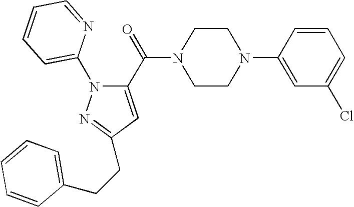 | 473.96 | 2.3 | 6.0900 |
| E62 | 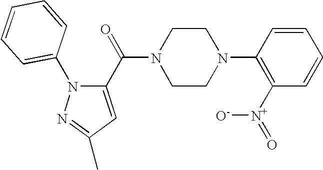 | 391.43 | 13.5 | |
| E63 | 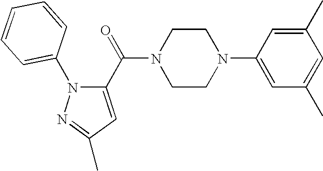 | 374.49 | 0.6 | 0.0022 |
| E64 | 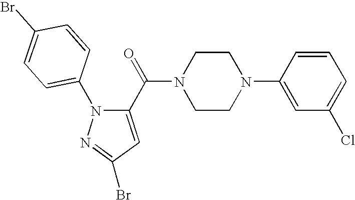 | 524.64 | 3.5 | |
| E65 | 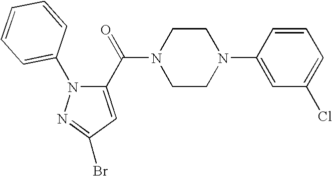 | 445.75 | 1.7 | 0.0106 |
| E66 | 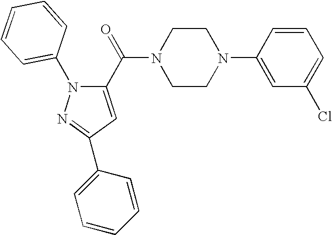 | 442.95 | 3.7 | 1.828 |
| E67 | 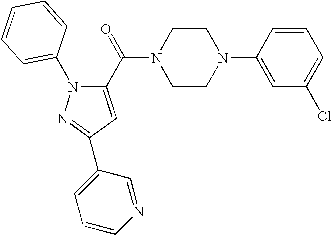 | 443.94 | 2.3 | 0.2370 |
| E68 | 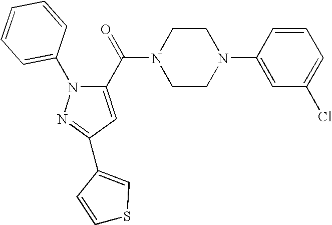 | 448.98 | 1.4 | 0.3390 |
| E69 | 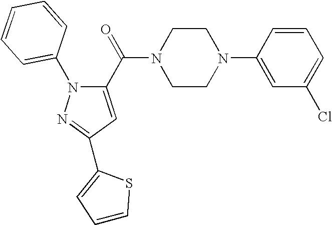 | 448.98 | 1.7 | 6.1540 |
| E70 | 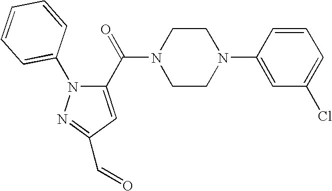 | 394.86 | 1.3 | 0.0108 |
| E71 | 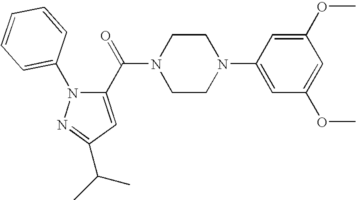 | 434.54 | 3.74 | 0.2000 |
| E72 | 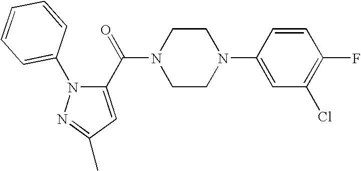 | 398.87 | 1.55 | 0.0066 |
| E73 | 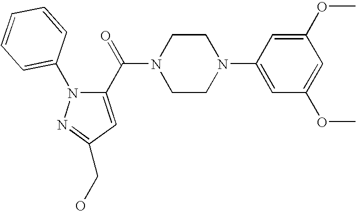 | 422.48 | 0.99 | 0.0069 |
| E74 | 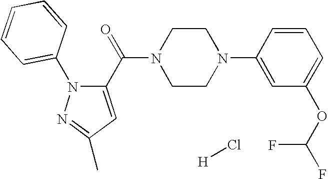 | 448.90 | | |
| E75 | 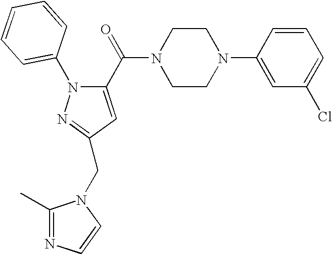 | 460.97 | 5.1 | 0.0612 |
| E76 | 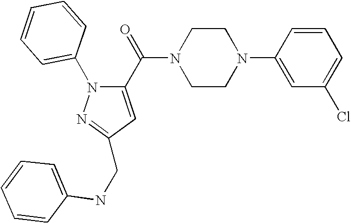 | 471.99 | 1.9 | 0.1375 |
| E77 | 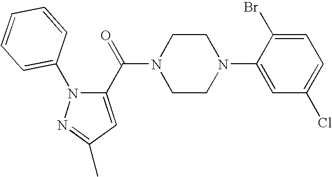 | 466.76 | 0.7 | 0.0267 |
| E78 | 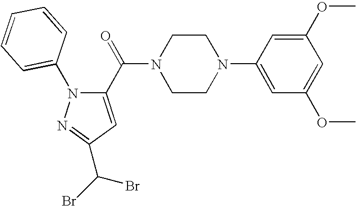 | 564.28 | 13.98 | 0.3180 |
| E79 | 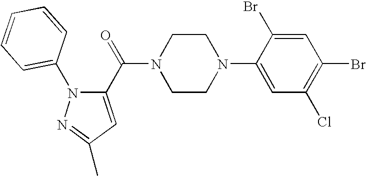 | 545.66 | 2.2 | 1.0380 |
| E80 | 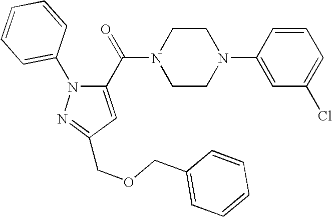 | 487.00 | 4.4 | 1.1260 |
| E81 | 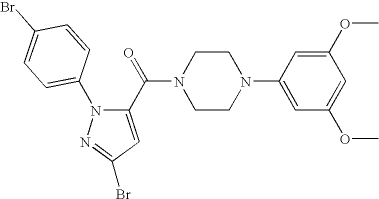 | 550.25 | 17.1 | |
| E82 | 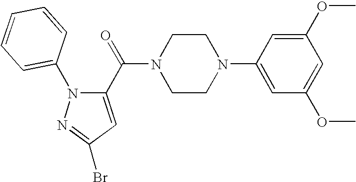 | 471.34 | 0.94 | 0.0018 |
| E83 | 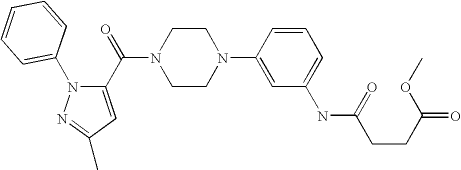 | 475.55 | 20 | |
| E84 | 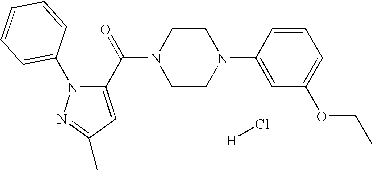 | 426.94 | 0.65 | 0.0029 |
| E85 | 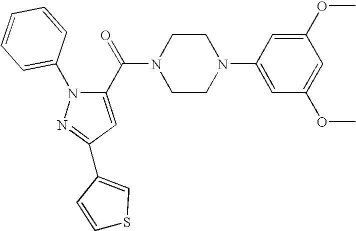 | 474.58 | 1.39 | 0.1807 |
| E86 | 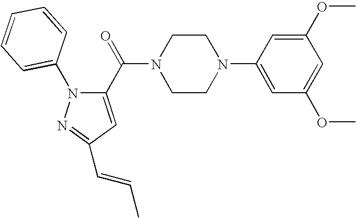 | 432.52 | 0.87 | 0.0068 |
| E87 | 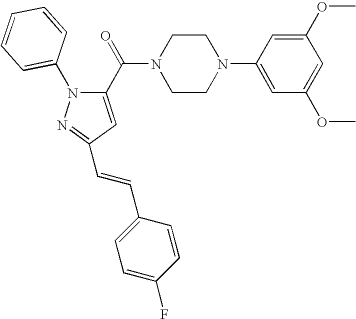 | 512.58 | 1.26 | 1.2830 |
| E88 | 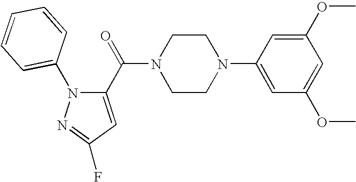 | 486.54 | 4.53 | 1.2570 |
| E89 | 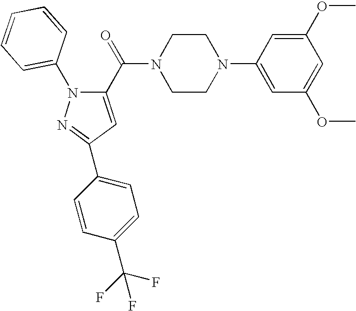 | 536.55 | 3.42 | 1.0980 |
| E90 | 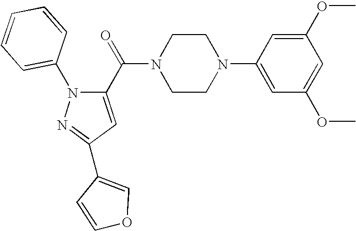 | 458.53 | 1.73 | 0.0188 |
| E91 | 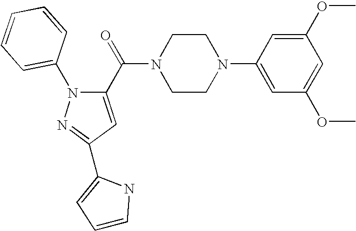 | 457.53 | 1 | 0.0105 |
| E92 | 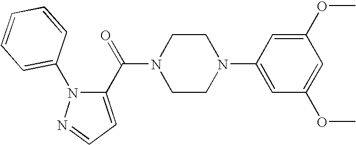 | 392.46 | 1.16 | 0.0010 |
| E93 | 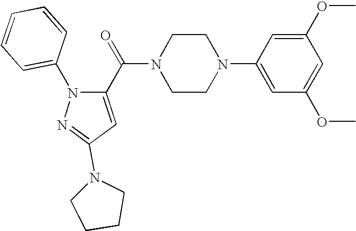 | 461.56 | 2.02 | 0.0063 |
| E94 | 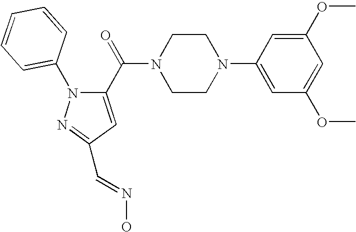 | 435.48 | 0.9 | 0.0020 |
| E95 | 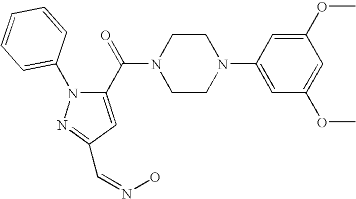 | 435.48 | 0.8 | 0.00017 |
| E96 | 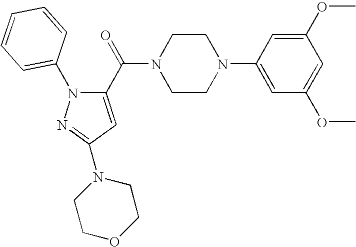 | 477.56 | 11.37 | 0.0733 |
| E97 | 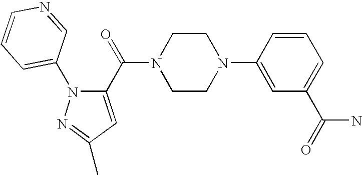 | 390.44 | 3.6 | 0.0811 |
| E98 | 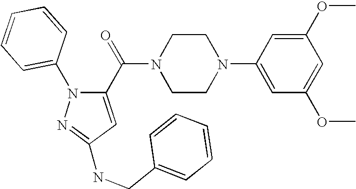 | 497.60 | 1.4 | 0.2230 |
| E99 | 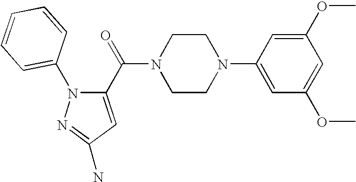 | 407.47 | 1 | 0.0024 |
| E100 | 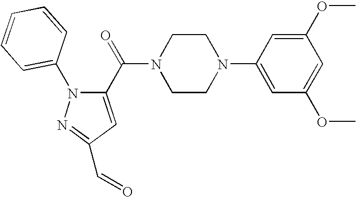 | 420.47 | 0.5 | 0.0041 |
| E101 | 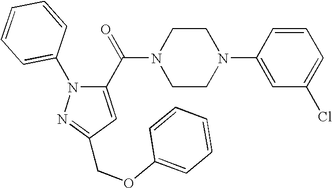 | 472.97 | 25 | |
| E102 | 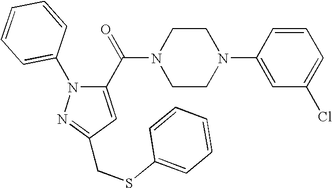 | 489.04 | 18 | |
| E103 | 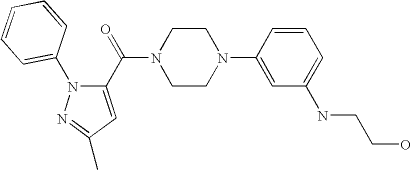 | 405.50 | 1 | 0.0167 |
| E104 | 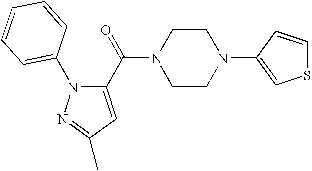 | 352.46 | 8 | 0.8389 |
| E105 | 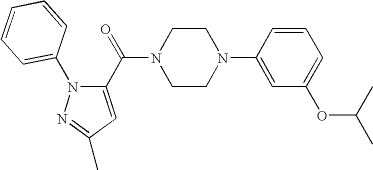 | 404.51 | 3.6 | 1.6510 |
| E106 | 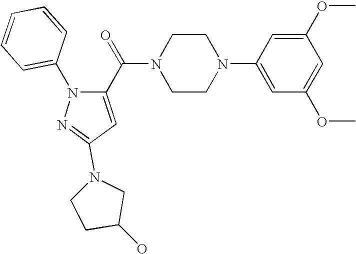 | 477.56 | 10 | 0.1419 |
| E107 | 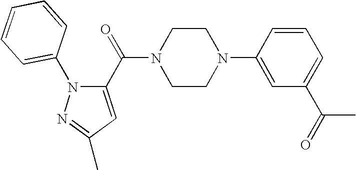 | 388.47 | 1.4 | 0.0104 |
| E108 | 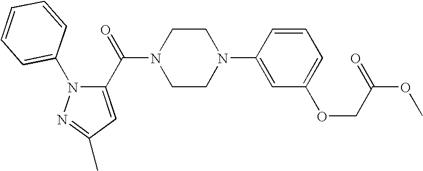 | 434.49 | 0.9 | |
| E109 | 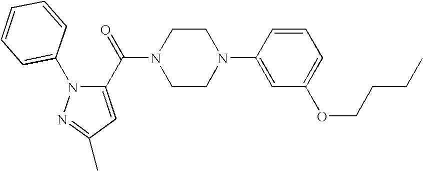 | 418.54 | 5.2 | 8.0450 |
| E110 | 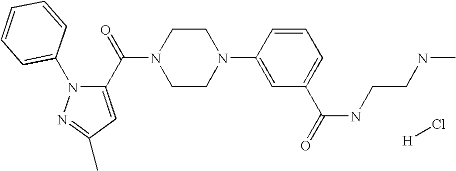 | 483.01 | 12 | 1.1661 |
| E111 | 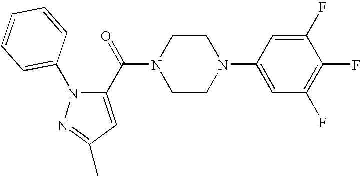 | 400.40 | 2.33 | |
| E112 | 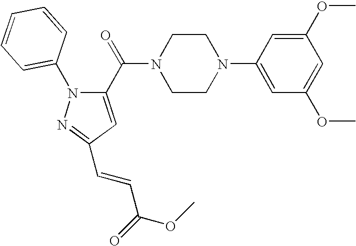 | 490.56 | 3 | |
| E113 | 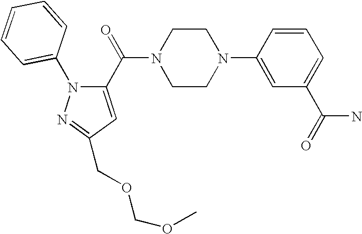 | 449.51 | 9.2 | |
| E114 | 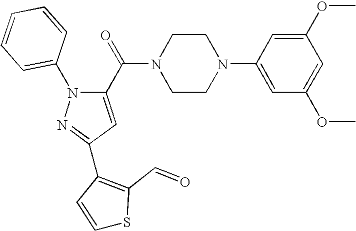 | 502.59 | 12.9 | |
| E115 | 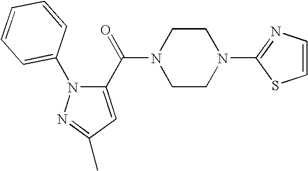 | 353.45 | <25 | 3.2370 |
| E116 | 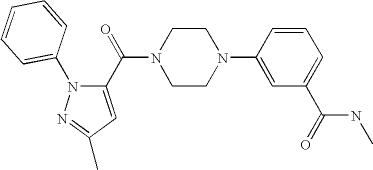 | 403.48 | 2.07 | 0.0113 |
| E117 | 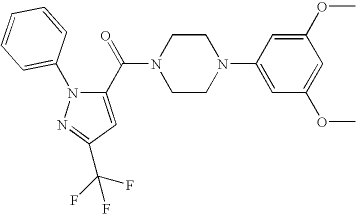 | 460.45 | 7.9 | 0.0284 |
| E118 | 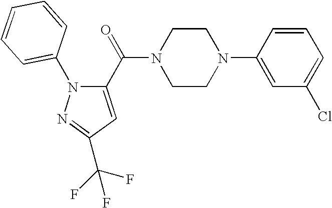 | 434.85 | 2.3 | 0.4030 |
| E119 | 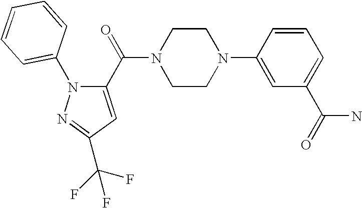 | 443.43 | 1.2 | 0.0103 |
| E120 | 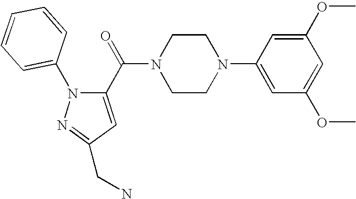 | 421.50 | 17.9 | |
| E121 | 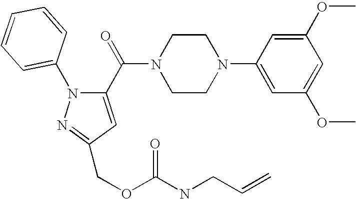 | 505.57 | 0.9 | |
| E122 | 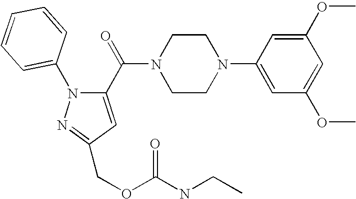 | 493.56 | 1.2 | |
| E123 | 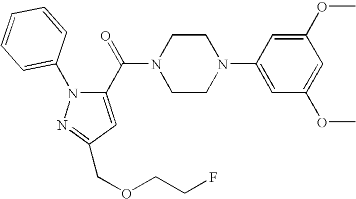 | 468.53 | 2.5 | |
| E124 | 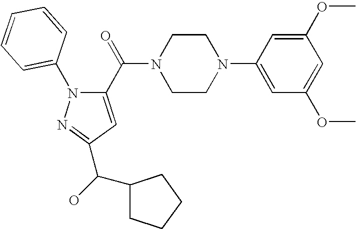 | 490.60 | <25 | |
| E125 | 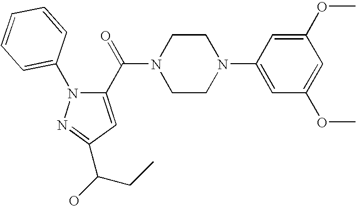 | 450.54 | 4.1 | |
| E126 | 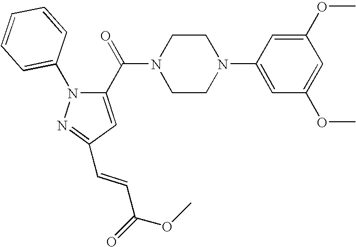 | 476.53 | 0.8 | |
| E127 | 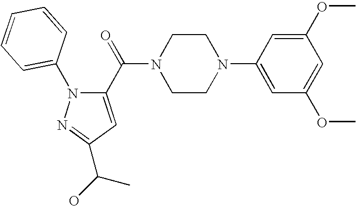 | 436.51 | 2.3 | |
| E128 | 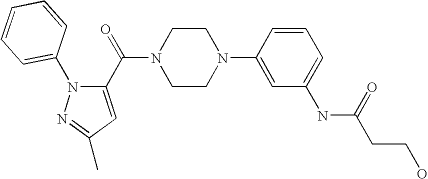 | 433.51 | 3.0 | 0.1196 |
| E129 | 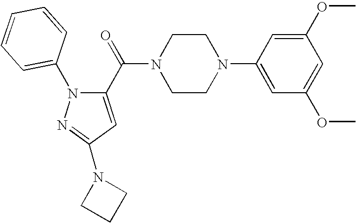 | 447.54 | 0.9 | |
| E130 | 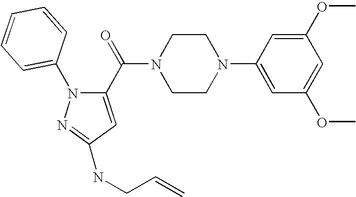 | 447.54 | 1.5 | |
| E131 | 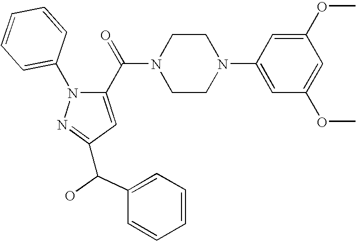 | 498.58 | <25 | |
| E132 | 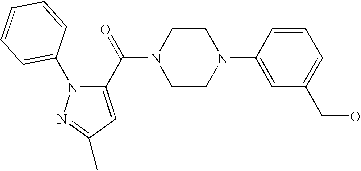 | 376.48 | 0.9 | |
| E133 | 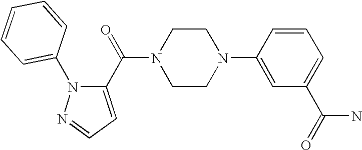 | 375.43 | 1 | |
| E134 | 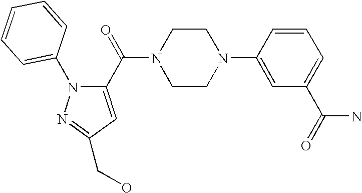 | 405.46 | 3.2 | |
| E135 | 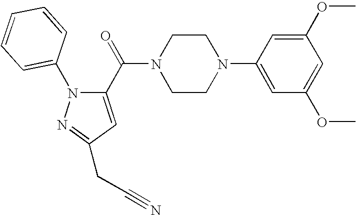 | 431.95 | 0.5 | |
| E136 | 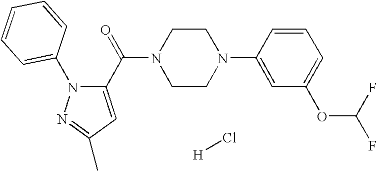 | 448.89 | 1.3 | |
| E137 | 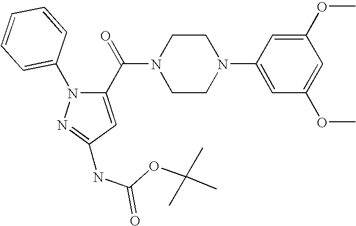 | 537.66 | 3.2 | |
| E138 | 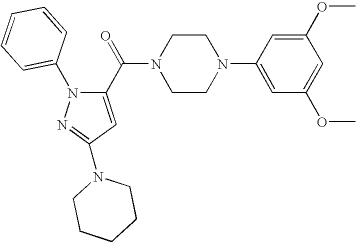 | 475.59 | 22 | |
| E139 | 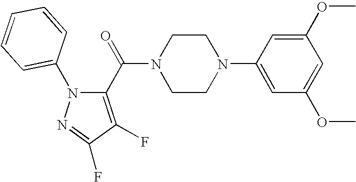 | 428.43 | 2.9 | |
| E140 | 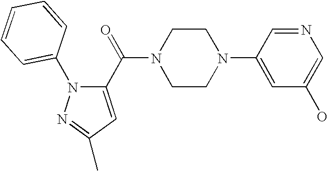 | 363.41 | 16.5 | |

1. A product corresponding to Formula (I) below: 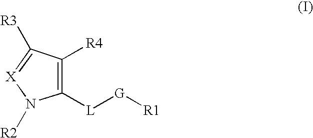
in which:
R1 and R2 are selected independently from the group consisting of aryl, heteroaryl, substituted aryl and substituted heteroaryl; R2 may also be chosen from the group C5-C7 cycloalkyl;
L is selected from the group consisting of C(R7)(R8), C═O, C═S, and C═N(R7);
R3 is selected from the group consisting of H, halogen, CF3, alkyl, substituted alkyl, alkylene, substituted alkylene, alkynyl, substituted alkynyl, cycloalkyl, cycloalkylene, heterocyclyl, substituted heterocyclyl, CO—R7, C(R7)═N—O(R8), COOH, CONH-aryl, CONH-heteroaryl, CONH—R7, CON(R7)(R8), CO—N(R7)-aryl, CO—N(R7)-heteroaryl, C(OR7)═NH, C[N(R7)(R8)]═NH, NH2, NH-aryl, NH-heteroaryl, NH(R7), N(R7)(R8), NH—CO—R7, N(R7)—CO-aryl, N(R7)—CO-heteroaryl, NH—SO2—R7, NH—SO2-aryl, NH—SO2-heteroaryl, NH—CH2—CO2(R7), NH—CH2-aryl, NH—CH2-heteroaryl, NH—COO—(C1-C4)alkyl, NH—CH2—(C2-C3)alkylene, NH—CH2—(C2-C3)alkynyl, N(R7)—N(R8)(R12), N—N═C(R7)(R8), CN, O—R7, O—CH2-aryl, O—CH2-heteroaryl, S—R7, SO—R7, SO₂—R7, aryl, heteroaryl, substituted cycloalkyl, substituted aryl and substituted heteroaryl;
R4 is selected from the group consisting of H, (C1-C3)alkyl, cyclopropyl, (C2-C3)alkylene, (C2-C3)alkynyl, O(C1-C3)alkyl, S—(C1-C3)alkyl, F, Cl and Br;
X is N or CH;
R7, R8 and R12 are independently selected from the group consisting of H, (C1-C3)alkyl and substituted (C1-C3)alkyl;
L-G-R1 is selected from the group consisting of: 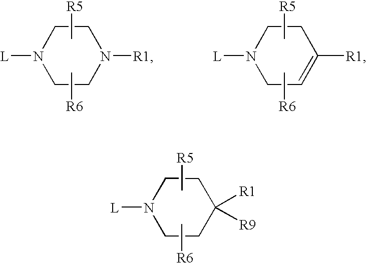
L and R1 are as defined above;
R5 and R6 are selected independently from the group consisting of H, (C1-C3)alkyl, oxo and halogen; and
R9 is (C1-C3)alkyl;
in racemic form, enriched in one enantiomer, enriched in one diastereoisomer, its tautomers, its prodrugs and its pharmaceutically acceptable salts, with the proviso that the product of formula (I) is not one of the following compounds:

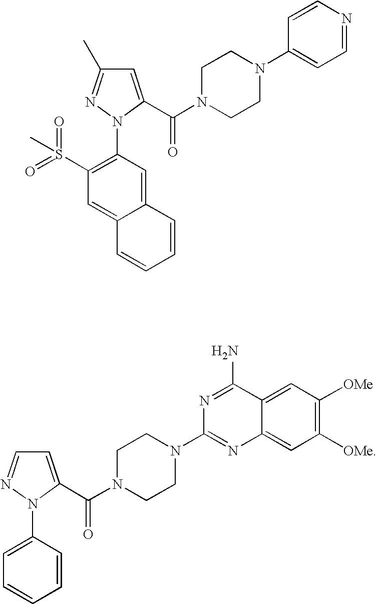

2. The product as claimed in claim 1, characterized in that X is N.

3. The product according to claim 1 or 2, characterized in that L-G-R1 is 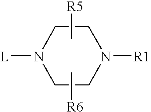
in which R5 and R6 are each H.

4. The product as claimed in any one of claims 1 to 3, characterized in that R1 is chosen from:
phenyl;
phenyl substituted with at least one radical chosen from halogen, (C1-C3)alkyl, CON(R10)(R11), O—R10, S—R10 and N(R10)(R11);
pyridyl;
pyridyl substituted with at least one radical chosen from halogen, (C1-C3)alkyl, CON(R10)(R11), O—R10, S—R10 and N(R10)(R11), in which R10 and R11 are chosen independently from H, (C1-C3)alkyl and halogenated (C1-C3)alkyl.

5. The product as claimed in claim 4, characterized in that R1 is chosen from:
phenyl substituted with halogen, (C1-C3)alkyl, (C1-C3)alkoxy or carboxamide,
2-pyridyl or 3-pyridyl,
2-pyridyl or 3-pyridyl substituted with halogen or (C1-C3)alkyl.

6. The product as claimed in claim 5, characterized in that R1 is phenyl substituted with:
a chloro radical, or
one or two methoxy radicals, or
a carboxamide radical.

7. The product as claimed in any one of claims 4 to 6, characterized in that R1 is chosen from 2,3-disubstituted phenyl, 2,5-disubstituted phenyl, 3-substituted phenyl, 3,5-disubstituted phenyl and 3,4-disubstituted phenyl.

8. The product as claimed in claim 5, characterized in that R1 is chosen from 4-substituted 2-pyridyl, 6-substituted 2-pyridyl, 4,6-disubstituted 2-pyridyl, 2-substituted 3-pyridyl and 5-substituted 3-pyridyl.

9. The product as claimed in claim 1, characterized in that R2 is chosen from phenyl, 3-pyridyl, phenyl substituted with at least one radical chosen from halogen, alkyl, O—R10, S—R10 and N(R10)(R11), in which R10 and R11 are independently chosen from H, (C1-C3)alkyl and halogenated (C1-C3)alkyl.

10. The product as claimed in claim 1, characterized in that R3 is chosen from H, (C1-C3)alkyl, CF3, hydroxymethyl, amino, azetidino and pyrrolidino.

11. The product as claimed in claim 10, characterized in that R3 is chosen from methyl, hydroxymethyl, CF3 and amino.

12. The product as claimed in claim 1, characterized in that R4 is H.

13. The product as claimed in claim 1, characterized in that it is chosen from:
(3,5-Dimethyl-1-phenyl-1H-pyrazol-4-yl)(4-phenylpiperazin-1-yl)methanone
(3,5-Dimethyl-1-phenyl-1H-pyrazol-4-yl)[4-(2-methoxyphenyl)piperazin-1-yl]methanone
(3,5-Dimethyl-1-phenyl-1H-pyrazol-4-yl)[4-(3-trifluoromethylphenyl)piper-azin-1-yl]methanone
(3,5-Dimethyl-1-phenyl-1H-pyrazol-4-yl)[4-(4-fluorophenyl)piperazin-1-yl]-methanone
1-{4-[4-(3,5-Dimethyl-1-phenyl-1H-pyrazole-4-carbonyl)piperazin-1-yl]-phenyl}ethanone
[4-(2,4-Dimethylphenyl)piperazin-1-yl](3,5-dimethyl-1-phenyl-1H-pyrazol-4-yl)methanone
[4-(3,4-Dichlorophenyl)piperazin-1-yl](3,5-dimethyl-1-phenyl-1H-pyrazol-4-yl)methanone
[4-(3,4-Dimethylphenyl)piperazin-1-yl](3,5-dimethyl-1-phenyl-1H-pyrazol-4-yl)methanone
(3,5-Dimethyl-1-phenyl-1H-pyrazol-4-yl)(4-o-tolylpiperazin-1-yl)methanone
[4-(2,3-Dimethylphenyl)piperazin-1-yl](3,5-dimethyl-1-phenyl-1H-pyrazol-4-yl)methanone
(3,5-Dimethyl-1-phenyl-1H-pyrazol-4-yl)[4-(2-ethylphenyl)piperazin-1-yl]-methanone
[4-(3-Chlorophenyl)piperazin-1-yl](3,5-dimethyl-1-phenyl-1H-pyrazol-4-yl)-methanone
(3,5-Dimethyl-1-phenyl-1H-pyrazol-4-yl)(4-m-tolylpiperazin-1-yl)methanone
(3,5-Dimethyl-1-phenyl-1H-pyrazol-4-yl)[4-(4-methoxyphenyl)piperazin-1-yl]-methanone
[4-(2,4-Dimethoxyphenyl)piperazin-1-yl](3,5-dimethyl-1-phenyl-1H-pyrazol-4-yl)methanone
(3,5-Dimethyl-1-phenyl-1H-pyrazol-4-yl)[4-(2-methylsulfanylphenyl)piper-azin-1-yl]methanone
[4-(4-Chlorophenyl)piperazin-1-yl](3,5-dimethyl-1-phenyl-1H-pyrazol-4-yl)-methanone
(3,5-Dimethyl-1-phenyl-1H-pyrazol-4-yl)(4-naphthalen-1-ylpiperazin-1-yl)-methanone
[4-(5-Chloro-2-methylphenyl)piperazin-1-yl](3,5-dimethyl-1-phenyl-1H-pyrazol-4-yl)methanone
(3,5-Dimethyl-1-phenyl-1H-pyrazol-4-yl)[4-(2-ethoxyphenyl)piperazin-1-yl]-methanone
[4-(2,3-Dichlorophenyl)piperazin-1-yl](3,5-dimethyl-1-phenyl-1H-pyrazol-4-yl)methanone
(3,5-Dimethyl-1-phenyl-1H-pyrazol-4-yl)[4-(4-trifluoromethylphenyl)piper-azin-1-yl]methanone
[4-(5-Chloro-2-methoxyphenyl)piperazin-1-yl](3,5-dimethyl-1-phenyl-1H-pyrazol-4-yl)methanone
[4-(4-Benzyloxyphenyl)piperazin-1-yl](3,5-dimethyl-1-phenyl-1H-pyrazol-4-yl)methanone
(3,5-Dimethyl-1-phenyl-1H-pyrazol-4-yl)[4-(2-trifluoromethylphenyl)piper-azin-1-yl]methanone
[4-(2,3-Dihydrobenzo[1,4]dioxin-6-yl)piperazin-1-yl](3,5-dimethyl-1-phenyl-1H-pyrazol-4-yl)methanone
[2-(4-Methoxyphenyl)-5-methyl-2H-pyrazol-3-yl](4-phenylpiperazin-1-yl)-methanone
[2-(4-Methoxyphenyl)-5-methyl-2H-pyrazol-3-yl][4-(2-methoxyphenyl)piper-azin-1-yl]methanone
[2-(4-Methoxyphenyl)-5-methyl-2H-pyrazol-3-yl][4-(3-trifluoromethylphenyl)-piperazin-1-yl]methanone
[4-(4-Fluorophenyl)piperazin-1-yl][2-(4-methoxyphenyl)-5-methyl-2H-pyrazol-3-yl]methanone
1-(4-{4-[2-(4-Methoxyphenyl)-5-methyl-2H-pyrazole-3-carbonyl]piperazin-1-yl}phenyl)ethanone
[4-(2,4-Dimethylphenyl)piperazin-1-yl][2-(4-methoxyphenyl)-5-methyl-2H-pyrazol-3-yl]methanone
[4-(3,4-Dichlorophenyl)piperazin-1-yl][2-(4-methoxyphenyl)-5-methyl-2H-pyrazol-3-yl]methanone
[4-(3,4-Dimethylphenyl)piperazin-1-yl][2-(4-methoxyphenyl)-5-methyl-2H-pyrazol-3-yl]methanone
[2-(4-Methoxyphenyl)-5-methyl-2H-pyrazol-3-yl](4-o-tolylpiperazin-1-yl)-methanone
[4-(2,3-Dimethylphenyl)piperazin-1-yl][2-(4-methoxyphenyl)-5-methyl-2H-pyrazol-3-yl]methanone
[4-(2-Ethylphenyl)piperazin-1-yl][2-(4-methoxyphenyl)-5-methyl-2H-pyrazol-3-yl]methanone
[4-(3-Chlorophenyl)piperazin-1-yl][2-(4-methoxyphenyl)-5-methyl-2H-pyrazol-3-yl]methanone
[2-(4-Methoxyphenyl)-5-methyl-2H-pyrazol-3-yl](4-m-tolylpiperazin-1-yl)-methanone
[2-(4-Methoxyphenyl)-5-methyl-2H-pyrazol-3-yl][4-(4-methoxyphenyl)-piperazin-1-yl]methanone
[4-(2,4-Dimethoxyphenyl)piperazin-1-yl][2-(4-methoxyphenyl)-5-methyl-2H-pyrazol-3-yl]methanone
[2-(4-Methoxyphenyl)-5-methyl-2H-pyrazol-3-yl][4-(2-methylsulfanylphenyl)-piperazin-1-yl]methanone
[4-(4-Chlorophenyl)piperazin-1-yl][2-(4-methoxyphenyl)-5-methyl-2H-pyrazol-3-yl]methanone
[2-(4-Methoxyphenyl)-5-methyl-2H-pyrazol-3-yl](4-naphthalen-1-ylpiperazin-1-yl)methanone
[4-(5-Chloro-2-methylphenyl)piperazin-1-yl][2-(4-methoxyphenyl)-5-methyl-2H-pyrazol-3-yl]methanone
[4-(2-Ethoxyphenyl)piperazin-1-yl][2-(4-methoxyphenyl)-5-methyl-2H-pyrazol-3-yl]methanone
[4-(2,3-Dichlorophenyl)piperazin-1-yl][2-(4-methoxyphenyl)-5-methyl-2H-pyrazol-3-yl]methanone
[2-(4-Methoxyphenyl)-5-methyl-2H-pyrazol-3-yl][4-(4-trifluoromethylphenyl)-piperazin-1-yl]methanone
[4-(5-Chloro-2-methoxyphenyl)piperazin-1-yl][2-(4-methoxyphenyl)-5-methyl-2H-pyrazol-3-yl]methanone
[4-(4-Benzyloxyphenyl)piperazin-1-yl][2-(4-methoxyphenyl)-5-methyl-2H-pyrazol-3-yl]methanone
[2-(4-Methoxyphenyl)-5-methyl-2H-pyrazol-3-yl][4-(2-trifluoromethylphenyl)-piperazin-1-yl]methanone
[4-(2,3-Dihydrobenzo[1,4]dioxin-6-yl)piperazin-1-yl][2-(4-methoxyphenyl)-5-methyl-2H-pyrazol-3-yl]methanone
[2-(3-Methoxyphenyl)-5-methyl-2H-pyrazol-3-yl](4-phenylpiperazin-1-yl)-methanone
2-(3-Methoxyphenyl)-5-methyl-2H-pyrazol-3-yl][4-(2-methoxyphenyl)piper-azin-1-yl]methanone
[2-(3-Methoxyphenyl)-5-methyl-2H-pyrazol-3-yl][4-(3-trifluoromethylphenyl)-piperazin-1-yl]methanone
[4-(4-Fluorophenyl)piperazin-1-yl][2-(3-methoxyphenyl)-5-methyl-2H-pyrazol-3-yl]methanone
1-(4-{4-[2-(3-Methoxyphenyl)-5-methyl-2H-pyrazole-3-carbonyl]piperazin-1-yl}phenyl)ethanone
[4-(2,4-Dimethylphenyl)piperazin-1-yl][2-(3-methoxyphenyl)-5-methyl-2H-pyrazol-3-yl]methanone
[4-(3,4-Dichlorophenyl)piperazin-1-yl][2-(3-methoxyphenyl)-5-methyl-2H-pyrazol-3-yl]methanone
[4-(3,4-Dimethylphenyl)piperazin-1-yl][2-(3-methoxyphenyl)-5-methyl-2H-pyrazol-3-yl]methanone
[2-(3-Methoxyphenyl)-5-methyl-2H-pyrazol-3-yl](4-o-tolylpiperazin-1-yl)-methanone
[4-(2,3-Dimethylphenyl)piperazin-1-yl][2-(3-methoxyphenyl)-5-methyl-2H-pyrazol-3-yl]methanone
[4-(2-Ethylphenyl)piperazin-1-yl][2-(3-methoxyphenyl)-5-methyl-2H-pyrazol-3-yl]methanone
[4-(3-Chlorophenyl)piperazin-1-yl][2-(3-methoxyphenyl)-5-methyl-2H-pyrazol-3-yl]methanone
[2-(3-Methoxyphenyl)-5-methyl-2H-pyrazol-3-yl](4-m-tolylpiperazin-1-yl)-methanone
[2-(3-Methoxyphenyl)-5-methyl-2H-pyrazol-3-yl][4-(4-methoxyphenyl)piper-azin-1-yl]methanone
[4-(2,4-Dimethoxyphenyl)piperazin-1-yl][2-(3-methoxyphenyl)-5-methyl-2H-pyrazol-3-yl]methanone
[2-(3-Methoxyphenyl)-5-methyl-2H-pyraol-3-yl][4-(2-methylsulfanylphenyl)-piperazin-1-yl]methanone
[4-(4-Chlorophenyl)piperazin-1-yl][2-(3-methoxyphenyl)-5-methyl-2H-pyrazol-3-yl]methanone
[2-(3-Methoxyphenyl)-5-methyl-2H-pyrazol-3-yl](4-naphthalen-1-ylpiperazin-1-yl)methanone
[4-(5-Chloro-2-methylphenyl)piperazin-1-yl][2-(3-methoxyphenyl)-5-methyl-2H-pyrazol-3-yl]methanone
[4-(2-Ethoxyphenyl)piperazin-1-yl][2-(3-methoxyphenyl)-5-methyl-2H-pyrazol-3-yl]methanone
[4-(2,3-Dichlorophenyl)piperazin-1-yl][2-(3-methoxyphenyl)-5-methyl-2H-pyrazol-3-yl]methanone
[2-(3-Methoxyphenyl)-5-methyl-2H-pyrazol-3-yl][4-(4-trifluoromethylphenyl)-piperazin-1-yl]methanone
[4-(5-Chloro-2-methoxyphenyl)piperazin-1-yl][2-(3-methoxyphenyl)-5-methyl-2H-pyrazol-3-yl]methanone
[4-(4-Benzyloxyphenyl)piperazin-1-yl][2-(3-methoxyphenyl)-5-methyl-2H-pyrazol-3-yl]methanone
[2-(3-Methoxyphenyl)-5-methyl-2H-pyrazol-3-yl][4-(2-trifluoromethylphenyl)-piperazin-1-yl]methanone
[4-(2,3-Dihydrobenzo[1,4]dioxin-6-yl)piperazin-1-yl][2-(3-methoxyphenyl)-5-methyl-2H-pyrazol-3-yl]methanone
(5-Methyl-2-phenyl-2H-pyrazol-3-yl)(4-phenylpiperazin-1-yl)methanone
[4-(2-Methoxyphenyl)piperazin-1-yl](5-methyl-2-phenyl-2H-pyrazol-3-yl)-methanone
(5-Methyl-2-phenyl-2H-pyrazol-3-yl)[4-(3-trifluoromethylphenyl)piperazin-1-yl]methanone
[4-(4-Fluorophenyl)piperazin-1-yl](5-methyl-2-phenyl-2H-pyrazol-3-yl)methanone
1-{4-[4-(5-Methyl-2-phenyl-2H-pyrazole-3-carbonyl)piperazin-1-yl]-phenyl)-ethanone
[4-(2,4-Dimethylphenyl)piperazin-1-yl](5-methyl-2-phenyl-2H-pyrazol-3-yl)-methanone
[4-(3,4-Dichlorophenyl)piperazin-1-yl](5-methyl-2-phenyl-2H-pyrazol-3-yl)-methanone
(5-Methyl-2-phenyl-2H-pyrazol-3-yl)(4-o-tolylpiperazin-1-yl)methanone
[4-(2,3-Dimethylphenyl)piperazin-1-yl](5-methyl-2-phenyl-2H-pyrazol-3-yl)-methanone
[4-(2-Ethylphenyl)piperazin-1-yl](5-methyl-2-phenyl-2H-pyrazol-3-yl)methanone
[4-(3-Chlorophenyl)piperazin-1-yl](5-methyl-2-phenyl-2H-pyrazol-3-yl)methanone
[4-(3-Methoxyphenyl)piperazin-1-yl](5-methyl-2-phenyl-2H-pyrazol-3-yl)-methanone
(5-Methyl-2-phenyl-2H-pyrazol-3-yl)(4-m-tolylpiperazin-1-yl)methanone
[4-(4-Methoxyphenyl)piperazin-1-yl](5-methyl-2-phenyl-2H-pyrazol-3-yl)-methanone
[4-(2,4-Dimethoxyphenyl)piperazin-1-yl](5-methyl-2-phenyl-2H-pyrazol-3-yl)-methanone
(5-Methyl-2-phenyl-2H-pyrazol-3-yl)[4-(2-methylsulfanylphenyl)piperazin-1-yl]methanone
[4-(4-Chlorophenyl)piperazin-1-yl](5-methyl-2-phenyl-2H-pyrazol-3-yl)methanone
(5-Methyl-2-phenyl-2H-pyrazol-3-yl)(4-naphthalen-1-ylpiperazin-1-yl)methanone
[4-(5-Chloro-2-methylphenyl)piperazin-1-yl](5-m ethyl-2-phenyl-2H-pyrazol-3-yl)methanone
[4-(2-Ethoxyphenyl)piperazin-1-yl](5-methyl-2-phenyl-2H-pyrazol-3-yl)methanone
[4-(2,3-Dichlorophenyl)piperazin-1-yl](5-methyl-2-phenyl-2H-pyrazol-3-yl)-methanone
(5-Methyl-2-phenyl-2H-pyrazol-3-yl)[4-(4-trifluoromethylphenyl)piperazin-1-yl]methanone
[4-(5-Chloro-2-methoxyphenyl)piperazin-1-yl](5-methyl-2-phenyl-2H-pyrazol-3-yl)methanone
[4-(4-Benzyloxyphenyl)piperazin-1-yl](5-methyl-2-phenyl-2H-pyrazol-3-yl)-methanone
[4-(2,3-Dihydrobenzo[1,4]dioxin-6-yl)piperazin-1-yl](5-methyl-2-phenyl-2H-pyrazol-3-yl)methanone
N-{4-[4-(5-Methyl-2-phenyl-2H-pyrazole-3-carbonyl)piperazin-1-yl]-phenyl)-methanesulfonamide
[2-(2-Fluorophenyl)-5-methyl-2H-pyrazol-3-yl](4-phenylpiperazin-1-yl)methanone
[2-(2-Fluorophenyl)-5-methyl-2H-pyrazol-3-yl][4-(2-methoxyphenyl)piperazin-1-yl]methanone
[2-(2-Fluorophenyl)-5-methyl-2H-pyrazol-3-yl][4-(4-fluorophenyl)piperazin-1-yl]methanone
1-(4-{4-[2-(2-Fluorophenyl)-5-methyl-2H-pyrazole-3-carbonyl]piperazin-1-yl}-phenyl)ethanone
[4-(2,4-Dimethylphenyl)piperazin-1-yl][2-(2-fluorophenyl)-5-methyl-2H-pyrazol-3-yl]methanone
[4-(3,4-Dichlorophenyl)piperazin-1-yl][2-(2-fluorophenyl)-5-methyl-2H-pyrazol-3-yl]methanone
[4-(3,4-Dimethylphenyl)piperazin-1-yl][2-(2-fluorophenyl)-5-methyl-2H-pyrazol-3-yl]methanone
[2-(2-Fluorophenyl)-5-methyl-2H-pyrazol-3-yl](4-o-tolylpiperazin-1-yl)-methanone
[4-(2,3-Dimethylphenyl)piperazin-1-yl][2-(2-fluorophenyl)-5-methyl-2H-pyrazol-3-yl]methanone
[4-(2-Ethylphenyl)piperazin-1-yl][2-(2-fluorophenyl)-5-methyl-2H-pyrazol-3-yl]methanone
[4-(3-Chlorophenyl)piperazin-1-yl][2-(2-fluorophenyl)-5-methyl-2H-pyrazol-3-yl]methanone
[2-(2-Fluorophenyl)-5-methyl-2H-pyrazol-3-yl][4-(3-methoxyphenyl)piperazin-1-yl]methanone
[2-(2-Fluorophenyl)-5-methyl-2H-pyrazol-3-yl](4-m-tolylpiperazin-1-yl)methanone
[2-(2-Fluorophenyl)-5-methyl-2H-pyrazol-3-yl][4-(4-methoxyphenyl)piperazin-1-yl]methanone
[4-(2,4-Dimethoxyphenyl)piperazin-1-yl][2-(2-fluorophenyl)-5-methyl-2H-pyrazol-3-yl]methanone
[2-(2-Fluorophenyl)-5-methyl-2H-pyrazol-3-yl][4-(2-methylsulfanylphenyl)-piperazin-1-yl]methanone
[4-(4-Chlorophenyl)piperazin-1-yl][2-(2-fluorophenyl)-5-methyl-2H-pyrazol-3-yl]methanone
[2-(2-Fluorophenyl)-5-methyl-2H-pyrazol-3-yl](4-naphthalen-1-ylpiperazin-1-yl)methanone
[4-(5-Chloro-2-methylphenyl)piperazin-1-yl][2-(2-fluorophenyl)-5-methyl-2H-pyrazol-3-yl]methanone
[4-(2-Ethoxyphenyl)piperazin-1-yl][2-(2-fluorophenyl)-5-methyl-2H-pyrazol-3-yl]methanone
[4-(2,3-Dichlorophenyl)piperazin-1-yl][2-(2-fluorophenyl)-5-methyl-2H-pyrazol-3-yl]methanone
[2-(2-Fluorophenyl)-5-methyl-2H-pyrazol-3-yl][4-(4-trifluoromethylphenyl)-piperazin-1-yl]methanone
[4-(5-Chloro-2-methoxyphenyl)piperazin-1-yl][2-(2-fluorophenyl)-5-methyl-2H-pyrazol-3-yl]methanone
[2-(2-Fluorophenyl)-5-methyl-2H-pyrazol-3-yl][4-(2-trifluoromethylphenyl)-piperazin-1-yl]methanone
[4-(2,3-Dihydrobenzo[1,4]dioxin-6-yl)piperazin-1-yl][2-(2-fluorophenyl)-5-methyl-2H-pyrazol-3-yl]methanone
N-(4-{4-[2-(2-Fluorophenyl)-5-methyl-2H-pyrazole-3-carbonyl]piperazin-1-yl}phenyl)methanesulfonamide
[2-(3-Fluorophenyl)-5-methyl-2H-pyrazol-3-yl](4-phenylpiperazin-1-yl)methanone
[2-(3-Fluorophenyl)-5-methyl-2H-pyrazol-3-yl][4-(2-methoxyphenyl)piperazin-1-yl]methanone
[2-(3-Fluorophenyl)-5-methyl-2H-pyrazol-3-yl][4-(3-trifluoromethylphenyl)-piperazin-1-yl]methanone
[2-(3-Fluorophenyl)-5-methyl-2H-pyrazol-3-yl][4-(4-fluorophenyl)piperazin-1-yl]methanone
1-(4-{4-[2-(3-Fluorophenyl)-5-methyl-2H-pyrazole-3-carbonyl]piperazin-1-yl}-phenyl)ethanone
[4-(2,4-Dimethylphenyl)piperazin-1-yl][2-(3-fluorophenyl)-5-methyl-2H-pyrazol-3-yl]methanone
[4-(3,4-Dichlorophenyl)piperazin-1-yl][2-(3-fluorophenyl)-5-methyl-2H-pyrazol-3-yl]methanone
[4-(3,4-Dimethylphenyl)piperazin-1-yl][2-(3-fluorophenyl)-5-methyl-2H-pyrazol-3-yl]methanone
[2-(3-Fluorophenyl)-5-methyl-2H-pyrazol-3-yl](4-o-tolylpiperazin-1-yl)methanone
[4-(2,3-Dimethylphenyl)piperazin-1-yl][2-(3-fluorophenyl)-5-methyl-2H-pyrazol-3-yl]methanone
[4-(2-Ethylphenyl)piperazin-1-yl][2-(3-fluorophenyl)-5-methyl-2H-pyrazol-3-yl]methanone
[4-(3-Chlorophenyl)piperazin-1-yl][2-(3-fluorophenyl)-5-methyl-2H-pyrazol-3-yl]methanone
[2-(3-Fluorophenyl)-5-methyl-2H-pyrazol-3-yl][4-(3-methoxyphenyl)piperazin-1-yl]methanone
[2-(3-Fluorophenyl)-5-methyl-2H-pyrazol-3-yl](4-m-tolylpiperazin-1-yl)methanone
[2-(3-Fluorophenyl)-5-methyl-2H-pyrazol-3-yl][4-(4-methoxyphenyl)piperazin-1-yl]methanone
[4-(2,4-Dimethoxyphenyl)piperazin-1-yl][2-(3-fluorophenyl)-5-methyl-2H-pyrazol-3-yl]methanone
12-(3-Fluorophenyl)-5-methyl-2H-pyrazol-3-yl][4-(2-methylsulfanylphenyl)-piperazin-1-yl]methanone
[4-(4-Chlorophenyl)piperazin-1-yl][2-(3-fluorophenyl)-5-methyl-2H-pyrazol-3-yl]methanone
[2-(3-Fluorophenyl)-5-methyl-2H-pyrazol-3-yl](4-naphthalen-1-ylpiperazin-1-yl)methanone
[4-(5-Chloro-2-methylphenyl)piperazin-1-yl][2-(3-fluorophenyl)-5-methyl-2H-pyrazol-3-yl]methanone
[4-(2-Ethoxyphenyl)piperazin-1-yl][2-(3-fluorophenyl)-5-methyl-2H-pyrazol-3-yl]methanone
[4-(2,3-Dichlorophenyl)piperazin-1-yl][2-(3-fluorophenyl)-5-methyl-2H-pyrazol-3-yl]methanone
[2-(3-Fluorophenyl)-5-methyl-2H-pyrazol-3-yl][4-(4-trifluoromethylphenyl)-piperazin-1-yl]methanone
[4-(5-Chloro-2-methoxyphenyl)piperazin-1-yl][2-(3-fluorophenyl)-5-methyl-2H-pyrazol-3-yl]methanone
[4-(4-Benzyloxyphenyl)piperazin-1-yl][2-(3-fluorophenyl)-5-methyl-2H-pyrazol-3-yl]methanone
[2-(3-Fluorophenyl)-5-methyl-2H-pyrazol-3-yl][4-(2-trifluoromethylphenyl)-piperazin-1-yl]methanone
[4-(2,3-Dihydrobenzo[1,4]dioxin-6-yl)piperazin-.1-yl][2-(3-fluorophenyl)-5-methyl-2H-pyrazol-3-yl]methanone
N-(4-{4-[2-(3-Fluorophenyl)-5-methyl-2H-pyrazole-3-carbonyl]piperazin-1-ylphenyl)methanesulfonamide
[2-(4-Fluorophenyl)-5-methyl-2H-pyrazol-3-yl](4-phenylpiperazin-1-yl)methanone
[2-(4-Fluorophenyl)-5-methyl-2H-pyrazol-3-yl][4-(2-methoxyphenyl)piperazin-1-yl]methanone
[2-(4-Fluorophenyl)-5-methyl-2H-pyrazol-3-yl][4-(3-trifluoromethylphenyl)-piperazin-1-yl]methanone
[2-(4-Fluorophenyl)-5-methyl-2H-pyrazol-3-yl][4-(4-fluorophenyl)piperazin-1-yl]methanone
1-(4-{4-[2-(4-Fluorophenyl)-5-methyl-2H-pyrazole-3-carbonyl]piperazin-1-yl}-phenyl)ethanone
[4-(2,4-Dimethylphenyl)piperazin-1-yl][2-(4-fluorophenyl)-5-methyl-2H-pyrazol-3-yl]methanone
[4-(3,4-Dichlorophenyl)piperazin-1-yl][2-(4-fluorophenyl)-5-methyl-2H-pyrazol-3-yl]methanone
[4-(3,4-Dimethylphenyl)piperazin-1-yl][2-(4-fluorophenyl)-5-methyl-2H-pyrazol-3-yl]methanone
[2-(4-Fluorophenyl)-5-methyl-2H-pyrazol-3-yl](4-o-tolylpiperazin-1-yl)methanone
[4-(2,3-Dimethylphenyl)piperazin-1-yl][2-(4-fluorophenyl)-5-methyl-2H-pyrazol-3-yl]methanone
[4-(2-Ethylphenyl)piperazin-1-yl][2-(4-fluorophenyl)-5-methyl-2H-pyrazol-3-yl]methanone
[4-(3-Chlorophenyl)piperazin-1-yl][2-(4-fluorophenyl)-5-methyl-2H-pyrazol-3-yl]methanone
[2-(4-Fluorophenyl)-5-methyl-2H-pyrazol-3-yl][4-(3-methoxyphenyl)piperazin-1-yl]methanone
[2-(4-Fluorophenyl)-5-methyl-2H-pyrazol-3-yl](4-m-tolylpiperazin-1-yl)methanone
[2-(4-Fluorophenyl)-5-methyl-2H-pyrazol-3-yl][4-(4-methoxyphenyl)piperazin-1-yl]methanone
[4-(2,4-Dimethoxyphenyl)piperazin-1-yl][2-(4-fluorophenyl)-5-methyl-2H-pyrazol-3-yl]methanone
[2-(4-Fluorophenyl)-5-methyl-2H-pyrazol-3-yl][4-(2-methylsulfanylphenyl)-piperazin-1-yl]methanone
[4-(4-Chlorophenyl)piperazin-1-yl][2-(4-fluorophenyl)-5-methyl-2H-pyrazol-3-yl]methanone
[2-(4-Fluorophenyl)-5-methyl-2H-pyrazol-3-yl](4-naphthalen-1-ylpiperazin-1-yl)methanone
[4-(5-Chloro-2-methylphenyl)piperazin-1-yl][2-(4-fluorophenyl)-5-methyl-2H-pyrazol-3-yl]methanone
[4-(2-Ethoxyphenyl)piperazin-1-yl][2-(4-fluorophenyl)-5-methyl-2H-pyrazol-3-yl]methanone
[4-(2,3-Dichlorophenyl)piperazin-1-yl][2-(4-fluorophenyl)-5-methyl-2H-pyrazol-3-yl]methanone
[2-(4-Fluorophenyl)-5-methyl-2H-pyrazol-3-yl][4-(4-trifluoromethylphenyl)-piperazin-1-yl]methanone
[4-(5-Chloro-2-methoxyphenyl)piperazin-1-yl][2-(4-fluorophenyl)-5-methyl-2H-pyrazol-3-yl]methanone
[4-(4-Benzyloxyphenyl)piperazin-1-yl][2-(4-fluorophenyl)-5-methyl-2H-pyrazol-3-yl]methanone
[2-(4-Fluorophenyl)-5-methyl-2H-pyrazol-3-yl][4-(2-trifluoromethylphenyl)-piperazin-1-yl]methanone
[4-(2,3-Dihydrobenzo[1,4]dioxin-6-yl)piperazin-1-yl][2-(4-fluorophenyl)-5-methyl-2H-pyrazol-3-yl]methanone
[2-(4-Methanesulfonylphenyl)-5-methyl-2H-pyrazol-3-yl](4-phenylpiperazin-1-yl)methanone
[2-(4-Methanesulfonylphenyl)-5-methyl-2H-pyrazol-3-yl][4-(3-trifluoromethyl-phenyl)piperazin-1-yl]methanone
1-(4-{4-[2-(4-Methanesulfonylphenyl)-5-methyl-2H-pyrazole-3-carbonyl]-piperazin-1-yl}phenyl)ethanone
[4-(2,4-Dimethylphenyl)piperazin-1-yl][2-(4-methanesulfonylphenyl)-5-methyl-2H-pyrazol-3-yl]methanone
[2-(4-Methanesulfonylphenyl)-5-methyl-2H-pyrazol-3-yl](4-o-tolylpiperazin-1-yl)methanone
[4-(3-Chlorophenyl)piperazin-1-yl][2-(4-methanesulfonylphenyl)-5-methyl-2H-pyrazol-3-yl]methanone
[2-(4-Methanesulfonylphenyl)-5-methyl-2H-pyrazol-3-yl][4-(3-methoxy-phenyl)piperazin-1-yl]methanone
[2-(4-Methanesulfonylphenyl)-5-methyl-2H-pyrazol-3-yl](4-m-tolylpiperazin-1-yl)methanone
[4-(2,4-Dimethoxyphenyl)piperazin-1-yl][2-(4-methanesulfonylphenyl)-5-methyl-2H-pyrazol-3-yl]methanone
[2-(4-Methanesulfonylphenyl)-5-methyl-2H-pyrazol-3-yl][4-(2-methylsulfanyl-phenyl)piperazin-1-yl]methanone
[4-(4-Chlorophenyl)piperazin-1-yl][2-(4-methanesulfonylphenyl)-5-methyl-2H-pyrazol-3-yl]methanone
[4-(5-Chloro-2-methylphenyl)piperazin-1-yl][2-(4-methanesulfonylphenyl)-5-methyl-2H-pyrazol-3-yl]methanone
[4-(2-Ethoxyphenyl)piperazin-1-yl][2-(4-methanesulfonylphenyl)-5-methyl-2H-pyrazol-3-yl]methanone
[4-(2,3-Dichlorophenyl)piperazin-1-yl][2-(4-methanesulfonylphenyl)-5-methyl-2H-pyrazol-3-yl]methanone
[2-(4-Methanesulfonylphenyl)-5-methyl-2H-pyrazol-3-yl][4-(4-trifluoromethyl-phenyl)piperazin-1-yl]methanone
[4-(4-Benzyloxyphenyl)piperazin-1-yl][2-(4-methanesulfonylphenyl)-5-methyl-2H-pyrazol-3-yl]methanone
[2-(4-Methanesulfonylphenyl)-5-methyl-2H-pyrazol-3-yl][4-(2-trifluoromethyl-phenyl)piperazin-1-yl]methanone
[4-(2,3-Dihydrobenzo[1,4]dioxin-6-yl)piperazin-1-yl][2-(4-methanesulfonyl-phenyl)-5-methyl-2H-pyrazol-3-yl]methanone
[2-(3-Chlorophenyl)-5-methyl-2H-pyrazol-3-yl](4-phenylpiperazin-1-yl)methanone
[2-(3-Chlorophenyl)-5-methyl-2H-pyrazol-3-yl][4-(2-methoxyphenyl)piperazin-1-yl]methanone
[2-(3-Chlorophenyl)-5-methyl-2H-pyrazol-3-yl][4-(3-trifluoromethylphenyl)-piperazin-1-yl]methanone
[2-(3-Chlorophenyl)-5-methyl-2H-pyrazol-3-yl][4-(2,4-dimethylphenyl)piperazin-1-yl]methanone
[2-(3-Chlorophenyl)-5-methyl-2H-pyrazol-3-yl][4-(3,4-dimethylphenyl)piperazin-1-yl]methanone
[2-(3-Chlorophenyl)-5-methyl-2H-pyrazol-3-yl](4-o-tolylpiperazin-1-yl)methanone
[2-(3-Chlorophenyl)-5-methyl-2H-pyrazol-3-yl][4-(2,3-dimethylphenyl)piperazin-1-yl]methanone
[2-(3-Chlorophenyl)-5-methyl-2H-pyrazol-3-yl][4-(3-methoxyphenyl)piperazin-1-yl]methanone
[2-(3-Chlorophenyl)-5-methyl-2H-pyrazol-3-yl](4-m-tolylpiperazin-1-yl)methanone
[2-(3-Chlorophenyl)-5-methyl-2H-pyrazol-3-yl][4-(2,4-dimethoxyphenyl)piperazin-1-yl]methanone
[2-(3-Chlorophenyl)-5-methyl-2H-pyrazol-3-yl][4-(2-methylsulfanylphenyl)-piperazin-1-yl]methanone
[2-(3-Chlorophenyl)-5-methyl-2H-pyrazol-3-yl][4-(4-chlorophenyl)piperazin-1-yl]methanone
[2-(3-Chlorophenyl)-5-methyl-2H-pyrazol-3-yl](4-naphthalen-1-ylpiperazin-1-yl)methanone
[2-(3-Chlorophenyl)-5-methyl-2H-pyrazol-3-yl][4-(2-ethoxyphenyl)piperazin-1-yl]methanone
[2-(3-Chlorophenyl)-5-methyl-2H-pyrazol-3-yl][4-(2,3-dichlorophenyl)piperazin-1-yl]methanone
[2-(3-Chlorophenyl)-5-methyl-2H-pyrazol-3-yl][4-(4-trifluoromethylphenyl)-piperazin-1-yl]methanone
[4-(5-Chloro-2-methoxyphenyl)piperazin-1-yl][2-(3-chlorophenyl)-5-methyl-2H-pyrazol-3-yl]methanone
[4-(4-Benzyloxyphenyl)piperazin-1-yl][2-(3-chlorophenyl)-5-methyl-2H-pyrazol-3-yl]methanone
[2-(3-Chlorophenyl)-5-methyl-2H-pyrazol-3-yl][4-(2-trifluoromethylphenyl)-piperazin-1-yl]methanone
[2-(3-Chlorophenyl)-5-methyl-2H-pyrazol-3-yl][4-(2,3-dihydrobenzo[1,4]-dioxin-6-yl)piperazin-1-yl]methanone
[2-(4-Chlorophenyl)-5-methyl-2H-pyrazol-3-yl](4-phenylpiperazin-1-yl)methanone
[2-(4-Chlorophenyl)-5-methyl-2H-pyrazol-3-yl][4-(2-methoxyphenyl)piperazin-1-yl]methanone
[2-(4-Chlorophenyl)-5-methyl-2H-pyrazol-3-yl][4-(3-trifluoromethylphenyl)-piperazin-1-yl]methanone
[2-(4-Chlorophenyl)-5-methyl-2H-pyrazol-3-yl][4-(4-fluorophenyl)piperazin-1-yl]methanone
1-(4-{4-[2-(4-Chlorophenyl)-5-methyl-2H-pyrazole-3-carbonyl]piperazin-1-yl}-phenyl)ethanone
[2-(4-Chlorophenyl)-5-methyl-2H-pyrazol-3-yl][4-(3,4-dichlorophenyl)piperazin-1-yl]methanone
[2-(4-Chlorophenyl)-5-methyl-2H-pyrazol-3-yl][4-(3,4-dimethylphenyl)piperazin-1-yl]methanone
[2-(4-Chlorophenyl)-5-methyl-2H-pyrazol-3-yl](4-o-tolylpiperazin-1-yl)methanone
[2-(4-Chlorophenyl)-5-methyl-2H-pyrazol-3-yl][4-(2,3-dimethylphenyl)piperazin-1-yl]methanone
[2-(4-Chlorophenyl)-5-methyl-2H-pyrazol-3-yl][4-(2-ethylphenyl)piperazin-1-yl]methanone
[2-(4-Chlorophenyl)-5-methyl-2H-pyrazol-3-yl][4-(3-chlorophenyl)piperazin-1-yl]methanone
[2-(4-Chlorophenyl)-5-methyl-2H-pyrazol-3-yl](4-m-tolylpiperazin-1-yl)methanone
[2-(4-Chlorophenyl)-5-methyl-2H-pyrazol-3-yl][4-(4-methoxyphenyl)piperazin-1-yl]methanone
[2-(4-Chlorophenyl)-5-methyl-2H-pyrazol-3-yl][4-(4-chlorophenyl)piperazin-1-yl]methanone
[2-(4-Chlorophenyl)-5-methyl-2H-pyrazol-3-yl](4-naphthalen-1-ylpiperazin-1-yl)methanone
[4-(5-Chloro-2-methylphenyl)piperazin-1-yl][2-(4-chlorophenyl)-5-methyl-2H-pyrazol-3-yl]methanone
[2-(4-Chlorophenyl)-5-methyl-2H-pyrazol-3-yl][4-(2,3-dichlorophenyl)piperazin-1-yl]methanone
[4-(5-Chloro-2-methoxyphenyl)piperazin-1-yl][2-(4-chlorophenyl)-5-methyl-2H-pyrazol-3-yl]methanone
[4-(4-Benzyloxyphenyl)piperazin-1-yl][2-(4-chlorophenyl)-5-methyl-2H-pyrazol-3-yl]methanone
[2-(4-Chlorophenyl)-5-methyl-2H-pyrazol-3-yl][4-(2,3-dihydrobenzo[1,4]-dioxin-6-yl)piperazin-1-yl]methanone
(5-Methyl-2-p-tolyl-2H-pyrazol-3-yl)(4-phenylpiperazin-1-yl)methanone
(5-Methyl-2-p-tolyl-2H-pyrazol-3-yl)[4-(3-trifluoromethylphenyl)piperazin-1-yl]methanone
1-{4-[4-(5-Methyl-2-p-tolyl-2H-pyrazole-3-carbonyl)piperazin-1-yl]-phenyl)-ethanone
[4-(3,4-Dichlorophenyl)piperazin-1-yl](5-methyl-2-p-tolyl-2H-pyrazol-3-yl)-methanone
(5-Methyl-2-p-tolyl-2H-pyrazol-3-yl)(4-o-tolylpiperazin-1-yl)methanone
[4-(2,3-Dimethylphenyl)piperazin-1-yl](5-methyl-2-p-tolyl-2H-pyrazol-3-yl)-methanone
[4-(2-Ethylphenyl)piperazin-1-yl](5-methyl-2-p-tolyl-2H-pyrazol-3-yl)methanone
[4-(3-Chlorophenyl)piperazin-1-yl](5-methyl-2-p-tolyl-2H-pyrazol-3-yl)methanone
[4-(3-Methoxyphenyl)piperazin-1-yl](5-methyl-2-p-tolyl-2H-pyrazol-3-yl)methanone
(5-Methyl-2-p-tolyl-2H-pyrazol-3-yl)(4-m-tolylpiperazin-1-yl)methanone
[4-(4-Methoxyphenyl)piperazin-1-yl](5-methyl-2-p-tolyl-2H-pyrazol-3-yl)methanone
[4-(2,4-Dimethoxyphenyl)piperazin-1-yl](5-methyl-2-p-tolyl-2H-pyrazol-3-yl)-methanone
[4-(2-Methylsulfanylphenyl)piperazin-1-yl](5-methyl-2-p-tolyl-2H-pyrazol-3-yl)methanone
[4-(4-Chlorophenyl)piperazin-1-yl](5-methyl-2-p-tolyl-2H-pyrazol-3-yl)methanone
(5-Methyl-2-p-tolyl-2H-pyrazol-3-yl)(4-naphthalen-1-ylpiperazin-1-yl)methanone
[4-(5-Chloro-2-methylphenyl)piperazin-1-yl](5-methyl-2-p-tolyl-2H-pyrazol-3-yl)methanone
[4-(2-Ethoxyphenyl)piperazin-1-yl](5-methyl-2-p-tolyl-2H-pyrazol-3-yl)methanone
[4-(2,3-Dichlorophenyl)piperazin-1-yl](5-methyl-2-p-tolyl-2H-pyrazol-3-yl)-methanone
(5-Methyl-2-p-tolyl-2H-pyrazol-3-yl )[4-(4-trifluoromethylphenyl)piperazin-1-yl]methanone
[4-(5-Chloro-2-methoxyphenyl)piperazin-1-yl](5-methyl-2-p-tolyl-2H-pyrazol-3-yl)methanone
[4-(4-Benzyloxyphenyl )piperazin-1-yl](5-methyl-2-p-tolyl-2H-pyrazol-3-yl)-methanone
(5-Methyl-2-p-tolyl-2H-pyrazol-3-yl)[4-(2-trifluoromethylphenyl)piperazin-1-yl]methanone
[4-(2,3-Dihydrobenzo[1,4]dioxin-6-yl)piperazin-1-yl](5-methyl-2-p-tolyl-2H-pyrazol-3-yl)methanone
N-(4-[4-(5-Methyl-2-p-tolyl-2H-pyrazole-3-carbonyl)piperazin-1-yl]-phenyl)-methanesulfonamide
(5-Methyl-2-m-tolyl-2H-pyrazol-3-yl)(4-phenylpiperazin-1-yl)methanone
[4-(2-Methoxyphenyl)piperazin-1-yl](5-methyl-2-m-tolyl-2H-pyrazol-3-yl)methanone
(5-Methyl-2-m-tolyl-2H-pyrazol-3-yl)[4-(3-trifluoromethylphenyl)piperazin-1-yl]methanone
[4-(4-Fluorophenyl)piperazin-1-yl](5-methyl-2-m-tolyl-2H-pyrazol-3-yl)methanone
1-{4-[4-(5-Methyl-2-m-tolyl-2H-pyrazole-3-carbonyl)piperazin-1-yl]-phenyl)-ethanone
[4-(2,4-Dimethylphenyl)piperazin-1-yl](5-methyl-2-m-tolyl-2H-pyrazol-3-yl)-methanone
[4-(3,4-Dichlorophenyl)piperazin-1-yl](5-methyl-2-m-tolyl-2H-pyrazol-3-yl)-methanone
[4-(3,4-Dimethylphenyl)piperazin-1-yl](5-methyl-2-m-tolyl-2H-pyrazol-3-yl)-methanone
(5-Methyl-2-m-tolyl-2H-pyrazol-3-yl)(4-o-tolylpiperazin-1-yl)methanone
[4-(2,3-Dimethylphenyl)piperazin-1-yl](5-methyl-2-m-tolyl-2H-pyrazol-3-yl)-methanone
[4-(2-Ethylphenyl)piperazin-1-yl](5-methyl-2-m-tolyl-2H-pyrazol-3-yl)methanone
[4-(3-Chlorophenyl)piperazin-1-yl](5-methyl-2-m-tolyl-2H-pyrazol-3-yl)methanone
[4-(3-Methoxyphenyl)piperazin-1-yl](5-methyl-2-m-tolyl-2H-pyrazol-3-yl)-methanone
(5-Methyl-2-m-tolyl-2H-pyrazol-3-yl)(4-m-tolylpiperazin-1-yl)methanone
[4-(4-Methoxyphenyl)piperazin-1-yl](5-methyl-2-m-tolyl-2H-pyrazol-3-yl)-methanone
[4-(2,4-Dimethoxyphenyl)piperazin-1-yl](5-methyl-2-m-tolyl-2H-pyrazol-3-yl)-methanone
[4-(2-Methylsulfanylphenyl)piperazin-1-yl](5-methyl-2-m-tolyl-2H-pyrazol-3-yl)methanone
[4-(4-Chlorophenyl)piperazin-1-yl](5-methyl-2-m-tolyl-2H-pyrazol-3-yl)methanone
(5-Methyl-2-m-tolyl-2H-pyrazol-3-yl)(4-naphthalen-1-ylpiperazin-1-yl)methanone
[4-(5-Chloro-2-methylphenyl)piperazin-1-yl](5-methyl-2-m-toly-2H-pyrazol-3-yl)methanone
[4-(2-Ethoxyphenyl)piperazin-1-yl](5-methyl-2-m-tolyl-2H-pyrazol-3-yl)methanone
[4-(2,3-Dichlorophenyl)piperazin-1-yl](5-methyl-2-m-tolyl-2H-pyrazol-3-yl)-methanone
(5-Methyl-2-m-tolyl-2H-pyrazol-3-yl)14-(4-trifluoromethylphenyl)piperazin-1-yl]methanone
[4-(5-Chloro-2-methoxyphenyl)piperazin-1-yl](5-methyl-2-m-tolyl-2H-pyrazol-3-yl)methanone
[4-(4-Benzyloxyphenyl)piperazin-1-yl](5-methyl-2-m-tolyl-2H-pyrazol-3-yl)-methanone
(5-Methyl-2-m-tolyl-2H-pyrazol-3-yl)[4-(2-trifluoromethylphenyl)piperazin-1-yl]methanone
[4-(2,3-Dihydrobenzo[1,4]dioxin-6-yl)piperazin-1-yl](5-methyl-2-m-tolyl-2H-pyrazol-3-yl)methanone
N-{4-[4-(5-Methyl-2-m-tolyl-2H -pyrazole-3-carbonyl)piperazin-1-yl]-phenyl)-methanesulfonamide
(2H-Indazol-3-yl)(4-phenylpiperazin-1-yl)methanone
(2H-Indazol-3-yl)[4-(2-methoxyphenyl)piperazin-1-yl]methanone
(2H-Indazol-3-yl)[4-(3-trifluoromethylphenyl)piperazin-1-yl]methanone
[4-(4-Fluorophenyl)piperazin-1-yl](2H-indazol-3-yl)methanone
1-{4-[4-(2H-Indazole-3-carbonyl)piperazin-1-yl]-phenyl}ethanone
[4-(2,4-Dimethylphenyl)piperazin-1-yl](2H-indazol-3-yl)methanone
[4-(3,4-Dichlorophenyl)piperazin-1-yl](2H-indazol-3-yl)methanone
[4-(3,4-Dimethylphenyl)piperazin-1-yl](2H-indazol-3-yl)methanone
(2H-Indazol-3-yl)(4-o-tolylpiperazin-1-yl)methanone
[4-(2,3-Dimethylphenyl)piperazin-1-yl](2H-indazol-3-yl)methanone
[4-(2-Ethylphenyl)piperazin-1-yl](2H-indazol-3-yl)methanone
[4-(3-Chlorophenyl)piperazin-1-yl](2H-indazol-3-yl)methanone
(2H-Indazol-3-yl)[4-(3-methoxyphenyl)piperazin-1-yl]methanone
(2H-Indazol-3-yl)(4-m-tolylpiperazin-1-yl)methanone
(2H-Indazol-3-yl)[4-(4-methoxyphenyl)piperazin-1-yl]methanone
[4-(2,4-Dimethoxyphenyl)piperazin-1-yl](2H-indazol-3-yl)methanone
(2H-Indazol-3-yl)[4-(2-methylsulfanylphenyl)piperazin-1-yl]methanone
[4-(4-Chlorophenyl)piperazin-1-yl](2H-indazol-3-yl)methanone
(2H-Indazol-3-yl)(4-naphthalen-1-ylpiperazin-1-yl)methanone
[4-(5-Chloro-2-methylphenyl)piperazin-1-yl](2H-indazol-3-yl)methanone
[4-(2-Ethoxyphenyl)piperazin-1-yl](2H-indazol-3-yl)methanone
[4-(2,3-Dichlorophenyl)piperazin-1-yl](2H-indazol-3-yl)methanone
(2H-Indazol-3-yl)[4-(4-trifluoromethylphenyl)piperazin-1-yl]methanone
[4-(5-Chloro-2-methoxyphenyl)piperazin-1-yl](2H-indazol-3-yl)methanone
[4-(4-Benzyloxyphenyl)piperazin-1-yl](2H-indazol-3-yl)methanone
(2H-Indazol-3-yl)[4-(2-trifluoromethylphenyl)piperazin-1-yl]methanone
[4-(2,3-Dihydrobenzo[1,4]dioxin-6-yl)piperazin-1-yl](2H-indazol-3-yl)methanone
N-{4-[4-(2H-Indazole-3-carbonyl)piperazin-1-yl]-phenyl}methanesulfonamide
(2-Methyl-1-quinolin-2-yl-1H-pyrrol-3-yl)(4-phenylpiperazin-1-yl)methanone
[4-(3,4-Dichlorophenyl)piperazin-1-yl](2-methyl-1-quinolin-2-yl-1H-pyrrol-3-yl)methanone
[4-(2-Methoxyphenyl)piperazin-1-yl](2-methyl-1-quinolin-2-yl-1H-pyrrol-3-yl)-methanone
[4-(3,4-Dimethylphenyl)piperazin-1-yl](2-methyl-1-quinolin-2-yl-1H-pyrrol-3-yl)methanone
[4-(3-Chlorophenyl)piperazin-1-yl](2-methyl-1-quinolin-2-yl-1H-pyrrol-3-yl)-methanone
[4-(3,4-Dimethylphenyl)piperazin-1-yl](1-isoquinolin-1-yl-2-methyl-1H-pyrrol-3-yl)methanone
[4-(3-Chlorophenyl)piperazin-1-yl](1-isoquinolin-1-yl-2-methyl-1H-pyrrol-3-yl)methanone
(3,5-Dimethyl-1-phenyl-1H-pyrazol-4-yl)[4-(3-methoxyphenyl)piperazin-1-yl]-methanone
N-{4-[4-(3,5-Dimethyl-1-phenyl-1H-pyrazole-4-carbonyl)piperazin-1-yl]-phenyl)methanesulfonamide
[2-(4-Methoxyphenyl)-5-methyl-2H-pyrazol-3-yl][4-(3-methoxyphenyl)piperazin-1-yl]methanone
N-(4-{4-[2-(4-Methoxyphenyl)-5-methyl-2H-pyrazole-3-carbonyl]piperazin-1-yl}phenyl)methanesulfonamide
[2-(3-Methoxyphenyl)-5-methyl-2H-pyrazol-3-yl][4-(3-methoxyphenyl)piperazin-1-yl]methanone
N-(4-{4-[2-(3-Methoxyphenyl)-5-methyl-2H-pyrazole-3-carbonyl]piperazin-1-yl}phenyl)methanesulfonamide
(5-Methyl-2-phenyl-2H-pyrazol-3-yl)[4-(2-trifluoromethylphenyl)piperazin-1-yl]methanone
[4-(4-Benzyloxyphenyl)piperazin-1-yl][2-(2-fluorophenyl)-5-methyl-2H-pyrazol-3-yl]methanone
N-(4-{4-[2-(4-Fluorophenyl)-5-methyl-2H-pyrazole-3-carbonyl]piperazin-1-yl-phenyl)methanesulfonamide
[2-(4-Methanesulfonylphenyl)-5-methyl-2H-pyrazol-3-yl][4-(2-methoxy-phenyl)piperazin-1-yl]methanone
[4-(4-Fluorophenyl)piperazin-1-yl][2-(4-methanesulfonylphenyl)-5-methyl-2H-pyrazol-3-yl]methanone
[4-(3,4-Dichlorophenyl)piperazin-1-yl][2-(4-methanesulfonylphenyl)-5-methyl-2H-pyrazol-3-yl]methanone
[4-(3,4-Dimethylphenyl)piperazin-1-yl][2-(4-methanesulfonylphenyl)-5-methyl-2H-pyrazol-3-yl]methanone
[4-(2,3-Dimethylphenyl)piperazin-1-yl][2-(4-methanesulfonylphenyl)-5-methyl-2H-pyrazol-3-yl]methanone
[2-(4-Methanesulfonylphenyl)-5-methyl-2H-pyrazol-3-yl][4-(4-methoxy-phenyl)piperazin-1-yl]methanone
[2-(4-Methanesulfonylphenyl)-5-methyl-2H-pyrazol-3-yl](4-naphthalen-1-yl-piperazin-1-yl)methanone
[4-(5-Chloro-2-methoxyphenyl)piperazin-1-yl][2-(4-methanesulfonylphenyl)-5-methyl-2H-pyrazol-3-yl]methanone
N-(4-{4-[2-(4-Methanesulfonylphenyl)-5-methyl-2H-pyrazole-3-carbonyl]-piperazin-1-yl)phenyl)methanesulfonamide
[2-(3-Chlorophenyl)-5-methyl-2H-pyrazol-3-yl][4-(4-fluorophenyl)piperazin-1-yl]methanone
[2-(3-Chlorophenyl)-5-methyl-2H-pyrazol-3-yl][4-(3,4-dichlorophenyl)piperazin-1-yl]methanone
[2-(3-Chlorophenyl)-5-methyl-2H-pyrazol-3-yl][4-(2-ethylphenyl)piperazin-1-yl]methanone
[2-(3-Chlorophenyl)-5-methyl-2H-pyrazol-3-yl][4-(3-chlorophenyl)piperazin-1-yl]methanone
[2-(3-Chlorophenyl)-5-methyl-2H-pyrazol-3-yl][4-(4-methoxyphenyl)piperazin-1-yl]methanone
[4-(5-Chloro-2-methylphenyl)piperazin-1-yl][2-(3-chlorophenyl)-5-methyl-2H-pyrazol-3-yl]methanone
N-(4-{4-[2-(3-Chlorophenyl)-5-methyl-2H-pyrazole-3-carbonyl]piperazin-1-yl}phenyl)methanesulfonamide
[2-(4-Chlorophenyl)-5-methyl-2H-pyrazol-3-yl][4-(2,4-dimethylphenyl)piperazin-1-yl]methanone
[2-(4-Chlorophenyl)-5-methyl-2H-pyrazol-3-yl][4-(3-methoxyphenyl)piperazin-1-yl]methanone
[2-(4-Chlorophenyl)-5-methyl-2H-pyrazol-3-yl][4-(2,4-dimethoxyphenyl)piperazin-1-yl]methanone
[2-(4-Chlorophenyl)-5-methyl-2H-pyrazol-3-yl][4-(2-methylsulfanylphenyl)-piperazin-1-yl]methanone
[2-(4-Chlorophenyl)-5-methyl-2H-pyrazol-3-yl][4-(2-ethoxyphenyl)piperazin-1-yl]methanone
[2-(4-Chlorophenyl)-5-methyl-2H-pyrazol-3-yl][4-(4-trifluoromethylphenyl)-piperazin-1-yl]methanone
[2-(4-Chlorophenyl)-5-methyl-2H-pyrazol-3-yl][4-(2-trifluoromethylphenyl)-piperazin-1-yl]methanone
N-(4-{4-[2-(4-Chlorophenyl)-5-methyl-2H-pyrazole-3-carbonyl]piperazin-1-yl}phenyl)methanesulfonamide
[4-(2-Methoxyphenyl)piperazin-1-yl](5-methyl-2-p-tolyl-2H-pyrazol-3-yl)methanone
[4-(4-Fluorophenyl)piperazin-1-yl](5-methyl-2-p-tolyl-2H-pyrazol-3-yl)methanone
[4-(2,4-Dimethylphenyl)piperazin-1-yl](5-methyl-2-p-tolyl-2H-pyrazol-3-yl)-methanone
[4-(3,4-Dimethylphenyl)piperazin-1-yl](5-methyl-2-p-tolyl-2H-pyrazol-3-yl)-methanone

14. The product as claimed in claim 1, characterized in that it is chosen from:
[4-(3-Chlorophenyl)piperazin-1-yl](5-methyl-2-phenyl-2H-pyrazol-3-yl)methanone (Example 1)
[4-(3,4-Dimethylphenyl)piperazin-1-yl](5-methyl-2-phenyl-2H-pyrazol-3-yl)-methanone (Example 2)
[4-(3,5-Dichlorophenyl)piperazin-1-yl](5-methyl-2-phenyl-2H-pyrazol-3-yl)-methanone (Example E2)
[4-(Quinolin-4-yl)piperazin-1-yl](5-methyl-2-phenyl-2H-pyrazol-3-yl)methanone (Example E17)
[4-(3-Chlorophenyl)piperazin-1-yl](5-hydroxymethyl-2-phenyl-2H-pyrazol-3-yl)methanone (Example E60)
[4-(3,4-Methylenedioxyphenyl)piperazin-1-yl](5-methyl-2-phenyl-2H-pyrazol-3-yl)methanone (Example E30)
[4-(3,5-Dimethoxyphenyl)piperazin-1-yl](5-methyl-2-phenyl-2H-pyrazol-3-yl)methanone (Example E20)
[4-(3,5-Dimethylphenyl)piperazin-1-yl](5-methyl-2-phenyl-2H-pyrazol-3-yl)-methanone (Example E63)
[4-(3-Difluoromethoxyphenyl)piperazin-1-yl](5-methyl-2-phenyl-2H-pyrazol-3-yl)methanone (Example E74)
[4-(3-Chlorophenyl)piperazin-1-yl][5-(2-methylimidazol-1-yl-methyl)-2-phenyl-2H-pyrazol-3-yl]methanone (Example E75)
[4-(3,5-Dimethoxyphenyl)piperazin-1-yl][5-(1H-pyrrol-2-yl)methyl-2-phenyl-2H-pyrazol-3-yl]methanone (Example E91)
[4-(3,5-Dimethoxyphenyl)piperazin-1-yl][5-(pyrrolidin-1-yl)methyl-2-phenyl-2H-pyrazol-3-yl]methanone (Example E93)
[4-(3-Carboxamidophenyl)piperazin-1-yl](5-trifluoromethyl-2-phenyl-2H-pyrazol-3-yl)methanone (Example El 19)
[5-(Azetidin-1-yl)-2-phenyl-2H-pyrazol-3-yl][4-(3,5-dimethoxyphenyl)piperazin-1-yl]methanone (Example E129)
[4-(3-Carboxamidophenyl)piperazin-1-yl](2-phenyl-2H-pyrazol-3-yl)methanone (Example E133)
[4-(3-Carboxamidophenyl)piperazin-1-yl](5-hydroxymethyl-2-phenyl-2H-pyrazol-3-yl)methanone (Example E134)
[4-(3-Carboxamidophenyl)piperazin-1-yl](5-methyl-2-phenyl-2H-pyrazol-3-yl)methanone (Example E23)
[4-(3,5-Dimethoxyphenyl)piperazin-1-yl](5-hydroxymethyl-2-phenyl-2H-pyrazol-3-yl)methanone (Example E73)
(5-Amino-2-phenyl-2H-pyrazol-3-yl)[4-(3,5-dimethoxyphenyl)piperazin-1-yl]-methanone (Example E99).

15. The product as claimed in claim 14, characterized in that it is chosen from:
[4-(3-Carboxamidophenyl)piperazin-1-yl](5-methyl-2-phenyl-2H-pyrazol-3-yl)methanone (Example E23)
[4-(3,5-Dimethoxyphenyl)piperazin-1-yl](5-hydroxymethyl-2-phenyl-2H-pyrazol-3-yl)methanone (Example E73)
(5-Amino-2-phenyl-2H-pyrazol-3-yl)[4-(3,5-dimethoxyphenyl)piperazin-1-yl]-methanone (Example E99).

16. A pharmaceutical composition comprising a product as claimed in any one of the preceding claims, in combination with a pharmaceutically acceptable excipient.

17. The use of a product as claimed in any one of claims 1 to 15, as an agent for inhibiting tubulin polymerization.

18. The use of a product as claimed in any one of claims 1 to 15, as an agent for inhibiting the proliferation of tumoral cells.

19. The use of a product as claimed in any one of claims 1 to 15, for promoting the disintegration of lumps of cells originating from vascular tissue.

20. The use of a product as claimed in any one of claims 1 to 15, for the manufacture of a medicinal product that is useful for treating a pathological condition.

21. The use as claimed in claim 20, in which the pathological condition is cancer.
